(19) United States
(12) Patent Application Publication
(10) Pub. No.: US 2024/0226104 A1
(43) Pub. Date: Jul. 11, 2024

(54) PI3K INHIBITORS, NANOFORMULATIONS, AND USES THEREOF

(71) Applicant: THE REGENTS OF THE UNIVERSITY OF MICHIGAN, Ann Arbor, MI (US)

(72) Inventors: Wei Gao, Ann Arbor, MI (US); Duxin Sun, Ann Arbor, MI (US); Fei Wen, Ann Arbor, MI (US); Ruiting Li, Ann Arbor, MI (US); Hongxiang Hu, Ann Arbor, MI (US); Luke F. Bugada, Ann Arbor, MI (US); Yudong Song, Ann Arbor, MI (US); Mahamadou Djibo, Ann Arbor, MI (US)

(21) Appl. No.: 18/556,250

(22) PCT Filed: Apr. 20, 2022
(86) PCT No.: PCT/US22/25520

§ 371 Date: Oct. 19, 2023

Related U.S. Application Data
(60) Provisional application No. 63/176,930, filed on Apr. 20, 2021.

(51) Int. Cl. A61K 31/519; A61K 9/00; A61K 9/51; A61K 31/337; A61P 35/04

(52) U.S. Cl. CPC A61K 31/519; A61K 9/0019; A61K 9/5169; A61K 31/337; A61P 35/04

(57) ABSTRACT

The present disclosure provides phosphatidylinositol 3-kinase (PI3K) inhibitors, and compositions, nanoformulations and methods for treating diseases or disorders (e.g., breast cancer, pancreatic cancer, lung cancer, and lymphoma) with PBK inhibitors or composition thereof. Disclosed herein is a composition comprising: an effective amount of a PI3K inhibitor or a pharmaceutically acceptable salt thereof; and an albumin nanoparticle.

### CROSS-REFERENCE TO RELATED APPLICATIONS

This application claims priority to and the benefit of U.S. Provisional Patent Application No. 63/176,930, filed on Apr. 20, 2021, which is incorporated herein by reference in its entirety.

### FIELD

The present disclosure provides phosphatidylinositol 3-kinase (PI3K) inhibitors, and compositions, nanoformulations, and methods for treating diseases or disorders (e.g., breast cancer, pancreatic cancer, lung cancer, lymphoma) with PI3K inhibitors or compositions thereof.

### BACKGROUND

The phosphatidylinositol 3-kinase (PI3K)/protein kinase B (AKT) signaling pathway is involved in many cellular functions including cell growth, proliferation, migration, differentiation, and apoptosis. The PI3K pathway is among the most frequently activated in human cancers, impacting almost 50% of the malignancies. Class IA isoforms PI3Kα, β and δ are particularly strongly associated with cancer. Despite considerable efforts to date, the clinical outcome of PI3K inhibitor-based treatments has achieved limited success. Reasons include Jack of tissue targeting, drug resistance such as that resulting from phosphatase and tensin homolog (PTEN) suppression, and lack of specificity that leads to dose limiting toxicity.

### SUMMARY

In one aspect, disclosed herein is a composition comprising: an effective amount of a phosphatidylinositol 3-kinase (PI3K) inhibitor, or a pharmaceutically acceptable salt thereof; and an albumin nanoparticle.

In some embodiments, the PI3K inhibitor is a Class I PI3K inhibitor. In some embodiments, the PI3K inhibitor is an isoform-selective PI3K inhibitor. In some embodiments, the PI3K inhibitor is selected from IPI-549, idelalisib, copanlisib, duvelisib, alpelisib, leniolisib, umbralisib, buparlisib, taselisib, pictilisib, PX-886, pilaralisib, BEZ235, GSK2126458, GSK2636771, AZD8186, SAR260301, gedatolisib, apitolisib, PQR309, MLN1117, and perifosine.

In some embodiments, the PI3K inhibitor is a compound of formula (III):

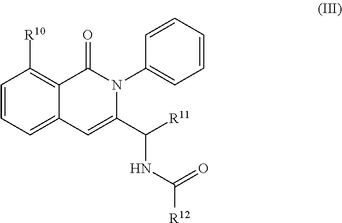

or a pharmaceutically acceptable salt thereof, wherein:
R¹⁰ is selected from —C≡C—R^{x}, C₁-C₄ alkyl, C₂-C₄ alkenyl, C₂-C₄ alkynyl, halo, cyano, C₁-C₄ alkoxy, C₁-C₄ alkylamino, and di-C₁-C₄-alkylamino;
R¹¹ is selected from C₁-C₄ alkyl and hydrogen;
R¹² is an 8- to 10-membered bicyclic heteroaryl having 1, 2, or 3 nitrogen atoms, wherein the heteroaryl is optionally substituted with 1 or 2 substituents selected from amino, C₁-C₄ alkyl, and halo; and

R^{x} is selected from a 5- or 6-membered monocyclic heteroaryl having 1 or 2 heteroatoms independently selected from N and S, aryl, hydrogen, and C₁-C₄ alkyl, wherein the heteroaryl and aryl are optionally substituted with 1 or 2 substituents selected from C₁-C₄ alkyl.

In some embodiments, R¹⁰ is —C≡C—R^{a}, and R^{a} is a 5-membered monocyclic heteroaryl having two nitrogen atoms, which is substituted with one C₁-C₄ alkyl. In some embodiments, R¹¹ is methyl. In some embodiments, R¹² is a pyrazolo[1,5-a]pyrimidine substituted with one amino group. In some embodiments, the compound of formula (III) is:

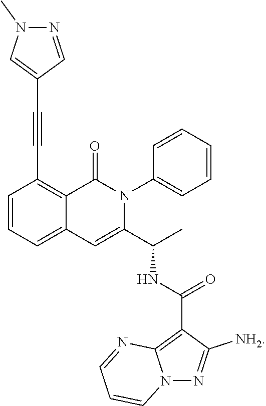 [SMILES: C[C@H](NC(=O)c1c(N)nn2cccnc12)c1cc2cccc(C#Cc3cnn(C)c3)c2c(=O)n1-c1ccccc1]

In some embodiments, the nanoparticle has a diameter between 50 and 200 nm. In some embodiments, the albumin nanoparticle encapsulates the PI3K inhibitor. In some embodiments, the albumin is human serum albumin or albumin from animal species.

In some embodiments, the composition further comprises a chemotherapeutic agent. In some embodiments, the albumin nanoparticle encapsulates the chemotherapeutic agent. In some embodiments, the chemotherapeutic agent is paclitaxel.

In another aspect, disclosed herein is a compound of formula (I):

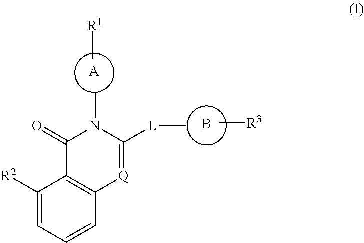

or a pharmaceutically acceptable salt thereof, wherein:
Q is CH or N;
A is aryl or a 5- or 6-membered monocyclic heteroaryl having 1, 2, 3, or 4 heteroatoms independently selected from N, O, S, and P;
R¹ is selected from hydrogen, halo, C₁-C₄ alkyl, C₃-C₆ cycloalkyl, C₁-C₄ haloalkyl, —OR^{a1}, —N(R^{b1})(R^{c1}), —SO₂R^{d1}, —SO₂N(R^{e1})(R^{f1}), and —NHSO₂R^{g1}, wherein R^{a1}, R^{b1}, R^{c1}, R^{d1}, R^{e1}, R^{f1}, and R^{g1} are each independently selected from hydrogen, C₁-C₄ alkyl, and C₁-C₄ haloalkyl;
R² is selected from hydrogen, halo, C₁-C₄ alkyl, C₁-C₄ haloalkyl, C₁-C₄ alkoxy, C₁-C₄ haloalkoxy, and a group of formula (II):

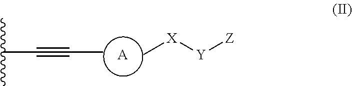

wherein:
   D is a monocyclic heteroaryl or monocyclic heterocyclyl, each of which is optionally substituted with a C₁-C₄ alkyl group;
      X is a bond, —C(O)—, —NH—, or —C(O)NH—;
      Y is —(CR^{a2}₂)ₙ-G²-, wherein each R^{a2} is independently selected from H and C₁-C₄ alkyl, or wherein two R^{a2} together with the carbon atom(s) to which they are attached form a C₃-C₇ cycloalkyl; G² is a bond, cycloalkylene, or heterocyclylene; and
   n is 0, 1, 2, or 3;
      Z is —OR^{b2}, —SR^{c2}, —N(R^{d2})(R^{b2}), or —CH₃, wherein Rb?, R^{e2}, R^{d2}, and R^{e2} are each independently selected from hydrogen, aryl, arylalkyl, C₁-C₄ alkyl, —C(O)—C₁-C₄₀ alkyl, —C(O)—C₂-C₄₀ alkenyl, and a group of formula (IIa):

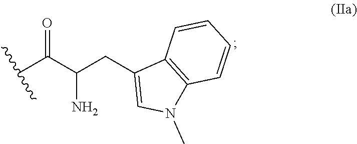 [SMILES: *C(=O)C(N)Cc1cn(C)c2ccccc12]

and
R³ is selected from hydrogen and a group -L3-E, wherein:
   L³ is a bond. C₁-C₂ alkylene, —CH═CH—, —C≡C—, —C(O)—, —O—, —NH—, —S—, —C(O)O—, —C(O)NH—, —C(O)S—, arylene, cycloalkylene, heteroarylene, or heterocyclylene, or wherein L³ comprises a combination of any two of such groups; and
   E is a bicyclic heterocyclyl or bicyclic heteroaryl, each of which is optionally substituted with 1, 2, 3, 4, or 5 substituents independently selected from halo, C₁-C₄ alkyl, C₃-C₆ cycloalkyl, C₃-C₆-cycloalkyl-C₁-C₄-alkyl, C₁-C₄ haloalkyl, oxo, —OR^{a3}, —N(R^{b3})(R^{b3}), —SO₂R^{d3}, —SO₂N(R^{e3})(R^{f3}), and —NHSO₂R^{b3}, wherein R^{a3}, R^{b3}, R^{c3}, R^{d3}, R^{e3}, R^{f3}, and R^{g3} are each independently selected from hydrogen, C₁-C₄ alkyl, and C₁-C₄ haloalkyl;
L is —(CR^{a4}R^{b4})ₘ-G⁴-, wherein:
   R^{a4} and R^{b4} are independently selected from hydrogen and C₁-C₄ alkyl;
   m is 0, 1, or 2; and
   G⁴ is a bond, —NHC(O)—, —NH—, —O—, or —S—; and
B is a bicyclic heteroaryl or bicyclic heterocyclyl, each of which is optionally substituted with 1, 2, 3, 4, or 5 substituents independently selected from halo, C₁-C₄ alkyl, C₃-C₆ cycloalkyl, C₁-C₄ haloalkyl, optionally substituted aryl, —OR^{a5}, —N(R^{b5})(R^{c5}), —SO₂R^{d5}, —SO₂N(R^{c5})(R^{f5}), and —NHSO₂R^{e5}, wherein R^{a5}, R^{b5}, R^{c5}, R^{d5}, R^{e5}, R^{f5}, and R^{g5} are each independently selected from hydrogen, C₁-C₄ alkyl, and C₁-C₄ haloalkyl;
wherein when R³ is hydrogen. R² is a group of formula (II) and Z is not —CH₃; and
wherein when R² is hydrogen, halo, C₁-C₄ alkyl, C₁-C₄ haloalkyl, C₁-C₄ alkoxy, or C₁-C₄ haloalkoxy, R³ is a group -L³-E.

In some embodiments, the compound is a compound of formula (Ia):

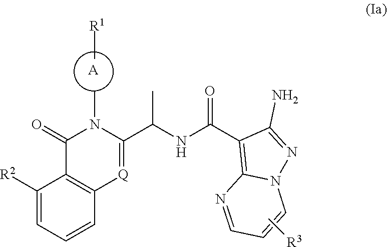

or a pharmaceutically acceptable salt thereof. In some embodiments, A is phenyl and R¹ is hydrogen.

In some embodiments, the compound is a compound of formula (Ib):

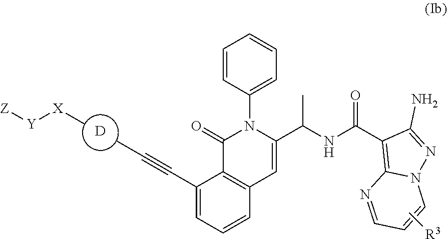

or a pharmaceutically acceptable salt thereof.

In some embodiments, D is a five-membered monocyclic heteroaryl or a 4- to 6-membered monocyclic heterocyclyl, each of which independently comprises 1, 2, 3, or 4 heteroatoms independently selected from N, O, S, and P. In some embodiments, D is selected from pyrrole, pyrazole, imidazole, imidazoline, oxazole, oxathiazole, oxadiazole, azetidine, pyrroline, pyrrolidine, and piperidine. In some embodiments, D has a structure selected from:

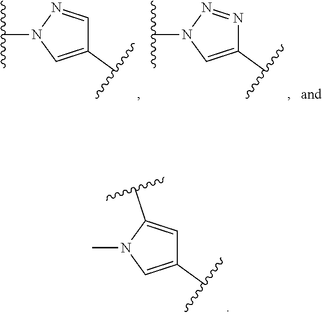

In some embodiments, X is a bond or —C(O)—.

In some embodiments, Y is —(CR^{a2}₂)ₙ—CH₂—, wherein n is 0 or 1, and wherein each R^{a2} is hydrogen, or wherein the two R^{a2} groups, together with the carbon atom to which they are attached, form a cyclopropylene ring.

In some embodiments, the group —X—Y—Z has a formula selected from:

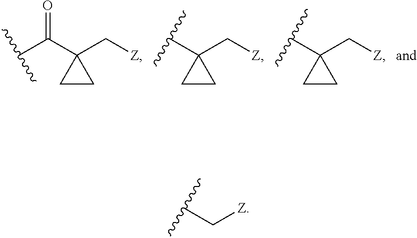

In some embodiments, Z is —OR^{b2}, wherein R^{b2} is selected from hydrogen, —C(O)—C₁-C₄₀ alkyl, —C(O)—C₂-C₄₀ alkenyl, and a group of formula (IIa). In some embodiments, Z is —OR^{b2}, wherein R^{b2} is selected from hydrogen, —C(O)—C₁₅-C₂₀ alkyl, —C(O)—C₁₅-C₂₀ alkenyl, and a group of formula (IIa).

In some embodiments, compound is a compound of formula (Ic):

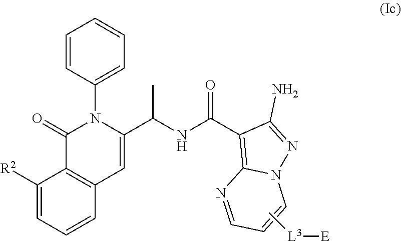

or a pharmaceutically acceptable salt thereof.

In some embodiments, R² is selected from halo and a group of formula (II). In some embodiments, R² is halo.

In some embodiments, L³ is a bond, —CH₂—CH₂—, —CH═CH—, —C≡C—, —C(O)NH—, or a 5-membered heteroarylene having 1, 2, or 3 nitrogen atoms.

In some embodiments, E has a formula:

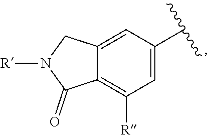

wherein R′ and R″ are independently selected from C₁-C₄ alkyl, C₃-C₆-cycloalkyl-C₁-C₄-alkyl, C₁-C₄ haloalkyl, and —NHSO₂R^{g3}, wherein R^{g3} is C₁-C₄ alkyl. In some embodiments, R′ is C₃-C₆-cycloalkyl-C₁-C₄-alkyl, and R″ is selected from C₁-C₄ alkyl, C₁-C₄ haloalkyl, and —NHSO₂R^{g3} wherein R^{g3} is C₁-C₄ alkyl.

In some embodiments, the compound is selected from:

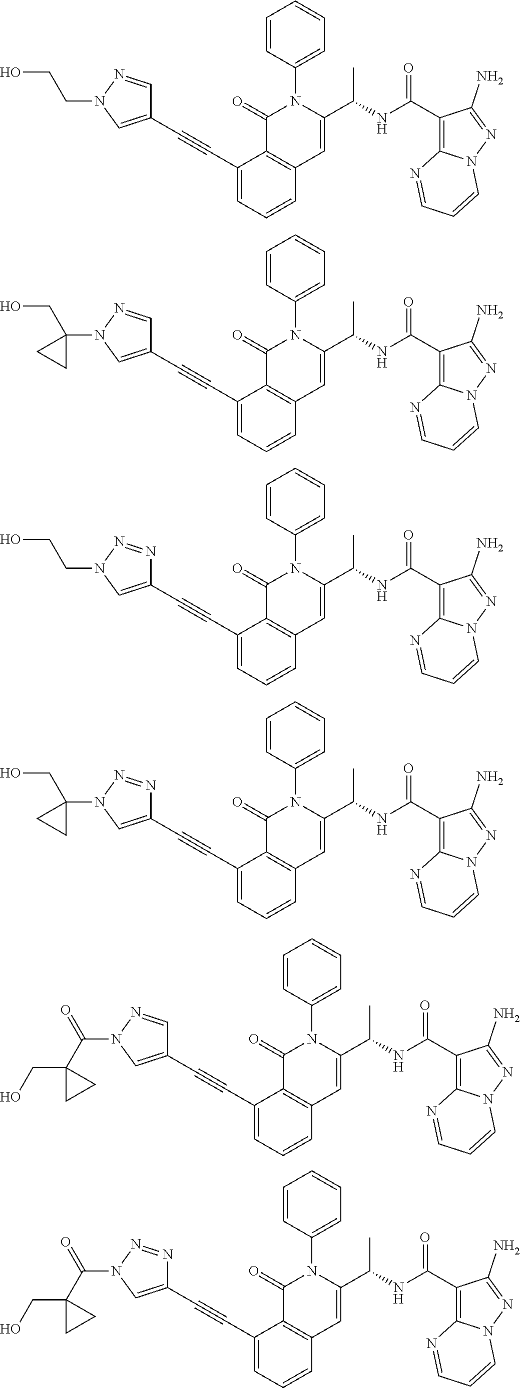

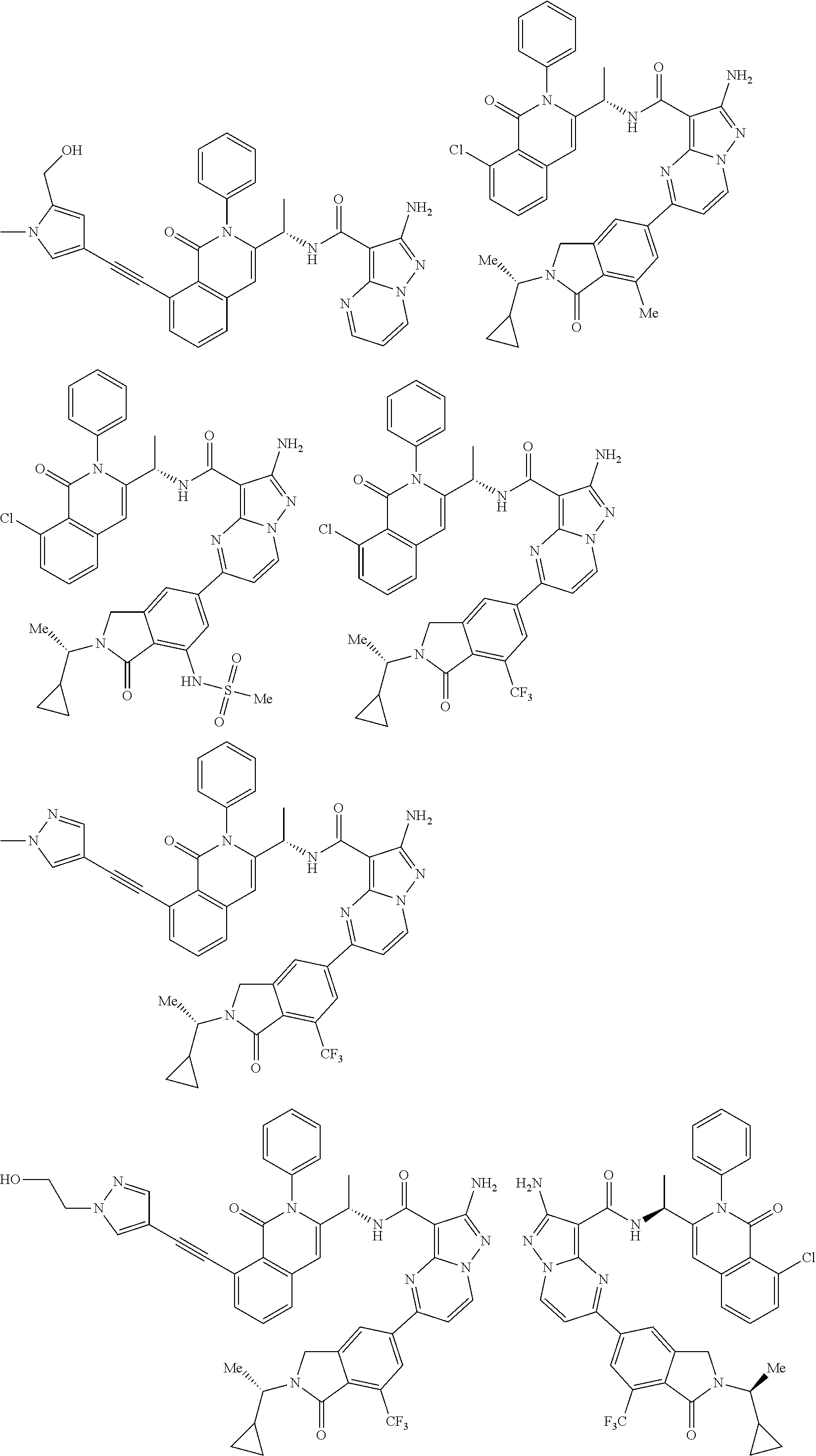

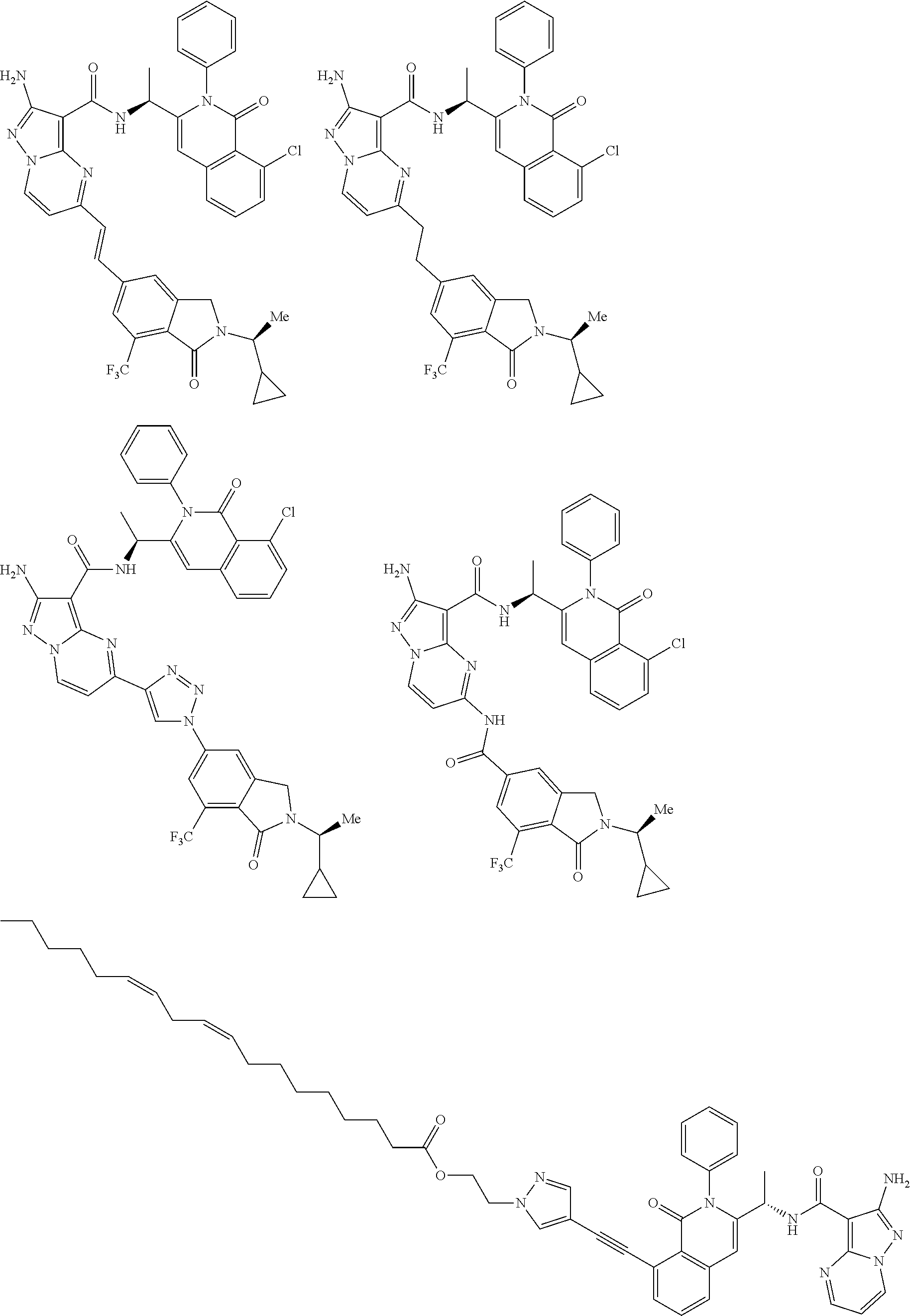

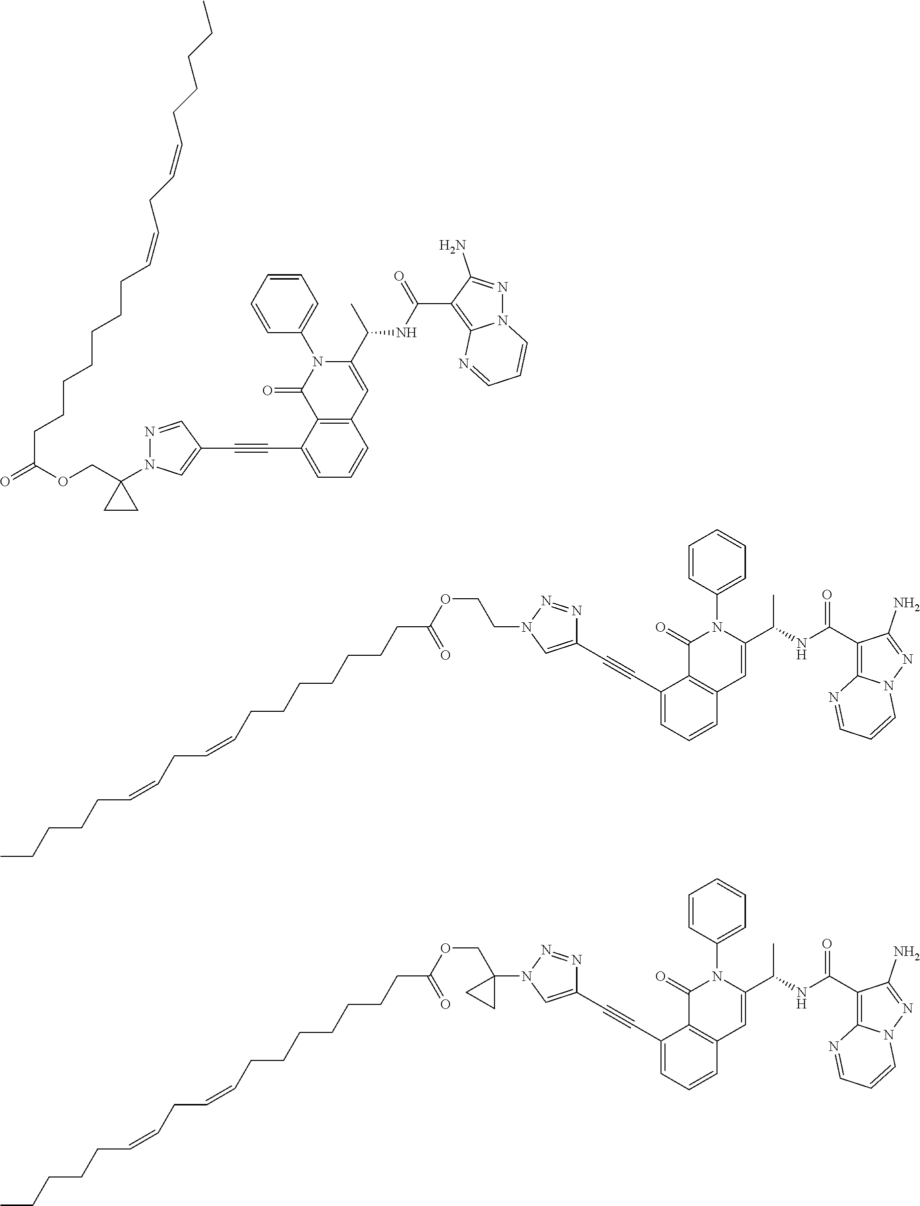 [SMILES: CCCCC/C=C\C/C=C\CCCCCCCC(=O)OCC1(n2cc(C#Cc3cccc4cc([C@H](C)NC(=O)c5c(N)nn6cccnc56)n(-c5ccccc5)c(=O)c34)cn2)CC1 ; CCCCC/C=C\C/C=C\CCCCCCCC(=O)OCCn1cc(C#Cc2cccc3cc([C@H](C)NC(=O)c4c(N)nn5cccnc45)n(-c4ccccc4)c(=O)c23)nn1 ; CCCCC/C=C\C/C=C\CCCCCCCC(=O)OCC1(n2cc(C#Cc3cccc4cc([C@H](C)NC(=O)c5c(N)nn6cccnc56)n(-c5ccccc5)c(=O)c34)nn2)CC1]

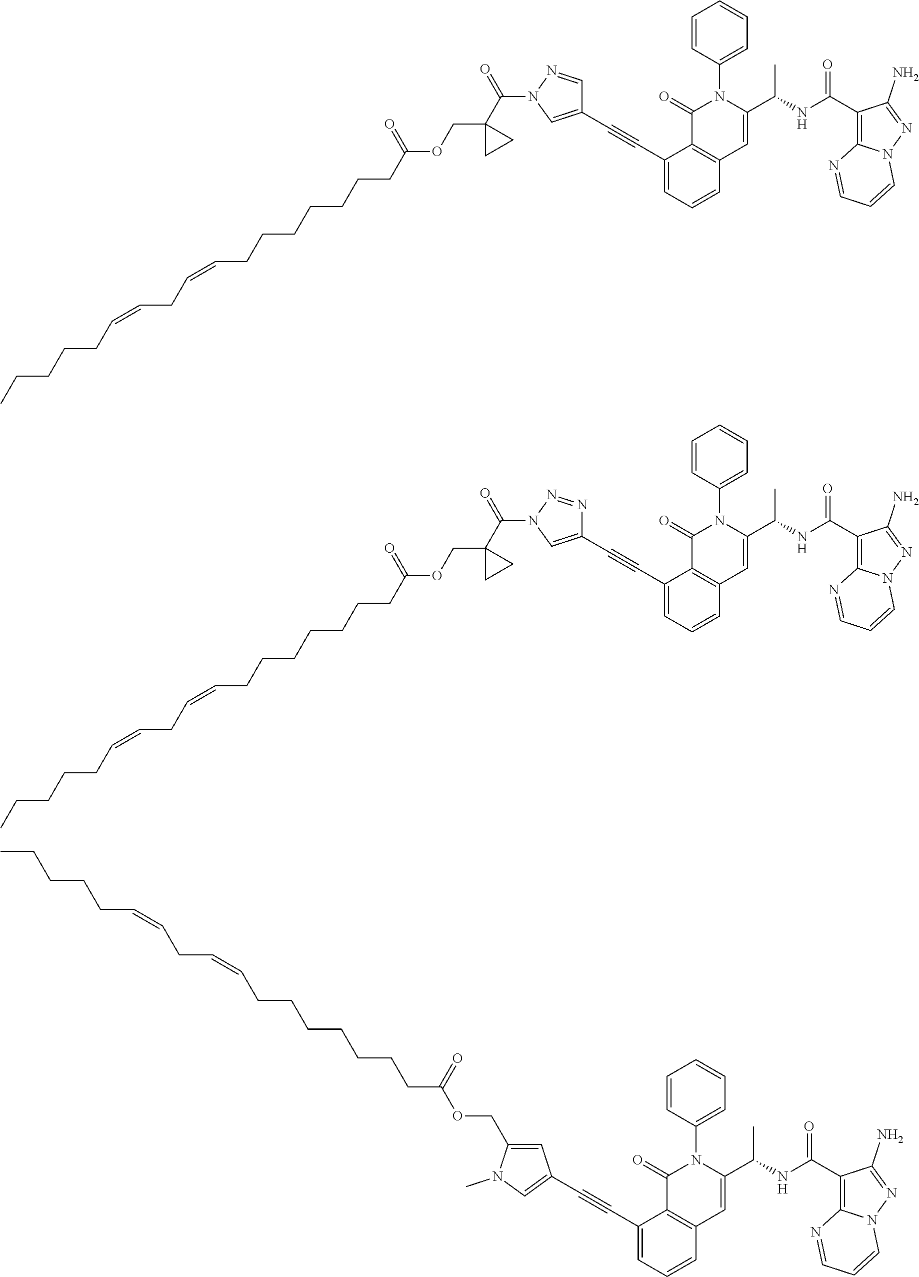

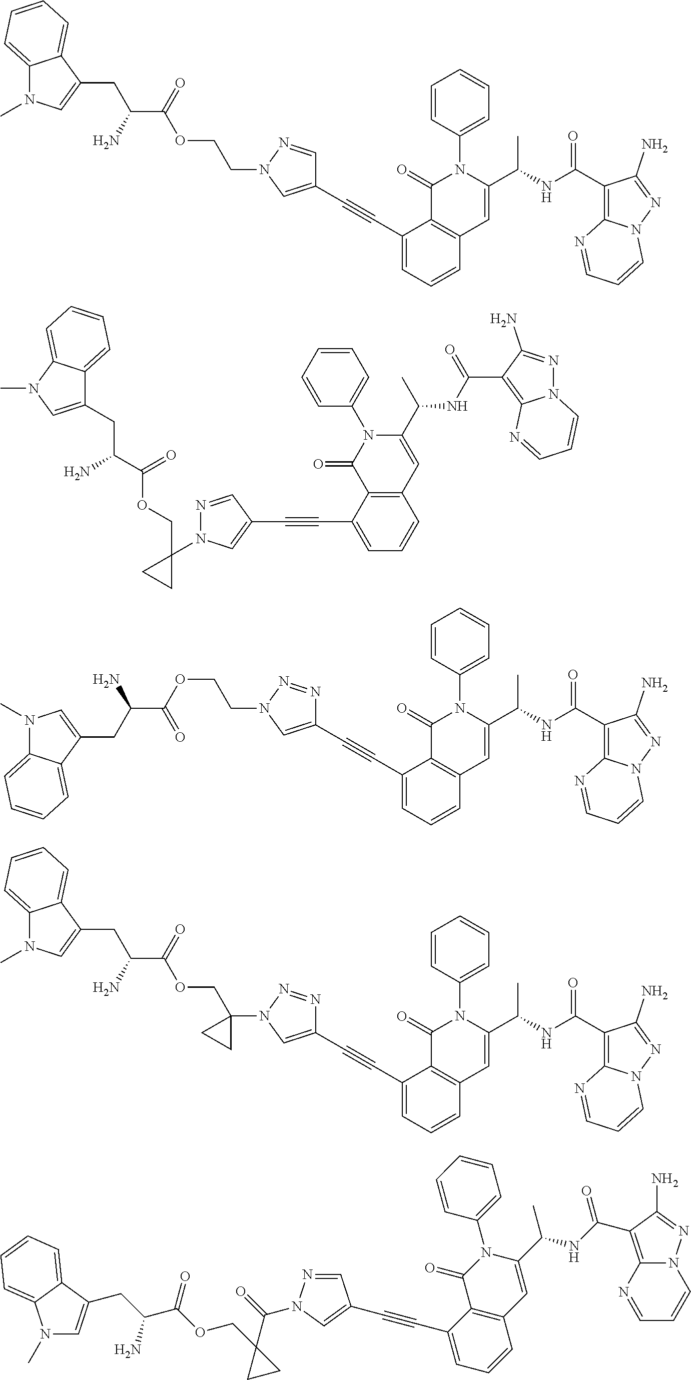

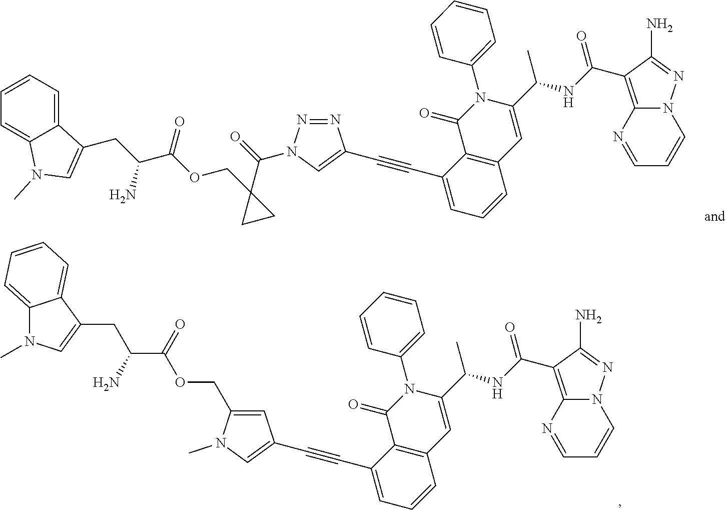 [SMILES: C[C@H](NC(=O)c1c(N)nn2cccnc12)c1cc2cccc(C#Cc3cn(C(=O)C4(COC(=O)[C@H](N)Cc5cn(C)c6ccccc56)CC4)nn3)c2c(=O)n1-c1ccccc1 ; C[C@H](NC(=O)c1c(N)nn2cccnc12)c1cc2cccc(C#Cc3cc(COC(=O)[C@H](N)Cc4cn(C)c5ccccc45)n(C)c3)c2c(=O)n1-c1ccccc1]

and pharmaceutically acceptable salts thereof.

In another aspect, disclosed herein is a pharmaceutical composition comprising a compound of formula (I), or a pharmaceutically acceptable salt thereof. In some embodiments, the compositions further comprise an albumin nanoparticle. In some embodiments, the albumin nanoparticle encapsulates the compound of formula (I). In some embodiments, the albumin nanoparticle has a diameter between 50 and 200 nm. In some embodiments, the albumin is human serum albumin or albumin from animal species.

In some embodiments, the compositions further comprise a liposome, a PLGA or PLA nanoparticle, a lipid nanoparticle, or a micelle.

In some embodiments, the compositions further comprise a chemotherapeutic agent in combination. In some embodiments, the chemotherapeutic agent is paclitaxel. In some embodiments, the chemotherapeutic agents are encapsulated in the albumin nanoparticle, liposome, PLGA nanoparticle, lipid nanoparticle, or micelle.

In a further aspect, disclosed herein are methods for treating or preventing a disease or disorder in a subject (e.g., a human) comprising administering to the subject an effective amount of a compound disclosed herein, or a pharmaceutically acceptable salt thereof, or a composition as disclosed herein.

In some embodiments, the methods further comprise administration of an immunotherapy. In some embodiments, the immunotherapy comprises administration of a PD-1 or PD-L1 antibody. In some embodiments, the immunotherapy is administered at the same time, preceding, or following the compound or the composition. In some embodiments, the immunotherapy is administered by subcutaneous injection.

In some embodiments, the disease or disorder comprises cancer, an autoimmune disease or disorder, or an inflammatory disease or disorder. In some embodiments, the disease or disorder is cancer. In some embodiments, the cancer comprises a solid tumor or hematological cancer. In some embodiments, the cancer is metastatic cancer. In select embodiments, the disease or disorder is breast cancer, pancreatic cancer, lung cancer, or lymphoma. In some embodiments, the methods suppress or eliminate cancer metastasis, decrease tumor growth, prevent tumor recurrences, or any combination thereof. In some embodiments, the compound or the composition is administered by subcutaneous injection.

Other aspects and embodiments of the disclosure will be apparent in light of the following detailed description and accompanying figures.

### BRIEF DESCRIPTION OF THE DRAWINGS

FIGS. 1A-1K show data demonstrating increased M2 macrophage infiltration in tumors and lymph nodes in tumor-bearing mice, and combination of IPI-549 and PTX enhanced M2 to M1 macrophage repolarization to inhibit MCTs growth. FIG. 1A and FIG. 1B show representative images of flow cytometry and quantification of F4/80⁺CD11b⁺ (macrophages), CD80⁺CD206⁺ (M1 phenotype), CD80⁺CD206⁺ (M2 phenotype) in tumor (PyMT-tumor), normal fat pad (N-fat pad), normal lymph nodes (N-LNs), and lymph nodes in tumor bearing mice (PyMT-LNs), n=3. Data are presented as mean±SD. ***P<0.001. FIG. 1C and FIG. 1D show confocal microscopy images of macrophages (red) with M2 phenotype (green) in tumor and lymph nodes of PyMT mice. The nuclei were stained with DAPI (blue). Scale bar: 100 μm. FIGS. 1E-1H show concentrations of TNF-α, IL-12, IL-10, and TGF-β in M2 macrophages cell culture medium (derived from the bone marrow derived macrophages), as measured by ELISA, after treatment with PTX (5 μM), IPI (IPI-549, 5 μM). DOX (doxorubicin, 5 μM), GEM (gemcitabine, 5 μM), PI (PTX, 2.5 μM plus IPI-549, 2.5 M), DI (doxorubicin, 2.5 μM plus IPI-549, 2.5 μM), G1 (gemcitabine, 2.5 μM plus IPI-549, 2.5 μM), n=3. Data are presented as mean±SD. *P<0.05. **P<0.01. ***P<0.001. FIG. 11 is a schematic of 3D multicellular tumor spheroids (MTCs). FIGS. 1J-1K show tumor growth curves and images of the 3D MTCs showing anticancer effect of single-drug treatment with PTX, IPI, DOX or GEM at the concentration of 5 μM, and the combination treatment with PI (PTX, 2.5 μM plus IPI-549, 2.5 μM). DI (doxorubicin, 2.5 μM plus IPI-549, 2.5 μM) or G1 (gemcitabine, 2.5 μM plus IPI-549, 2.5 M). PBS treatment served as the control, n=3. Data are presented as mean±SD. ^{###}P<0.001. Significant differences as compared with PBS treated group. (***P<0.001).

| FIGS. 2A-2F show Nano-PI characterization and enhanced accumulation in both tumors and lymph nodes in MMTV-PyMT transgenic mice. FIG. 2A shows size distribution of Nano-PI (PTX, 0.2 mg/ml) detected by dynamic light scattering (n=3). FIG. 2B shows transmission electron microscopy (TEM) imaging of Nano-PI. Scale bar: 200 nm. FIGS. 2C-2D show stability of Nano-PI as measured by size distribution with different dilutions (PTX concentration from 2×10⁻³ to 2×10⁻⁵). FIGS. 2E-2F show PTX and IPI-549 concentration in plasma, lymph nodes, tumor, and fat pad after intravenous injection of free PTX and IPI-549 (PTX/IPI), intravenous injection of albumin formulation of PTX (Nano-P) plus oral or intraperitoneal injection of IPI-549 (Nano-P+IPI (P.O.) or Nano-P+IPI (I.P.)), and Nano-PI at the dose of PTX 5 mg/kg and IPI-549 2.5 mg/kg into 14-15 weeks old MMTV-PyMT transgenic mice with spontaneous breast cancer (three mice per group, 10 tumors, and 8 fat pad tissues were analyzed for each mouse). Data are shown as mean #SD. *P<0.05, **P<0.01. ***P<0.001. Significant difference as compared with PTX/IPI (free drug) group.

FIGS. 3A-3H show that Nano-PI enhanced delivery to macrophages in both tumors and lymph nodes of MMTV-PyMT transgenic mice. FIG. 3A shows representative tumor confocal microscopic images and 3D MFI plot (area within dashed box) show the drug (green) distribution, and pixel-by-pixel Pearson's correlation R-value (means±SD, n=4) of drug colocalization with blood vessels (α-CD31) and macrophages (α-F4/80). Tumor samples were collected 4 h post intravenous injection of Nano-PI encapsulated with fluorescent PTX-OG488 and IPI-549 (F-Nano-PI) or free PTX-OG488 plus IPI-549 (F-PTX/IPI) in MMTV-PyMT mice. The macrophages, blood vessels, and nucleus were stained with F4/80 (red). CD31 (cyan), and DAPI (blue). Bar: 200 μm. Area within dashed box to show the overlay of F-PTX/IPI with blood vessels (white) or macrophages (yellow). FIG. 3B shows quantification of overlay of drug with blood vessel and macrophages in tumors. F-PTX/IPI (n=8). Nano-PI (n=7). FIG. 3C shows representative images of flow cytometry and quantifications of drug (PTX-OG488) distribution in TAMs (F4/80) and tumor cells (CD44) within tumor tissues collected 4 h post intravenous injection of F-Nano-PI and F-PTX/IPI in MMTV-PyMT mice (n=4). FIG. 3D shows representative lymph nodes confocal images and 3D mean fluorescent Intensity (MFI) plot show the drug (green) distribution, and pixel-by-pixel Pearson's correlation R-value (means±SD, n=4) of drug with blood vessels (α-CD31) and macrophages (α-F4/80). Lymph node samples were collected 4 h post intravenous injection of F-Nano-PI and F-PTX/IPI in MMTV-PyMT mice. The macrophages, blood vessels, and nucleus were stained with F4/80 (red), CD31 (cyan), and DAPI (blue). Bar represents 200 μm. Area within dashed box to show the overlay of F-PTX/IPI with blood vessels (white) or macrophages (yellow). FIG. 3E shows quantification of overlay of drug with blood vessels (α-CD31) and macrophages (α-F4/80) in lymph nodes. (n=5). FIG. 3F shows representative images of flow cytometry and quantifications of drug (PTX-OG488) in SSM (CD169) and MCM/MSM (F4/80) cells within lymph nodes 4 h post intravenous injection of F-Nano-PI and F-PTX/IPI in MMTV-PyMT mice, n=4 independent experiments. FIG. 3G shows confocal microscopic imaging showing the drug (green) distribution within lymph nodes, and pixel-by-pixel Pearson's correlation R-value (means±SD, n=6) of drug colocalization with B cells, T cells, and macrophages (area within dashed box). FIG. 3H shows quantification of colocalization of drug with B cells, T cells, and macrophages, 4 h post administration of F-Nano-PI and F-PTX/IPI in MMTV-PyMT mice (n=3). The macrophages. B cells, T cells, and nucleus were stained with F4/80 and CD169. CD19, CD3, and DAPI, respectively. Data are presented as mean±SD. *P<0.05, **P<0.01, ***P<0.001.

FIGS. 4A-4J show that Nano-PI combined with α-PD1 achieved long-term complete remission and eliminated lung metastasis in MMTV-PyMT mice. FIG. 4A is an illustration of a dosing scheme in which MMTV-PyMT mice were administrated with different treatment at day 66 after birth and observed for 183 days after birth. FIG. 4B shows total tumor volume changes as calculated by the sum of all tumors in each mouse (n=10) after different treatments; mouse serum albumin (vehicle, I.V.), Nano-P (10 mg/kg. I.V.), Nano-P (10 mg/kg. I. V.) plus α-PD1. IPI-549 (15 mg/kg. P.O.) plus α-PD1, Nano-P (5 mg/kg. I V.) plus IPI549 (5 mg/kg. P.O.) and α-PD1, Nano-PI (PTX 10 mg/kg, IPI-549 5 mg/kg), Nano-PI (PTX 10 mg/kg. IPI-549 5 mg/kg) plus α-PD1. The drug treatment was given intravenously once every three days for five doses. α-PD1 were administered intraperitonially once every three days for 3 doses (100 μg/mouse). FIG. 4C shows H&E and Bouin's staining and quantification of metastatic nodules in the lung on the 183rd day after birth. The red circle shows the metastatic lesions. FIG. 4D shows survival rate of MMTV-PyMT mice after treatment (n=10). Data are shown as mean±SD (n=3). ^{#}P<0.05, ^{##}P<0.01. Statistically significant differences as compared with Nano-P plus α-PD group (**P<0.01, ***P<0.001). FIG. 4E is a schematic depicting treatment schedule for tumor re-challenge in the MMTV-PyMT mice with tumor remission after treatment of Nano-PI plus α-PD1 on 210 days after birth as described in FIG. 4A and wild-type FVB/NJ female mice were served as control. FIG. 4F shows changes in tumor volumes were measured for 38 days after tumor inoculation. FIG. 4G is an illustration of dosing scheme showing that MMTV-PyMT mice were treated with different treatment at 80 days after birth. FIG. 4H shows total tumor volume changes as calculated by the sum of all tumors in each mouse (n=10) after intravenous injection of vehicle, PTX/IPI-549 (PTX, 5 mg/kg. IPI-549 2.5 mg/kg, I.V.) plus α-PD1 and Nano-PI (PTX 5 mg/kg, IPI-549 2.5 mg/kg, I.V.) for 5 times plus α-PD1 (100 μg/mouse, IP) for 3 times. FIG. 4I shows H&E and Bouin's staining and quantification analysis show metastatic nodules of the lung on the 113th day after birth. Red circles show the metastasis lesions. Data are shown as mean±SD (n=3). *P<0.05. Statistically significant differences as compared with PTX/IPI-549 plus α-PD1 group (**P<0.01, ***P<0.001). FIG. 4J shows survival rate after treatment of different formulations at a low dose (n=10).

FIGS. 5A-5F show that Nano-PI plus α-PD1 remolded tumor immune microenvironment in MMTV-PyMT transgenic mice. FIG. 5A shows the tSNE visualization and quantification of all immune cells in tumor by Cytometry by time of flight (Cy TOF) test from MMTV-PyMT transgenic mice 10 days after treatment with vehicle (I.V.), a combination of Nano-P (10 mg/kg. I.V.) and IPI549 (5 mg/kg. I.P.) plus α-PD1, and Nano-PI (PTX, 10 mg/kg, IPI-549, 5 mg/kg) plus α-PD1 for 5 times. α-PD1 by I.P, at 100 μg/mouse for 3 times. Data are shown as mean±SD. (n=3). The relative gating scheme is described in Example 4 and the relative panels are listed in Table 1. FIG. 5B shows tSNE visualization of the expression of CD206, CD115, IL-4, and IL10 in tumor from MMTV-PyMT transgenic mice following the same treatment and CyTOF analysis in FIG. 5A. FIGS. 5C-5E show flow cytometry quantification of M1 (F4/80⁺CD80⁺) and M2 (F4/80⁺CD206⁺) macrophage percentage among total macrophages and M1/M2 ratio in tumor of MMTV-PyMT transgenic mice following the same treatment in FIG. 5A. Data are shown as mean±SD (n=3). ^{#}P<0.05. Statistically significant differences as compared with vehicle group (*P<0.05. **P<0.01, ***P<0.001). Representative images are shown in FIG. 19A. FIG. 5F shows confocal microscopic imaging showing the changes of macrophage phenotypes in tumor tissues after the same treatment in FIG. 4B. The total macrophages, M1 macrophages, M2 macrophages and nucleus were stained with F4/80 (red), CD80 (cyan). CD206 (green) and DAPI (blue). Bar represents 400 μm.

FIGS. 6A-6F show that Nano-PI plus α-PD1 prevents T cell exhaustion and activates DCs in tumors of MMTV-PyMT transgenic mice. FIG. 6A shows tSNE visualization of the expression of CTLA-4. PD1. TIM-3. FR4 of T cells in tumor from MMTV-PyMT mice following the same treatment and CyTOF analysis as in FIG. 5A. FIG. 5B shows tSNE visualization of the expression of CD103 in DCs in tumors from MMTV-PyMT mice following the same treatment and CyTOF analysis as in FIG. 4A. FIG. 5C shows representative flow cytometric images and quantification of DCs (CD11C⁺CD103⁺) and activated DCs (CD80⁺CD86⁺) in tumor of MMTV-PyMT mice. Data are shown as mean±SD. (n=4). Statistically significant differences as compared with the vehicle group (**P<0.01, ***P<0.001 vs vehicle, *P<0.05). FIGS. 5D-5F show data from ELISA analysis, showing the granzyme B. IL-12, and IFN-γ in tumor of MMTV-PyMT mice with the same dosage regimen in FIG. 6C. Data are shown as mean±SD. (n=9). ^{##}P<0.01. ^{###}P<0.001, Statistically significant differences as compared with vehicle group (**P<0.01, ***P<0.001).

FIGS. 7A-7H show that Nano-PI combined with α-PD1 remodels the immune microenvironment in lymph nodes of PyMT mice. FIG. 7A shows Cy TOF analysis of all immune cells in lymph nodes of MMTV-PyMT mice following the same treatment as in FIG. 6A. Data are shown as mean±SD. (n=3). The relative gating scheme is described in Example 4. FIGS. 7A-7D show flow cytometry quantification of M1 (F4/80⁺CD80⁺) and M2 (F4/80⁺CD206⁺) macrophage populations in lymph nodes of MMTV-PyMT transgenic mice with the same dosage regimen as described in FIG. 6A. Data are shown as mean±SD (n=3). *P<0.05, **P<0.01. Significant differences as compared with vehicle group (**P<0.01, ***P<0.001). The flow cytometric images are in FIG. 19B. FIG. 7E shows whole lymph nodes scanning by a Nikon Alsi confocal microscopy showing the changes of macrophages phenotypes in lymph nodes following the treatment in FIG. 4B. The total macrophages, M1 macrophages. M2 macrophages and nucleus were stained with F4/80 (red), CD80 (cyan). CD206 (green) and DAPI (blue). Bar represents 400 μm. FIGS. 7F-7H show data from ELISA analyses, showing the amount of granzyme B. IL-12, and IFN-γ in the lymph nodes of MMTV-PyMT transgenic mice following the treatment in FIG. 4B. Data are shown as mean±SD. (n=4). (&P<0.05, &&P<0.01), (**P<0.01, *** P<0.001) Significant differences as compared with vehicle group (**P<0.01. ***P<0.001).

FIGS. 8A-8E show polarization of Macrophage to M1 or M2 phenotype. ELISA analysis (FIG. 8A) and Western blot (FIG. 8B) show the amount of cytokines (TGF-β, TNF-α, and IL-10) and expression of cell surface markers (CD80 and CD206) in the PBS-treated (M0), LPS/IFN-γ-treated (M1), and IL-4/IL-13-treated (M2) macrophages that were generated from bone marrow derived macrophages (BMDMs). The values are mean±SD (n=3). ^{$}P<0.05, ^{$$$}P<0.001. Statistically significant differences as compared with PBS group (**P<0.01, ***P<0.001). FIG. 8C shows INOS and CD206 expression in the PBS-treated (M0). LPS/IFN-γ-treated (M1), and IL-4/IL-13-treated (M2) macrophages that were generated from RAW264.7 macrophages. FIG. 8D shows macrophage morphology after treatment with PBS (M0), LPS/IFN-γ (M1), and IL-4/IL-13 (M2) observed using an inverted fluorescence microscope. Scale bars, 20 μm. FIG. 8E shows data from a transwell invasion assay for determining the invasion 4T1 breast cancer cells after incubation with the conditioned medium of M2 macrophages generated from RAW264.7 macrophages. Scale bars, 100 μm.

FIGS. 9A-9G show the inhibitory effect of PTX and IPI-549 treatment on 3D tumor spheroids and 3D MCTs growth. FIGS. 9A-9B show growth curves and images of the 3D tumor spheroids that established by 4T1 cells alone show the inhibitory effect of single treatment of PTX, IPI-549, doxorubicin (DOX), gemcitabine (GEM) at the concentration of 5 μM, and the combination treatment of PI (PTX 2.5 μM+IPI 2.5 μM), DI (DOX 2.5 μM plus IPI 2.5 μM), G1 (GEM 2.5 μM plus IPI 2.5 μM). PBS treatment served as the control, n=3. Data are presented as mean±SD. ###P<0.001. Statistically significant differences as compared with PBS treated group (***P<0.001). FIGS. 9C-9D show representative images of MCTs (co-culture of 4T1 cells and M2 macrophages). FIGS. 9E-9F show growth curves of MCTs showing an inhibitory effect of single-drug and combination treatment with PTX and/or IPI-549 at the concentration of 0.1, 1, 2, 5, 10 μM. PBS and drug solvent treatment served as the control, n=3. Data are presented as mean±SD. FIG. 9G shows the synergistic inhibitory effect of PTX and IPI-549 on MCT growth (red dot) mapped on Cartesian axes and connected by an isobole (line of additivity) in the isobologram.

FIGS. 10A-10C show that PTX and IPI-549 promote M2 to M1-macrophage repolarization and inhibit cancer cell growth. FIGS. 10A and 10B show ELISA analysis showing the amount of TNF-α and IL-12 in the medium supernatant of M2 macrophages (derived from BMDMs) after treatment with PTX (1, 5, and 10 μM), IPI (IPI-549, 1, 5, and 10 μM) as well as the combination between any two concentrations of PTX with IPI-549. The values are mean±SD (n=3). FIG. 10C shows cell viabilities after treatment with PTX (1, 5, and 10 μM), IPI (IPI-549, 1, 5, and 10 μM) as well as the combination between any two concentrations of PTX with IPI-549 detected by methyl tetrazolium (MTT) assays. Cells treated with PBS were served control with 100% cell viability. The values are mean±SD (n=3).

FIGS. 11A-11C show stability and drug release of Nano-PI in vitro. FIG. 11A shows stability of Nano-PI at different dilutions (PTX concentration from 2× 10-3 to 2× 10-+mg/mL) as measured by size distribution with dynamic light scattering (n=3). Size distribution of Nano-PI was detected immediately after dilution with PBS containing 10% fetal bovine serum (FBS). Size distribution at the range of 2×10⁻³ to 4×10⁻⁴ mg/mL (PTX concentration) remained the same with the main peak of 142 nm. FIGS. 11B and 11C show in vitro cumulative release profiles of IPI-549 (FIG. 11B) and PTX (FIG. 11C) from Nano-PI in plasma at 37° C. Data are shown as mean±SD (n=3).

FIGS. 12A-12E show that Nano-PI inhibited M2 macrophage polarization, tumor growth, and invasion. FIG. 12A shows morphology of RAW 274.7 derived macrophages after treatment with PBS (M0 macrophages), LPS/IFN-γ (M1 macrophages), or IL-4/IL-13 (M2 macrophages). Nano-PI was treated after cells pretreated with IL-4/IL-13 turning to M2 macrophages. Scale bars, 20 μm. FIGS. 12B and 12C show results of an ELISA assay showing the concentration of TGF-β (FIG. 12B) and IL-12 (FIG. 12C) in M2 macrophages cell cultured medium (derived from RAW 274.7) following treatment with PTX, IPI-549 (IPI), PTX plus IPI, Nano-P, and Nano-PI at the PTX and IPI concentration of 10 μM and 5 μM, respectively. FIG. 12D shows cell viability of 4T1 cells as detected by MTT assay after incubation with PTX. IPI, and Nano-PI for 24 h. FIG. 12E shows data from a transwell invasion assay for determining the invasion 4T1 breast cancer cells after incubation with the conditioned medium of PTX, IPI, and Nano-PI treated M2 macrophages (generated from RAW264.7 macrophages). Scale bars, 100 μm. The values are mean±SD (n=3). *P<0.05. **P<0.01, ***P<0.001.

FIGS. 13A-13B show pharmacokinetics and tissue distributions of Nano-PI in PyMT transgenic mice. PTX (FIG. 13A) and IPI-549 (FIG. 13B) concentration in the liver, spleen, and lung after intravenous injection of free PTX and IPI-549 in combination, albumin formulation of paclitaxel (Nano-P) plus oral IPI-549, and Nano-PI at the dose of PTX (5 mg/kg) and IPI-549 (2.5 mg/kg) on MMTV-PyMT mice (n=3). Data are shown as mean±SD.

FIGS. 14A-14B show mass spectrometry (MS) images of IPI-549 and PTX distribution. MS imaging shows the drug distribution in tumor and lymph nodes that were delivered by Nano-PI. MMTV-PyMT mice (female, 10-11 weeks old) were administrated with Nano-PI (IV) at the dosage of PTX 100 mg/kg and IPI-549 50 mg/kg, and tumors and lymph nodes were dissected and prepared the frozen sections for MS imaging 4 hours post treatment. PTX is shown in green. IPI-549 is shown in red. Overlay imaging (yellow) suggests colocalization of PTX and IPI-549.

FIGS. 15A-15D show Nano-PI distribution in the lymph nodes. Lymph nodes were collected 4 h post intravenous injection of Nano-PI encapsulated with fluorescent PTX-OG488 and IPI-549 (F-Nano-PI) in MMTV-PyMT mice. Confocal microscopic imaging shows the drug (green) distribution within lymph nodes, and pixel-by-pixel Pearson's correlation shows drug colocalization with B cells, T cells, and macrophages (area within dashed box). The macrophages. B cells, T cells, and nucleus were stained with F4/80 and CD169, CD19, CD3, and DAPI, respectively. FIG. 15A shows the whole lymph nodes. Scale bar: 500 μm. FIG. 15B shows magnification of the area of drug distribution in lymph nodes and FIG. 15C shows drug distribution with B cells, T cells and macrophages. Scale bar: 200 μm. FIG. 15D shows pixel-by-pixel Pearson's correlation R-value (mean±SD, n=6) of drug distribution in B cells, T cells, and macrophages within lymph nodes, 4 h post administration.

FIGS. 16A-16F show the in vivo antitumor efficacy of Nano-PI on PyMT transgenic mice. FIGS. 16A-16B show the total tumor volume (FIG. 16A) and tumor numbers (FIG. 16B) calculated by sum of all tumors in each group (n=10 mice) of the same test in FIG. 4A (main text). FIG. 16C is an illustration of a dosing scheme in which MMTV-PyMT transgenic mice were administered vehicle (IV), Nano-P. (5 mg/kg, I.V.) plus IPI-549 (15 mg/kg, I.P.) and α-PD1. Nano-PI (PTX, 10 mg/kg, IPI-549, 5 mg/kg) plus α-PD1 for 5 times. α-PD1 were dosed by I.P, once every three days for a total of 3 times at the dosage of 100 μg/mouse. FIGS. 16D-16F show total tumor volume changes calculated by sum of all tumors in each mouse (n≥10 tumors).

FIGS. 17A-17J show total memory related T and B cell population after the treatment, from flow cytometry analysis of memory-related T cells including TCM (CD3⁺CD197⁺), TEM (CD3⁺CD44⁺), and TRM (CD3⁺CD103⁺) (FIGS. 17A-17E), as well as memory B (MB) cells including MB1 (CD19⁺CD73⁺CD80⁺). MB2 (CD19⁺CD73⁺PD-L2⁺), MB3 (CD19⁺PD-L2⁺CD80⁺), and MB4 (CD19⁺CD73⁺CD80-PD-L2⁺) (FIGS. 17F-17J) in the lymph nodes, spleen, bone marrow, blood, and lung at the end of the tumor rechallenge test. Data were shown as mean±SD (n=3). *P<0.05, **P<0.01. ***P<0.001.

FIGS. 18A-18E show the in vivo antitumor efficacy of Nano-PI on 4T1 orthotopic breast cancer model. FIG. 18A is an illustration of the dosing scheme for treatment of 3 doses in 4T1 tumor bearing mice. FIG. 18B shows the tumor growth curves of breast cancer-bearing mice after different treatments; mouse serum albumin (vehicle, I.V.). Nano-P (PTX, 10 mg/kg. I.V.), Nano-P (PTX, 10 mg/kg, I.V.) plus α-PD1. IPI-549 (5 mg/kg, P.O.) plus α-PD1. Nano-P (PTX: 5 mg/kg, I.V) plus IPI549 (5 mg/kg, P.O.) and α-PD1, Nano-P (PTX: 5 mg/kg, I.V.) plus IPI549 (5 mg/kg, I.P.) and α-PD1, and Nano-PI (PTX, 10 mg/kg, IPI-549, 5 mg/kg) plus α-PD1 for 3 times. α-PD1 were dosed by I.P, once every three days for a total of 3 times at the dosage of 100 μg/mouse. The values are mean±SD (n=8). **P<0.01. ***P<0.001. FIG. 18C shows tumor weights. *P<0.05. **P<0.01 as compared with the vehicle group (*P<0.05, **P<0.01, ***P<0.001). FIG. 18D shows photographs of the excised tumors at the 24th day. FIG. 18E shows H&E section of lung tissues showing lung metastasis of breast cancer-bearing mice. Bar represents 100 μm.

FIGS. 19A-19B show representative images of flow cytometric analysis. M1 (F4/80*CD80⁺) (FIG. 19A) and M2 (F4/80*CD206+) macrophage (FIG. 19B) percentages among total macrophages in tumor and lymph node from MMTV-PyMT transgenic mice 10 days after treatment with mouse serum albumin (vehicle, I.V.), a combination of Nano-P (10 mg/kg. I.V.) and IPI549 (5 mg/kg, IP) plus α-PD1, and Nano-PI (PTX, 10 mg/kg, IPI-549, 5 mg/kg) plus α-PD1 for 5 times, α-PD1 by IP at 100 μg/mouse for 3 times (n=3).

FIGS. 20A-20C show that Nano-PI combined with α-PD1 remodeled immune microenvironment in tumors of 4T1 breast cancer mice and MMTV-PyMT transgenic mice. FIG. 20A shows quantification from a flow cytometry analysis of MHC II CD206 (M1 macrophages). MHC II⁻CD206⁺ (M2 macrophages) in 4T1 tumors after treatment with mouse serum albumin (vehicle, I.V.). Nano-P. (PTX: 10 mg/kg, I.V.). Nano-P (PTX: 10 mg/kg. I.V.) plus α-PD1. IPI-549 (5 mg/kg. P.O.) plus α-PD1, Nano-P (5 mg/kg, I.V.) plus IPI549 (15 mg/kg, P.O.) and α-PD1, Nano-P (PTX: 5 mg/kg. I.V) plus IP1549 (15 mg/kg, I.P.) and α-PD1, and Nano-PI (PTX, 10 mg/kg. IPI-549, 5 mg/kg) plus α-PD1 for 3 times. α-PD1 were dosed by I.P, once every three days for a total of 3 times at the dosage of 100 μg/mouse. Data are shown as mean±SD (n=3). FIGS. 20B-20C show quantification from flow cytometry analyses of CD3 T cells, CD4 T cells. CD8 T cells, and Tregs (CD4⁺Foxp3⁺) following the same treatment as described in (FIG. 20A). Data are shown as mean±SD (n=3). ^{#}P<0.05. ^{##}P<0.01, ^{###}P<0.001 Statistically significant differences as compared with the vehicle group (*P<0.05, **P<0.01, ***P<0.001).

FIGS. 21A-21B show quantification from a flow cytometry analysis of total immune cells (CD45⁺) as well as the macrophages (CD45⁺F4/80⁺), T cells (CD45⁺CD3⁺), and B cells (CD45⁺CD19) out of 2 million single cells of each tumor sample from different MMTV-PyMT transgenic mice. MMTV-PyMT transgenic mice were treated with vehicle, Nano-P plus IPI549 (I.P.) and α-PD1. Nano-PI plus α-PD1 every three days and in total 5 times at the PTX and IPI-549 dosages of 10 mg/kg and 5 mg/kg, respectively. *P<0.05, **P<0.01, ***P<0.001.

FIGS. 22A-22B show total immune cell populations in lymph nodes, quantified from a flow cytometry analysis of total immune cells (CD45⁺) as well as the macrophages (CD45 F4/80⁺), T cells (CD45⁺CD3⁺). B cells (CD45⁺CD19), and NK cells (CD45⁺CD335⁺) out of 2 million single cells of each tumor sample from different MMTV-PyMT transgenic mice. MMTV-PyMT transgenic mice were treated with mouse serum albumin (vehicle), Nano-P plus IPI549 (IP) and α-PD1, Nano-PI plus α-PD1 every three days and in total 5 times at the PTX and IPI-549 dosages of 10 mg/kg and 5 mg/kg, respectively. *P<0.05, **P<0.01. ***P<0.001.

FIG. 23 shows that Nano-PI combined with α-PD1 improved survival of KPC mice with metastatic pancreatic cancer. FIG. 23 shows survival rate of KPC mice with pancreatic cancer after treatment (n=10); mouse serum albumin (vehicle), Abraxane (Abrax, IV 10 mg/kg)+IPI549 (IP, 15 mg/kg)+α-PD1 (PD1. IP, 100 μg); and Nano-PI (PTX 10 mg/kg. IPI-549 5 mg/kg) plus α-PD1 (PD-1, IP 100 μg). The Nano-PI was given intravenously once every three days for five doses. α-PD1 were administered intraperitonially once every three days for 3 doses (100 μg/mouse). IPI-549 was given intraperitoneally once every three days for five doses.

### DETAILED DESCRIPTION

Described herein are phosphatidylinositol 3-kinase (PI3K) inhibitors and compositions thereof. PI3K inhibitor compositions comprising a chemotherapeutic agent are shown to remodel the immune microenvironment in lymph nodes and tumors, and when combined with αPD-1 achieve complete remission with 100% survival and complete elimination of metastasis in transgenic mice with spontaneous metastatic breast cancer and pancreatic cancer (at >200 days).

Section headings as used in this section and the entire disclosure herein are merely for organizational purposes and are not intended to be limiting.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular. All publications, patent applications, patents and other references mentioned herein are incorporated by reference in their entirety.

As used herein, “treat,” “treating” and the like means a slowing, stopping, or reversing of progression of a disease or disorder when provided a compound or composition described herein to an appropriate control subject. The term also means a reversing of the progression of such a disease or disorder to a point of eliminating or greatly reducing the symptoms. As such, “treating” means an application or administration of the compositions described herein to a subject, where the subject has a disease or a symptom of a disease, where the purpose is to cure, heal, alleviate, relieve, alter, remedy, ameliorate, improve, or affect the disease or symptoms of the disease.

A “subject” or “patient” may be human or non-human and may include, for example, animal strains or species used as “model systems” for research purposes, such a mouse model as described herein. Likewise, patient may include either adults or juveniles (e.g., children) Moreover, patient may mean any living organism, preferably a mammal (e.g., humans and non-humans) that may benefit from the administration of compositions contemplated herein. Examples of mammals include, but are not limited to, any member of the Mammalian class; humans, non-human primates such as chimpanzees, and other apes and monkey species; farm animals such as cattle, horses, sheep, goats, swine, domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

As used herein, the terms “providing,” “administering,” “introducing.” are used interchangeably herein and refer to the placement of the compositions of the disclosure into a subject by a method or route which results in at least partial localization of the composition to a desired site. The compositions can be administered by any appropriate route which results in delivery to a desired location in the subject.

Definitions of specific functional groups and chemical terms are described in more detail below. For purposes of this disclosure, the chemical elements are identified in accordance with the Periodic Table of the Elements. CAS version, Handbook of Chemistry and Physics, 75^{th} Ed., inside cover, and specific functional groups are generally defined as described therein. Additionally, general principles of organic chemistry, as well as specific functional moieties and reactivity, are described in Sorrell, Organic Chemistry, 2^{nd} edition, University Science Books. Sausalito, 2006; Smith, March's Advanced Organic Chemistry: Reactions, Mechanism, and Structure, 7th Edition, John Wiley & Sons, Inc., New York, 2013: Larock, Comprehensive Organic Transformations, 3rd Edition. John Wiley & Sons, Inc., New York, 2018; and Carruthers. Some Modern Methods of Organic Synthesis, 3rd Edition, Cambridge University Press, Cambridge, 1987; the entire contents of each of which are incorporated herein by reference.

The term “alkyl,” as used herein, means a straight or branched, saturated hydrocarbon chain. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, 4,4-dimethylpentan-2-yl, n-heptyl, n-octyl, n-nonyl, n-decyl, undecyl, dodecyl, tridecyl, tetradecyl, pentadecyl, hexadecyl, heptadecyl, octadecyl, nonadecyl and icosyl.

The term “alkenyl,” as used herein, means a straight or branched hydrocarbon chain containing at least one carbon-carbon double bond. The double bond(s) may be located at any positions with the hydrocarbon chain. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkynyl” as used herein, means a straight or branched hydrocarbon chain containing at least one carbon-carbon triple bond. The triple bond(s) may be located at any positions with the hydrocarbon chain Representative examples of alkynyl include, but are not limited to, ethynyl, propynyl, and butynyl.

The term “alkylene,” as used herein, refers to a divalent group derived from a straight or branched chain hydrocarbon, for example, of 1 to 10 carbon atoms. Representative examples of alkylene include, but are not limited to, —CH₂CH₂—, —CH₂CH₂CH₂—, —CH₂CH(CH₃)CH₂—, —CH₂CH₂CH₂CH₂—, —CH₂CH(CH₃)CHCH₂—, —CH₂CH₂CH₂CH₂CH₂—, —CH₂CH₂CH₂CH₂CH₂CH₂—, —CH₂CH₂CH₂CH₂CH₂ CH₂CH₂—, —CH₂(CH₂)₆CH₂—, —CH₂(CH₂)—CH₂—, and —CH₂(CH₂)₂CH₂—.

The term “alkoxy,” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy and tert-butoxy.

The term “amino,” as used herein, refers to an —NH₂ group. The term “alkylamino.” as used herein, refers to a group —NHR, wherein R is an alkyl group as defined herein. The term “dialkylamino.” as used herein, refers to a group —NR₂, wherein each R is independently an alkyl group as defined herein.

The term “aryl,” as used herein, refers to an aromatic carbocyclic ring system having a single ring (monocyclic) or multiple rings (bicyclic or tricyclic) including fused ring systems, and zero heteroatoms. As used herein, aryl contains 6-20 carbon atoms (C₆-C₂₀ aryl), 6 to 14 ring carbon atoms (C₆-C₁₄ aryl), 6 to 12 ring carbon atoms (C₆-C₁₂ aryl), or 6 to 10 ring carbon atoms (C₆-C₁₀ aryl). Representative examples of aryl groups include, but are not limited to, phenyl, naphthyl, anthracenyl, and phenanthrenyl.

As used herein, the term “arylene” refers to a divalent aryl group. Representative examples of arylene groups include, but are not limited to, phenylene groups (e.g., 1,2-phenylene, 1,3-phenylene, and 1,4-phenylene).

The term “arylalkyl,” as used herein, refers to an alkyl group, as defined herein, in which at least one hydrogen atom is replaced with an aryl group, as defined herein. Representative arylalkyl groups include, but are not limited to, benzyl, phenethyl, diphenylmethyl, and trityl. The term “cyano,” as used herein, means a —CN group.

The term “cycloalkyl,” as used herein, refers to a saturated carbocyclic ring system containing three to ten carbon atoms and zero heteroatoms. The cycloalkyl may be monocyclic, bicyclic, bridged, fused, or spirocyclic. Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, bicyclo[2.2.1]heptanyl, bicyclo[3.2.1]octanyl, and bicyclo[5.2.0]nonanyl.

The term “cycloalkylene,” as used herein, refers to a divalent cycloalkyl group.

The term “halogen” or “halo,” as used herein, means F, Cl, Br, or I.

The term “haloalkyl.” as used herein, means an alkyl group, as defined herein, in which at least one hydrogen atom (e.g., one, two, three, four, five, six, seven or eight hydrogen atoms) is replaced with a halogen. In some embodiments, each hydrogen atom of the alkyl group is replaced with a halogen. Representative examples of haloalkyl include, but are not limited to, fluoromethyl, difluoromethyl, trifluoromethyl, 2,2,2-trifluoroethyl, and 3,3,3-trifluoropropyl.

As used herein, the term “haloalkoxy” means a haloalkyl group, as defined herein, is appended to the parent molecular moiety through an oxygen atom. Representative examples of haloalkoxy include, but are not limited to, difluoromethoxy, trifluoromethoxy, and 2,2,2-trifluoroethoxy.

As used herein, the term “heteroaryl” refers to an aromatic group having a single ring (monocyclic) or multiple rings (bicyclic or tricyclic), and having one or more ring heteroatoms independently selected from N, O, S, and P. The aromatic monocyclic rings are five- or six-membered rings containing at least one heteroatom independently selected from N, O, S, and P (e.g., 1, 2, 3, or 4 heteroatoms independently selected from N, O, S, and P). The five-membered aromatic monocyclic rings have two double bonds, and the six-membered aromatic monocyclic rings have three double bonds. The bicyclic heteroaryl groups are exemplified by a monocyclic heteroaryl ring appended fused to a monocyclic aryl group, as defined herein, or a monocyclic heteroaryl group, as defined herein. The tricyclic heteroaryl groups are exemplified by a monocyclic heteroaryl ring fused to two rings independently selected from a monocyclic aryl group, as defined herein, and a monocyclic heteroaryl group as defined herein. Representative examples of monocyclic heteroaryl include, but are not limited to, pyridinyl (including pyridin-2-yl, pyridin-3-yl, pyridin-4-yl), pyrimidinyl, pyrazinyl, pyridazinyl, pyrrolyl, benzopyrazolyl, 1,2,3-triazolyl, 1,3,4-thiadiazolyl, 1,2,4-thiadiazolyl, 1,3,4-oxadiazolyl, 1,2,4-oxadiazolyl, imidazolyl, thiazolyl, isothiazolyl, thienyl, furanyl, oxazolyl, isoxazolyl, 1,2,4-triazinyl, and 1,3,5-triazinyl. Representative examples of bicyclic heteroaryl include, but are not limited to, benzimidazolyl, benzodioxolyl, benzofuranyl, benzooxadiazolyl, benzopyrazolyl, benzothiazolyl, benzothienyl, benzotriazolyl, benzoxadiazolyl, benzoxazolyl, chromenyl, imidazopyridine, imidazothiazolyl, indazolyl, indolyl, isobenzofuranyl, isoindolyl, isoquinolinyl, naphthyridinyl, purinyl, pyridoimidazolyl, quinazolinyl, quinolinyl, quinoxalinyl, thiazolopyridinyl, thiazolopyrimidinyl, thienopyrrolyl, and thienothienyl. Representative examples of tricyclic heteroaryl include, but are not limited to, dibenzofuranyl and dibenzothienyl. The monocyclic, bicyclic, and tricyclic heteroaryls are connected to the parent molecular moiety through any carbon atom or any nitrogen atom contained within the rings.

As used herein, the term “heteroarylene” refers to a divalent heteroaryl group.

As used herein, the term “heterocycle” or “heterocyclic” refers to a saturated or partially unsaturated non-aromatic cyclic group having one or more ring heteroatoms independently selected from N, O, S, and P, means a monocyclic heterocycle, a bicyclic heterocycle, or a tricyclic heterocycle. The monocyclic heterocycle is a three-, four-, five-, six-, seven-, or eight-membered ring containing at least one heteroatom independently selected from N, O, S, and P. The three- or four-membered ring contains zero or one double bond, and one heteroatom selected from N, O, S, and P. The five-membered ring contains zero or one double bond and one, two or three heteroatoms selected from N, O, S, and P. The six-membered ring contains zero, one, or two double bonds and one, two, or three heteroatoms selected from N, O, S, and P. The seven- and eight-membered rings contains zero, one, two, or three double bonds and one, two, or three heteroatoms selected from N, O, S, and P. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, oxetanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. Representative examples of bicyclic heterocycles include, but are not limited to, benzopyranyl, benzothiopyranyl, chromanyl, 2,3-dihydrobenzofuranyl, 2,3-dihydrobenzothienyl, 2,3-dihydroisoquinoline, 2-azaspiro[3.3]heptan-2-yl, azabicyclo[2.2.1]heptyl (including 2-azabicyclo[2,2,1]hept-2-yl), 2,3-dihydro-1H-indolyl, isoindolinyl, octahydrocyclopenta[c]pyrrolyl, octahydropyrrolopyridinyl, and tetrahydroisoquinolinyl. Tricyclic heterocycles are exemplified by a bicyclic heterocycle fused to a phenyl group, or a bicyclic heterocycle fused to a monocyclic cycloalkyl, or a bicycle heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms.

Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3,3,1,1^{3,7}]decane), and oxa-adamantane (2-oxatricyclo[3,3,1,1^{3,7}]decane). The monocyclic, bicyclic, and tricyclic heterocycles are connected to the parent molecular moiety through any carbon atom or any nitrogen atom contained within the rings.

As used herein, the term “heterocyclylene” refers to a divalent heterocyclyl group.

As used herein, the term “substituent” refers to a group substituted on an atom of the indicated group.

When a group or moiety can be substituted, the term “substituted” indicates that one or more (e.g., 1, 2, 3, 4, 5, or 6; in some embodiments 1, 2, or 3; and in other embodiments 1 or 2) hydrogen atoms on the group indicated in the expression using “substituted” can be replaced with a selection of recited indicated groups or with a suitable substituent group known to those of skill in the art (e.g., one or more of the groups recited below), provided that the designated atom's normal valence is not exceeded. Substituent groups include, but are not limited to, alkyl, alkenyl, alkynyl, alkoxy, acyl, amino, amido, amidino, aryl, azido, carbamoyl, carboxyl, carboxyl ester, cyano, cycloalkyl, cycloalkenyl, guanidino, halo, haloalkyl, haloalkoxy, heteroalkyl, heteroaryl, heterocyclyl, hydroxy, hydrazino, imino, oxo, nitro, phosphate, phosphonate, sulfonic acid, sulfonamido, thiol, thione, thioxo, or combinations thereof.

As used herein, in chemical structures the indication:

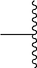

represents a point of attachment of one moiety to another moiety.

In some instances, the number of carbon atoms in a hydrocarbyl substituent (e.g., alkyl alkenyl) is indicated by the prefix “Cₓ-C_{y}”, wherein x is the minimum and y is the maximum number of carbon atoms in the substituent. Thus, for example, “C₁-C₃alkyl” refers to an alkyl substituent containing from 1 to 3 carbon atoms.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

Where substituent groups are specified by their conventional chemical formulae, written from left to right, they optionally encompass substituents resulting from writing the structure from right to left, e.g., —CH₂O— is intended to encompass —OCH₂—, and —C(O)NH— is intended to encompass —NHC(O)—.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Compounds

In one aspect, disclosed is a compound of formula (I):

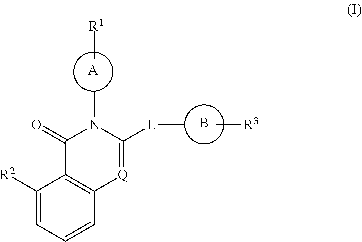

or a pharmaceutically acceptable salt thereof, wherein:
Q is CH or N;
A is aryl or a 5- or 6-membered monocyclic heteroaryl having 1, 2, 3, or 4 heteroatoms independently selected from N, O, S, and P;
R¹ is selected from hydrogen, halo, C₁-C₄ alkyl, C₃-C₆ cycloalkyl, C₁-C₄ haloalkyl, —OR^{a1}, —N(R^{b1})(R^{c1}), —SO₂R^{d1}, —SO₂N(R^{e1})(R^{f1}), and —NHSO₂R&1, wherein R^{a1}, R^{b1}, R^{c1}, R^{d1}, R^{e1}, R^{f1}, and R^{g1} are each independently selected from hydrogen, C₁-C₄ alkyl, and C₁-C₄ haloalkyl;
R² is selected from hydrogen, halo, C₁-C₄ alkyl, C₁-C₄ haloalkyl, C₁-C₄ alkoxy, C₁-C₄ haloalkoxy, and a group of formula (II):

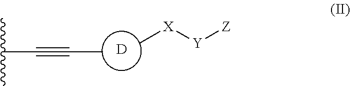

wherein:
   D is a monocyclic heteroaryl or monocyclic heterocyclyl, each of which is optionally substituted with a C₁-C₄ alkyl group;
   X is a bond, —C(O)—, —NH—, or —C(O)NH—;
   Y is —(CR^{a2})ₙ-G²-, wherein each R^{a2} is independently selected from H and C₁-C₄ alkyl, or wherein two R^{a2} together with the carbon atom(s) to which they are attached form a C₃-C₇ cycloalkylene ring; G2 is a bond, cycloalkylene, or heterocyclylene; and n is 0, 1, 2, or 3;
   Z is —OR^{b2}, —SR^{c2}, —N(R^{d2})(R^{e2}), or —CHs, wherein R^{b2}, R^{c2}, R^{d2}, and R^{e2} are each independently selected from hydrogen, aryl, arylalkyl, C₁-C₄ alkyl, —C(O)—C₁-C₄₀ alkyl, —C(O)—C₂-C₄₀ alkenyl, and a group of formula (IIa):

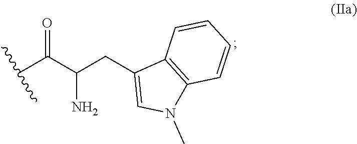 [SMILES: *C(=O)C(N)Cc1cn(C)c2ccccc12]

and
R³ is selected from hydrogen and a group -L³-E, wherein:
   L³ is a bond, C₁-C₂ alkylene, —CH═CH—, —C≡C—, —C(O)—, —O—, —NH—, —S—, —C(O)O)—, —C(O)NH—, —C(O)S—, arylene, cycloalkylene, heteroarylene, or heterocyclylene, or wherein L³ comprises a combination of any two of such groups; and
   E is a bicyclic heterocyclyl or bicyclic heteroaryl, each of which is optionally substituted with 1, 2, 3, 4, or 5 substituents independently selected from halo, C₁-C₄ alkyl, C₃-C₆ cycloalkyl, C₃-C₆-cycloalkyl-C₁-C₄-alkyl, C₁-C₄ haloalkyl, oxo, —OR^{a3}, —N(R^{b3})(R^{c3}), —SO₂R^{d3}, —SO₂N(R^{e3})(R^{f3}), and —NHSO₂R^{g3}, wherein R^{a3}, R^{b3}, R^{c3}, R^{d3}, R^{e3}, R^{f3}, and R^{g3} are each independently selected from hydrogen, C₁-C₄ alkyl, and C₁-C₄ haloalkyl;
L is —(CR^{a4}R^{b4})ₘ-G⁴-, wherein:
   R^{a4} and R^{b4} are independently selected from hydrogen and C₁-C₄ alkyl;
   m is 0 1, or 2; and
   G⁴ is a bond, —NHC(O)—, —NH—, —O— or —S—; and
B is a bicyclic heteroaryl or bicyclic heterocyclyl, each of which is optionally substituted with 1, 2, 3, 4, or 5 substituents independently selected from halo, C₁-C₄ alkyl, C₃-C₆ cycloalkyl, C₁-C₄ haloalkyl, optionally substituted aryl, —OR^{a5}, —N(R^{b5})(R^{c5}), —SO₂R^{d5}, —SO₂N(R^{e5})(R^{f5}), and —NHSO₂R^{g5}, wherein R^{a5}, R^{b5}, R_{c5}, R^{d5}, R^{e5}, R^{f5}, and R^{g5} are each independently selected from hydrogen, C₁-C₄ alkyl, and C₁-C₄ haloalkyl:
wherein when R³ is hydrogen, R² is a group of formula (II) and Z is not —CH₃; and
wherein when R² is hydrogen, halo, C₁-C₄ alkyl, C₁-C₄ haloalkyl, C₁-C₅ alkoxy, or C₁-C₄ haloalkoxy, R³ is a group -L³-E.

In some embodiments, L is —(CR^{a4}R^{b4})ₘ-G⁴-, wherein m is 0, 1, or 2, R^{a4} and R^{b4} are independently selected from hydrogen and methyl, and G4 is a bond, —NHC(O)—, —NH—, —O—, or —S—. In some embodiments, L has a formula selected from:

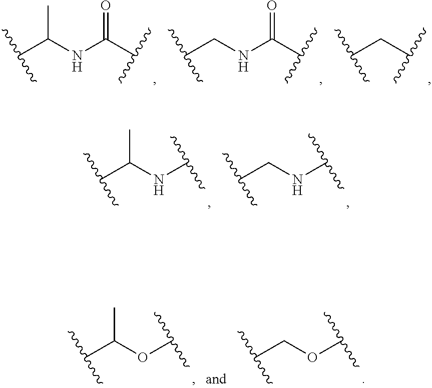

In some embodiments, B is a nine-membered bicyclic heteroaryl or a nine-membered bicyclic heterocyclyl having 1, 2, 3, or 4 heteroatoms independently selected from N, O, S, and P. In some embodiments, B is a nine-membered bicyclic heteroaryl or a nine-membered bicyclic heterocyclyl having 1, 2, 3, or 4 heteroatoms independently selected from N, O, and S. In some embodiments, B is a nine-membered bicyclic heteroaryl or a nine-membered bicyclic heterocyclyl having 1, 2, 3, or 4 nitrogen atoms. In some embodiments, B is a pyrazolopyrimidine. In some embodiments, in addition —R³, B is optionally substituted with 1, 2, 3, 4, or 5 substituents independently selected from halo, C₁-C₄ alkyl, C₁-C₄ haloalkyl, —OR^{a5}, —N(R^{b5})(R^{c5}), —SO?R^{d5}, —SO₂N(R^{e5})(R^{f5}), and —NHSO₂R^{g5}, wherein R^{a5}, R^{b5}, R^{c5}, R^{d5}, R^{e5}, R^{f5}, and R^{g5} are each independently selected from hydrogen, C₁-C₄ alkyl, and C₁-C₄ haloalkyl. In some embodiments, in addition —R³, B is substituted with one substituent selected from halo, C₁-C₄ alkyl, C₁-C₄ haloalkyl, —OR^{a5}, —N(R^{b5})(R^{c5}), —SO₂R^{d5}, —SO₂N(R^{e5})(R^{f5}), and —NHSO₂R^{g5}. In some embodiments, in addition to —R³, B is substituted with one substituent selected from halo, methyl, trifluoromethyl, —OR^{a5}, —N(R^{b5})(R^{c5}), —SO₂R^{d5}, —SO₂N(R^{e5})(R^{f5}), and —NHSO₂R^{g5}, wherein R^{a5}, R^{b5}, R^{c5}, R^{d5}, R^{e5}, R^{f5}, and R^{g5} are each independently selected from hydrogen, methyl, ethyl, isopropyl, t-butyl, and trifluoromethyl.

In some embodiments, the compound is a compound of formula (Ia):

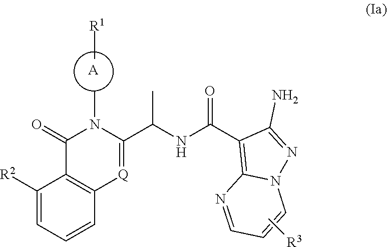

or a pharmaceutically acceptable salt thereof.

In some embodiments. A is phenyl or a monocyclic heteroaryl having 1, 2, 3, or 4 heteroatoms independently selected from N, O, S, and P. In some embodiments, A is phenyl or a monocyclic heteroaryl having 1, 2, 3, or 4 heteroatoms independently selected from N, O, and S. In some embodiments, A is phenyl or a monocyclic heteroaryl having one heteroatom selected from N, O, and S. In some embodiments, A is selected from phenyl, pyridyl, furan, and thiophene. In some embodiments, R¹ is hydrogen. In some embodiments, A is phenyl and R¹ is hydrogen.

In some embodiments, R² is selected from hydrogen, halo, C₁-C₄ alkyl, C₁-C₄ haloalkyl, C₁-C₄ alkoxy. C₁-C₄ haloalkoxy, and a group of formula (II). In some embodiments, R¹ is selected from hydrogen, fluoro, chloro, bromo, methyl, trifluoromethyl, methoxy, trifluoromethoxy, a group of formula (II). In some embodiments, R² is chloro. In some embodiments, R² is a group of formula (II).

In some embodiments, the compound is a compound of formula (Ib):

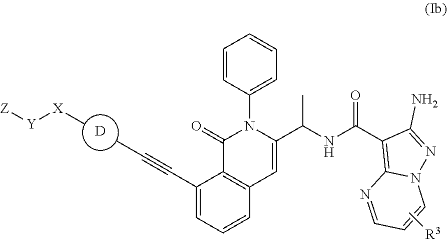

or a pharmaceutically acceptable salt thereof.

In some embodiments, D is a five-membered monocyclic heteroaryl or a 4- to 6-membered monocyclic heterocyclyl, each of which independently comprises 1, 2, 3, or 4 heteroatoms independently selected from N, O, S, and P. In some embodiments, D is a five-membered monocyclic heteroaryl or a 4- to 6-membered monocyclic heterocyclyl, each of which independently comprises 1, 2, 3, or 4 heteroatoms independently selected from N, O, and S. In some embodiments, D is selected from pyrrole, pyrazole, imidazole, imidazoline, oxazole, oxathiazole, oxadiazole, azetidine, pyrroline, pyrrolidine, and piperidine. In some embodiments, D has a structure selected from:

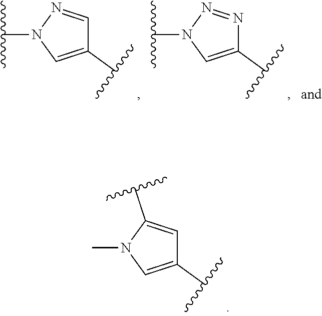

In some embodiments, X is a bond or —C(O)—. In some embodiments, X is a bond. In some embodiments, X is —C(O)—.

In some embodiments, Y is —(CR^{a2}₂)ₙ—CH₂—, wherein n is 0 or 1, and wherein each R^{a2} is hydrogen, or wherein the two R^{a2} groups, together with the carbon atom to which they are attached, form a C₃-C₆ cycloalkyl. In some embodiments, Y is —(CR^{a2}₂)ₙ—CH₂—, wherein n is 0 or 1, and wherein each R^{a2} is hydrogen, or wherein the two R^{a2} groups, together with the carbon atom to which they are attached, form a cyclopropylene ring. In some embodiments, Y is —(CR^{a2}₂)ₙ-G²-, wherein each R^{a2} is independently selected from H and C₁-C₄ alkyl, or wherein two R^{a2} together with the carbon atom(s) to which they are attached form a C₃-C₇ cycloalkylene ring: G² is a cycloalkylene or heterocyclylene, and n is 0, 1, 2, or 3. In some embodiments, G² is cyclobutylene, cyclopentylene, or cyclohexylene.

In some embodiments, the group —X—Y—Z has a formula selected from:

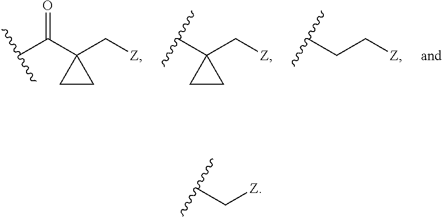

In some embodiments, Z is —OR^{b2}, —SR^{c2}, or —N(R^{d2})(R^{e2}), wherein R^{b2}, R^{c2}, R^{d2}, and R^{e2} are each independently selected from hydrogen, methyl, ethyl, isopropyl, t-butyl, phenyl, benzyl, —C(O)—C₁-C₄₀ alkyl, —C(O)—C₂-C₄₀ alkenyl, and a group of formula (IIa). In some embodiments, Z is —OR^{b2}, wherein R^{b2} is selected from hydrogen, —C(O)—C₁-C₄₀ alkyl, —C(O)—C₂-C₄₀ alkenyl, and a group of formula (IIa). In some embodiments, Z is —OR^{b2}, wherein R^{b2} is selected from hydrogen, —C(O)—C₁₅-C₂₀ alkyl, —C(O)—C₁₅-C₂₀ alkenyl, and a group of formula (IIa).

In some embodiments, Z is —OR^{b2}, wherein R^{b2} is selected from C(O)—C₁-C₄₀ alkyl, —C(O)—C₂-C₄₀ alkenyl, wherein the C₁-C₄₀ alkyl or the C₂-C₄₀ alkenyl group corresponds to the lipid tail of a saturated or unsaturated fatty acid, such as crotonic acid, myristoleic acid, palmitoleic acid, sapienic acid, oleic acid, elaidic acid, vaccenic acid, gadoleic acid, eicosenoic acid, erucic acid, nervonic acid, eicosadienoic acid, docosadienoic acid, linolenic acid, γ-linolenic acid, linolelaidic acid, pinolenic acid, eleostearic acid, mead acid, paullinic acid, gondoic acid, dihomo-γ-linolenic acid, eicosatrienoic acid, stearidonic acid, arachidonic acid, eicosatetraenoic acid, adrenic acid, bosseopentaenoic acid, eicosapentaenoic acid, ozubondo acid, docosahexaenoic acid, docosatetraenoic acid, tetracosanoic acid, pentanoic acid, butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosanoic acid, lignoceric acid, pentacosylic acid, cerotic acid, carboceric acid, montanic acid, nonacosylic acid, melissic acid, hentriacontylic acid, lacccroic acid, psyllic acid, geddic acid, ceroplastic acid, bexatriacontylic acid, heptatriacontylic acid, octatriacontylic acid, nonatriacontylic acid, and tetracontylic acid.

In some embodiments, Z is —OR^{b2}, wherein R^{b2} is a group of formula (IIa).

In some embodiments, the group —X—Y—Z has a formula selected from

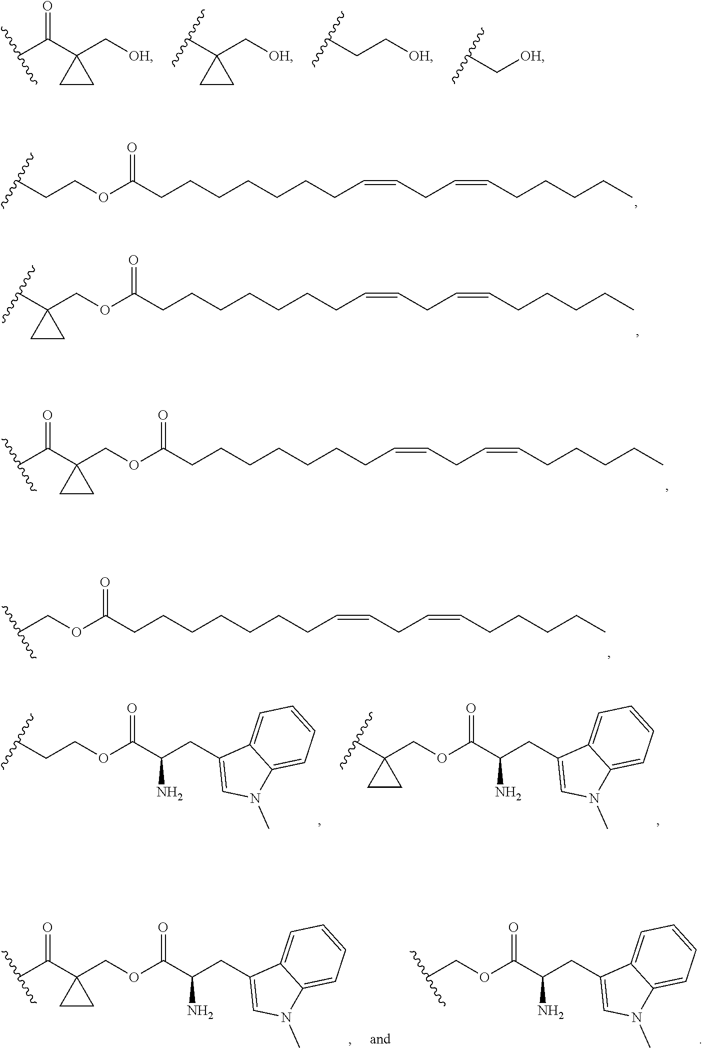

In some embodiments, compound is a compound of formula (Ic):

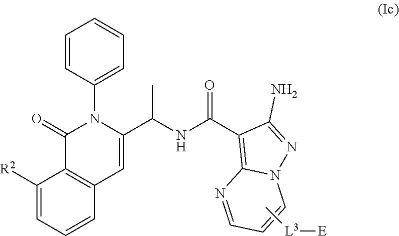

or a pharmaceutically acceptable salt thereof.

In some embodiments, R² is selected from hydrogen, halo, C₁-C₄ alkyl, C₁-C₄ haloalkyl, C₁-C₄ alkoxy, C₁-C₄ haloalkoxy, and a group of formula (II). In some embodiments, R² is selected from hydrogen, fluoro, chloro, bromo, methyl, trifluoromethyl, methoxy, trifluoromethoxy, a group of formula (II). In some embodiments, R² is chloro. In some embodiments, R² is a group of formula (II).

In some embodiments, L³ is a bond, —CH₂—CH₂—, —CH═CH—, —C≡C—, —C(O)NH—, or a 5-membered monocyclic heteroarylene having 1, 2, 3, or 4 heteroatoms independently selected from N, O, S, and P. In some embodiments, L′ is a bond, —CH₂—CH₂—, —CH═CH—, —C≡C—, —C(O)NH—, or a 5-membered monocyclic heteroarylene having 1, 2, or 3 nitrogen atoms. In some embodiments, L³ is a bond. In some embodiments, L³ is —CH₂—CH₂—. In some embodiments, L³ is —CH═CH—. In some embodiments, L³ is —C≡C—. In some embodiments, L³ is —C(O)NH—. In some embodiments, L³ is 5-membered monocyclic heteroarylene having 1, 2, or 3 nitrogen atoms. In some embodiments, L³ has formula:

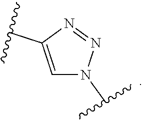 [SMILES: *c1cn(*)nn1]

In some embodiments, E is a 8-10 membered bicyclic heterocyclyl or bicyclic heteroaryl, each of which is optionally substituted with 1, 2, 3, 4, or 5 substituents independently selected from halo, C₁-C₄ alkyl, C₃-C₆ cycloalkyl, C₃-C₆-cycloalkyl-C₁-C₄-alkyl, C₁-C₄ haloalkyl, oxo, —OR^{a3}, —N(R^{b3})(R^{c3}), —SO₂R^{d3}, —SO₂N(R^{e3})(R^{f3}), and —NHSO₂R^{g3}, wherein R^{a3}, R^{b3}, R^{c3}, R^{d3}, R^{e3}, R^{f3}, and R^{g3} are each independently selected from hydrogen, C₁-C₄ alkyl, and C₁-C₄ haloalkyl. In some embodiments, E is a bicyclic heterocyclyl or bicyclic heteroaryl, in which one ring of the bicyclic ring is a phenyl, pyridyl, pyridazinyl, pyrimidinyl, or pyrazinyl, and the other ring of the bicyclic ring is a pyrrolidinone, a pyrrolidine-dione, a piperidine-dione, a pyrrole, a pyrazole, an imidazole, an imidazoline, an oxazole, an oxathiazole, an oxadiazole, a pyrroline, a pyrrolidine, a piperidine, or an azetidine, any of which is optionally substituted as described above. In some embodiments, E is isoindolinone.

In some embodiments, E has a formula:

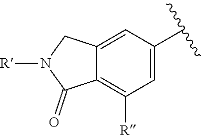

wherein R′ and R″ are independently selected from C₁-C₄ alkyl, C₃-C₆-cycloalkyl-C₁-C₄-alkyl, C₁-C₄ haloalkyl, and —NHSO₂R^{g3}, wherein Re is C₁-C₄ alkyl. In some embodiments, R′ is C₃-C₆-cycloalkyl-C₁-C₄-alkyl, and R″ is selected from C₁-C₄ alkyl, C₁-C₄ haloalkyl, and —NHSO₂R^{g3}, wherein R^{g3} is C₁-C₄ alkyl. In some embodiments, E has a formula selected from:

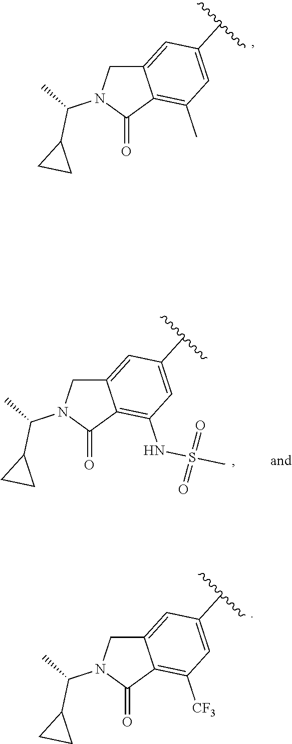 [SMILES: *c1cc(C)c2c(c1)CN([C@@H](C)C1CC1)C2=O ; *c1cc2c(c(NS(C)(=O)=O)c1)C(=O)N([C@@H](C)C1CC1)C2 ; *c1cc2c(c(C(F)(F)F)c1)C(=O)N([C@@H](C)C1CC1)C2]

In some embodiments, the compound is selected from:

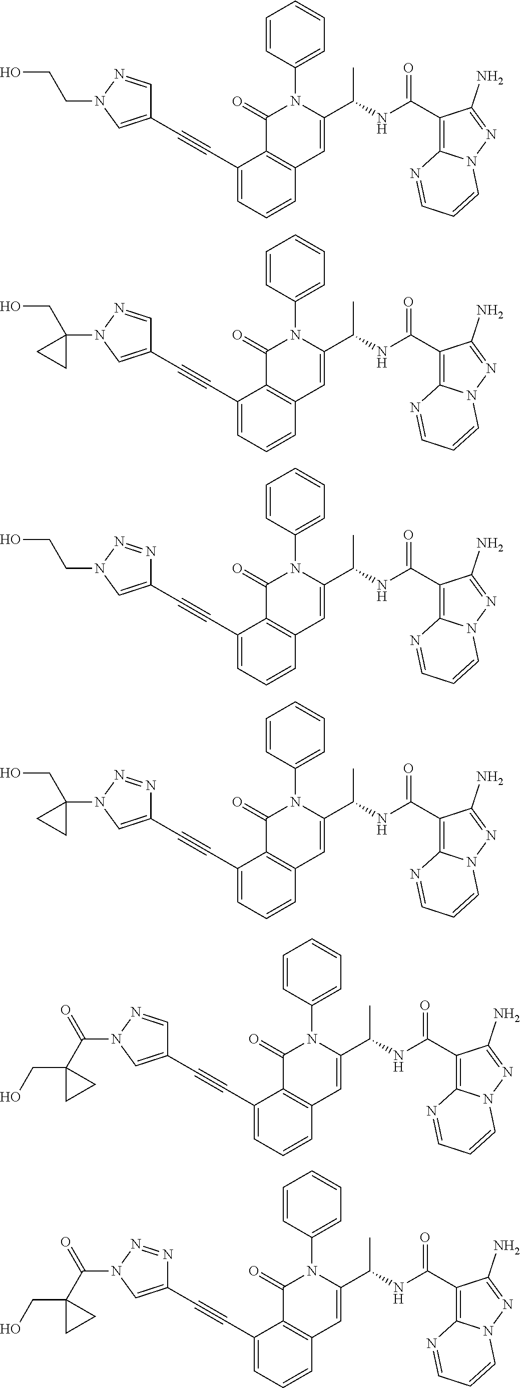

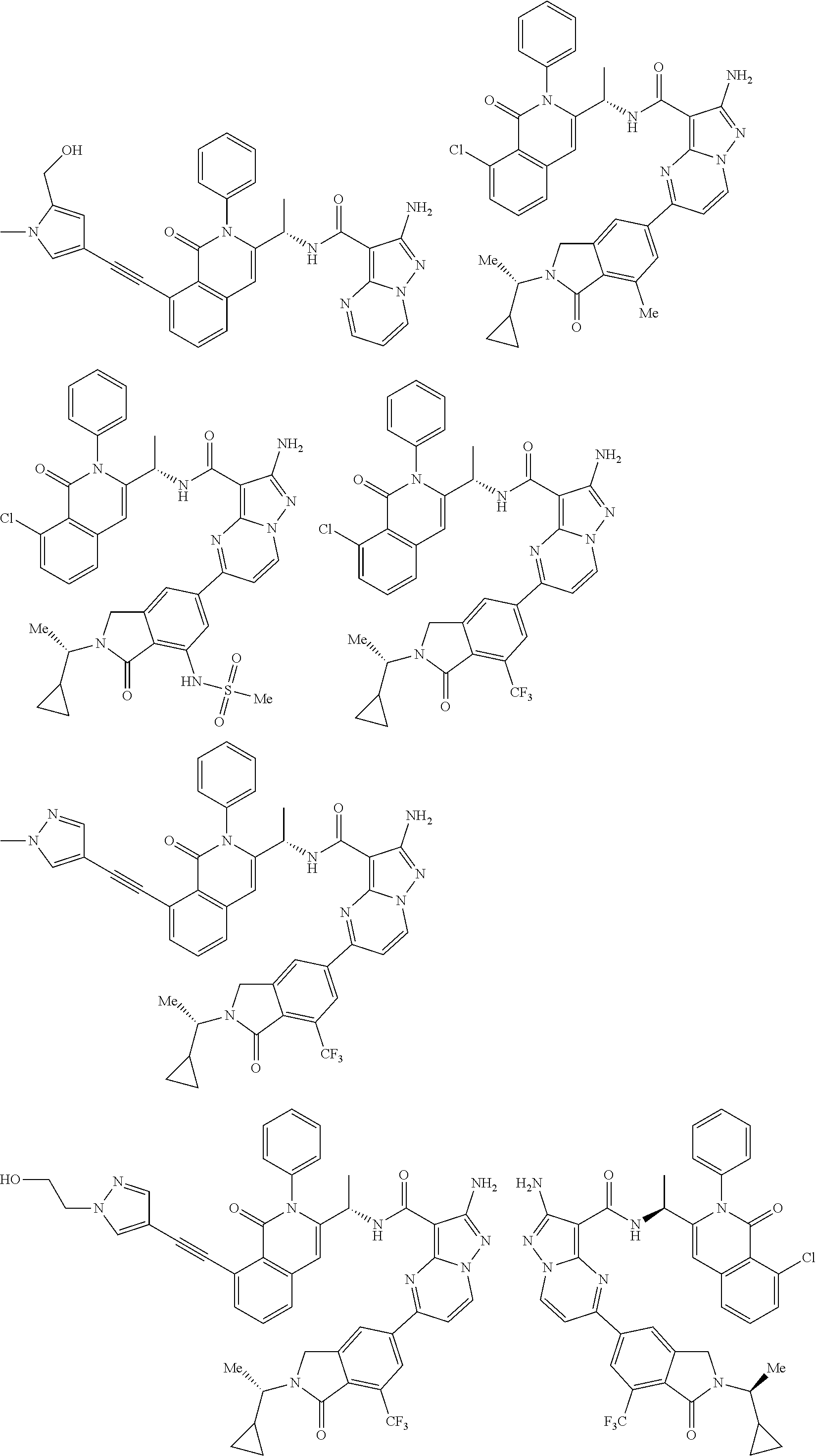

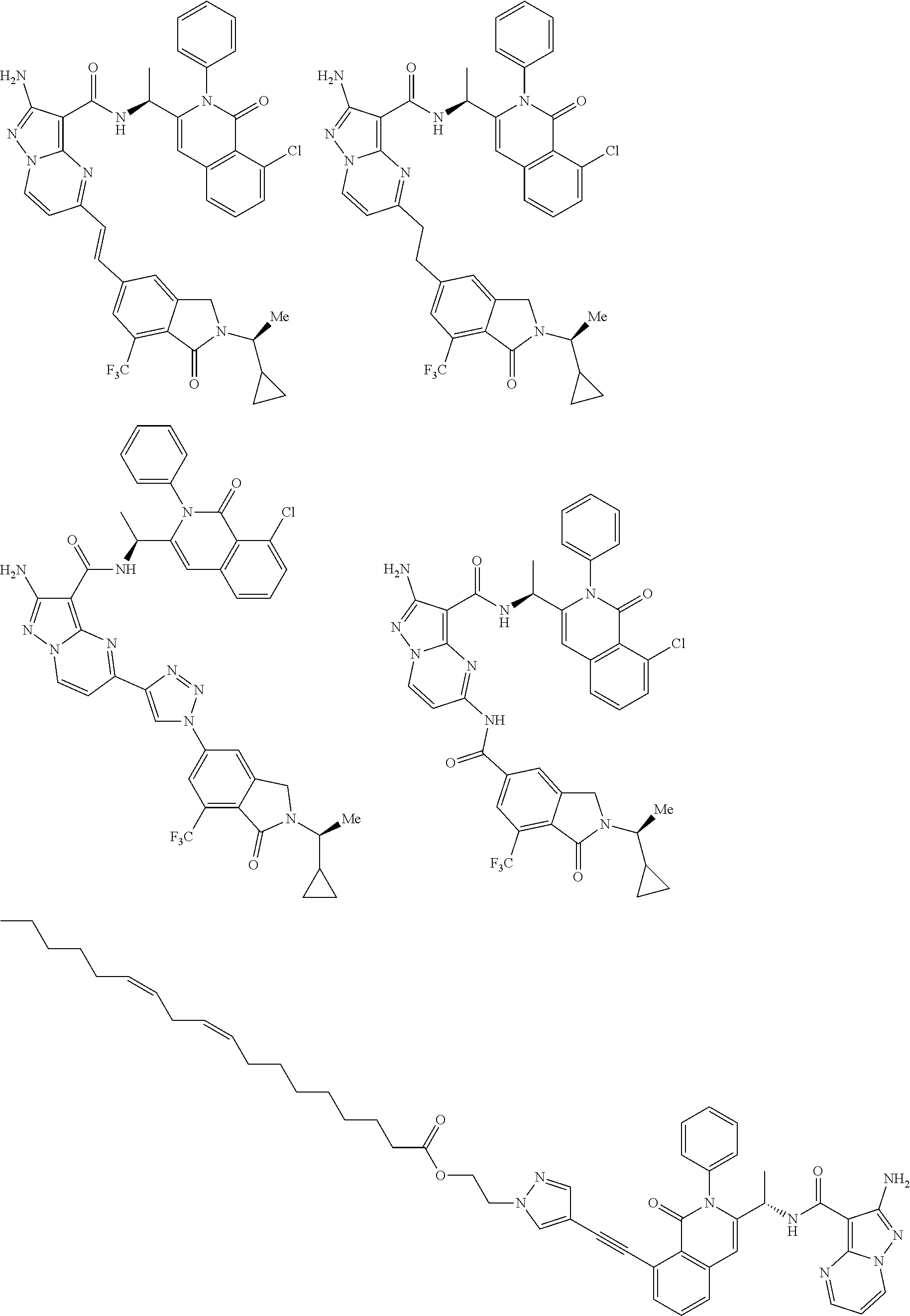

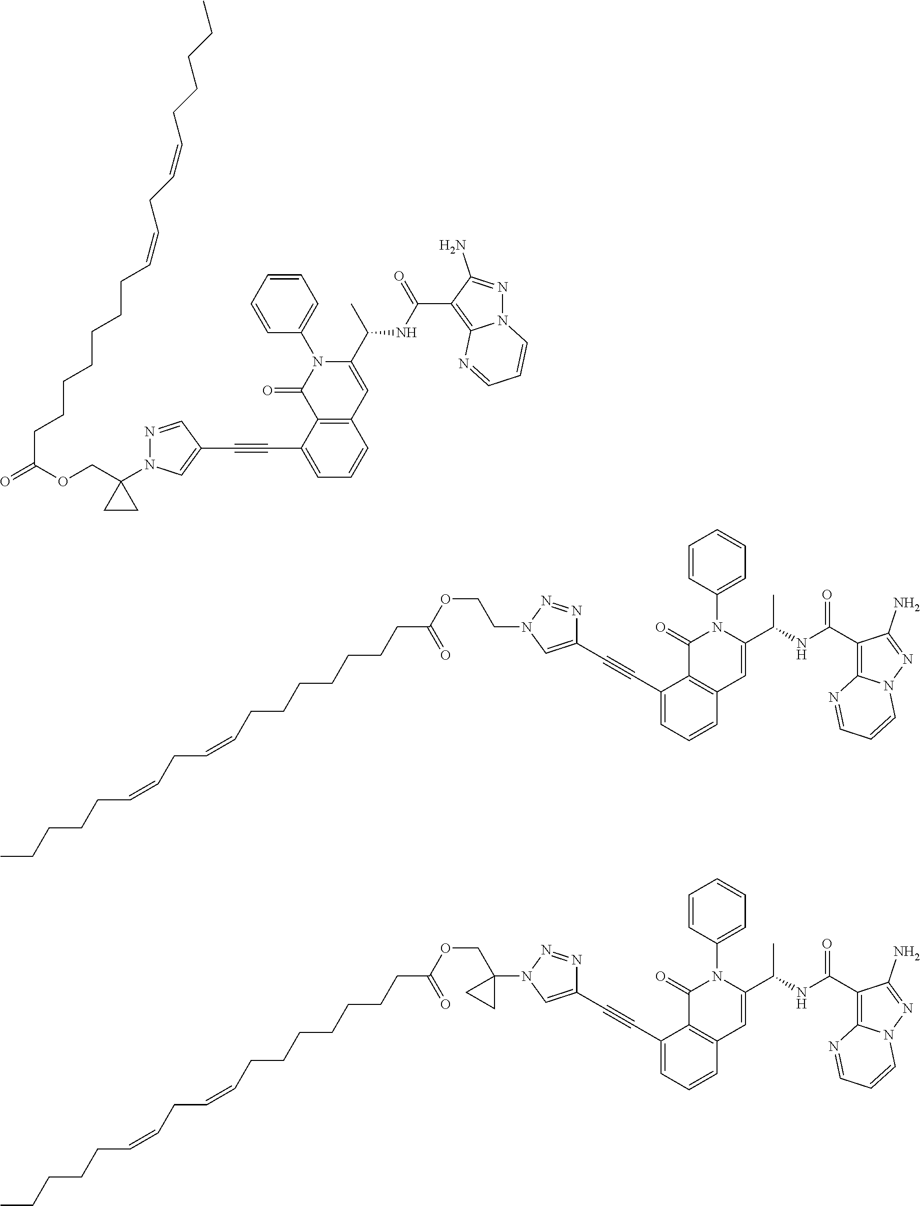 [SMILES: CCCCC/C=C\C/C=C\CCCCCCCC(=O)OCC1(n2cc(C#Cc3cccc4cc([C@H](C)NC(=O)c5c(N)nn6cccnc56)n(-c5ccccc5)c(=O)c34)cn2)CC1 ; CCCCC/C=C\C/C=C\CCCCCCCC(=O)OCCn1cc(C#Cc2cccc3cc([C@H](C)NC(=O)c4c(N)nn5cccnc45)n(-c4ccccc4)c(=O)c23)nn1 ; CCCCC/C=C\C/C=C\CCCCCCCC(=O)OCC1(n2cc(C#Cc3cccc4cc([C@H](C)NC(=O)c5c(N)nn6cccnc56)n(-c5ccccc5)c(=O)c34)nn2)CC1]

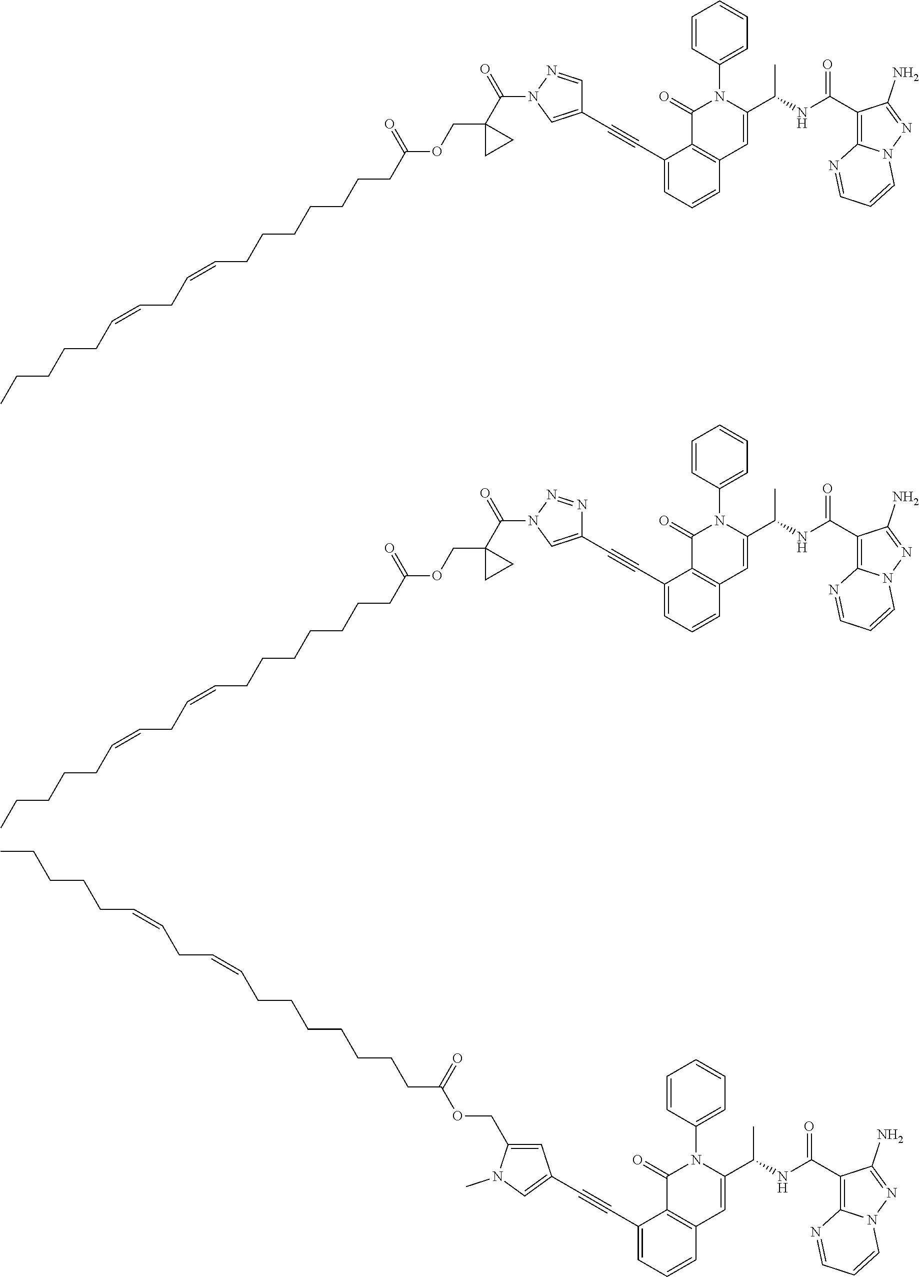

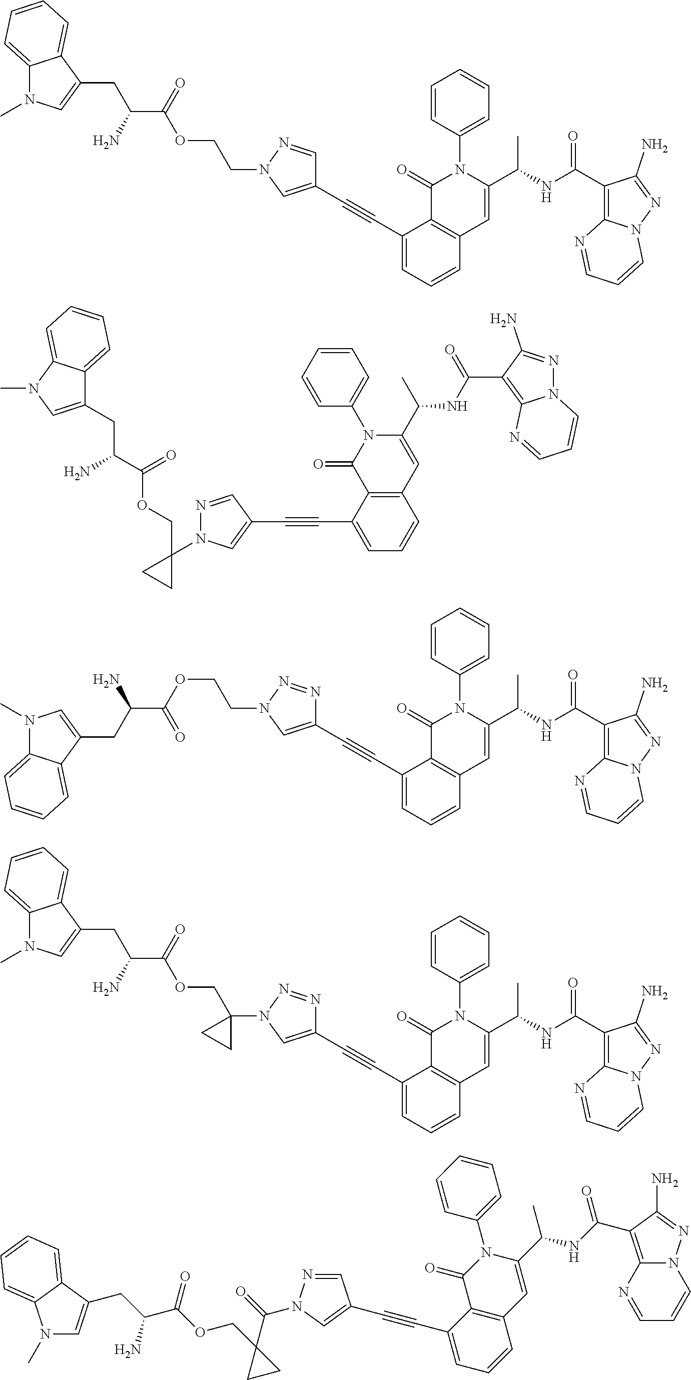

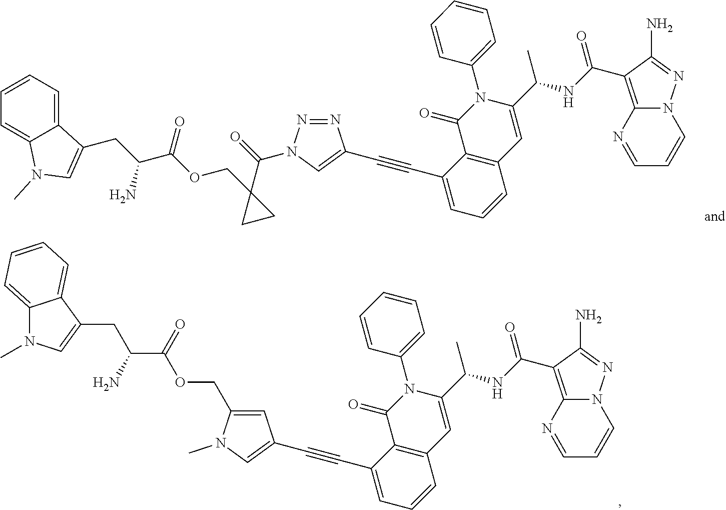 [SMILES: C[C@H](NC(=O)c1c(N)nn2cccnc12)c1cc2cccc(C#Cc3cn(C(=O)C4(COC(=O)[C@H](N)Cc5cn(C)c6ccccc56)CC4)nn3)c2c(=O)n1-c1ccccc1 ; C[C@H](NC(=O)c1c(N)nn2cccnc12)c1cc2cccc(C#Cc3cc(COC(=O)[C@H](N)Cc4cn(C)c5ccccc45)n(C)c3)c2c(=O)n1-c1ccccc1]

and pharmaceutically acceptable salts thereof.

Additional PI3K inhibitor compounds, which may be used in pharmaceutical compositions such as those comprising an albumin nanoparticle, include compounds of formula (III):

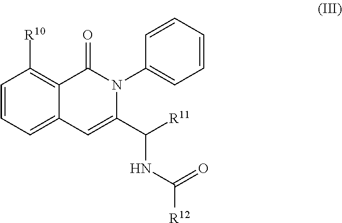

or a pharmaceutically acceptable salt thereof, wherein:
R¹⁰ is selected from —C≡C—R^{x}, C₁-C₄ alkyl, C₂-C₄ alkenyl, C₂-C₄ alkynyl, halo, cyano, C₁-C₄ alkoxy, C₁-C₄ alkylamino, and di-C₁-C₄-alkylamino;
R¹¹ is selected from C₁-C₄ alkyl and hydrogen;
R¹² is an 8- to 10-membered bicyclic heteroaryl having 1, 2, or 3 nitrogen atoms, wherein the heteroaryl is optionally substituted with 1 or 2 substituents selected from amino, C₁-C₄ alkyl, and halo; and
R^{x} is selected from a 5- or 6-membered monocyclic heteroaryl having 1 or 2 heteroatoms independently selected from N and S, aryl, hydrogen, and C₁-C₄ alkyl, wherein the heteroaryl and aryl are optionally substituted with 1 or 2 substituents selected from C₁-C₄ alkyl.

In some embodiments, R¹⁰ is selected from methyl, fluoro, chloro, cyano, methoxy, methylamino, dimethylamino, and —C≡C—R^{x}, wherein R^{x} is selected from pyrazolyl, thiazolyl, pyridyl, and phenyl, each of which is optionally substituted with one methyl group. In some embodiments, R¹⁰ is —C≡C—R^{x}, and R^{x} is a 5-membered monocyclic heteroaryl having two nitrogen atoms, which is substituted with one C₁-C₄ alkyl. In some embodiments, R¹⁰ is —C≡C—R^{x}, and R^{x} is pyrazolyl substituted with a methyl group.

In some embodiments, R¹¹ is C₁-C₄ alkyl. In some embodiments, R¹¹ is methyl.

In some embodiments, R¹² is a 9- or 10-membered bicyclic heteroaryl having 2 or 3 nitrogen atoms (e.g., pyrazolo[1,5-a]pyrimidinyl, pyrazolo[1,5-a]pyridinyl, quinolinyl, or naphthyridinyl), wherein the heteroaryl is optionally substituted with one amino group. In some embodiments, R¹² is a pyrazolo[1,5-a]pyrimidine substituted with one amino group.

In some embodiments, the compound of formula (III) is selected from:

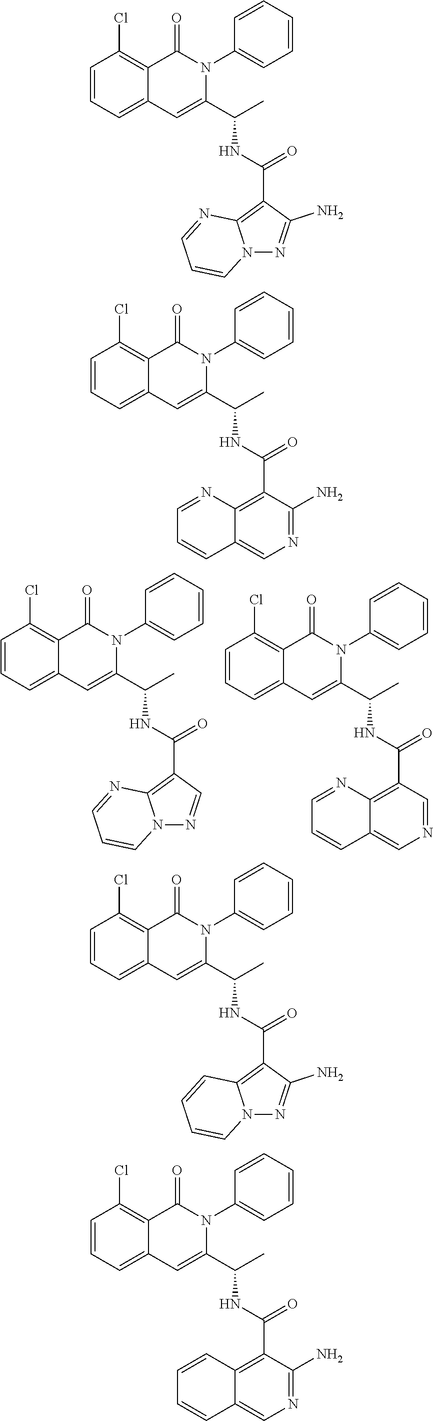

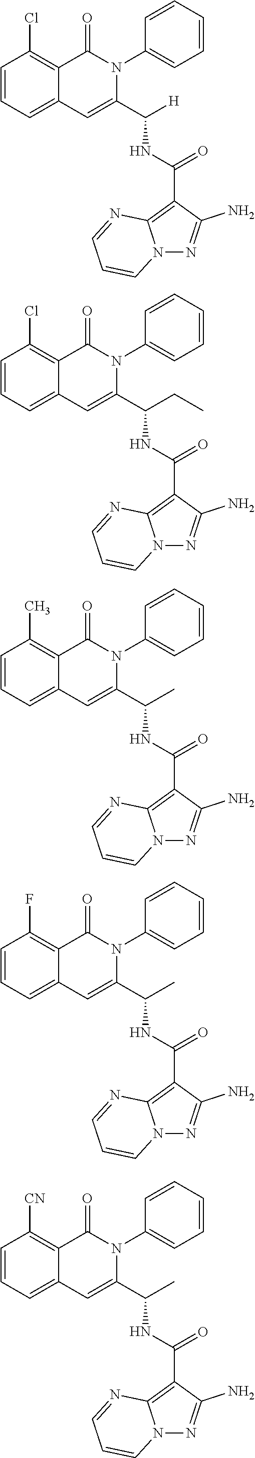

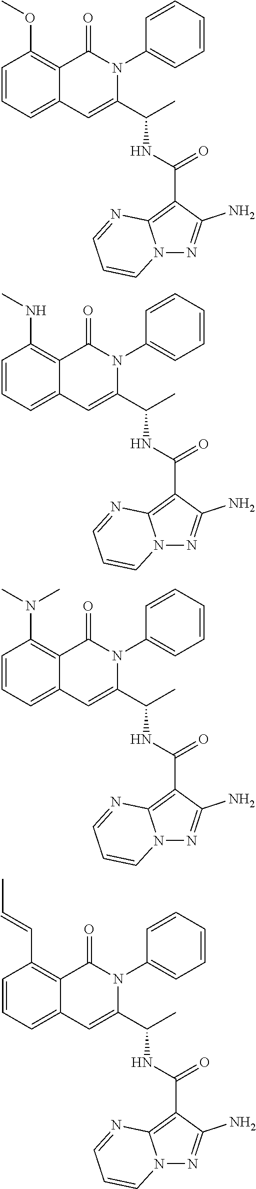

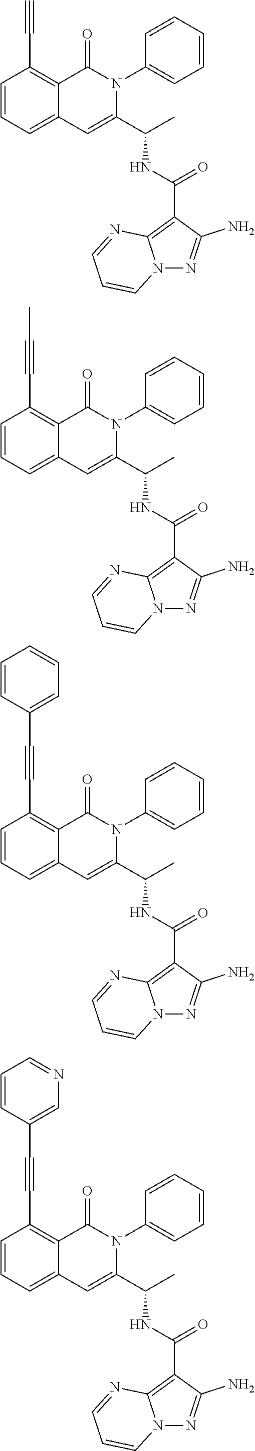 [SMILES: C#Cc1cccc2cc([C@H](C)NC(=O)c3c(N)nn4cccnc34)n(-c3ccccc3)c(=O)c12 ; CC#Cc1cccc2cc([C@H](C)NC(=O)c3c(N)nn4cccnc34)n(-c3ccccc3)c(=O)c12 ; C[C@H](NC(=O)c1c(N)nn2cccnc12)c1cc2cccc(C#Cc3ccccc3)c2c(=O)n1-c1ccccc1 ; C[C@H](NC(=O)c1c(N)nn2cccnc12)c1cc2cccc(C#Cc3cccnc3)c2c(=O)n1-c1ccccc1]

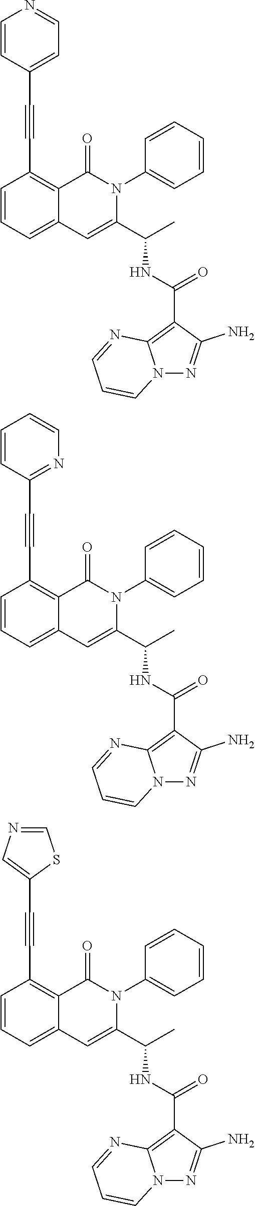 [SMILES: C[C@H](NC(=O)c1c(N)nn2cccnc12)c1cc2cccc(C#Cc3ccncc3)c2c(=O)n1-c1ccccc1 ; C[C@H](NC(=O)c1c(N)nn2cccnc12)c1cc2cccc(C#Cc3ccccn3)c2c(=O)n1-c1ccccc1 ; C[C@H](NC(=O)c1c(N)nn2cccnc12)c1cc2cccc(C#Cc3cncs3)c2c(=O)n1-c1ccccc1]

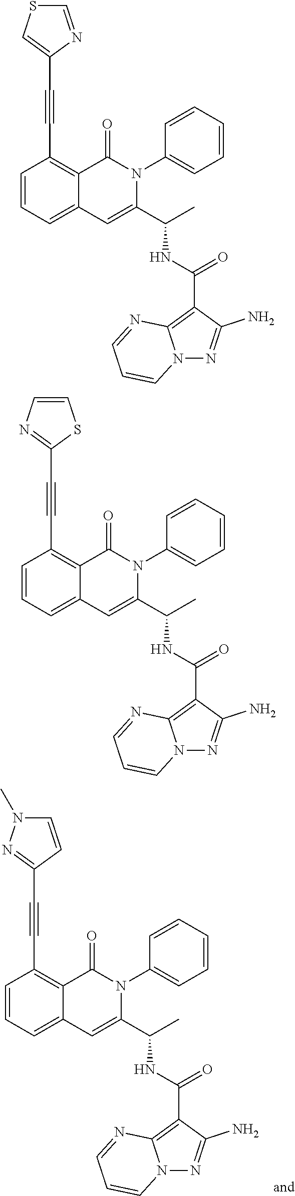 [SMILES: C[C@H](NC(=O)c1c(N)nn2cccnc12)c1cc2cccc(C#Cc3cscn3)c2c(=O)n1-c1ccccc1 ; C[C@H](NC(=O)c1c(N)nn2cccnc12)c1cc2cccc(C#Cc3nccs3)c2c(=O)n1-c1ccccc1 ; C[C@H](NC(=O)c1c(N)nn2cccnc12)c1cc2cccc(C#Cc3ccn(C)n3)c2c(=O)n1-c1ccccc1]

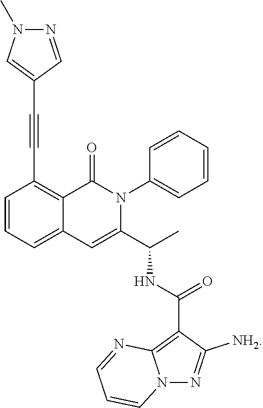 [SMILES: C[C@H](NC(=O)c1c(N)nn2cccnc12)c1cc2cccc(C#Cc3cnn(C)c3)c2c(=O)n1-c1ccccc1]

In some embodiments, the compound of formula (III) is:

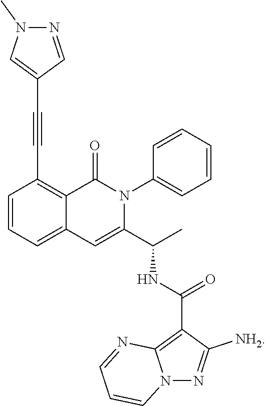 [SMILES: C[C@H](NC(=O)c1c(N)nn2cccnc12)c1cc2cccc(C#Cc3cnn(C)c3)c2c(=O)n1-c1ccccc1]

The compounds may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E. Fundamental Stereochemistry, in Pure Appl. Chem., 1976, 45: 13-30. The disclosure contemplates various stereoisomers and mixtures thereof and these are specifically included within the scope of this disclosure. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry,” 5th edition (1989), Longman Scientific & Technical, Essex CM20 2JE. England (or more recent versions thereof), or (2) direct separation of the mixture of optical enantiomers on chiral chromatographic columns, or (3) fractional recrystallization methods.

It should be understood that the compounds may possess tautomeric forms, as well as geometric isomers, and that these also constitute embodiments of the disclosure.

The present disclosure also includes isotopically-labeled compounds, which is identical to those recited in formula (I) or formula (III), but for the fact that one or more atoms are replaced by an atom having an atomic mass or mass number different from the atomic mass or mass number usually found in nature. Examples of isotopes include those for hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³Cl, respectively. Substitution with heavier isotopes such as deuterium, for example, ²H, can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated into the compounds are ¹¹C, ¹³N, ¹⁵O, and ¹⁸F Isotopically-labeled compounds can generally be prepared by conventional techniques known to those skilled in the art or by processes analogous to those described in the accompanying Examples using appropriate isotopically-labeled reagent in place of non-isotopically-labeled reagent.

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides, and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine. N,N-dibenzylphenethylamine, 1-ephenamine and N,N′-dibenzylethylenediamine, ethylenediamine, ethanolamine, diethanolamine, piperidine, piperazine, and the like.

Compounds may be synthesized according to a variety of methods, including those illustrated in the Examples. Reaction conditions and reaction times for each individual step can vary depending on the particular reactants employed and substituents present in the reactants used. Specific procedures are provided in the Examples section. Reactions can be worked up in the conventional manner, e.g., by eliminating the solvent from the residue and further purified according to methodologies generally known in the art such as, but not limited to, crystallization, distillation, extraction, trituration, and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the disclosure. Suitable protecting groups and the methods for protecting and deprotecting different substituents using such suitable protecting groups are well known to those skilled in the art; examples of which can be found in P G M Wuts and T W Greene, in Greene's book titled Protective Groups in Organic Synthesis (4th ed.), John Wiley & Sons, NY (2006), which is incorporated herein by reference in its entirety. Synthesis of the compounds of the disclosure can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization, or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the disclosure as it is defined in the appended claims. All alternatives, modifications, and equivalents of the synthetic methods and specific examples are included within the scope of the claims.

### 3. Compositions

The disclosed compounds, along with other PI3K inhibitor compounds, may be incorporated into compositions that may be suitable for administration to a subject (such as a patient, which may be a human or non-human).

a. Pharmaceutical Compositions

The compounds may be incorporated into pharmaceutically acceptable compositions. The pharmaceutical compositions may include a “therapeutically effective amount” or a “prophylactically effective amount” of the compound(s). A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the disclosure (e.g., a compound of formula (I)) are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The pharmaceutical compositions and formulations may include pharmaceutically acceptable carriers. The term “pharmaceutically acceptable carrier,” as used herein, means a non-toxic, inert solid, semi-solid or liquid filler, diluent, encapsulating material, surfactant, cyclodextrins or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; tale; excipients such as, but not limited to, cocoa butter and suppository waxes; oils such as, but not limited to, peanut oil, cottonseed oil, safflower oil, sesame oil, olive oil, corn oil and soybean oil; surfactants such as, but not limited to, cremophor EL, cremophor RH 60, Solutol HS 15 and polysorbate 80; cyclodextrins such as, but not limited to, alpha-CD, beta-CD, gamma-CD, HP-beta-CD, SBE-beta-CD; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The route by which the disclosed compounds are administered and the form of the composition will dictate the type of carrier to be used. The composition may be in a variety of forms, suitable, for example, for systemic administration (e.g., oral, rectal, nasal, sublingual, buccal, implants, or parenteral injections) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, cyclodextrins combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, dimethyl sulfoxide, N-Methyl-2-Pyrrolidone, dimethylacetamide and phosphate (or other suitable buffer). The amount of solvent(s) in a systemic or topical composition is typically from about 0 to about 100%.

Suitable suspending agents include AVICEL RC-591 (from FMC Corporation of Philadelphia, Pa.) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Del. Suitable surfactants include those disclosed in the C.T.F.A. Cosmetic Ingredient Handbook, 1992, pp, 587-592; Remington's Pharmaceutical Sciences, 15th Ed, 1975, pp, 335-337; and McCutcheon's Volume 1, Emulsifiers & Detergents, 1994, North American Edition, pp, 236-239. The amount of surfactant(s) in the systemic or topical composition is typically about 0.1% to about 5%.

Suitable cyclodextrins include alpha-CD, beta-CD, gamma-CD, hydroxypropyl betadex (HP-beta-CD), sulfobutyl-ether β-cyclodextrin (SBE-beta-CD). The amount of cyclodextrins in the systemic or topical composition is typically about 0% to about 40%.

Although the amounts of components in the systemic compositions may vary depending on the type of systemic composition prepared, in general, systemic compositions include 0.01% to 50% of an active compound (e.g., a compound of formula (I) or formula (III)) and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose, and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound (e.g., a compound of formula (I) or formula (III)), and a carrier including one or more diluents disclosed above in a capsule comprising gelatin. Granules typically comprise a disclosed compound, and preferably glidants such as silicon dioxide to improve flow characteristics Implants can be of the biodegradable or the non-biodegradable type.

The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose. EUDRAGIT® coatings (available from Evonik Industries of Essen, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms Such compositions typically include one or more of soluble filler substances such as diluents including sucrose, sorbitol, and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include; a disclosed compound (e.g., a compound of formula (I) or formula (III)), and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the compound. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.5%, particularly, about 0.001% to about 0.1%.

Suitable pH adjusting additives include HCl or NaOH in amounts sufficient to adjust the pH of a topical pharmaceutical composition.

b. Albumin Nanoparticle Compositions

The disclosure further provides compositions comprising an effective amount of a phosphatidylinositol 3-kinase (PI3K) inhibitor, or a pharmaceutically acceptable salt thereof, and albumin nanoparticles. A variety of PI3K inhibitors can be used in conjunction with the albumin nanoparticles to form suitable compositions. In some embodiments, the PI3K inhibitor is a compound of formula (I), such as a compound of formula (I) disclosed herein. In some embodiments, the PI3K inhibitor is a compound of formula (III), such as a compound of formula (III) disclosed herein. In some embodiments, the PI3K inhibitor is selected from IPI-549, idelalisib, copanlisib, duvelisib, alpelisib, leniolisib, umbralisib, buparlisib, taselisib, pictilisib, PX-886, pilaralisib. BEZ235, GSK2126458, GSK2636771, AZD8186, SAR260301, gedatolisib, apitolisib, PQR309, MLN1117, and perifosine. The albumin nanoparticles compositions and formulations may also include pharmaceutically acceptable carriers, as described above

In some embodiments, the albumin nanoparticle encapsulates, e.g., forms a shell surrounding, the PI3K inhibitor. In certain embodiments, the nanoparticle composition achieves a high encapsulate efficiency (>70-90%) and good stability.

Albumins include the most abundant plasma proteins in mammals and albumins from a large and diverse number of mammals have been characterized by biochemical methods and/or by sequence information. Any natural, synthetic, or engineered albumin may be used in the context of the nanoparticle compositions described herein. In some embodiments, the albumin is human serum albumin or albumin from animal species (e.g., bovine serum albumin, porcine serum albumin, or the like). In some embodiments, the albumin is human serum albumin.

The compositions may contain albumin and the PI3K inhibitor in different molar ratios. The molar ratio of albumin to disclosed compounds ranges from 1:20 to 20:1

In some embodiments, the diameter of each albumin nanoparticle is in the range of 50 to 200 nm. The diameter of the nanoparticle may be about 50 nm, about 75 nm, about 100 nm, about 125 nm, about 150 nm, about 175 nm, or about 200 nm.

c. Additional Nanoformulations

In some embodiments, the PI3K inhibitors are incorporated into compositions comprising poly(lactic acid) (PLA) and/or poly(lactic-co-glycolic acid) (PLGA) nanoparticles, a liposome, lipid nanoparticle, or a micelle. In some embodiments, the PI3K inhibitors are encapsulated in the PLA or PLGA nanoparticle, the liposome, the lipid nanoparticle, or the micelle. The nanoformulations may also include pharmaceutically acceptable carriers, as described above.

In some embodiments, the disclosed compounds are incorporated into liposomal compositions comprising one or more vesicle forming lipids. Methods of making liposomal compositions include, for example, lipid film hydration, optionally coupled with sonication or extrusion, solvent evaporation (e.g., ethanol injection, ether injection, or reverse phase evaporation) or detergent removal methods. The disclosed compounds can be combined with the lipid(s) before formation of the vesicles (passive loading) or after vesicle formation (active loading). The liposome compositions may prolong circulation time in vivo, increase stability of the compound, and prevent degradation in the bloodstream. The liposomal composition may increase the distribution of the compounds within the lung, breast, pancreas, and spleen.

Any naturally occurring or synthetic vesicle forming lipid or combinations thereof can be used. The one or more vesicle forming lipids may be selected from di-aliphatic chain lipids, such as phospholipids; diglycerides; di-aliphatic glycolipids; single lipids such as sphingomyelin or glycosphingolipid; steroidal lipids; hydrophilic polymer derivatized lipids; or mixtures thereof.

The liposomes may contain other non-vesicle forming lipids or other moieties, including but not limited to amphiphilic polymers, polyanions, sterols, and surfactants. The liposomes contained in the liposome composition can also be targeting liposomes, e.g., liposomes containing one or more targeting moieties or biodistribution modifiers on the surface of the liposomes. A targeting moiety can be any agent that is capable of specifically binding or interacting with a desired target and are generally known in the art, for example ligands such as folic acid, proteins, antibody or antibody fragments, and the like).

The liposomes can have any liposome structure, e.g., structures having an inner space sequestered from the outer medium by one or more lipid bilayers, or any microcapsule that has a semi-permeable membrane with a lipophilic central part where the membrane sequesters an interior. In some embodiments, the liposome may be a unilamellar liposome, having a single lipid layer. The disclosed compounds may be completely or partially located in the interior space of the liposome or completely or partially within the bilayer membrane of the liposome. In some embodiments, the lipids for a micelle.

In some embodiments, the disclosed compounds are incorporated into nanoformulations comprising PLA and/or PLGA. PLA or PLGA nanoformulations may be prepared by various methods known in the art such as single/double emulsion-solvent evaporation technique, spray drying, spray freeze drying, supercritical fluid drying, and nanoprecipitation.

d. Additional Therapeutic Agents

The compositions disclosed herein may further comprise at least one additional therapeutic agent. In some embodiments, the at least one additional therapeutic agent comprises at least one chemotherapeutic agent. As used herein, the term “chemotherapeutic” or “anti-cancer drug” includes any small molecule or other drug used in cancer treatment or prevention. Chemotherapeutics include, but are not limited to, cyclophosphamide, methotrexate, 5-fluorouracil, doxorubicin, docetaxel, daunorubicin, bleomycin, vinblastine, dacarbazine, cisplatin, paclitaxel, raloxifene hydrochloride, tamoxifen citrate, abemacicilib, afinitor (Everolimus), alpelisib, anastrozole, pamidronate, anastrozole, exemestane, capecitabine, epirubicin hydrochloride, cribulin mesylate, toremifene, fulvestrant, letrozole, gemcitabine, goserelin, ixabepilone, emtansine, lapatinib, olaparib, megestrol, neratinib, palbociclib, ribociclib, talazoparib, thiotepa, toremifene, methotrexate, and tucatinib. In select embodiments, the chemotherapeutic agent is paclitaxel.

In some embodiments, the chemotherapeutic is added to the pharmaceutical composition comprising the compounds disclosed herein. In some embodiments, the compositions of the chemotherapeutic agent are incorporated into the compositions comprising a PI3K inhibitor and an albumin nanoparticle. In some embodiments, the albumin nanoparticle encapsulates, e.g., forms a shell surrounding, both the chemotherapeutic agent and the PI3K inhibitor. In some embodiments, the chemotherapeutic agent is incorporated into the compositions comprising a PI3K inhibitor and a PLGA and/or PLA nanoparticle, a liposome, a lipid nanoparticle, or a micelle. In some embodiments, the chemotherapeutic agent is encapsulated in the PLGA and/or PLA nanoparticle, liposome, lipid nanoparticle, or micelle.

### 4. Methods of Use

The disclosure further provides methods for treating a disease or disordering comprising administration of a PI3K inhibitor, or a composition thereof, to a subject in need thereof. In some embodiments, the subject is a human.

The PI3K inhibitors may target any class of PI3K, including Class I (e.g., IA and IB), Class II, or Class III. In some embodiments, the PI3K inhibitors is a compound as disclosed herein. In some embodiments, the PI3K inhibitors comprises isoform-selective PI3K inhibitors, dual pan-Class I PI3K/m-TOR inhibitors, and pan-Class I PI3K inhibitors without significant m-TOR activity PI3K inhibitors useful in the present compositions, nanoformulations and methods include, but are not limited to, IPI-549, idelalisib, copanlisib, duvelisib, alpelisib, leniolisib, umbralisib, buparlisib, taselisib, pictilisib, PX-886, pilaralisib, BEZ235, GSK2126458, GSK2636771, AZD8186, SAR260301, gedatolisib, apitolisib, PQR309, MLN1117, and perifosine

The disease or disorder may comprise cancer, autoimmune, and inflammatory diseases.

In some embodiments, the disease or disorder is an inflammatory disease or disorder. Inflammatory diseases are characterized by activation of the immune system in a tissue or an organ to abnormal levels that may lead to abnormal function and/or disease in the tissue or organ. The inflammatory diseases and disorders that may be treated by the methods of the present invention include, but are not limited to, arthritis, rheumatoid arthritis, asthma, inflammatory bowel disease (Crohn's disease or ulcerative colitis), chronic obstructive pulmonary disease (COPD), allergic rhinitis, vasculitis (polyarteritis nodosa, temporal arteritis, Wegener's granulomatosis, Takayasu's arteritis, or Behcet's syndrome), inflammatory neuropathy, psoriasis, systemic lupus erythematosus (SLE), chronic thyroiditis. Hashimoto's thyroiditis, Addison's disease, polymyalgia rheumatica. Sjogren's syndrome, or Churg-Strauss syndrome. In some important embodiments, the inflammatory disease is rheumatoid arthritis.

In some embodiments, the disease or disorder is an autoimmune disease or disorder. Autoimmune diseases and disorders refer to conditions in a subject characterized by cellular, tissue and/or organ injury caused by an immunologic reaction of the subject to its own cells, tissues and/or organs. Autoimmune diseases and disorders that may be treated by the methods of the present invention include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy. Churg-Strauss syndrome, cicatricial pemphigoid, CREST syndrome, cold agglutinin disease, Crohn's disease, discoid lupus, essential mixed cryoglobulinemia, fibromyalgia-fibromyositis, glomerulonephritis. Graves' disease. Guillain-Barre, Hashimoto's thyroiditis, idiopathic pulmonary fibrosis, idiopathic thrombocytopenia purpura (ITP), irritable bowel disease (IBD), IgA neuropathy, juvenile arthritis, lichen planus, lupus erythematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychondritis, polyglandular syndromes, polymyalgia rheumatics, polymyositis and dermatomyositis, primary agammaglobulinemia, primary biliary cirrhosis, psoriasis, psoriatic arthritis. Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, takayasu arteritis, temporal arteritis/giant cell arteritis, ulcerative colitis, uveitis, vasculitides such as dermatitis herpetiformis vasculitis, vitiligo, and Wegener's granulomatosis.

Some autoimmune disorders are also associated with an inflammatory condition. Examples of inflammatory disorders which are also autoimmune disorders that can be prevented, treated or managed in accordance with the methods of the invention include, but are not limited to, asthma, encephalitis, inflammatory bowel disease, chronic obstructive pulmonary disease (COPD), allergic disorders, pulmonary fibrosis, undifferentiated spondyloarthropathy, undifferentiated arthropathy, arthritis, inflammatory osteolysis, and chronic inflammation resulting from chronic viral or bacterial infections. Examples of the types of psoriasis which can be treated in accordance with the compositions and methods of the invention include, but are not limited to, plaque psoriasis, pustular psoriasis, erythrodermic psoriasis, guttate psoriasis and inverse psoriasis.

In some embodiments, the disease or disorder is cancer. In some embodiments, the cancer comprises a solid tumor. In some embodiments, the cancer comprises a blood cancer or lymphoma. In some embodiments, the cancer is metastatic cancer. In some embodiments, the disclosed compounds, compositions, or methods result in suppression of elimination of metastasis. In some embodiments, the disclosed compounds, compositions, or methods result in decreased tumor growth. In some embodiments, the disclosed compounds, compositions, or methods prevent tumor recurrence.

PI3K inhibitors may be useful to treat a wide variety of cancers including carcinoma, sarcoma, lymphoma, leukemia, melanoma, mesothelioma, multiple myeloma, or seminoma. The cancer may be a cancer of the bladder, blood, bone, brain, breast, cervix, colon/rectum, endometrium, head and neck, kidney, liver, lung, lymph nodes, muscle tissue, ovary, pancreas, prostate, skin, spleen, stomach, testicle, thyroid, or uterus In select embodiments, the cancer may comprise breast cancer.

The PI3K inhibitor, or a composition thereof, may be administered to a subject by a variety of methods. In any of the uses or methods described herein, administration may be by various routes known to those skilled in the art, including without limitation oral, inhalation, intravenous, intramuscular, topical, subcutaneous, systemic, and/or intraperitoneal administration to a subject in need thereof. In some embodiments, the PI3K inhibitor, or a composition thereof, as disclosed herein may be administered by parenteral administration (including, but not limited to, subcutaneous, intramuscular, intravenous, intraperitoneal, intracardiac and intraarticular injections). In some embodiments, the PI3K inhibitor, or a composition thereof, as disclosed herein may be administered by oral administration.

The amount of the PI3K inhibitor, or a composition thereof, of the present disclosure required for use in treatment or prevention will vary not only with the particular compound selected but also with the route of administration, the nature and/or symptoms of the disease and the age and condition of the patient and will be ultimately at the discretion of the attendant physician or clinician. The determination of effective dosage levels, that is the dosage levels necessary to achieve the desired result, can be accomplished by one skilled in the art using routine methods, for example, human clinical trials, in vivo studies, and in vitro studies. For example, useful dosages of a PI3K inhibitor, or a composition thereof, can be determined by comparing their in vitro activity, and in vivo activity in animal models.

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data Dosages necessary to achieve the MEC will depend on individual characteristics and route of administration. However, FIPLC assays or bioassays can be used to determine plasma concentrations. Dosage intervals can also be determined using MEC value. Compositions should be administered using a regimen, which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the symptoms to be treated and the route of administration. Further, the dose, and perhaps dose frequency, will also vary according to the age, body weight, and response of the individual patient. A program comparable to that discussed above may be used in veterinary medicine.

PI3K inhibitors, or compositions thereof, disclosed herein can be evaluated for efficacy and toxicity using known methods. For example, the toxicology of a particular compound, a subset of the compounds sharing certain chemical moieties, or a composition comprising a PI3K inhibitor, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs, or monkeys, may be determined using known methods. Efficacy may be established using several recognized methods, such as in vitro methods, animal models, or human clinical trials. When selecting a model to determine efficacy, the skilled artisan can be guided by the state of the art to choose an appropriate model, dose, route of administration and/or regime.

A therapeutically effective amount of a PI3K inhibitor or compound disclosed herein, or compositions thereof, may be administered alone or in combination with a therapeutically effective amount of at least one additional therapeutic agent. In some embodiments, effective combination therapy is achieved with a single composition or pharmacological formulation that includes both agents, or with two distinct compositions or formulations, administered at the same time, wherein one composition includes a compound of this invention, and the other includes the second agent(s).

In some embodiments, the at least one additional therapeutic agent comprises at least one chemotherapeutic agent. As used herein, the term “chemotherapeutic” or “anti-cancer drug” includes any small molecule or other drug used in cancer treatment or prevention. Chemotherapeutics include, but are not limited to, cyclophosphamide, methotrexate, 5-fluorouracil, doxorubicin, docetaxel, daunorubicin, bleomycin, vinblastine, dacarbazine, cisplatin, paclitaxel, raloxifene hydrochloride, tamoxifen citrate, abemaciclib, everolimus, alpelisib, anastrozole, pamidronate, anastrozole, exemestane, capecitabine, epirubicin hydrochloride, eribulin mesylate, toremifene, fulvestrant, letrozole, gemcitabine, goserelin, ixabepilone, emtansine, lapatinib, olaparib, megestrol, neratinib, palbociclib, ribociclib, talazoparib, thiotepa, toremifene, methotrexate, and tucatinib. In select embodiments, the chemotherapeutic agent comprises paclitaxel.

The chemotherapeutic agent (e.g., paclitaxel) may be provided separated or in a single composition with the PI3K inhibitor. In some embodiments, the single composition of the chemotherapeutic agent is simultaneously incorporated into compositions comprising an albumin nanoparticle In some embodiments, the albumin nanoparticle encapsulates, e.g., forms a shell surrounding, both the chemotherapeutic agent and the PI3K inhibitor.

A wide range of second therapies may be used in conjunction with the compounds of the present disclosure. The second therapy may be administration of an additional therapeutic agent or may be a second therapy not connected to administration of another agent. Such second therapies include, but are not limited to, surgery, immunotherapy, radiotherapy, or an additional chemotherapeutic or anti-cancer agent.

The second therapy (e.g., an immunotherapy) may be administered at the same time as the initial therapy, either in the same composition or in a separate composition administered at substantially the same time as the first composition. In some embodiments, the second therapy may precede or follow the treatment of the first therapy by time intervals ranging from hours to months.

In some embodiments, the second therapy includes immunotherapy. Immunotherapies include chimeric antigen receptor (CAR) T-cell or T-cell transfer therapies, cytokine therapy, immunomodulators, cancer vaccines, or administration of antibodies (e.g., monoclonal antibodies).

In some embodiments, the immunotherapy comprises administration of antibodies. The antibodies may target antigens either specifically expressed by tumor cells or antigens shared with normal cells. In some embodiments, the immunotherapy may comprise an antibody targeting, for example, CD20, CD33, CD52, CD30, HER (also referred to as erbB or EGFR), VEGF, CTLA-4 (also referred to as CD152), epithelial cell adhesion molecule (EpCAM, also referred to as CD326), and PD-1/PD-L1. Suitable antibodies include, but are not limited to, rituximab, blinatumomab, trastuzumab, gemtuzumab, alemtuzumab, ibritumomab, tositumomab, bevacizumab, cetuximab, panitumumab, ofatumumab, ipilimumab, brentuximab, pertuzumab and the like). In some embodiments, the additional therapeutic agent may comprise anti-PD-1/PD-L1 antibodies, including, but not limited to, pembrolizumab, nivolumab, cemiplimab, atezolizumab, avelumab, durvalumab, and ipilimumab. The antibodies may also be linked to a chemotherapeutic agent. Thus, in some embodiments, the antibody is an antibody-drug conjugate.

The immunotherapy (e.g., administration of antibodies) may be administered to a subject by a variety of methods. In any of the uses or methods described herein, administration may be by various routes known to those skilled in the art, including without limitation oral, inhalation, intravenous, intramuscular, topical, subcutaneous, systemic, and/or intraperitoneal administration to a subject in need thereof. In some embodiments, the immunotherapy may be administered in the same or different manner than the PI3K inhibitor, or composition thereof. The immunotherapy may be administered by parenteral administration (including, but not limited to, subcutaneous, intramuscular, intravenous, intraperitoneal, intracardiac and intraarticular injections).

### 5. Kits

In another aspect, the disclosure provides kits comprising at least one disclosed compound or a pharmaceutically acceptable salt thereof, or a composition comprising the compound or a pharmaceutically acceptable salt thereof, and instructions for using the compound or composition.

The kits can also comprise other agents and/or products co-packaged, co-formulated, and/or co-delivered with other components. For example, a drug manufacturer, a drug reseller, a physician, a compounding shop, or a pharmacist can provide a kit comprising a disclosed compound and/or product and another agent (e.g., a chemotherapeutic, a monoclonal antibody, a pain reliever, an anti-seizure medicine, a steroid, an anti-emetic) for delivery to a patient

The kits can also comprise instructions for using the components of the kit. The instructions are relevant materials or methodologies pertaining to the kit. The materials may include any combination of the following; background information, list of components, brief or detailed protocols for using the compositions, trouble-shooting, references, technical support, and any other related documents. Instructions can be supplied with the kit or as a separate member component, either as a paper form or an electronic form which may be supplied on computer readable memory device or downloaded from an internet website, or as recorded presentation.

It is understood that the disclosed kits can be employed in connection with the disclosed methods. The kit may further contain containers or devices for use with the methods or compositions disclosed herein. The kits optionally may provide additional components such as buffers and disposable single-use equipment (e.g., pipettes, cell culture plates or flasks).

The kits provided herein are in suitable packaging. Suitable packaging includes, but is not limited to, vials, bottles, jars, flexible packaging, and the like. Individual member components of the kits may be physically packaged together or separately.

### 6. Examples

Abbreviations used in the schemes and examples that follow are: AcOH is acetic acid; BPO is benzoyl peroxide: DCM is dichloromethane: DIPEA is N, N-diisopropylethylamine: DMAP is 4-dimethylaminopyridine: DMF is dimethylformamide; DMSO is dimethyl sulfoxide: EDC is 1-ethyl-3-(3-dimethylaminopropyl)carbodiimide; eq is equivalent: Et₃N or NEt₃ is triethylamine: Et₂O is diethyl ether: EtOAc is ethyl acetate; EtOH is ethanol; Et₃SiH is triethylsilane: HATU is 1-[Bis(dimethylamino)methylene]-1H-1,2,3-triazolo[4,5-b]pyridinium 3-oxid hexafluorophosphate, N-[(Dimethylamino)-1H-1,2,3-triazolo-[4,5-b]pyridin-1-ylmethylene]-N-methylmethanaminium hexafluorophosphate N-oxide: HCl is hydrochloric acid; MeOH is methanol: MsCl is methanesulfonyl chloride: NBS is N-bromosuccinimide; ON is overnight: Pd/C is palladium on carbond; PMBNH₂ is p-methoxybenzylamine: RT is room temperature; TBAF is tetra-n-butylammonium fluoride; TBAI is tetrabutylammonium idodide; THF is tetrahydrofuran; and TFA is trifluoroacetic acid.

All air and moisture sensitive manipulations were performed under either argon or in vacuo using standard Schlenk techniques. Anhydrous solvents (Et₂O, THF, Dioxane, DMSO, DMF. DCM and Toluene) were purchased from Fischer Scientific. All chemicals were purchased from Fischer Scientific. Sigma Aldrich, TCI WUXI Apptec and DC Chemicals Europe and were used without further purification unless mentioned otherwise.

Analytical thin layer chromatography (TLC) was performed with Merck SIL G/UV254 plates. Compounds were visualized by exposure to UV light or by dipping the plates in solutions of ninhydrin or potassium permanganate followed by heating or by staining with Iodine vapor in a wide jar chamber. Column chromatography was performed in air with silicagel 60 (Fluka). Column chromatography was performed with Merck Kieselgel 60 (200-500 mm). The solvent systems were given (s/s v:v).

NMR spectra 1H (300 MHz), 13C (75 MHz) were respectively recorded on an ARX 300 or an Avance II 500 Bruker spectrometer. Chemical shifts (8, ppm) are given with reference to residual 1H or 13C of deuterated solvents in the solvent indicated (CDC) 7.26, 77.00; (CD3)₂CO 2.05, 29.84 and 206.26, (CD₃)₂SO 2.50, 39.52)). ¹H- and ¹³C-NMR chemical shifts (8) are quoted in parts per million (ppm) relative to the TMS scale. Coupling constants J are quoted in Hz. The following abbreviations are used for the proton spectra multiplicities; s; singlet, d; doublet, t; triplet, q; quartet, qt; quintuplet, m; multiplet, br.; broad, dd; double doublet, dt; double triplet. Coupling constants (J) are reported in Hertz (Hz). Several signals could not be attributed will be represented by ArH (aromatic hydrogen).

Mass spectra (MS) were recorded with an LCQ-advantage (ThermoFinnigan) mass spectrometer with positive (ESI+) or negative (ESI−) electrospray ionization (ionization tension 4.5 kV, injection temperature 240° C.).

### Example 1

### Series 1

### Synthesis of Series 1 Building Blocks:

General synthetic strategy used for the synthesis of series 1 building blocks:

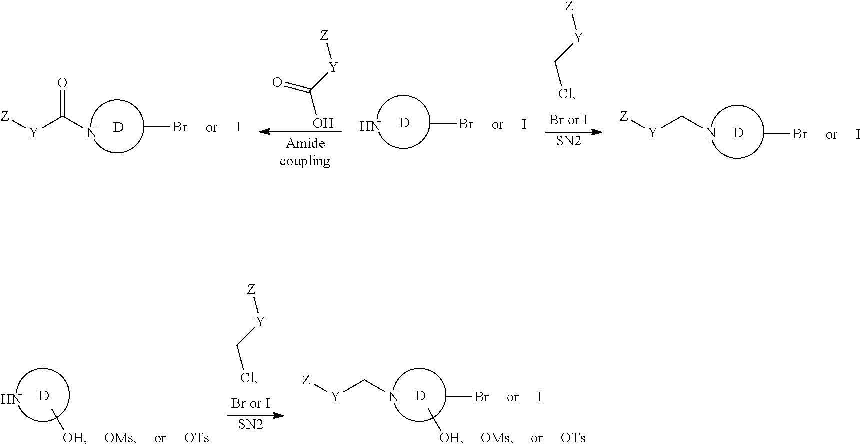

The 4-membered, 5-membered, and 6-membered rings are commercially available, e.g., from DC Chemicals. When synthesized in house, the following general procedures 1, 2, 3, 4 and 5 was used.

### General Procedure 1: Amide Coupling

Under argon atmosphere, a solution of the carboxylic acid (1 eq.) and HATU (0.95 eq.) in anhydrous DMF (0.05 M) was stirred at 0° C. for 0.5 h. Then a solution of the heterocyclic amine (I eq.) in DMF (0.1M) was added. The mixture was stirred at 0° C.-RT for 2 h (unless mentioned otherwise). Progress of the reaction was monitored by TLC using CH₂Cl₂-MeOH 9:1 mixture as eluent and 1H NMR using ARX 300 Brucker spectrometer. Then the mixture was extracted with dichloromethane (3×) and water, washed with brine, dried over anhydrous Na₂SO₄, concentrated in vacuo and purified by manual column chromatography using CH₂Cl₂-EtOAc or CH₂Cl₂-MeOH step gradient solvent system as an eluent.

### General Procedure 2: Esterification

Under argon atmosphere, a solution of the carboxylic acid (1 eq.), EDC·HCl (0.95 eq.), DMAP (10 mol %) in anhydrous THF (0.05 M) was stirred at 0° C. for 0.5 h. Then a solution of the alcohol (1 eq.) in THF (0.1M) was added. The mixture was stirred at 0° C. to RT overnight (unless mentioned otherwise) Progress of the reaction was monitored by TLC using CH₂Cl₂-MeOH 9:1 mixture as eluent and 1 H NMR using ARX 300 Brucker spectrometer. Then THE was removed and the mixture was extracted with dichloromethane (3×) and water, washed with brine, dried over anhydrous Na₂SO₄, concentrated in vacuo and purified by manual column chromatography using CH₂CL-EtOAc or CH₂Cl₂-MeOH step gradient solvent system as an eluent.

### General Procedure 3: N-Alkylation

To a solution of the amine (1 eq.) in anhydrous DMF (0.2 M) was added Potassium Carbonate (3 eq.) the mixture was stirred at RT for 5 minutes. Then the halogen derivative (1.2 eq.) was added and the mixture was stirred at 80° C., for overnight (when the halogen is a chlorine, 10 mol % of NaI was added to the reaction media). Progress of the reaction was monitored by TLC using CH₂Cl₂-MeOH 9:1 mixture as eluent and 1H NMR using ARX 300 Brucker spectrometer. Upon completion, the mixture was extracted with dichloromethane (3×) and water, washed with brine, dried over anhydrous Na₂SO₄, concentrated in vacuo and purified by manual column chromatography using CH₂Cl₂-EtOAc or CH₂Cl₂-MeOH step gradient solvent system as an eluent.

### General Procedure 4: N-Alkylation

The mixture of amine (1 eq), KI (1.5 eq), KOH (5 eq) and 22a (2 eq) in Toluene/H₂O (0.03 M, 1:1) were reacted at 80° ° C. for 16 h. After the reaction was complete, the mixture was cooled to rt and extracted with EtOAc. The combined organic phases were dried over Na₂SO₄, concentrated in vacuo, and purified by column chromatography using CH₂Cl₂-EtOAc or CH₂Cl2-MeOH step gradient solvent system as an eluent.

### General Procedure 5: N-Alkylation

A (0.3 M) DMF solution of amine (1 eq), 2-bromoethanol (1.1 eq), Cs₂CO₃ (1.5) and TBAI (0.2 eq) was heated at 80° ° C. for 2 h (unless mentioned otherwise). Reaction was monitored by TLC and 1H NMR. The reaction was then diluted into iced water and extracted with EtOAc (3×). The combined organic layer was washed with water, brine, dried over anhydrous Na₂SO₄, concentrated in vacuo, and purified by manual column chromatography using CH₂Cl₂-MeOH step gradient solvent to afford the desired product.

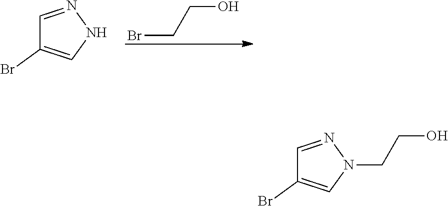

Following the General Procedure 5, 4-bromo-1H-pyrazole (1.00 g, 6.80 mmol, 1 eq) was reacted with DMF (20 mL, 0.3 M), 2-bromoethanol (0.93 g, 7.48 mmol, 1.1 eq), Cs₂CO₃ (3.32 g, 10.20 mmol, 1.5 eq) and TBAI (0.50 g, 1.36 mmol, 0.2 eq) which, after column purification (SiO₂, CH₂Cl₂-EtOAc 1:1 then CH₂Cl2-MeOH 9:1) afford the desired.

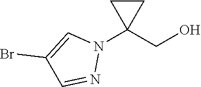 [SMILES: OCC1(n2cc(Br)cn2)CC1]

The building block (CAS=2241142-25-4, [1-(4-bromopyrazol-1-yl)cyclopropyl]methanol]) was ordered from click chemistry.

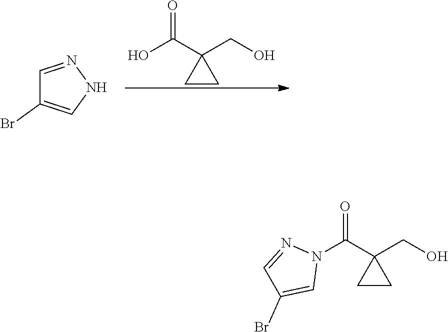 [SMILES: Brc1cn[nH]c1 ; O=C(O)C1(CO)CC1 ; O=C(n1cc(Br)cn1)C1(CO)CC1]

Following the General Procedure 1, 1-(hydroxymethyl)cyclopropane-1-carboxylic acid (1 g, 8.61 mmol, I eq.) DIPEA (2.3 mL, 13 mmol, 1.5 eq) and HATU (3.1 g, 8.18 mmol, 0.95 eq.) in anhydrous DMF (17 mL, 0.5 M) was stirred at 0° C. for 0.5 h. Then a solution of 4-bromo-1H-pyrazole (1.27 g, 8.61 mmol, 1 eq.) in DMF (17 mL, 0.5M) was added follow by DIPEA (1.5 mL, 8.61 mmol, I eq). The mixture was stirred at 0° C. to RT for 4 h. Progress of the reaction was monitored by TLC. The mixture was extracted with dichloromethane (3×) and iced water, washed with brine, dried over anhydrous Na₂SO₄, concentrated in vacuo and purified by manual column chromatography using CH₂Cl₂-EtOAc step gradient solvent system as an eluent to afford the desired product.

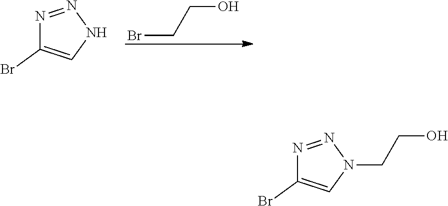

Following the General Procedure 5, 4-bromo-1H-1,2,3-triazole (1.01 g, 6.80 mmol, 1 eq) was reacted with DMF (20 mL, 0.3 M), 2-bromoethanol (0.93 g, 7.48 mmol, 1.1 eq), Cs₂CO₃ (3.32 g, 10.20 mmol, 1.5 eq) and TBAI (0.50 g, 1.36 mmol, 0.2 eq) which, after column purification (SiO₂, CH₂Cl₂-EtOAc step gradient solvent system as an eluent) afforded the desired product.

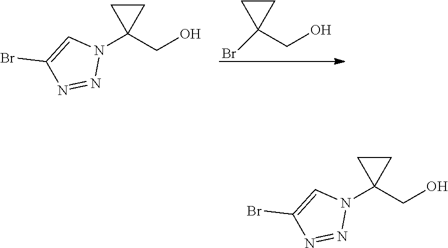 [SMILES: OCC1(n2cc(Br)nn2)CC1 ; OCC1(Br)CC1 ; OCC1(n2cc(Br)nn2)CC1]

According to General Procedure 5, 4-bromo-1H-1,2,3-triazole (1.01 g, 6.80 mmol, 1 eq) was reacted with DMF (20 mL, 0.3 M), (I-bromocyclopropyl)methanol (1.13 g, 7.48 mmol, 1.1 eq), Cs₂CO₃ (3.32 g, 10.20 mmol, 1.5 eq) and TBAI (0.50 g, 1.36 mmol, 0.2 eq) which, after column purification (SiO₂, CH₂Cl₂-EtOAc step gradient solvent system as an eluent) afforded the desired product as a yellow oil.

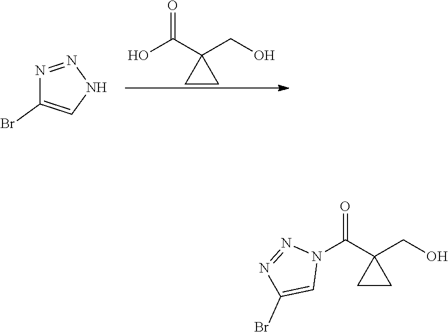 [SMILES: Brc1c[nH]nn1 ; O=C(O)C1(CO)CC1 ; O=C(n1cc(Br)nn1)C1(CO)CC1]

Following the General Procedure 1, 1-(hydroxymethyl)cyclopropane-1-carboxylic acid (1 g, 8.61 mmol, 1 eq.), DIPEA (2.3 mL, 13 mmol, 1.5 eq), and HATU (3.1 g, 8.18 mmol, 0.95 eq.) in anhydrous DMF (17 mL, 0.5 M) was stirred at 0° C. for 0.5 h. Then a solution of 4-bromo-1H-1,2,3-triazole (1.27 g, 8.61 mmol, I eq.) in DMF (17 mL, 0.5M) was added follow by DIPEA (1.5 mL, 8.61 mmol, I eq). The mixture was stirred at 0° C. to RT for 4 h. Progress of the reaction was monitored by TLC. The mixture was extracted with dichloromethane (3×) and iced water, washed with brine, dried over anhydrous Na₂SO₄, concentrated in vacuo and purified by manual column chromatography using CH₂Cl₂-EtOAc step gradient solvent system as an eluent to afford the desired product.

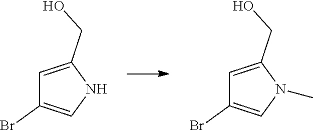

A suspension of (4-bromo-1H-pyrrol-2-yl)methanol (1 g, 5.68 mmol, 1 eq), K₂CO₃ (2 g, 14.2 mmol, 2.5 eq) and Mel (0.39 mL, 6.3 mmol, 1.1 eq) was stirred in THF (20 mL, 0.3 M) at 60° ° C. overnight. Then the reaction was filtrated and dried in vacuo to afford the desired product.

### Ligand Synthesis:

The alkyne derivative was purchased from Wuxi Apptec. The coupling of the alkyne to the 4-membered and the S-membered rings was performed using the following General Procedure 6 described below:

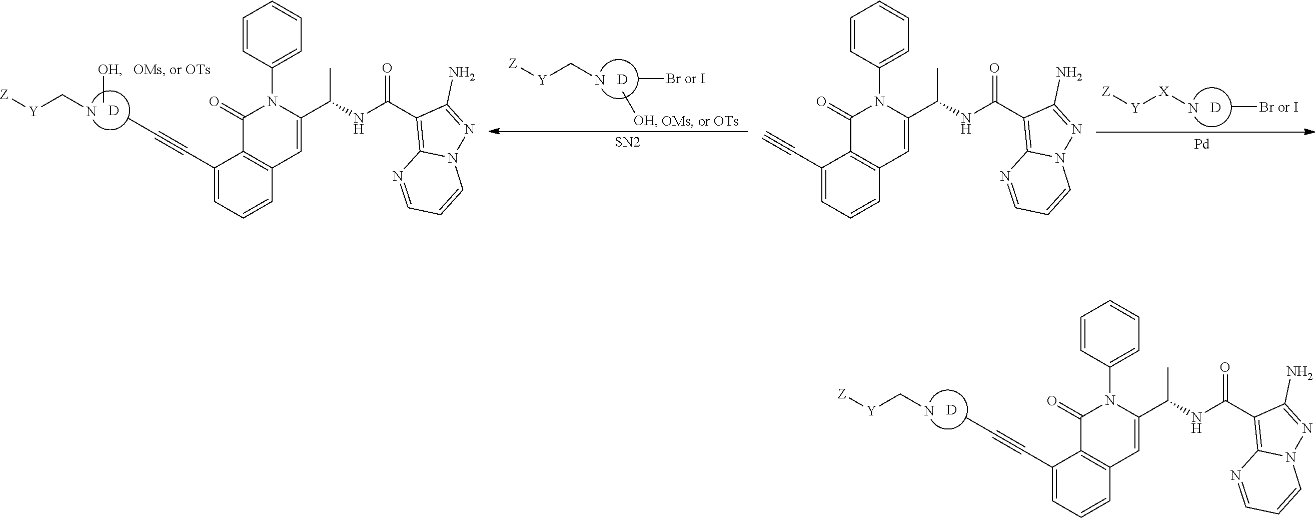

### General Procedure 6: Synthesis of Arylethylnyl Derivatives by Sonogashira Coupling.

Using a procedure of Rozhkov and co-workers, halogenoaryl derivative (1 equiv.), alkyne (2 equiv.), bis(diphenylphosphino)palladium dichloride (5 mol %), CuI (10 mol %) and anhydrous NEt₃ (8 equiv.) were dissolved in 2 mL of DMF and stirred at RT for 16 h (unless noted otherwise). Progress of the reaction was monitored by TLC using CH₂Cl₂-MeOH 9:1 mixture as eluent and 1H NMR using ARX 300 Brucker spectrometer. Upon completion of the reaction, solvent was evaporated in vacuo and solid residue was purified by manual column chromatography using CH₂Cl₂-EtOAc or CH₂Cl₂-MeOH step gradient solvent system as an eluent.

### (S)-2-amino-N-(1-(8-((1-(2-hydroxyethyl)-1H-pyrazol-4-yl)ethynyl)-1-oxo-2-phenyl-1,2-dihydroisoquinolin-3-yl)ethyl)pyrazolo[1,5-a]pyrimidine-3-carboxamide

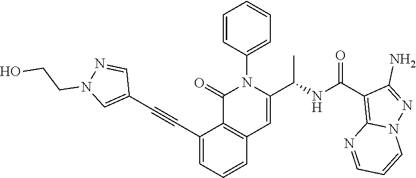 [SMILES: C[C@H](NC(=O)c1c(N)nn2cccnc12)c1cc2cccc(C#Cc3cnn(CCO)c3)c2c(=O)n1-c1ccccc1]

According to General Procedure 6, 2-(4-bromo-1H-pyrazol-1-yl)ethan-1-ol (50 mg, 0.26 mmol, 1.2 equiv.) in DMF (2.2 mL, 0.1 M) was reacted with (S)-2-amino-N-(1-(8-ethynyl-1-oxo-2-phenyl-1,2-dihydroisoquinolin-3-yl)ethyl)pyrazolo[1,5-a]pyrimidine-3-carboxamide (0.1 g, 0.22 mmol, 1.0 equiv.), bis(diphenylphosphino)palladium dichloride (8 mg, 0.011 mmol, 5 mol %), CuI (4.2 mg, 0.022 mmol, 10 mol %) and NEt₃ (0.25 mL, 1.76 mmol, 8 equiv.) after which column purification (SiO₂, CH₂Cl₂-MeOH step gradient as eluent system) afforded the desired product

### (S)-2-amino-N-(1-(8-((1-(1-(hydroxymethyl)cyclopropyl)-1H-pyrazol-4-yl)ethynyl)-1-oxo-2-phenyl-1,2-dihydroisoquinolin-3-yl)ethyl)pyrazolo[1,5-a]pyrimidine-3-carboxamide

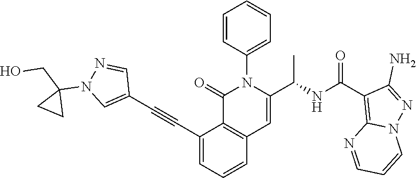 [SMILES: C[C@H](NC(=O)c1c(N)nn2cccnc12)c1cc2cccc(C#Cc3cnn(C4(CO)CC4)c3)c2c(=O)n1-c1ccccc1]

According to General Procedure 6, (1-(4-bromo-1H-pyrazol-1-yl)cyclopropyl)methanol (57 mg, 0.26 mmol, 1.2 equiv.) in DMF (2.2 mL, 0.1 M) was reacted with (S)-2-amino-N-(1-(8-ethynyl-1-oxo-2-phenyl-1,2-dihydroisoquinolin-3-yl)ethyl)pyrazolo[1,5-a]pyrimidine-3-carboxamide (0.1 g, 0.22 mmol, 1.0 equiv.), bis(diphenylphosphino)palladium dichloride (8 mg, 0.011 mmol, 5 mol %), CuI (4.2 mg, 0.022 mmol, 10 mol %) and NEt₃ (0.25 mL, 1.76 mmol, 8 equiv.) after which column purification (SiO₂, CH₂Cl₂-MeOH step gradient as eluent system) afforded the desired product.

### (S)-2-amino-N-(1-(8-((1-(2-hydroxyethyl)-1H-1,2,3-triazol-4-yl)ethynyl)-1-oxo-2-phenyl-1,2-dihydroisoquinolin-3-yl)ethyl)pyrazolo[1,5-a]pyrimidine-3-carboxamide

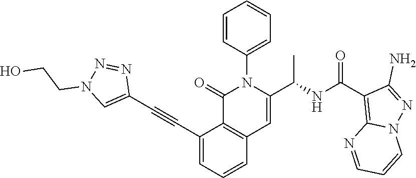 [SMILES: C[C@H](NC(=O)c1c(N)nn2cccnc12)c1cc2cccc(C#Cc3cn(CCO)nn3)c2c(=O)n1-c1ccccc1]

According to General Procedure 6, 2-(4-bromo-1H-1,2,3-triazol-1-yl)ethan-1-ol (50 mg, 0.26 mmol, 1.2 equiv.) in DMF (2.2 mL, 0.1 M) was reacted with (S)-2-amino-N-(1-(8-ethynyl-1-oxo-2-phenyl-1,2-dihydroisoquinolin-3-yl)ethyl)pyrazolo[1,5-a]pyrimidine-3-carboxamide (0.1 g, 0.22 mmol, 1.0 equiv.), bis(diphenylphosphino)palladium dichloride (8 mg, 0.011 mmol, 5 mol %), CuI (4.2 mg, 0.022 mmol, 10 mol %) and NEt₃ (0.25 mL, 1.76 mmol, 8 equiv.) after which column purification (SiO₂, CH₂Cl₂-MeOH step gradient as eluent system) afforded the desired product.

### (S)-2-amino-N-(1-(8-((1-(1-(hydroxymethyl)cyclopropyl)-1H-1,2,3-triazol-4-yl)ethynyl)-1-oxo-2-phenyl-1,2-dihydroisoquinolin-3-yl)ethyl)pyrazolo[1,5-a]pyrimidine-3-carboxamide

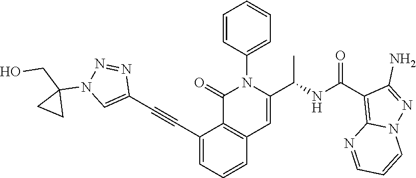 [SMILES: C[C@H](NC(=O)c1c(N)nn2cccnc12)c1cc2cccc(C#Cc3cn(C4(CO)CC4)nn3)c2c(=O)n1-c1ccccc1]

According to General Procedure 6, (1-(4-bromo-1H-1,2,3-triazol-1-yl)cyclopropyl)methanol (57 mg, 0.26 mmol, 1.2 equiv.) in DMF (2.2 mL, 0.1 M) was reacted with (S)-2-amino-N-(1-(8-ethynyl-1-oxo-2-phenyl-1,2-dihydroisoquinolin-3-yl)ethyl)pyrazolo[1,5-a]pyrimidine-3-carboxamide (0.1 g, 0.22 mmol, 1.0 equiv.), bis(diphenylphosphino)palladium dichloride (8 mg, 0.011 mmol, 5 mol %), CuI (4.2 mg, 0.022 mmol, 10 mol %) and NEt₃ (0.25 mL, 1.76 mmol, 8 equiv.) after which column purification (SiO₂, CH₂Cl₂-MeOH step gradient as eluent system) afforded the desired product.

### (S)-2-amino-N-(1-(8-((1-(1-(hydroxymethyl)cyclopropane-1-carbonyl)-1H-pyrazol-4-yl)ethynyl)-1-oxo-2-phenyl-1,2-dihydroisoquinolin-3-yl)ethyl)pyrazolo[1,5-a]pyrimidine-3-carboxamide

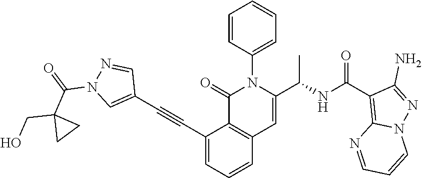 [SMILES: C[C@H](NC(=O)c1c(N)nn2cccnc12)c1cc2cccc(C#Cc3cnn(C(=O)C4(CO)CC4)c3)c2c(=O)n1-c1ccccc1]

According to General Procedure 6, (4-bromo-1H-pyrazol-1-yl)(1-(hydroxymethyl)-cyclopropyl)methanone (64 mg, 0.26 mmol, 1.2 equiv.) in DMF (2.2 mL, 0.1 M) was reacted with (S)-2-amino-N-(1-(8-ethynyl-1-oxo-2-phenyl-1,2-dihydroisoquinolin-3-yl)ethyl)pyrazolo[1,5-a]pyrimidine-3-carboxamide (0.1 g, 0.22 mmol, 1.0 equiv.), bis(diphenylphosphino)palladium dichloride (8 mg, 0.011 mmol, 5 mol %), CuI (4.2 mg, 0.022 mmol, 10 mol %) and NEt₃ (0.25 mL, 1.76 mmol, 8 equiv.) after which column purification (SiO₂, CH₂Cl₂-MeOH step gradient as eluent system) afforded the desired product.

### (S)-2-amino-N-(1-(8-((1-(1-(hydroxymethyl)cyclopropane-1-carbonyl)-1H-1,2,3-triazol-4-ylethynyl)-1-oxo-2-phenyl-1,2-dihydroisoquinolin-3-yl)ethyl)pyrazolo[1,5-a]pyrimidine-3-carboxamide

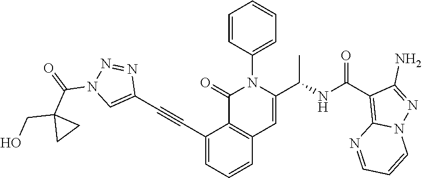 [SMILES: C[C@H](NC(=O)c1c(N)nn2cccnc12)c1cc2cccc(C#Cc3cn(C(=O)C4(CO)CC4)nn3)c2c(=O)n1-c1ccccc1]

According to General Procedure 6, (4-bromo-1H-1,2,3-triazol-1-yl)(1-(hydroxymethyl)-cyclopropyl)methanone (67 mg, 0.26 mmol, 1.2 equiv.) in DMF (2.2 mL, 0.1 M) was reacted with (S)-2-amino-N-(1-(8-ethynyl-1-oxo-2-phenyl-1,2-dihydroisoquinolin-3-yl)ethyl)pyrazolo[1,5-a]pyrimidine-3-carboxamide (0.1 g, 0.22 mmol, 1.0 equiv.), bis(diphenylphosphino)palladium dichloride (8 mg, 0.011 mmol, 5 mol %), CuI (4.2 mg, 0.022 mmol, 10 mol %) and NEt₃ (0.25 mL, 1.76 mmol, 8 equiv.) after which column purification (SiO₂, CH₂Cl₂-MeOH step gradient as eluent system) afforded the desired product.

### (S)-2-amino-N-(1-(8-((5-(hydroxymethyl)-1-methyl-1H-pyrrol-3-yl)ethynyl)-1-oxo-2-phenyl-1,2-dihydroisoquinolin-3-yl)ethyl)pyrazolo[1,5-a]pyrimidine-3-carboxamide

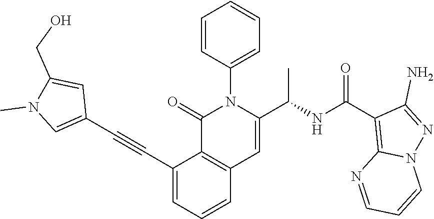 [SMILES: C[C@H](NC(=O)c1c(N)nn2cccnc12)c1cc2cccc(C#Cc3cc(CO)n(C)c3)c2c(=O)n1-c1ccccc1]

According to General Procedure 6, (4-bromo-1-methyl-1H-pyrrol-2-yl)methanol (50 mg, 0.26 mmol, 1.2 equiv.) in DMF (2.2 mL, 0.1 M) was reacted with (S)-2-amino-N-(1-(8-ethynyl-1-oxo-2-phenyl-1,2-dihydroisoquinolin-3-yl)ethyl)pyrazolo[1,5-a]pyrimidine-3-carboxamide (0.1 g, 0.22 mmol, 1.0 equiv.), bis(diphenylphosphino)palladium dichloride (8 mg, 0.011 mmol, 5 mol %), CuI (4.2 mg, 0.022 mmol, 10 mol %) and NEt₃ (0.25 mL, 1.76 mmol, 8 equiv.) after which column purification (SiO₂, CH₂Cl₂-MeOH step gradient as eluent system) afforded the desired product.

### Example 2

### Series 2

### Synthesis of the Building Blocks:

### 1. Isoindolinones

The isoindolinone derivatives were synthesized as describes in the general procedures below.

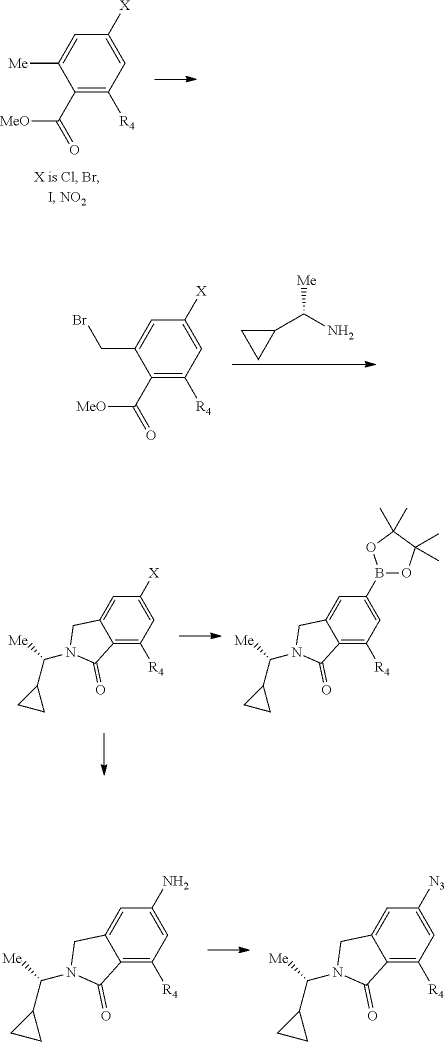

### General Procedure 8: Bromination

To a solution of 4-halo/or nitro-2-substituted-6-methylbenzoic acid methyl ester (1 eq) in CCl₄ (0.2 M) were added NBS (1.1 eq) and BPO (80 wt. %, 0.1 eq). The resulting mixture was stirred at reflux for 3 h. Upon completion, the reaction mixture was cooled to RT and filtered to remove the precipitate. The filtrate was concentrated in vacuo to afford the crude product that was used directly in the next step.

### General Procedure 9; Amide Coupling

A mixture of 4-halo/or nitro-2-substituted-6-methylbenzoic acid (1 eq), (S)-1-cyclopropylethan-1-amine (1.2 eq), HATU (1.2 eq) and diisopropylethylamine (3 eq) in anhydrous DMF (0.3 M) was stirred at 0° ° C. to RT for 3 h. The reaction mixture was quenched with saturated aqueous solution of NH₄Cl, extracted with EtOAc, dried over Na₂SO₄, concentrated in vacuo, and purified by manual column chromatography using CH₂Cl₂-EtOAc step gradient solvent system as an eluent to afford the desired product.

General Procedure 10: Isoindolinone Cyclization from Amide

The intermediate (1 eq) from the previous step was dissolved in THF (0.2 M) and cooled to −78° C., then sec-Butyllithium (1.4 M in cyclohexane, 2.5 eq) was added dropwise. The reaction mixture was stirred at −78° C., for 20 min, then DMF (5 eq) was added dropwise. After 1 h, the reaction was carefully quenched with saturated aqueous solution of NH₄Cl and extracted with EtOAc. After concentration in vacuo, the crude product was dissolved in DCM (0.32 M) and cooled to 0° C. Et₃SiH (1 eq) and TFA (0.62 M) were added and the reaction mixture was stirred at RT for 20 minutes. The reaction mixture was concentrated in vacuo, quenched with saturated aqueous solution of NaHCO₃, extracted with DCM, concentrated in vacuo, and purified by manual column chromatography using CH₂Cl₂-EtOAc step gradient solvent system as an eluent to afford the desired product.

General Procedure 11: Isoindolinone Cyclization from Methyl Ester

In a round-bottom flask equipped with a reflux condenser and a balloon of N2, crude 2-(bromomethyl)-benzoate (1 eq) was combined with (S)-1-cyclopropylethan-1-amine hydrochloride (2 eq), K₂CO₃ (3 eq) and B(OH)₃ (0.2 eq) in acetonitrile (0.25 M). The resulting mixture was stirred at 50° C., for 72 h. Upon completion, the reaction mixture was cooled to RT and 3/4 of the solvent was removed in vacuo. The mixture was partitioned between EtOAc and H₂O. The aqueous phase was separated and extracted with additional EtOAc. The combined organic extracts were washed with water and brine. The organic phase was dried over Na₂SO₄, concentrated in vacuo, and purified by manual column chromatography using CH₂C₁₂-EtOAc or CH₂Cl₂-MeOH step gradient solvent system as an eluent to afford the desired product.

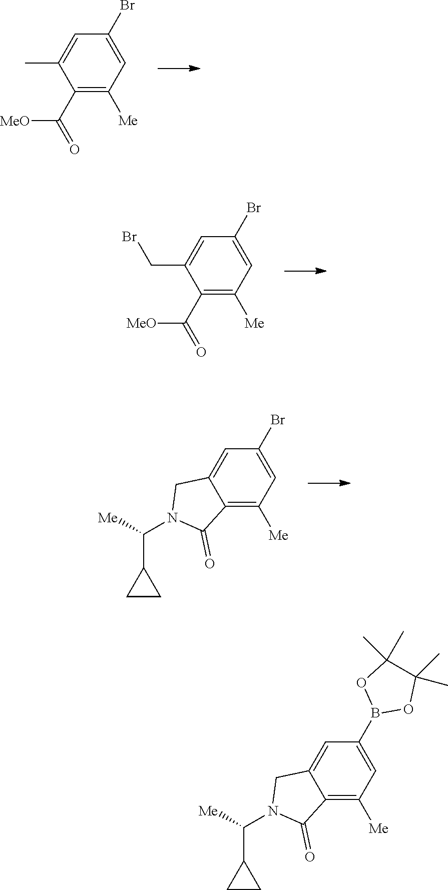 [SMILES: COC(=O)c1c(C)cc(Br)cc1C ; COC(=O)c1c(C)cc(Br)cc1CBr ; Cc1cc(Br)cc2c1C(=O)N([C@@H](C)C1CC1)C2 ; Cc1cc(B2OC(C)(C)C(C)(C)O2)cc2c1C(=O)N([C@@H](C)C1CC1)C2]

### Step 1; methyl 4-bromo-2-(bromomethyl)-6-methylbenzoate

According to general procedure 8, methyl 4-bromo-2,6-dimethylbenzoate (2 g, 8.23 mmol, 1 eq), NBS (1.611 g, 9.05 mmol, 1.1 eq), BPO (0.2 g, 0.823 mmol, 0.1 eq), and CCl₄ (41 mL, 0.2 M) were reacted together according to General Procedure 8 to afford the titled product which was used in the next step without further purification.

### Step 2: (S)-5-bromo-2-(1-cyclopropylethyl)-7-methylisoindolin-1-one

According to General Procedure 11, crude methyl 4-bromo-2-(bromomethyl)-6-methylbenzoate (2.65 g, 8.23 mmol, 1 eq), (S)-1-cyclopropylethan-1-amine hydrochloride (1.4 g, 16.46 mmol, 2 eq), K₂CO₃ (3.41 g, 24.69 mmol, 3 eq) and B(OH): (7 μL 0.16 mmol, 0.2 eq) in acetonitrile (35 mL, 0.25 M) was reacted together to afford the titled product.

### Step 3: (S)-2-(1-cyclopropylethyl)-7-methyl-5-(4,4,5,5-tetramethyl-1,3,2-dioxaborolan-2-yl)isoindolin-1-one

A Purged (3×) suspension of (S)-5-bromo-2-(1-cyclopropylethyl)-7-methylisoindolin-1-one (1 g, 3.4 mmol, 1 eq), bis(pinacolato)diboron (1 g, 4.1 mmol), potassium acetate (1.2 g, 8.5 mmol, 2.5 eq) and PdCl₂(dppf) (0.25 g, 0.34 mmol, 0.1 eq) in dioxane (10 mL, 0.3 M) was heated to 100° C., for 3 h. After cooling to RT, the reaction mixture was filtered through a pad of celite and the filter pad was washed with 15% MeOH/DCM (15 mL). Et₂O (100 mL) was added to the filtrate to precipitate. The solid residue was used in the next step without further purification.

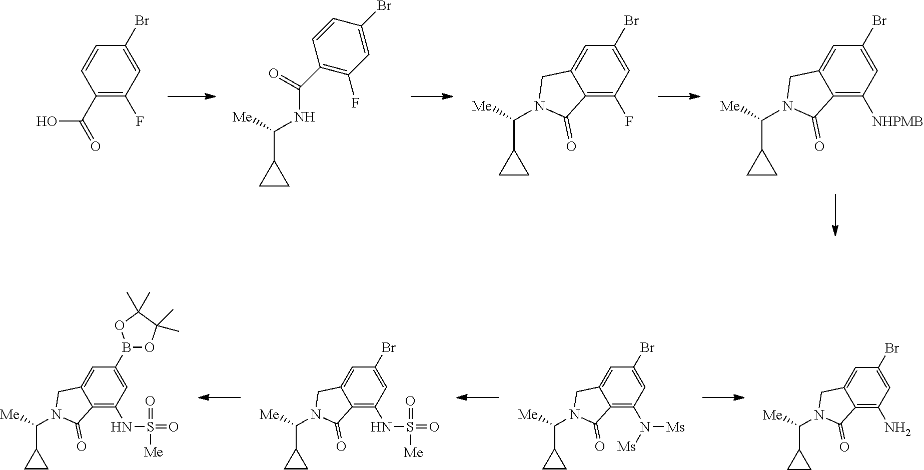

### Step 1: (S)-4-bromo-N-(1-cyclopropylethyl)-2-fluorobenzamide

A mixture of 4-bromo-2-fluorobenzoic acid (6 g, 18.4 mmol, 1 eq), (S)-1-cyclopropylethan-1-amine (2.82 g, 33 mmol, 1.2 eq), HATU (12.3 g, 33 mol, 1.2 eq) and diisopropylethylamine (14.5 mL, 82.2 mmol, 3 eq) in anhydrous DMF (37 mL, 0.5 M) was stirred at 0° ° C. to RT for 3 h. The reaction mixture was quenched with saturated aqueous solution of NH₄Cl, extracted with EtOAc, dried over Na₂SO₄, concentrated in vacuo, and purified by manual column chromatography using CH₂Cl₂-EtOAc step gradient solvent system as an eluent to afford the desired product.

### Step 2: (S)-5-bromo-2-(1-cyclopropylethyl)-7-fluoroisoindolin-1-one

(S)-4-bromo-N-(1-cyclopropylethyl)-2-fluorobenzamide (6 g, 20.4 mmol, 1 eq) was dissolved in THF (100 mL, 0.2 M) and cooled to −78° C., then sec-butyllithium (36 mL, 1.4 M in cyclohexane, 2.5 eq) was added dropwise. The reaction mixture was stirred at −78° C. for 20 min, then DMF (7.5 mL, 102 mmol, 5 eq) was added dropwise. After 1 h, the reaction was carefully quenched with saturated aqueous solution of NH₄Cl and extracted with EtOAc. After concentration in vacuo, the crude product was dissolved in DCM (60 mL, 0.32 M) and cooled to 0° C. Et₃SiH (3.3 mL, 6.8 mmol, 1 eq) and TFA (33 mL, 0.62 M) were added and the reaction mixture was stirred at RT for 20 minutes. The reaction mixture was concentrated in vacuo, quenched with saturated aqueous solution of NaHCO₃, extracted with DCM, concentrated in vacuo, and purified by manual column chromatography using CH₂Cl₂-EtOAc step gradient solvent system as an eluent to afford the desired product.

### Step 3: (S)-5-bromo-2-(1-cyclopropylethyl)-7-((4-methoxybenzyl)amino)isoindolin-1-one

(S)-5-bromo-2-(1-cyclopropylethyl)-7-fluoroisoindolin-1-one (3 g, 10 mmol, 1 eq) was combined with neat PMBNH₂ (4 mL) and heated to 100° C., for 14 h. The reaction mixture was cooled and partitioned between 10% aqueous solution of citric acid solution and EtOAc. The aqueous layer was separated and back extracted with additional EtOAc. The organic layers were combined and washed with additional 10% aqueous solution of citric acid solution, brine, dried over Na₂SO₄ and concentrated in vacuo to afford (S)-5-bromo-2-(1-cyclopropylethyl)-7-((4-methoxybenzyl)amino)isoindolin-1-one that was used crude in the next step

### Step 4: (S)-7-amino-5-bromo-2-(1-cyclopropylethyl)isoindolin-1-one

Crude (S)-5-bromo-2-(1-cyclopropylethyl)-7-((4-methoxybenzyl)amino)isoindolin-1-one was combined with TFA (15 mL) and stirred at 40 ºC for 3 h. The reaction mixture was concentrated under reduced pressure and quenched with saturated aqueous solution of NaHCO₃ and diluted with EtOAc. The combined organic phases were washed with brine and dried over Na₂SO₄, concentrated in vacuo and purified by manual column chromatography using Hexane-EtOAc step gradient solvent system as an eluent to afford the desired product to afford the desired product.

### Step 5: (S)—N-(6-bromo-2-(1-cyclopropylethyl)-3-oxoisoindolin-4-yl)-N-(methylsulfonyl) methanesulfonamide

(S)-7-amino-5-bromo-2-(1-cyclopropylethyl)isoindolin-1-one (1.00 g, 3.38 mmol) was dissolved in DCM (10 mL) and the mixture was cooled to 0° C. To this solution was added DMAP (40 mg, 0.34 mmol). DIPEA (1.6 mL, 10.1 mmol) and MsCl (0.7 mL, 8.5 mmol). The reaction mixture was warmed to RT and stirred for 1 h. The reaction was quenched with 1 M HCl (aq.) and diluted with EtOAc. The aqueous layer was separated and back extracted with additional EtOAc. The organic layers were combined, washed with brine, and dried over MgSO₄. Concentration under reduced pressure furnished bis-sulfonylated product that was taken crude into the next step.

### Step 6: (S)—N-(6-bromo-2-(1-cyclopropylethyl)-3-oxoisoindolin-4-yl)methanesulfonamide

Crude product from the previous step was dissolved in THE (5 mL) and TBAF (1.0 M in THE, 5.4 mL, 5.4 mmol) was added. An additional portion of TBAF (1.0 M in THE, 3.0 mL) was added after 15 min, followed by a final portion of TBAF (1.0 M in THF, 3.0 mL) after 2 h. The reaction mixture was stirred for an additional 1 h, then quenched with 1 M HCl (aq.) and diluted with EtOAc. The aqueous layer was separated and back extracted with additional EtOAc. The organic phase was dried over Na₂SO₄, concentrated in vacuo, and purified by manual column chromatography using CH₂Cl₂-EtOAc or CH₂Cl₂-MeOH step gradient solvent system as an eluent to afford the desired product

### Step 7: (S)—N-(2-(1-cyclopropylethyl)-3-oxo-6-(4,4,5,5-tetramethyl-1,3,2-dioxaborolan-2-yl) isoindolin-4-yl)methanesulfonamide

A purged (3×) suspension of (S)—N-(6-bromo-2-(1-cyclopropylethyl)-3-oxoisoindolin-4-yl)methanesulfonamide (0.5 g, 1.34 mmol, 1 eq), bis(pinacolato)diboron (0.2 g, 1.61 mmol, 1.2 eq), potassium acetate (0.48 g, 3.4 mmol, 2.5 eq) and PdCl₂(dppf) (0.1 g, 0.13 mmol, 0.1 eq) in dioxane (5 mL, 0.3 M) was heated to 100° C., for 5 h. After cooling to RT, the reaction mixture was filtered through a pad of celite and the filter pad was washed with THF. Et₂O (50 mL) was added to the filtrate to precipitate. The solid residue was used in the next step without further purification.

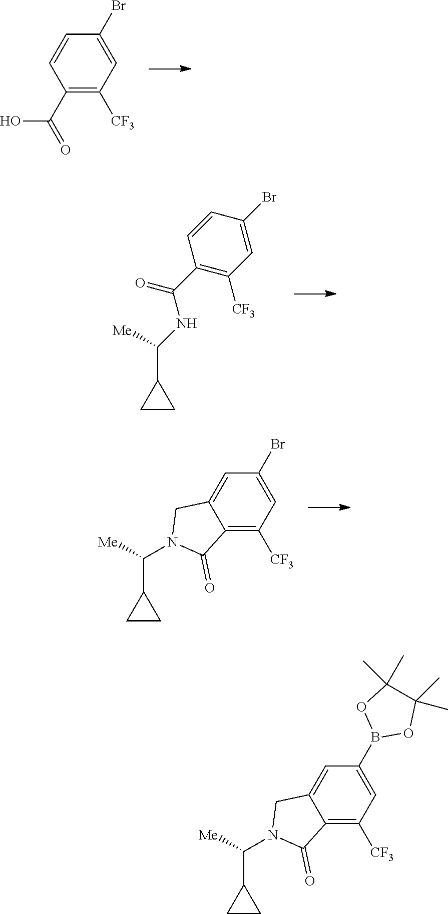 [SMILES: O=C(O)c1ccc(Br)cc1C(F)(F)F ; C[C@H](NC(=O)c1ccc(Br)cc1C(F)(F)F)C1CC1 ; C[C@@H](C1CC1)N1Cc2cc(Br)cc(C(F)(F)F)c2C1=O ; C[C@@H](C1CC1)N1Cc2cc(B3OC(C)(C)C(C)(C)O3)cc(C(F)(F)F)c2C1=O]

### Step 1: (S)-4-bromo-N-(1-cyclopropylethyl)-2-fluorobenzamide

A mixture of 4-bromo-2-(trifluoromethyl)benzoic acid (20 g, 74.3 mmol, 1 eq). (S)-1-cyclopropylethan-1-amine (7.62 g, 89.2 mmol, 1.2 eq), HATU (33.2 g, 89.2 mol, 1.2 eq) and diisopropylethylamine (43.5 mL, 246.6 mmol, 3 eq) in anhydrous DMF (100 mL, 0.74 M) was stirred at 0° C., to RT for 3 h. The reaction mixture was quenched with saturated aqueous solution of NH₄Cl, extracted with EtOAc, dried over Na₂SO₄, concentrated in vacuo, and purified by manual column chromatography using CH₂Cl₂-MeOH (9:1) as an eluent to afford the desired product.

### Step 2: (S)-5-bromo-2-(1-cyclopropylethyl)-7-(trifluoromethyl)isoindolin-1-one

(S)-4-bromo-N-(1-cyclopropylethyl)-2-(trifluoromethyl)benzamide (10 g, 29.7 mmol, 1 eq) was dissolved in THF (100 mL, 0.3 M) and cooled to −78° ° C., then sec-butyllithium (14.5 mL, 1.4 M in cyclohexane, 2.5 eq) was added dropwise. The reaction mixture was stirred at −78° C. for 20 min, then DMF (11.5 mL, 148.5 mmol, 5 eq) was added dropwise. After 1 h, the reaction was carefully quenched with saturated aqueous solution of NH₄Cl and extracted with EtOAc. After concentration in vacuo, the crude product was dissolved in DCM (90 mL, 0.32 M) and cooled to 0° C. Et₃SiH (4.8 mL, 29.7 mmol, 1 eq) and TFA (25 mL, 1.2 M) were added and the reaction mixture was stirred at RT for 20 minutes. The reaction mixture was concentrated in vacuo, quenched with saturated aqueous solution of NaHCO₃, extracted with DCM, concentrated in vacuo, and purified by manual column chromatography using CH₂Cl₂-EtOAc step gradient solvent system as an eluent to afford the desired product.

### Step 3: (S)-5-bromo-2-(1-cyclopropylethyl)-7-((4-methoxybenzyl)amino)isoindolin-1-one

A purged (3×) suspension of (S)-5-bromo-2-(1-cyclopropylethyl)-7-(trifluoromethyl)isoindolin-1-one (4 g, 11.5 mmol, 1 eq), bis(pinacolato)diboron (1.7 g, 13.7 mmol, 1.2 eq), potassium acetate (4.1 g, 28.75 mmol, 2.5 eq) and PdCl₂(dppf) (0.9 g, 1.2 mmol, 0.1 eq) in dioxane (40 mL, 0.3 M) was heated to 100° C. for 5 h. After cooling to RT, the reaction mixture was filtered through a pad of celite and the filter pad was washed with THF. Et₂O (100 mL) was added to the filtrate to precipitate. The solid residue was used in the next step without further purification.

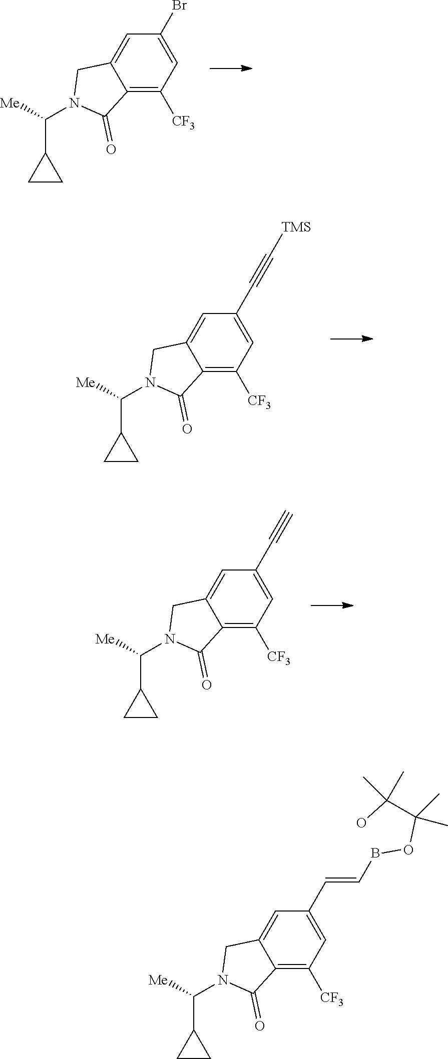

### Step 1: (S)-2-(1-cyclopropylethyl)-7-(trifluoromethyl)-5-((trimethylsilyl)ethynyl)isoindolin-1-one

A mixture of (S)-5-bromo-2-(1-cyclopropylethyl)-7-(trifluoromethyl)isoindolin-1-one (10 g, 27.4 mmol, 1 equiv.), ethynyltrimethylsilane (7.6 mL, 54.8 mmol, 2 equiv.), bis(diphenylphosphino)palladium dichloride (0.6 g, 0.78 mmol, 3 mol %), CuI (0.26 g, 1.4 mmol, 5 mol %) in dry NEt₃ (50 mL, 0.5 M) was heated at reflux for 16 h under argon. The reaction mixture was filtered over Celite® and washed with EtOAc. The filtrate was evaporated in vacuo, the residue was purified by column chromatography on silica gel eluting with a mixture of CH₂Cl₂-EtOAc step gradient solvent system as an eluent to afford the desired product.

### Step 2: (S)-2-(1-cyclopropylethyl)-5-ethynyl-7-(trifluoromethyl)isoindolin-1-one

Tetrabutylammonium fluoride (1.0 M in tetrahydrofuran, 25 mL, 1.2 equiv) was added to (S)-2-(1-cyclopropylethyl)-7-(trifluoromethyl)-5-((trimethylsilyl)ethynyl)isoindolin-1-one (7.4 g, 20.1 mmol, 1 eq). The reaction was stirred during 16 h after which the solvent was removed in vacuo and the crude residue was purified by column chromatography (SiO₂, CH₂Cl₂/-MeOH 9:1) to provide desired product.

### Step 3: (S,E)-2-(1-cyclopropylethyl)-5-(2-(4,4,5,5-tetramethyl-1,3,2-dioxaborolan-2-yl)vinyl)-7-(trifluoromethyl)isoindolin-1-one

An argon fluxed (3×) solution of (S)-2-(1-cyclopropylethyl)-5-ethynyl-7-(trifluoromethyl)isoindolin-1-one (2 g, 6.82 mmol, 1 equiv.) in anhydrous toluene (45 mL, 0.15 M) and RuHCl(CO)(PPh₃)₃ (0.32 g, 0.34 mmol, 5 mol %) was stirred for 5 minutes. Then 4,4,5,5-tetramethyl-1,3,2-dioxaborolane (1, 32, 10.3 mmol, 1.5 equiv.) was added. The mixture was stirred for 18 h at 50° C. The crude reaction was concentrated in vacuo then extracted with EtOAc and a saturated solution of NaHCO₃. The organic layer was concentrated in vacuo and precipitated with Et₂O. The crude was used in the next step without additional purification.

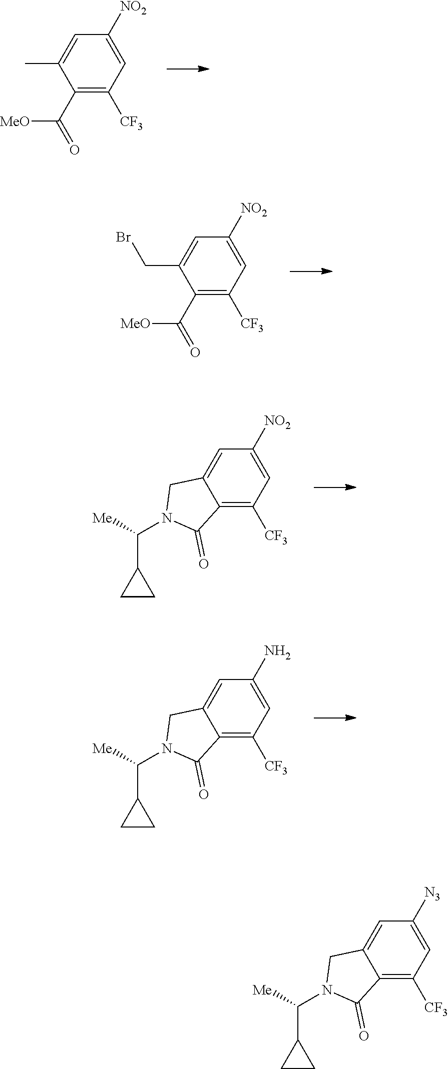 [SMILES: COC(=O)c1c(C)cc([N+](=O)[O-])cc1C(F)(F)F ; COC(=O)c1c(CBr)cc([N+](=O)[O-])cc1C(F)(F)F ; C[C@@H](C1CC1)N1Cc2cc([N+](=O)[O-])cc(C(F)(F)F)c2C1=O ; C[C@@H](C1CC1)N1Cc2cc(N)cc(C(F)(F)F)c2C1=O ; C[C@@H](C1CC1)N1Cc2cc(N=[N+]=[N-])cc(C(F)(F)F)c2C1=O]

### Step 1; methyl 2-(bromomethyl)-4-nitro-6-(trifluoromethyl)benzoate

According to General Procedure 8, methyl 2-methyl-4-nitro-6-(trifluoromethyl)benzoate (21.7 g, 82.3 mmol, 1 eq). NBS (16.11 g, 90.5 mmol, 1.1 eq). BPO (2 g, 82.3 mmol, 0.1 eq), and CCl₄ (410 mL, 0.2 M) were reacted together to afford the titled product which was used in the next step without further purification.

### Step 2: (S)-2-(1-cyclopropylethyl)-5-nitro-7-(trifluoromethyl)isoindolin-1-one

According to General Procedure 11, crude methyl 2-(bromomethyl)-4-nitro-6-(trifluoromethyl)benzoate (28.2 g, 82.3 mmol, 1 eq), (S)-1-cyclopropylethan-1-amine hydrochloride (14 g, 164.6 mmol, 2 eq), K₂CO₃ (34.1 g, 246.9 mmol, 3 eq) and B(OH): (70 μL 1.6 mmol, 0.2 eq) in acetonitrile (350 mL, 0.25 M) was reacted together to afford the titled product.

### Step 3: (S)-5-amino-2-(1-cyclopropylethyl)-7-(trifluoromethyl)isoindolin-1-one

To a solution of (S)-2-(1-cyclopropylethyl)-5-nitro-7-(trifluoromethyl)isoindolin-1-one (15 g, 47.8 mmol, 1 equiv.) in a mixture of H₂O-EtOH (1:1, 160 mL, 0.3 M) was added dropwise a suspension of NH₄Cl (2.2 g, 38.2 mmol, 0.8 equiv.) and Fe(0) (16 g, 287 mmol, 6 equiv.). The reaction mixture was refluxed during 2 h. After cooling, the resulting suspension was filtered through a pad of Celite® and washed with a solution of THE/EtOH. After evaporation in vacuo, the residue was purified by flash chromatography on silica gel eluting with DCM/MeOH (9:1) to yield the desired product

### Step 4: (S)-5-azido-2-(1-cyclopropylethyl)-7-(trifluoromethyl)isoindolin-1-one

To a solution of (S)-5-amino-2-(1-cyclopropylethyl)-7-(trifluoromethyl)isoindolin-1-one (10 g, 35.2 mmol, 1 equiv.) in HCl 17% (100 mL) was added dropwise at 0° C. a solution of NaNO₂ (3.6 g, 52.8 mmol, 1.5 equiv.) in water (12 mL). The reaction mixture was stirred at this temperature for 60 minutes then a solution of NaN₃ (3.4 g, 52.8 mmol, 1.5 equiv.) in water (20 mL) was added dropwise. The reaction mixture was warmed to room temperature and stirred for an additional 2 h. The resulting suspension was filtered then dried to yield titled product.

### Synthesis of Isoquinolinone Building Blocks:

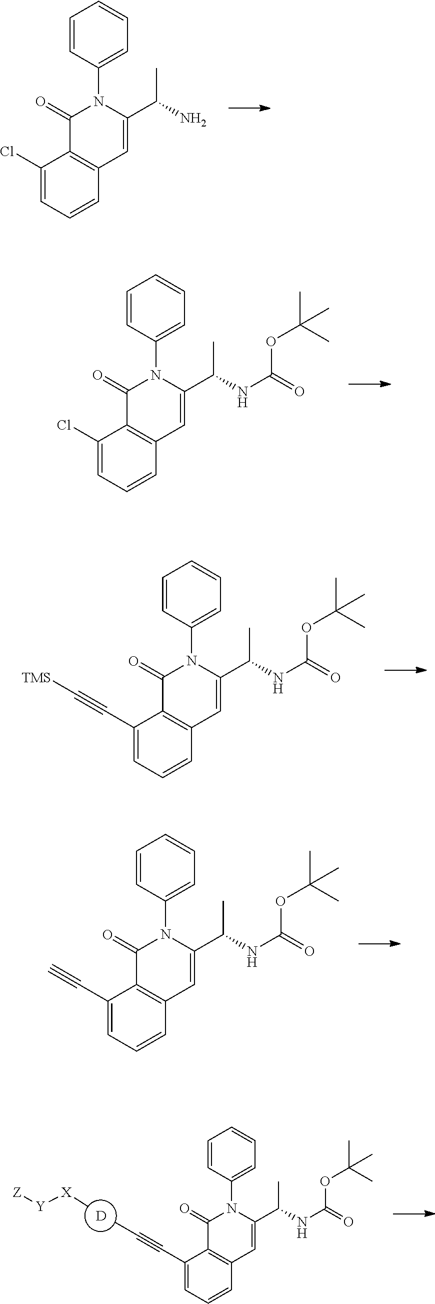

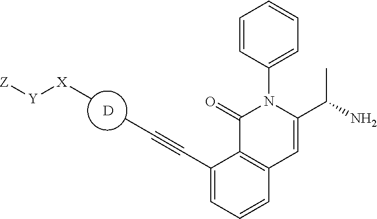

### Step 1; tert-butyl (S)-(1-(8-chloro-1-oxo-2-phenyl-1,2-dihydroisoquinolin-3-yl)ethyl)carbamate

An anhydrous DCM solution (70 ml, 0.5 M) of (S)-3-(1-aminoethyl)-8-chloro-2-phenylisoquinolin-1(2H)-one (10 g, 33.56 mmol, 1 eq) was treated by trimethylamine (11.7 mL, 83.9 mmol, 2.5 eq) and (Boc)₂O (5.2 g, 40.3 mmol, 1.05 eq) at 0° C., to RT over 16 h. The mixture was washed with 1 N HCl aqueous solution and brine to afford the desired product tert-butyl (S)-(1-(8-chloro-1-oxo-2-phenyl-1,2-dihydroisoquinolin-3-yl)ethyl)carbamate as a solid.

### Step 2; tert-butyl (S)-(1-(1-oxo-2-phenyl-8-((trimethylsilyl)ethynyl)-1,2-dihydroisoquinolin-3-yl)ethyl)carbamate

To an agron fluxed (3×) suspension of dichlorobis(acetonitrile)palladium (15 mol %), X-Phos (45 mol %), cesium carbonate (3.0 equiv) and tert-butyl (S)-(1-(8-chloro-1-oxo-2-phenyl-1,2-dihydroisoquinolin-3-yl)ethyl)carbamate (4 g, 10 mmol, 1.0 equiv) in propionitrile (100 mL, 0.1 M) was added ethynyl trimethylsilane (5.0 equiv., in 5 mL propionitrile). The resulting yellow mixture was stirred at RT for 20 min then heated to 75° ° C. for 3 h. The brownish-black mixture was cooled to RT, filtered through a pad of celite. The liquid phase was concentrated in vacuo and purified by manual column chromatography using CH₂Cl₂-EtOAc step gradient solvent system as an eluent to afford the desired product tert-butyl (S)-(1-(1-oxo-2-phenyl-8-((trimethylsilyl)ethynyl)-1,2-dihydroisoquinolin-3-yl)ethyl)carbamate.

### Step 3; tert-butyl (S)-(1-(8-ethynyl-1-oxo-2-phenyl-1,2-dihydroisoquinolin-3-yl)ethyl)carbamate

A solution of tert-butyl (S)-(1-(1-oxo-2-phenyl-8-((trimethylsilyl)ethynyl)-1,2-dihydroisoquinolin-3-yl)ethyl)carbamate (2.8 g, 6 mmol) and NaOH 10% (14.6 mL) in methanol (61 mL) was refluxed during 16 h. After cooling, the reaction mixture was evaporated to dryness. The residue was purified by flash chromatography on silica gel eluting with CH₂Cl: MeOH (9:1) to yield de title product tert-butyl (S)-(1-(8-ethynyl-1-oxo-2-phenyl-1,2-dihydroisoquinolin-3-yl)ethyl)carbamate as a solid.

### Step 4: General Procedure 6: Synthesis of Arylethylnyl Derivatives by Sonogashira Coupling

Using a procedure of Rozhkov and co-workers, halogenoaryl derivative (1 equiv.), alkyne (2 equiv.), bis(diphenylphosphino)palladium dichloride (5 mol %), CuI (10 mol %) and anhydrous NEt₃ (8 equiv.) were dissolved in 2 mL of DMF and stirred at RT for 16 h (unless noted otherwise). Progress of the reaction was monitored by TLC using CH₂Cl₂-MeOH 9:1 mixture as eluent and 1H NMR using ARX 300 Brucker spectrometer. Upon completion of the reaction, solvent was evaporated in vacuo and solid residue was purified by manual column chromatography using CH₂Cl₂-EtOAc or CH₂Cl₂-MeOH step gradient solvent system as an eluent.

### Step 5: General Procedure 7: Boc Deprotection

A solution of the Boc-protected amine in HCl (4 M in dioxane, 0.5 M) was stirred at 0° C. to RT during 4 h (unless mentioned otherwise). Then the mixture was dried in vacuo to afford the titled product.

### Synthesis of the Ligands:

Six type of linkers were used for the synthesis of the series 2 compounds; no-linker, ethyl alkyl linker, ethenyl alkenyl linker, ethynyl alkynyl linker, amidyl linker, triazolyl linker.

### No-Linker:

No-linker type of ligands are compounds in which the isoquinoline is directly bound to the aminopyrimidinopyridine moiety. The synthesis of the ligands is described below.

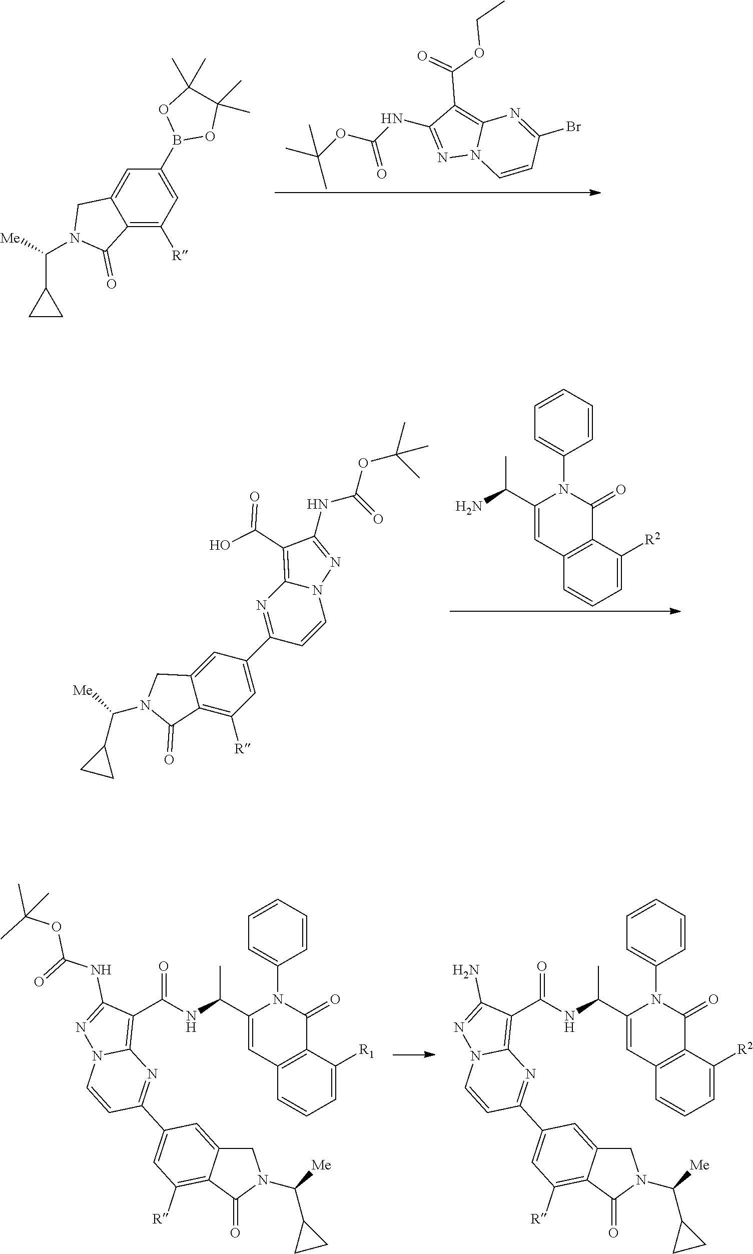

### Step 1: General Procedure 12: Suzuki Coupling

A mixture of boronic ester (68 mg, 0.173 mmol 1 eq), the aryl bromide (40 mg, 0.190 mmol 1.1 eq), PdCl₂(dppf) (10 mol %), Na₂CO₃ (5 eq) in dioxane/H₂O (4:1, 0.05 M) was heated at 95° C., for 15 min. Upon completion of the reaction, the reaction was partitioned between CH₂Cl₂ and H₂O. The aqueous layer was separated and back extracted with additional CH₂Cl. The organic layers were combined, dried over Na₂SO₄ evaporated in vacuo and purified by manual column chromatography using CH₂Cl₂-EtOAc or CH₂Cl₂-MeOH step gradient solvent system as an eluent.

### Step 2: General Procedure 1: Amide Coupling

Under argon atmosphere, a solution of the carboxylic acid (1 eq.) and HATU (0.95 eq.) in anhydrous DMF (0.05 M) was stirred at 0° ° C. for 0.5 h. Then a solution of the heterocyclic amine (1 eq.) in DMF (0.1M) was added. The mixture was stirred at 0° C. to RT for 2 h (unless mentioned otherwise). Progress of the reaction was monitored by TLC using CH₂Cl₂-MeOH 9:1 mixture as eluent and 1H NMR using ARX 300 Brucker spectrometer. Then the mixture was extracted with dichloromethane (3×) and water, washed with brine, dried over anhydrous Na₂SO₄, concentrated in vacuo and purified by manual column chromatography using CH₂Cl₂-EtOAc or CH₂Cl₂-MeOH step gradient solvent system as an eluent.

### Step 3: General Procedure 13 Boc Deprotection

A solution of the Boc-protected amine in HCl (4 M in dioxane, 0.05 M) was stirred at 0° C. to RT over 4 h (unless mentioned otherwise). Then the mixture was dried in vacuo to afford the titled product.

### 2-amino-N—((S)-1-(8-chloro-1-oxo-2-phenyl-1,2-dihydroisoquinolin-3-yl)ethyl)-5-(2-((S)-1-cyclopropylethyl)-7-methyl-1-oxoisoindolin-5-yl)pyrazolo[1,5-a]pyrimidine-3-carboxamide

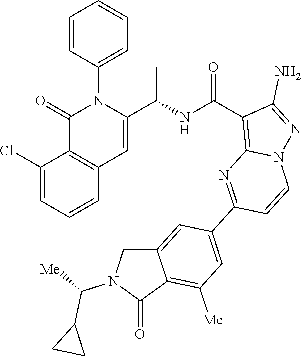 [SMILES: Cc1cc(-c2ccn3nc(N)c(C(=O)N[C@@H](C)c4cc5cccc(Cl)c5c(=O)n4-c4ccccc4)c3n2)cc2c1C(=O)N([C@@H](C)C1CC1)C2]

The desired product was synthesized in 3 steps following General Procedure 12 (Suzuki coupling, step 1), General Procedure 1 (amide coupling, step 2), and General Procedure 13 (boc deprotection, step 3)

### 2-amino-N—((S)-1-(8-chloro-1-oxo-2-phenyl-1,2-dihydroisoquinolin-3-yl)ethyl)-5-(2-((S)-1-cyclopropylethyl)-7-(methylsulfonamido)-1-oxoisoindolin-5-yl)pyrazolo[1,5-a]pyrimidine-3-carboxamide

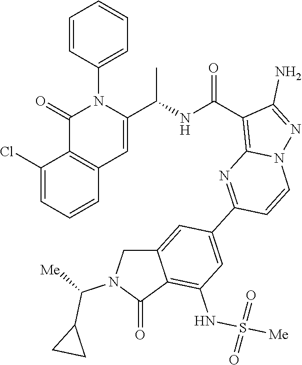 [SMILES: C[C@H](NC(=O)c1c(N)nn2ccc(-c3cc4c(c(NS(C)(=O)=O)c3)C(=O)N([C@@H](C)C3CC3)C4)nc12)c1cc2cccc(Cl)c2c(=O)n1-c1ccccc1]

The desired product was synthesized in 3 steps following General Procedure 12 (Suzuki coupling, step 1), General Procedure 1 (amide coupling, step 2), and General Procedure 13 (boc deprotection, step 3)

### 2-amino-N—((S)-1-(8-chloro-1-oxo-2-phenyl-1,2-dihydroisoquinolin-3-yl)ethyl)-5-(2-((S)-1-cyclopropylethyl)-1-oxo-7-(trifluoromethyl)isoindolin-5-yl)pyrazolo[1,5-a]pyrimidine-3-carboxamide

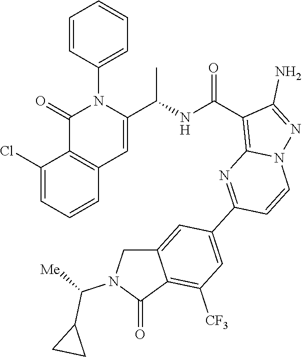 [SMILES: C[C@H](NC(=O)c1c(N)nn2ccc(-c3cc4c(c(C(F)(F)F)c3)C(=O)N([C@@H](C)C3CC3)C4)nc12)c1cc2cccc(Cl)c2c(=O)n1-c1ccccc1]

The desired product was synthesized in 3 steps following General Procedure 12 (Suzuki coupling, step 1), General Procedure 1 (amide coupling, step 2), and General Procedure 13 (boc deprotection, step 3)

### 2-amino-5-(2-((S)-1-cyclopropylethyl)-1-oxo-7-(trifluoromethyl)isoindolin-5-yl)-N—((S)-1-(8-((1-methyl-1H-pyrazol-4-yl)ethynyl)-1-oxo-2-phenyl-1,2-dihydroisoquinolin-3-yl)ethyl)pyrazolo[1,5-a]pyrimidine-3-carboxamide

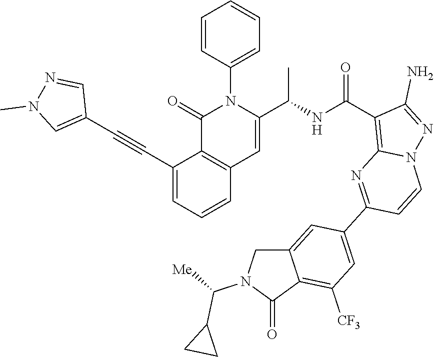 [SMILES: C[C@H](NC(=O)c1c(N)nn2ccc(-c3cc4c(c(C(F)(F)F)c3)C(=O)N([C@@H](C)C3CC3)C4)nc12)c1cc2cccc(C#Cc3cnn(C)c3)c2c(=O)n1-c1ccccc1]

The desired product was synthesized in 3 steps following General Procedure 12 (Suzuki coupling, step 1), General Procedure 1 (amide coupling, step 2), and General Procedure 13 (boc deprotection, step 3)

### 2-amino-5-(2-((S)-1-cyclopropylethyl)-1-oxo-7-(trifluoromethyl)isoindolin-5-yl)-N—((S)-1-(8-((1-(2-hydroxyethyl)-1H-pyrazol-4-yl)ethynyl)-1-oxo-2-phenyl-1,2-dihydroisoquinolin-3-yl)ethyl)pyrazolo[1,5-a]pyrimidine-3-carboxamide

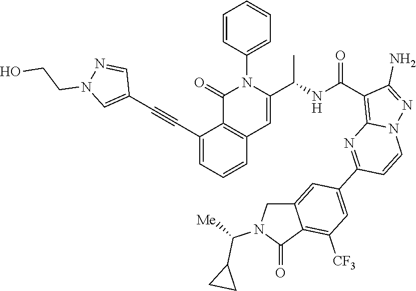 [SMILES: C[C@H](NC(=O)c1c(N)nn2ccc(-c3cc4c(c(C(F)(F)F)c3)C(=O)N([C@@H](C)C3CC3)C4)nc12)c1cc2cccc(C#Cc3cnn(CCO)c3)c2c(=O)n1-c1ccccc1]

The desired product was synthesized in 3 steps following General Procedure 12 (Suzuki coupling, step 1), General Procedure 1 (amide coupling, step 2), and General Procedure 13 (boc deprotection, step 3)

### Alkyne, Alkene, Alkane, Triazole and Amide Linker:

### 2-amino-N—((S)-1-(8-chloro-1-oxo-2-phenyl-1,2-dihydroisoquinolin-3-yl)ethyl)-5-((2-((S)-1-cyclopropylethyl)-1-oxo-7-(trifluoromethyl)isoindolin-5-yl)ethynyl)pyrazolo[1,5-a]pyrimidine-3-carboxamide

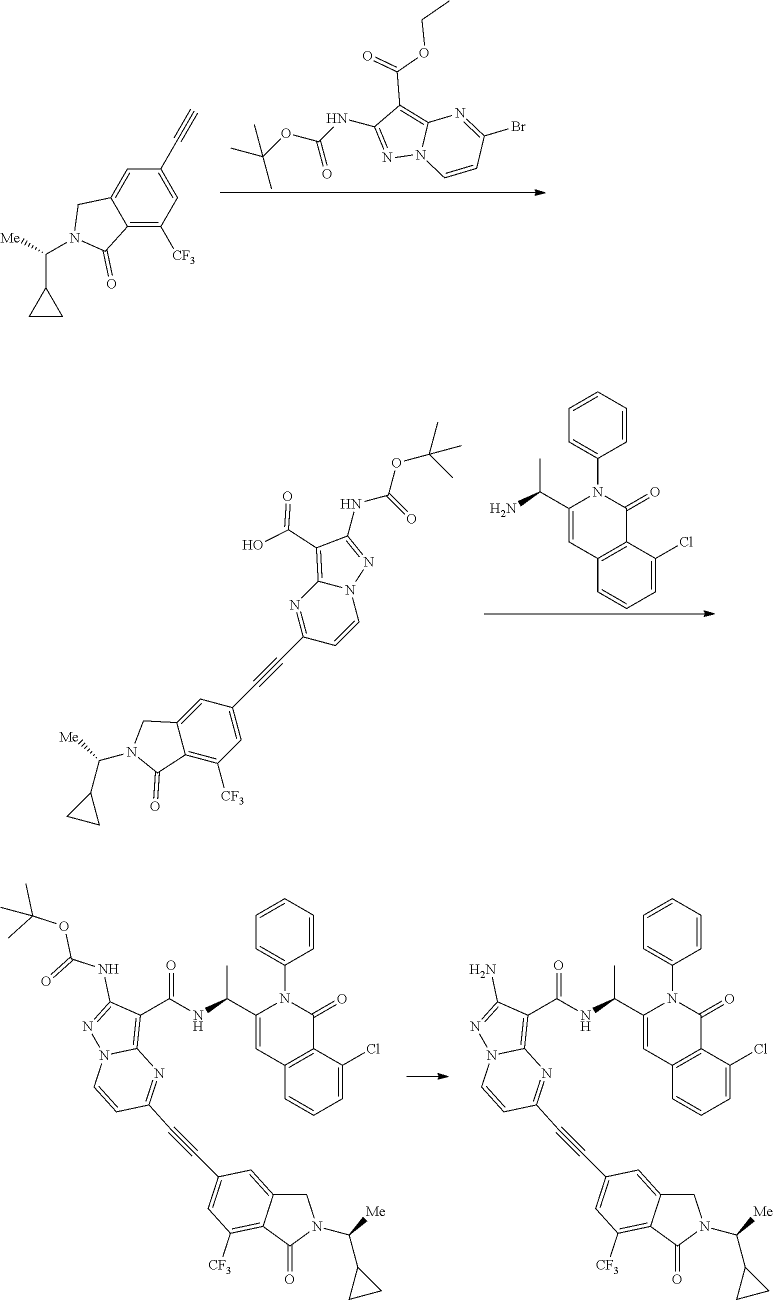 [SMILES: C#Cc1cc2c(c(C(F)(F)F)c1)C(=O)N([C@@H](C)C1CC1)C2 ; CCOC(=O)c1c(NC(=O)OC(C)(C)C)nn2ccc(Br)nc12 ; C[C@@H](C1CC1)N1Cc2cc(C#Cc3ccn4nc(NC(=O)OC(C)(C)C)c(C(=O)O)c4n3)cc(C(F)(F)F)c2C1=O ; C[C@H](N)c1cc2cccc(Cl)c2c(=O)n1-c1ccccc1 ; C[C@H](NC(=O)c1c(NC(=O)OC(C)(C)C)nn2ccc(C#Cc3cc4c(c(C(F)(F)F)c3)C(=O)N([C@@H](C)C3CC3)C4)nc12)c1cc2cccc(Cl)c2c(=O)n1-c1ccccc1 ; C[C@H](NC(=O)c1c(N)nn2ccc(C#Cc3cc4c(c(C(F)(F)F)c3)C(=O)N([C@@H](C)C3CC3)C4)nc12)c1cc2cccc(Cl)c2c(=O)n1-c1ccccc1]

The desired product was synthesized in 3 steps following General Procedure 6 (Sonogashira coupling, step 1), General Procedure 1 (amide coupling, step 2), and General Procedure 13 (boc deprotection, step 3)

### 2-amino-N—((S)-1-(8-chloro-1-oxo-2-phenyl-1,2-dihydroisoquinolin-3-yl)ethyl)-5-((E)-2-(2-((S)-1-cyclopropylethyl)-1-oxo-7-(trifluoromethyl)isoindolin-5-yl)vinyl)pyrazolo[1,5-a]pyrimidine-3-carboxamide

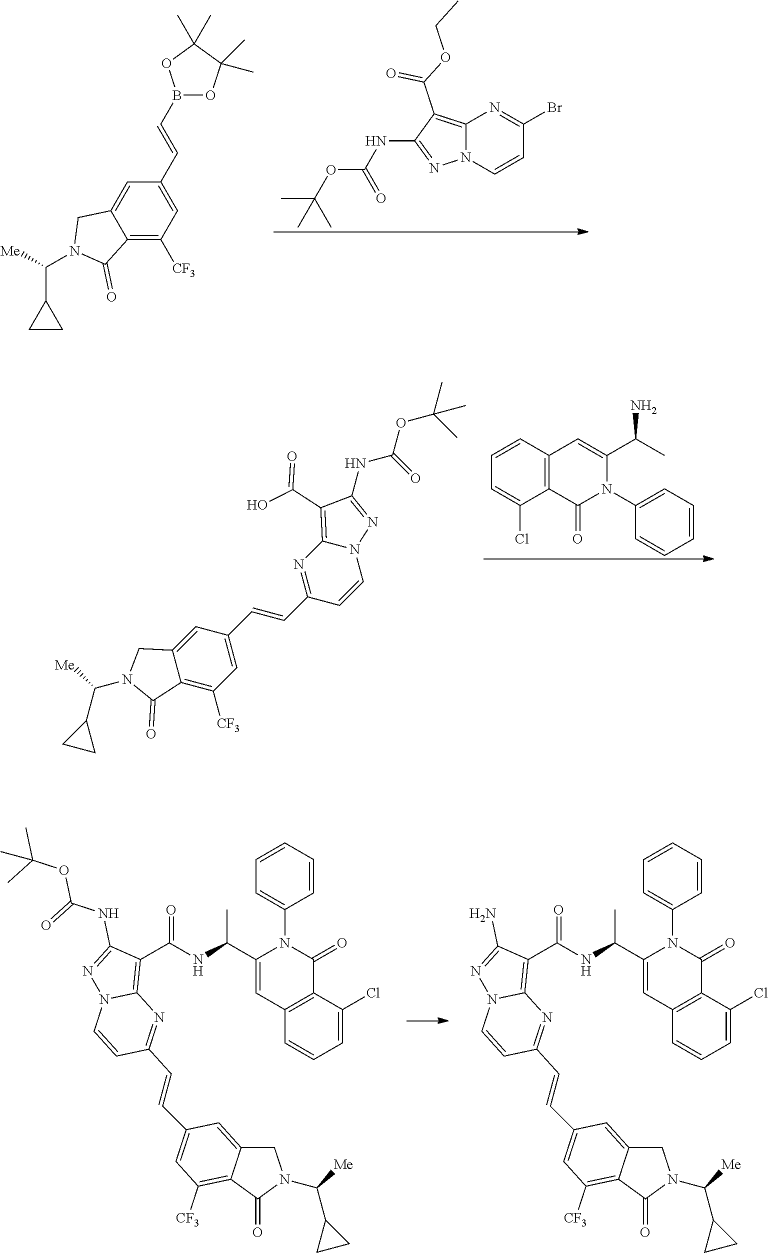

The desired product was synthesized in 3 steps following General Procedure 12 (Suzuki coupling, step 1), General Procedure 1 (amide coupling, step 2), and General Procedure 13 (boc deprotection, step 3).

### 2-amino-N—((S)-1-(8-chloro-1-oxo-2-phenyl-1,2-dihydroisoquinolin-3-yl)ethyl)-5-(2-(2-((S)-1-cyclopropylethyl)-1-oxo-7-(trifluoromethyl)isoindolin-5-yl)ethyl)pyrazolo[1,5-a]pyrimidine-3-carboxamide

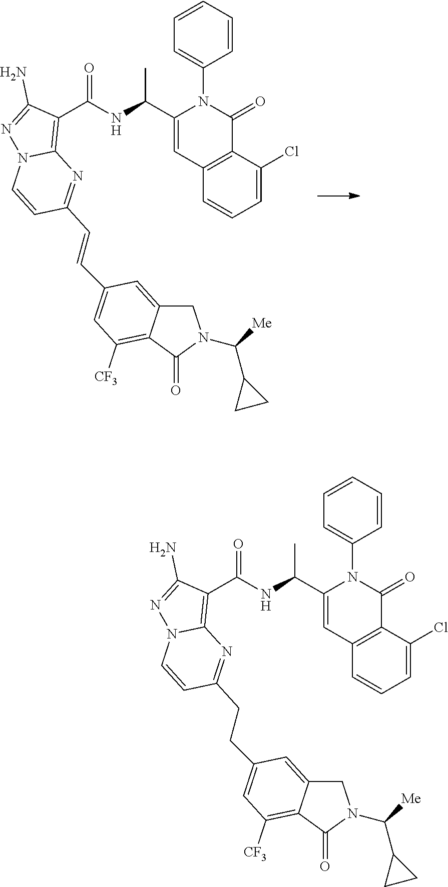 [SMILES: C[C@H](NC(=O)c1c(N)nn2ccc(/C=C/c3cc4c(c(C(F)(F)F)c3)C(=O)N([C@@H](C)C3CC3)C4)nc12)c1cc2cccc(Cl)c2c(=O)n1-c1ccccc1 ; C[C@H](NC(=O)c1c(N)nn2ccc(CCc3cc4c(c(C(F)(F)F)c3)C(=O)N([C@@H](C)C3CC3)C4)nc12)c1cc2cccc(Cl)c2c(=O)n1-c1ccccc1]

A solution of 2-amino-N—((S)-1-(8-chloro-1-oxo-2-phenyl-1,2-dihydroisoquinolin-3-yl)ethyl)-5-((E)-2-(2-((S)-1-cyclopropylethyl)-1-oxo-7-(trifluoromethyl)isoindolin-5-yl)vinyl)pyrazole [1,5-a]pyrimidine-3-carboxamide (1 eq) in THF (0.05 M) was titrated with Pd/C (20 mol %). Then the mixture was filtrated through a pad of celite. The liquid layer was concentrated in vacuo and purified by manual column chromatography using CH₂Cl₂-MeOH step gradient solvent system as an eluent to afford the desired product.

### 2-amino-N—((S)-1-(8-chloro-1-oxo-2-phenyl-1,2-dihydroisoquinolin-3-yl)ethyl)-5-(1-(2-((S)-1-cyclopropylethyl)-1-oxo-7-(trifluoromethyl)isoindolin-5-yl)-1H-1,2,3-triazol-4-yl)pyrazolo[1,5-a]pyrimidine-3-carboxamide

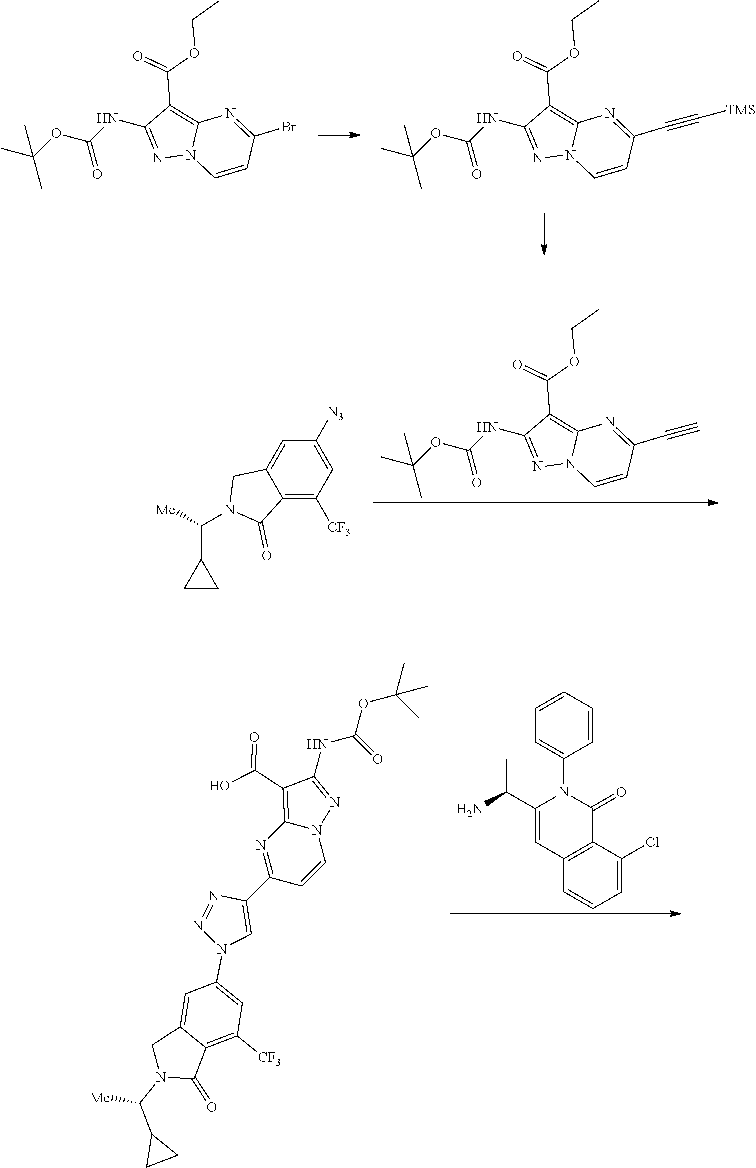 [SMILES: CCOC(=O)c1c(NC(=O)OC(C)(C)C)nn2ccc(Br)nc12 ; CCOC(=O)c1c(NC(=O)OC(C)(C)C)nn2ccc(C#C[Si](C)(C)C)nc12 ; C[C@@H](C1CC1)N1Cc2cc(N=[N+]=[N-])cc(C(F)(F)F)c2C1=O ; C#Cc1ccn2nc(NC(=O)OC(C)(C)C)c(C(=O)OCC)c2n1 ; C[C@@H](C1CC1)N1Cc2cc(-n3cc(-c4ccn5nc(NC(=O)OC(C)(C)C)c(C(=O)O)c5n4)nn3)cc(C(F)(F)F)c2C1=O ; C[C@H](N)c1cc2cccc(Cl)c2c(=O)n1-c1ccccc1]

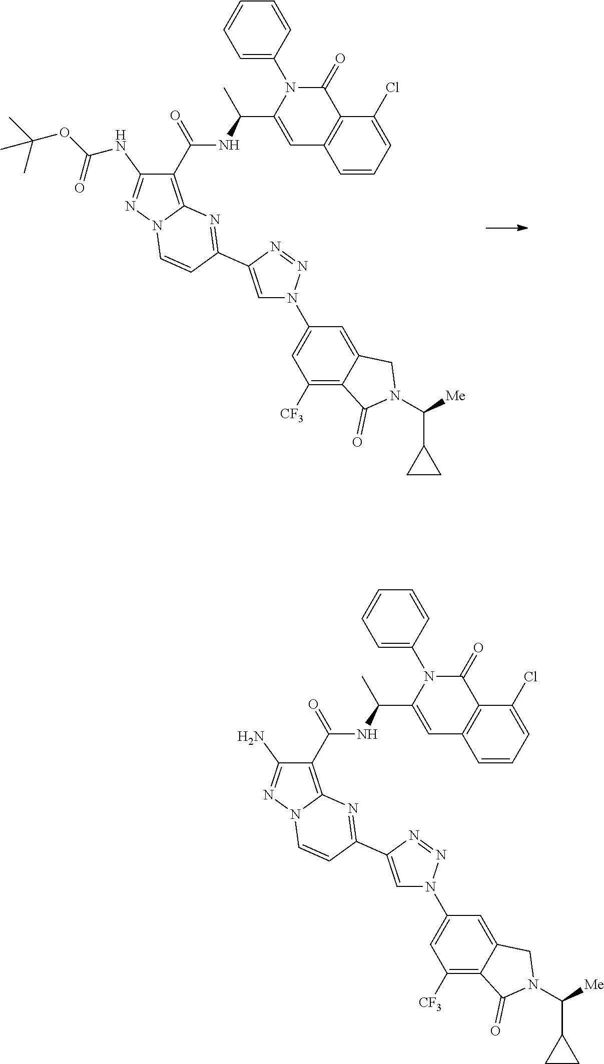 [SMILES: C[C@H](NC(=O)c1c(NC(=O)OC(C)(C)C)nn2ccc(-c3cn(-c4cc5c(c(C(F)(F)F)c4)C(=O)N([C@@H](C)C4CC4)C5)nn3)nc12)c1cc2cccc(Cl)c2c(=O)n1-c1ccccc1 ; C[C@H](NC(=O)c1c(N)nn2ccc(-c3cn(-c4cc5c(c(C(F)(F)F)c4)C(=O)N([C@@H](C)C4CC4)C5)nn3)nc12)c1cc2cccc(Cl)c2c(=O)n1-c1ccccc1]

### Step 1; ethyl 2-((tert-butoxycarbonyl)amino)-5-((trimethylsilyl)ethynyl)pyrazolo[1,5-a]pyrimidine-3-carboxylate

A mixture of ethyl 5-bromo-2-((tert-butoxycarbonyl)amino)pyrazolo[1,5-a]pyrimidine-3-carboxylate (1 equiv.), ethynyltrimethylsilane (2 equiv.), bis(diphenylphosphino)palladium dichloride (3 mol %), CuI (5 mol %) in dry NEt₃ (0.5 M) was heated at reflux for 16 h under argon. The reaction mixture was filtered over Celite® and washed with EtOAc. The filtrate was evaporated in vacuo, the residue was purified by column chromatography on silica gel eluting with a mixture of CH₂Cl2-MeOH step gradient solvent system as an eluent to afford the desired product.

### Step 2; ethyl 2-((tert-butoxycarbonyl)amino)-5-ethynylpyrazolo[1,5-a]pyrimidine-3-carboxylate

Tetrabutylammonium fluoride (1.0 M in tetrahydrofuran, 25 mL, 1.2 equiv.) was added to ethyl 2-((tert-butoxycarbonyl)amino)-5-((trimethylsilyl)ethynyl)pyrazolo[1,5-a]pyrimidine-3-carboxy late (1 eq). The reaction was stirred during 16 h after which the solvent was removed in vacuo and the crude residue was purified by column chromatography (SiO₂, CH₂Cl₂/-MeOH 9:1) to provide desired product.

### Step 3: (S)-2-((tert-butoxycarbonyl)amino)-5-(1-(2-(1-cyclopropylethyl)-1-oxo-7-(trifluoromethyl)isoindolin-5-yl)-1H-1,2,3-triazol-4-yl)pyrazolo[1,5-a]pyrimidine-3-carboxylic acid

(S)-5-azido-2-(1-cyclopropylethyl)-7-(trifluoromethyl)isoindolin-1-one (1 equiv, ethyl 2-((tert-butoxycarbonyl)amino)-5-ethynylpyrazolo[1,5-a]pyrimidine-3-carboxylate (1.2 equiv.), and CuI (10 mol %) were dissolved in degassed DMSO (0.5 M) and stirred at 60° C. for 16 h under argon. Upon completion of the reaction, water was added and the resulting suspension was removed by filtration. The residue was purified by flash chromatography using CH₂Cl2-MeOH step gradient solvent system as an eluent to afford the titled product.

### Step 4; tert-butyl (3-(((S)-1-(8-chloro-1-oxo-2-phenyl-1,2-dihydroisoquinolin-3-yl)ethyl)carbamoyl)-5-(1-(2-((S)-1-cyclopropylethyl)-1-oxo-7-(trifluoromethyl)isoindolin-5-yl)-1H-1,2,3-triazol-4-yl)pyrazolo[1,5-a]pyrimidin-2-yl)carbamate

According to general procedure 1. (S)-2-((tert-butoxycarbonyl)amino)-5-(1-(2-(1-cyclopropylethyl)-1-oxo-7-(trifluoromethyl)isoindolin-5-yl)-1H-1,2,3-triazol-4-yl)pyrazolo[1,5-a]pyrimidine-3-carboxylic acid (1 eq.), (S)-3-(1-aminoethyl)-8-chloro-2-phenylisoquinolin-1(2H)-one (1 eq) and HATU (0.95 eq.) in anhydrous DMF (0.05 M) was reacted together to afford the desired product.

### Step 5: 2-amino-N—((S)-1-(8-chloro-1-oxo-2-phenyl-1,2-dihydroisoquinolin-3-yl)ethyl)-5-(1-(2-((S)-1-cyclopropylethyl)-1-oxo-7-(trifluoromethyl)isoindolin-5-yl)-1H-1,2,3-triazol-4-yl)pyrazolo[1,5-a]pyrimidine-3-carboxamide

A solution of the tert-butyl (3-(((S)-1-(8-chloro-1-oxo-2-phenyl-1,2-dihydroisoquinolin-3-yl)ethyl)carbamoyl)-5-(1-(2-((S)-1-cyclopropylethyl)-1-oxo-7-(trifluoromethyl)isoindolin-5-yl)-1H-1,2,3-triazol-4-yl)pyrazolo[1,5-a]pyrimidin-2-yl)carbamate in HCl (4 M in dioxane, 0.05 M) was stirred at 0° C., to RT over 4 h. Then the mixture was dried in vacuo to afford the titled product.

### 2-amino-N—((S)-1-(8-chloro-1-oxo-2-phenyl-1,2-dihydroisoquinolin-3-yl)ethyl)-5-(2-((S)-1-cyclopropylethyl)-1-oxo-7-(trifluoromethyl)isoindoline-5-carboxamido)pyrazolo[1,5-a]pyrimidine-3-carboxamide

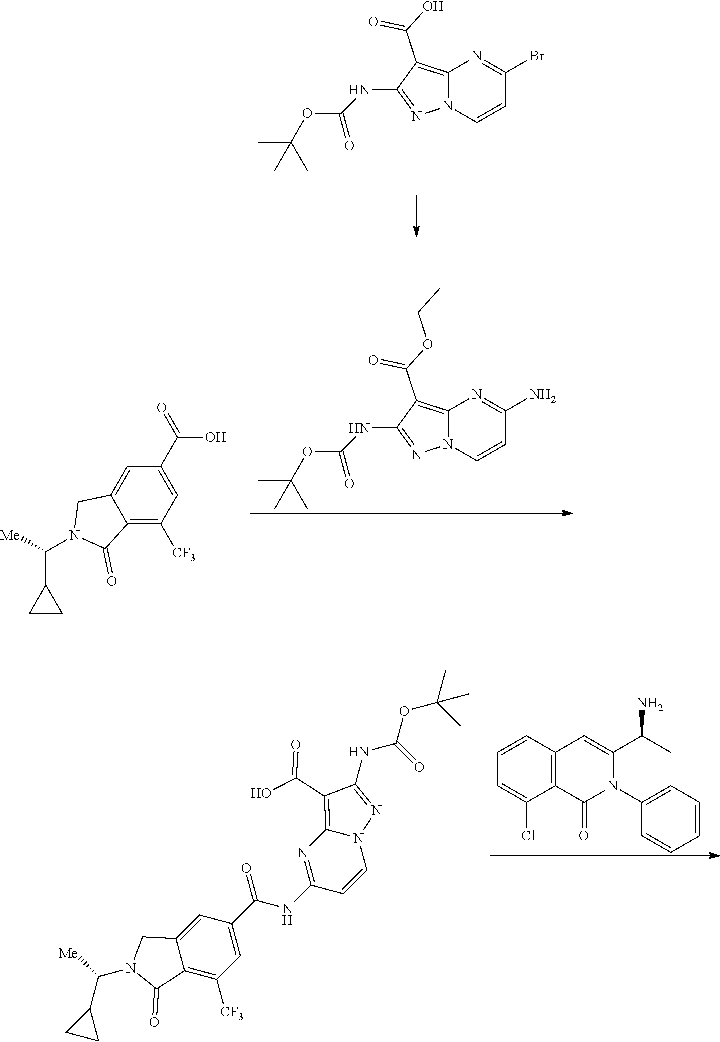

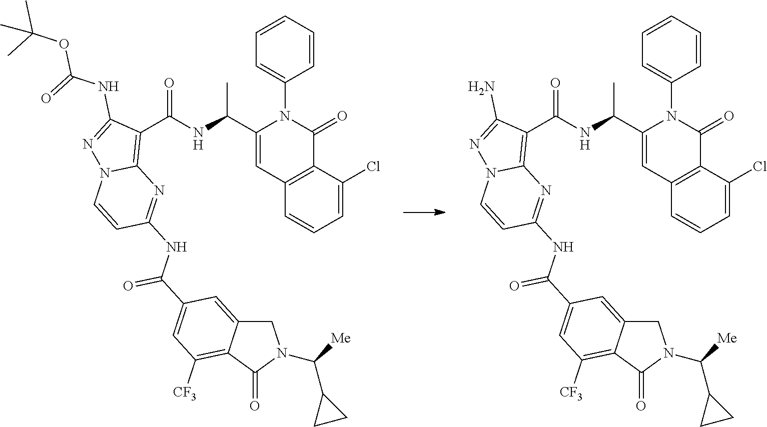 [SMILES: C[C@H](NC(=O)c1c(NC(=O)OC(C)(C)C)nn2ccc(NC(=O)c3cc4c(c(C(F)(F)F)c3)C(=O)N([C@@H](C)C3CC3)C4)nc12)c1cc2cccc(Cl)c2c(=O)n1-c1ccccc1 ; C[C@H](NC(=O)c1c(N)nn2ccc(NC(=O)c3cc4c(c(C(F)(F)F)c3)C(=O)N([C@@H](C)C3CC3)C4)nc12)c1cc2cccc(Cl)c2c(=O)n1-c1ccccc1]

### Step 1; ethyl 5-amino-2-((tert-butoxycarbonyl)amino)pyrazolo[1,5-a]pyrimidine-3-carboxylate

Ethyl 5-bromo-2-((tert-butoxycarbonyl)amino)pyrazolo[1,5-a]pyrimidine-3-carboxylate in ammonia (7 M in MeOH, 0.05 M) was refluxed for 16 h at 100° ° C. to afford the desired product.

### Step 2: (S)-2-((tert-butoxycarbonyl)amino)-5-(2-(1-cyclopropylethyl)-1-oxo-7-(trifluoromethyl)isoindoline-5-carboxamido)pyrazolo[1,5-a]pyrimidine-3-carboxylic acid

According to General Procedure 1 (S)-2-(1-cyclopropylethyl)-1-oxo-7-(trifluoromethyl)isoindoline-5-carboxylic acid (1 eq.), ethyl 5-amino-2-((tert-butoxycarbonyl)amino)pyrazolo[1,5-a]pyrimidine-3-carboxylate (1 eq) and HATU (0.95 eq.) in anhydrous DMF (0.05 M) was reacted together to afford the desired product.

### Step 3; tert-butyl (3-(((S)-1-(8-chloro-1-oxo-2-phenyl-1,2-dihydroisoquinolin-3-yl)ethyl)carbamoyl)-5-(2-((S)-1-cyclopropylethyl)-1-oxo-7-(trifluoromethyl)isoindoline-5-carboxamido)pyrazolo[1,5-a]pyrimidin-2-yl)carbamate

According to General Procedure 1 (S)-2-((tert-butoxycarbonyl)amino)-5-(2-(1-cyclopropylethyl)-1-oxo-7-(trifluoromethyl)isoindoline-5-carboxamido)pyrazolo[1,5-a]pyrimidine-3-carboxylic acid (1 eq.), ethyl (S)-3-(1-aminoethyl)-8-chloro-2-phenylisoquinolin-1(2H)-one (1 eq) and HATU (0.95 eq.) in anhydrous DMF (0.05 M) was reacted together to afford the desired product.

### Step 5: 2-amino-N—((S)-1-(8-chloro-1-oxo-2-phenyl-1,2-dihydroisoquinolin-3-yl)ethyl)-5-(2-((S)-1-cyclopropylethyl)-1-oxo-7-(trifluoromethyl)isoindoline-5-carboxamido)pyrazolo[1,5-a]pyrimidine-3-carboxamide

A solution of the tert-butyl (3-(((S)-1-(8-chloro-1-oxo-2-phenyl-1,2-dihydroisoquinolin-3-yl)ethyl)carbamoyl)-5-(2-((S)-1-cyclopropylethyl)-1-oxo-7-(trifluoromethyl)isoindoline-5-carboxamido)pyrazolo[1,5-a]pyrimidin-2-yl)carbamate in HCl (4 M in dioxane, 0.05 M) was stirred at 0° C. to RT during 4 h. Then the mixture was dried in vacuo to afford the titled product.

### Example 3

### Series 3 and 4

### General Procedure 7: Esterification

Under a nitrogen atmosphere, a solution of linoleic acid/or N^{α}-(tert-butoxycarbonyl)-1-methyl-D-tryptophan (1.5 eq.). EDC hydrochloride (1.4 eq.), and DMAP (0.5 eq.) in dry THF (0.1 M) was stirred at 0° C. for 0.5 h. Following, a solution of Arylethynyl-pyrazole (or pyrrole) (1 eq.) in dry THF (0.1 M) was added slowly by a syringe. The solution was stirred at 0° C. to RT for 16 h (unless noted otherwise). Progress of the reaction was monitored by TLC using CH₂Cl₂-MeOH 10:1 mixture as eluent and 1 H NMR using ARX 300 Brucker spectrometer. Upon completion of the reaction, the mixture was extracted with dichloromethane (3×). The combined organic layer and washed with 0.2 M HCl aqueous, saturated aqueous solution of NaHCO₃ and brine, respectively. Then, the solid residue was purified by manual column chromatography using CH₂Cl₂-EtOAc or CH₂Cl₂-MeOH step gradient solvent system as an eluent. Compounds 8, 9, 10 and 11 were prepared using this procedure.

### General Procedure 13 Boc Deprotection

A solution of the Boc-protected amine in HCl (4 M in dioxane, 0.05 M) was stirred at 0° C. to RT during 4 h (unless mentioned otherwise). Then the mixture was dried in vacuo to afford the titled product.

### 2-(4-((3-((S)-1-(2-aminopyrazolo[1,5-a]pyrimidine-3-carboxamido)ethyl)-1-oxo-2-phenyl-1,2-dihydroisoquinolin-8-yl)ethynyl)-1H-pyrazol-1-yl)ethyl (9Z,12Z)-octadeca-9,12-dienoate

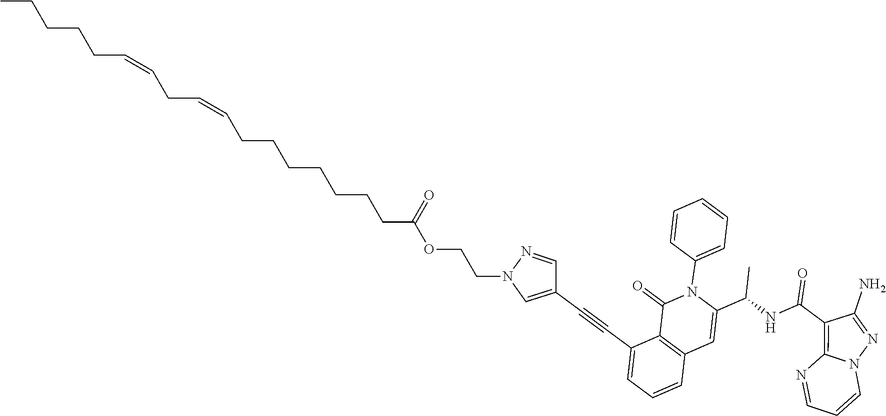 [SMILES: CCCCC/C=C\C/C=C\CCCCCCCC(=O)OCCn1cc(C#Cc2cccc3cc([C@H](C)NC(=O)c4c(N)nn5cccnc45)n(-c4ccccc4)c(=O)c23)cn1]

The desired product was obtained from (S)-2-amino-N-(1-(8-((1-(2-hydroxyethyl)-1H-pyrazol-4-yl)ethynyl)-1-oxo-2-phenyl-1,2-dihydroisoquinolin-3-yl)ethyl)pyrazolo[1,5-a]pyrimidine-3-carboxamide (40 mg, 0.072 mmol, 1 equiv.), linoleic acid (34 mL, 0.107 mmol, 1.5 eq.), EDC hydrochloride (19 mg, 0.101 mmol, 1.4 eq.), and DMAP (5 mg, 0.036 mmol, 0.5 eq.) in dry DMF (2.4 mL, 0.03 M) according to General Procedure 7.

### (1-(4-((3-((S)-1-(2-aminopyrazolo[1,5-a]pyrimidine-3-carboxamido)ethyl)-1-oxo-2-phenyl-1,2-dihydroisoquinolin-8-yl)ethynyl)-1H-pyrazol-1-yl)cyclopropyl)methyl (9Z,12Z)-octadeca-9,12-dienoate

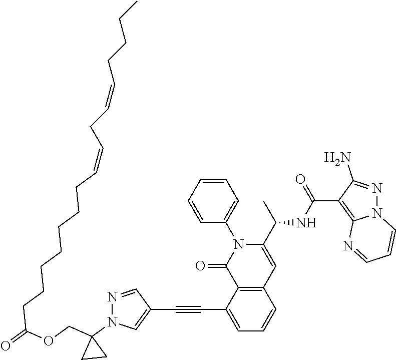 [SMILES: CCCC/C=C\C/C=C\CCCCCCCC(=O)OCC1(n2cc(C#Cc3cccc4cc([C@H](C)NC(=O)c5c(N)nn6cccnc56)n(-c5ccccc5)c(=O)c34)cn2)CC1]

The desired product was obtained as a solid from (S)-2-amino-N-(1-(8-((1-(1-(hydroxymethyl)cyclopropyl)-1H-pyrazol-4-yl)ethynyl)-1-oxo-2-phenyl-1,2-dihydroisoquinolin-3-yl)ethyl)pyrazolo[1,5-a]pyrimidine-3-carboxamide (42 mg, 0.072 mmol, 1 equiv.), linoleic acid (34 mL, 0.107 mmol, 1.5 eq.), EDC hydrochloride (19 mg, 0.101 mmol, 1.4 eq.), and DMAP (5 mg, 0.036 mmol, 0.5 eq.) in dry DMF (2.4 mL, 0.03 M) according to General Procedure 7.

### 2-(4-((3-((S)-1-(2-aminopyrazolo[1,5-a]pyrimidine-3-carboxamido)ethyl)-1-oxo-2-phenyl-1,2-dihydroisoquinolin-8-yl)ethynyl)-1H-1,2,3-triazol-1-yl)ethyl (9Z,12Z)-octadeca-9,12-dienoate

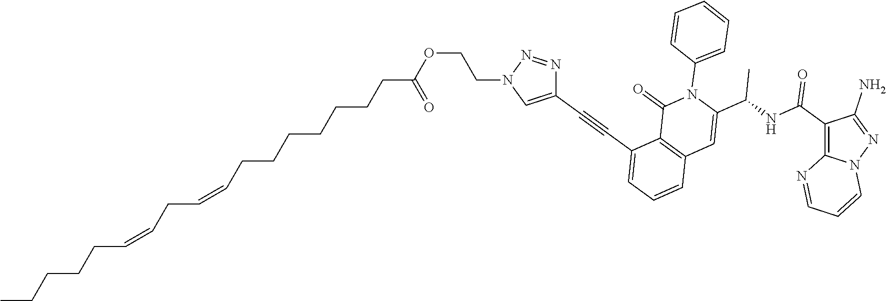 [SMILES: CCCCC/C=C\C/C=C\CCCCCCCC(=O)OCCn1cc(C#Cc2cccc3cc([C@H](C)NC(=O)c4c(N)nn5cccnc45)n(-c4ccccc4)c(=O)c23)nn1]

The desired product was obtained as a solid from (S)-2-amino-N-(1-(8-((1-(2-hydroxyethyl)-1H-1,2,3-triazol-4-yl)ethynyl)-1-oxo-2-phenyl-1,2-dihydroisoquinolin-3-yl)ethyl)pyrazolo[1,5-a]pyrimidine-3-carboxamide (41 mg, 0.072 mmol, 1 equiv.), linoleic acid (34 mL, 0.107 mmol, 1.5 eq.), EDC hydrochloride (19 mg, 0.101 mmol, 1.4 eq.), and DMAP (5 mg, 0.036 mmol, 0.5 eq.) in dry DMF (2.4 mL, 0.03 M) according to General Procedure 7.

### (1-(4-((3-((S)-1-(2-aminopyrazolo[1,5-a]pyrimidine-3-carboxamido)ethyl)-1-oxo-2-phenyl-1,2-dihydroisoquinolin-8-yl)ethynyl)-1H-1,2,3-triazol-1-yl)cyclopropyl)methyl (9Z,12Z)-octadeca-9,12-dienoate

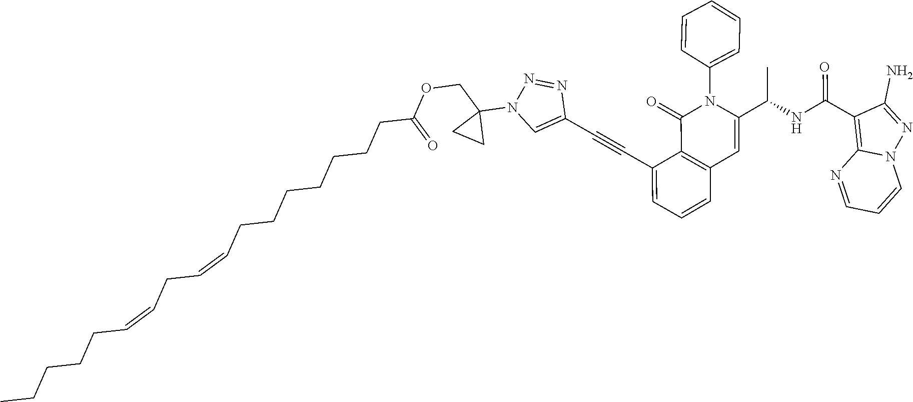 [SMILES: CCCCC/C=C\C/C=C\CCCCCCCC(=O)OCC1(n2cc(C#Cc3cccc4cc([C@H](C)NC(=O)c5c(N)nn6cccnc56)n(-c5ccccc5)c(=O)c34)nn2)CC1]

The desired product was obtained as a solid from (S)-2-amino-N-(1-(8-((1-(1-(hydroxymethyl)cyclopropyl)-1H-1,2,3-triazol-4-yl)ethynyl)-1-oxo-2-phenyl-1,2-dihydroisoquinolin-3-yl)ethyl)pyrazolo[1,5-a]pyrimidine-3-carboxamide (42 mg, 0.072 mmol, 1 equiv.), linoleic acid (34 mL, 0.107 mmol, 1.5 eq.), EDC hydrochloride (19 mg, 0.101 mmol, 1.4 eq.), and DMAP (5 mg, 0.036 mmol, 0.5 eq.) in dry DMF (2.4 mL, 0.03 M) according to General Procedure 7.

### (1-(4-((3-((S)-1-(2-aminopyrazolo[1,5-a]pyrimidine-3-carboxamido)ethyl)-1-oxo-2-phenyl-1,2-dihydroisoquinolin-8-yl)ethynyl)-1H-pyrazole-1-carbonyl)cyclopropyl)methyl (9Z,12Z)-octadeca-9,12-dienoate

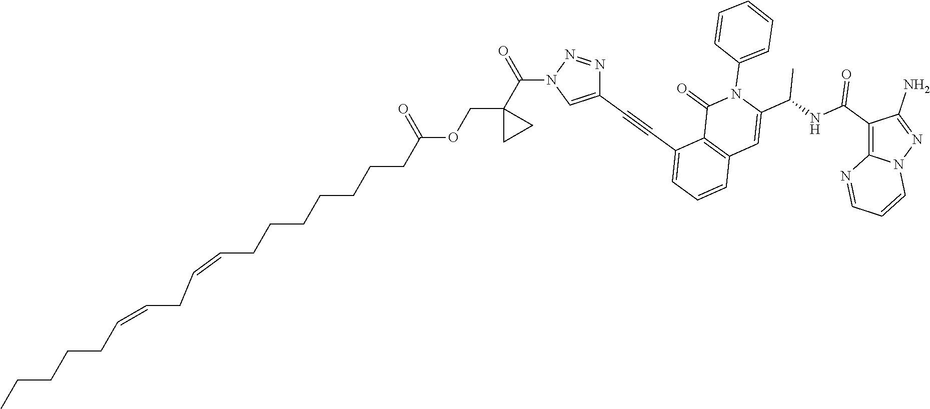 [SMILES: CCCCC/C=C\C/C=C\CCCCCCCC(=O)OCC1(C(=O)n2cc(C#Cc3cccc4cc([C@H](C)NC(=O)c5c(N)nn6cccnc56)n(-c5ccccc5)c(=O)c34)nn2)CC1]

The desired product was obtained as a solid from (S)-2-amino-N-(1-(8-((1-(1-(hydroxymethyl)cyclopropane-1-carbonyl)-1H-pyrazol-4-yl)ethynyl)-1-oxo-2-phenyl-1,2-dihydroisoquinolin-3-yl)ethyl)pyrazolo[1,5-a]pyrimidine-3-carboxamide (44 mg, 0.072 mmol, 1 equiv.), linoleic acid (34 mL, 0.107 mmol, 1.5 eq.), EDC hydrochloride (19 mg, 0.101 mmol, 1.4 eq.), and DMAP (5 mg, 0.036 mmol, 0.5 eq.) in dry DMF (2.4 mL, 0.03 M) according to General Procedure 7.

### (1-(4-((3-((S)-1-(2-aminopyrazolo[1,5-a]pyrimidine-3-carboxamido)ethyl)-1-oxo-2-phenyl-1,2-dihydroisoquinolin-8-yl)ethynyl)-1H-1,2,3-triazole-1-carbonyl)cyclopropyl)methyl (9Z,12Z)-octadeca-9,12-dienoate

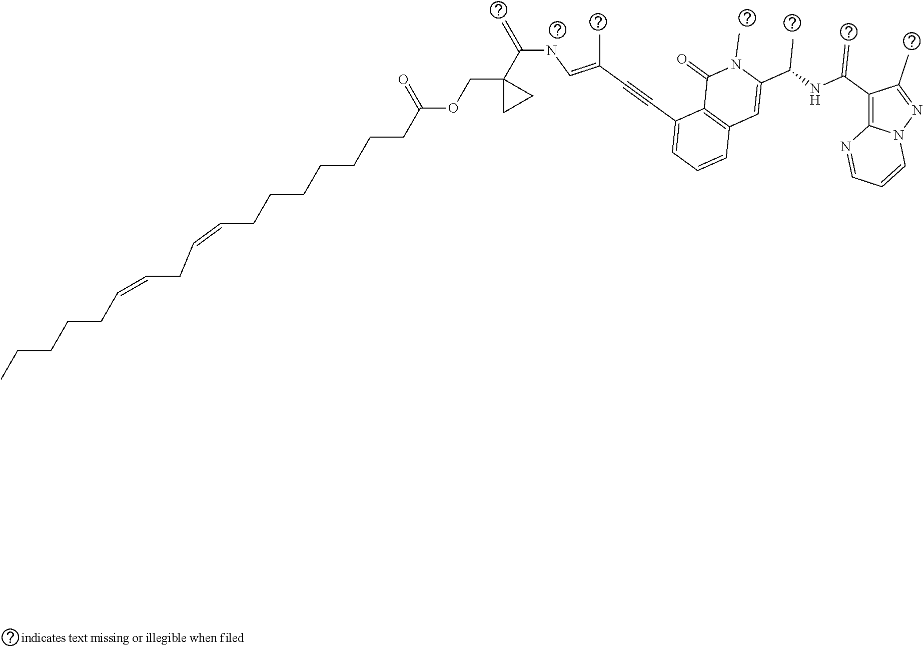 [SMILES: C=C(N[C@@H](C)c1cc2cccc(C#C/C(C)=C/NC(=C)C3(COC(=O)CCCCCCC/C=C\C/C=C\CCCCC)CC3)c2c(=O)n1C)c1c(C)nn2cccnc12]

Desired product was obtained as a solid from (S)-2-amino-N-(1-(8-((1-(1-(hydroxymethyl)cyclopropane-1-carbonyl)-1H-1,2,3-triazol-4-yl)ethynyl)-1-oxo-2-phenyl-1,2-dihydroisoquinolin-3-yl)ethyl)pyrazolo[1,5-a]pyrimidine-3-carboxamide (44 mg, 0.072 mmol, 1 equiv.), linoleic acid (34 mL, 0.107 mmol, 1.5 eq.), EDC hydrochloride (19 mg, 0.101 mmol, 1.4 eq.), and DMAP (5 mg, 0.036 mmol, 0.5 eq.) in dry DMF (2.4 mL, 0.03 M) according to General Procedure 7.

### (4-((3-((S)-1-(2-aminopyrazolo[1,5-a]pyrimidine-3-carboxamido)ethyl)-1-oxo-2-phenyl-1,2-dihydroisoquinolin-8-yl)ethynyl)-1-methyl-1H-pyrrol-2-yl)methyl (97,12Z)-octadeca-9,12-dienoate

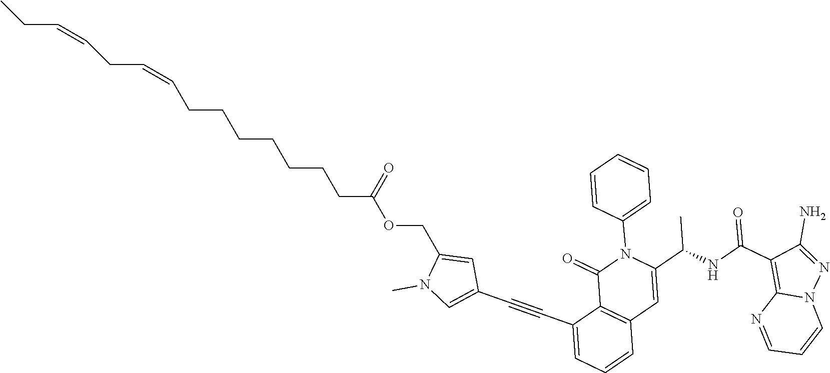 [SMILES: CC/C=C\C/C=C\CCCCCCCC(=O)OCc1cc(C#Cc2cccc3cc([C@H](C)NC(=O)c4c(N)nn5cccnc45)n(-c4ccccc4)c(=O)c23)cn1C]

The desired product was obtained as a solid from (S)-2-amino-N-(1-(8-((5-(hydroxymethyl)-1-methyl-1H-pyrrol-3-yl)ethynyl)-1-oxo-2-phenyl-1,2-dihydroisoquinolin-3-yl)ethyl)pyrazolo[1,5-a]pyrimidine-3-carboxamide (40 mg, 0.072 mmol, I equiv.), linoleic acid (34 mL, 0.107 mmol, 1.5 eq.), EDC hydrochloride (19 mg, 0.101 mmol, 1.4 eq.), and DMAP (5 mg, 0.036 mmol, 0.5 eq.) in dry DMF (2.4 mL, 0.03 M) according to General Procedure 7.

### 2-(4-((3-((S)-1-(2-aminopyrazolo[1,5-a]pyrimidine-3-carboxamido)ethyl)-1-oxo-2-phenyl-1,2-dihydroisoquinolin-8-yl)ethynyl)-1H-pyrazol-1-yl)ethyl 1-methyl-D-tryptophanate

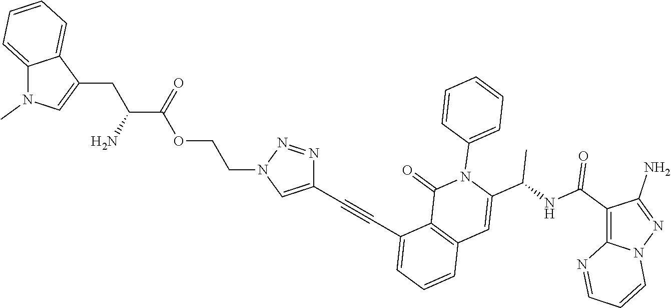 [SMILES: C[C@H](NC(=O)c1c(N)nn2cccnc12)c1cc2cccc(C#Cc3cn(CCOC(=O)[C@H](N)Cc4cn(C)c5ccccc45)nn3)c2c(=O)n1-c1ccccc1]

Step 1: Desired product was obtained as a solid from (S)-2-amino-N-(1-(8-((1-(2-hydroxyethyl)-1H-pyrazol-4-yl)ethynyl)-1-oxo-2-phenyl-1,2-dihydroisoquinolin-3-yl)ethyl)pyrazolo[1,5-a]pyrimidine-3-carboxamide (40 mg, 0.072 mmol, I equiv.), N^{α}-(tert-butoxycarbonyl)-1-methyl-D-tryptophan (35 mg, 0.107 mmol, 1.5 eq.), EDC hydrochloride (19 mg, 0.101 mmol, 1.4 eq.), and DMAP (5 mg, 0.036 mmol, 0.5 eq.) in dry DMF (2.4 mL, 0.03 M) according to General Procedure 7.

Step 2: Desired product was obtained as a solid from 2-(4-((3-((S)-1-(2-aminopyrazolo[1,5-a]pyrimidine-3-carboxamido)ethyl)-1-oxo-2-phenyl-1,2-dihydroisoquinolin-8-yl)ethynyl)-1H-pyrazol-1-yl)ethyl N^{α}-(tert-butoxycarbonyl)-1-methyl-D-tryptophanate after a 4 h stirring in HCl (4 M in dioxane, 0.05 M) according to general procedure 13.

### (1-(4-((3-((S)-1-(2-aminopyrazolo[1,5-a]pyrimidine-3-carboxamido)ethyl)-1-oxo-2-phenyl-1,2-dihydroisoquinolin-8-yl)ethynyl)-1H-pyrazol-1-yl)cyclopropyl)methyl 1-methyl-D-tryptophanate

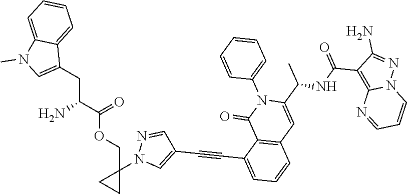 [SMILES: C[C@H](NC(=O)c1c(N)nn2cccnc12)c1cc2cccc(C#Cc3cnn(C4(COC(=O)[C@H](N)Cc5cn(C)c6ccccc56)CC4)c3)c2c(=O)n1-c1ccccc1]

Step 1: (S)-2-amino-N-(1-(8-((1-(1-(hydroxymethyl)cyclopropyl)-1H-pyrazol-4-yl)ethynyl)-1-oxo-2-phenyl-1,2-dihydroisoquinolin-3-yl)ethyl)pyrazolo[1,5-a]pyrimidine-3-carboxamide (42 mg, 0.072 mmol, I equiv.) was reacted with N^{α}-(tert-butoxycarbonyl)-1-methyl-D-tryptophan (35 mg, 0.107 mmol, 1.5 eq.), EDC hydrochloride (19 mg, 0.101 mmol, 1.4 eq.), and DMAP (5 mg, 0.036 mmol, 0.5 eq.) in dry DMF (2.4 mL, 0.03 M) according to General Procedure 7.

Step 2: The desired product was obtained as a solid from (1-(4-((3-((S)-1-(2-aminopyrazolo[1,5-a]pyrimidine-3-carboxamido)ethyl)-1-oxo-2-phenyl-1,2-dihydroisoquinolin-8-yl)ethynyl)-1H-pyrazol-1-yl)cyclopropyl)methyl N^{α}-(tert-butoxycarbonyl)-1-methyl-D-tryptophanate after a 4 h stirring in HCl (4 M in dioxane, 0.05 M) according to general procedure 13.

### 2-(4-((3-((S)-1-(2-aminopyrazolo[1,5-a]pyrimidine-3-carboxamido)ethyl)-1-oxo-2-phenyl-1,2-dihydroisoquinolin-8-yl)ethynyl)-1H-1,2,3-triazol-1-yl)ethyl 1-methyl-D-tryptophanate

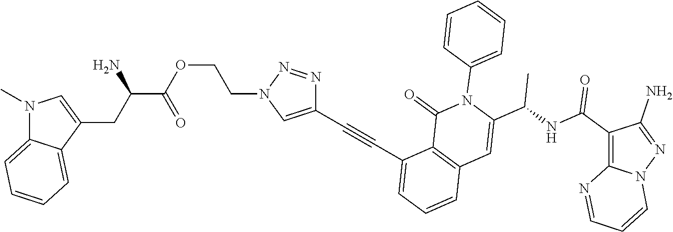 [SMILES: C[C@H](NC(=O)c1c(N)nn2cccnc12)c1cc2cccc(C#Cc3cn(CCOC(=O)[C@H](N)Cc4cn(C)c5ccccc45)nn3)c2c(=O)n1-c1ccccc1]

Step 1: (S)-2-amino-N-(1-(8-((1-(2-hydroxyethyl)-1H-1,2,3-triazol-4-yl)ethynyl)-1-oxo-2-phenyl-1,2-dihydroisoquinolin-3-yl)ethyl)pyrazolo[1,5-a]pyrimidine-3-carboxamide (41 mg, 0.072 mmol, 1 equiv.) was reacted with N^{α}-(tert-butoxycarbonyl)-1-methyl-D-tryptophan (35 mg, 0.107 mmol, 1.5 eq.), EDC hydrochloride (19 mg, 0.101 mmol, 1.4 eq.), and DMAP (5 mg, 0.036 mmol, 0.5 eq.) in dry DMF (2.4 mL, 0.03 M) according to General Procedure 7.

Step 2: The desired product was obtained as a solid from 2-(4-((3-((S)-1-(2-aminopyrazolo[1,5-a]pyrimidine-3-carboxamido)ethyl)-1-oxo-2-phenyl-1,2-dihydroisoquinolin-8-yl)ethynyl)-1H-1,2,3-triazol-1-yl)ethyl N^{α}-(tert-butoxycarbonyl)-1-methyl-D-tryptophanate after a 4 h stirring in HCl (4 M in dioxane, 0.05 M) according to general procedure 13.

### (1-(4-((3-((S)-1-(2-aminopyrazolo[1,5-a]pyrimidine-3-carboxamido)ethyl)-1-oxo-2-phenyl-1,2-dihydroisoquinolin-8-yl)ethynyl)-1H-1,2,3-triazol-1-yl)cyclopropyl)methyl 1-methyl-D-tryptophanate

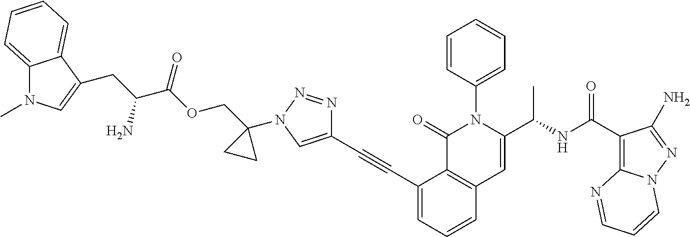 [SMILES: C[C@H](NC(=O)c1c(N)nn2cccnc12)c1cc2cccc(C#Cc3cn(C4(COC(=O)[C@H](N)Cc5cn(C)c6ccccc56)CC4)nn3)c2c(=O)n1-c1ccccc1]

Step 1: (S)-2-amino-N-(1-(8-((1-(1-(hydroxymethyl)cyclopropyl)-1H-1,2,3-triazol-4-yl)ethynyl)-1-oxo-2-phenyl-1,2-dihydroisoquinolin-3-yl)ethyl)pyrazolo[1,5-a]pyrimidine-3-carboxamide (42 mg, 0.072 mmol, I equiv.) was reacted with No-(tert-butoxycarbonyl)-1-methyl-D-tryptophan (35 mg, 0.107 mmol, 1.5 eq.), EDC hydrochloride (19 mg, 0.101 mmol, 1.4 eq.), and DMAP (5 mg, 0.036 mmol, 0.5 eq.) in dry DMF (2.4 mL, 0.03 M) according to General Procedure 7.

Step 2: The desired product was obtained as a solid (1-(4-((3-((S)-1-(2-aminopyrazolo[1,5-a]pyrimidine-3-carboxamido)ethyl)-1-oxo-2-phenyl-1,2-dihydroisoquinolin-8-yl)ethynyl)-1H-1,2,3-triazol-1-yl)cyclopropyl)methyl N^{α}-(tert-butoxycarbonyl)-1-methyl-D-tryptophanate after a 4 h stirring in HCl (4 M in dioxane, 0.05 M) according to general procedure 13.

### (1-(4-((3-((S)-1-(2-aminopyrazolo[1,5-a]pyrimidine-3-carboxamido)ethyl)-1-oxo-2-phenyl-1,2-dihydroisoquinolin-8-yl)ethynyl)-1H-pyrazole-1-carbonyl)cyclopropyl)methyl 1-methyl-D-tryptophanate

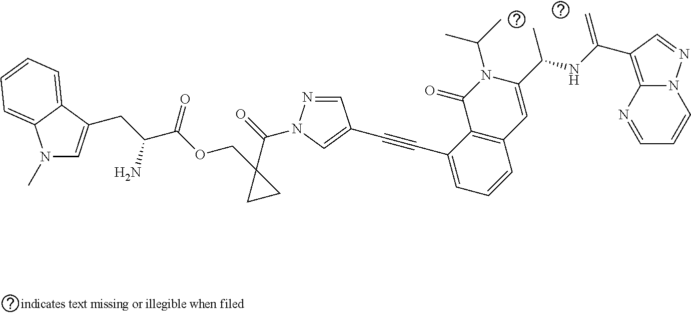 [SMILES: C=C(N[C@@H](C)c1cc2cccc(C#Cc3cnn(C(=O)C4(COC(=O)[C@H](N)Cc5cn(C)c6ccccc56)CC4)c3)c2c(=O)n1C(C)C)c1cnn2cccnc12]

Step 1: (S)-2-amino-N-(1-(8-((1-(1-(hydroxymethyl)cyclopropane-1-carbonyl)-1H-pyrazol-4-yl)ethynyl)-1-oxo-2-phenyl-1,2-dihydroisoquinolin-3-yl)ethyl)pyrazolo[1,5-a]pyrimidine-3-carboxamide (44 mg, 0.072 mmol, 1 equiv.) was reacted with N^{α}-(tert-butoxycarbonyl)-1-methyl-D-tryptophan (35 mg, 0.107 mmol, 1.5 eq.), EDC hydrochloride (19 mg, 0.101 mmol, 1.4 eq.), and DMAP (5 mg, 0.036 mmol, 0.5 eq.) in dry DMF (2.4 mL, 0.03 M) according to General Procedure 7.

Step 2: The desired product was obtained as a solid from (1-(4-((3-((S)-1-(2-aminopyrazolo[1,5-a]pyrimidine-3-carboxamido)ethyl)-1-oxo-2-phenyl-1,2-dihydroisoquinolin-8-yl)ethynyl)-1H-pyrazole-1-carbonyl)cyclopropyl)methyl Na-(tert-butoxycarbonyl)-1-methyl-D-tryptophanate after a 4 h stirring in HCl (4 M in dioxane, 0.05 M) according to general procedure 13.

### (1-(4-((3-((S)-1-(2-aminopyrazolo[1,5-a]pyrimidine-3-carboxamido)ethyl)-1-oxo-2-phenyl-1,2-dihydroisoquinolin-8-yl)ethynyl)-1H-1,2,3-triazole-1-carbonyl)cyclopropyl)methyl 1-methyl-D-tryptophanate

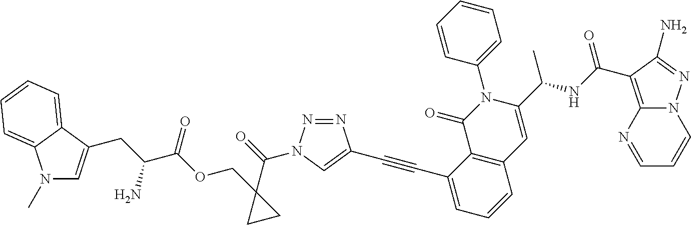 [SMILES: C[C@H](NC(=O)c1c(N)nn2cccnc12)c1cc2cccc(C#Cc3cn(C(=O)C4(COC(=O)[C@H](N)Cc5cn(C)c6ccccc56)CC4)nn3)c2c(=O)n1-c1ccccc1]

Step 1: (S)-2-amino-N-(1-(8-((1-(1-(hydroxymethyl)cyclopropane-1-carbonyl)-1H-1,2,3-triazol-4-yl)ethynyl)-1-oxo-2-phenyl-1,2-dihydroisoquinolin-3-yl)ethyl)pyrazolo[1,5-a]pyrimidine-3-carboxamide (44 mg, 0.072 mmol, I equiv.) was reacted with N^{α}-(tert-butoxycarbonyl)-1-methyl-D-tryptophan (35 mg, 0.107 mmol, 1.5 eq.), EDC hydrochloride (19 mg, 0.101 mmol, 1.4 eq.), and DMAP (5 mg, 0.036 mmol, 0.5 eq.) in dry DMF (2.4 mL, 0.03 M) according to General Procedure 7.

Step 2: The desired product was obtained as a solid from (1-(4-((3-((S)-1-(2-aminopyrazolo[1,5-a]pyrimidine-3-carboxamido)ethyl)-1-oxo-2-phenyl-1,2-dihydroisoquinolin-8-ylethynyl)-1H-1,2,3-triazole-1-carbonyl)cyclopropyl)methyl N^{α}-(tert-butoxycarbonyl)-1-methyl-D-tryptophanate after a 4 h stirring in HCl (4 M in dioxane, 0.05 M) according to general procedure 13.

### (4-((3-((S)-1-(2-aminopyrazolo[1,5-a]pyrimidine-3-carboxamido)ethyl)-1-oxo-2-phenyl-1,2-dihydroisoquinolin-8-yl)ethynyl)-1-methyl-1H-pyrrol-2-yl)methyl 1-methyl-D-tryptophanate

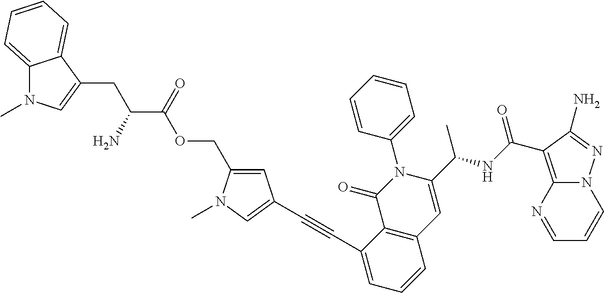 [SMILES: C[C@H](NC(=O)c1c(N)nn2cccnc12)c1cc2cccc(C#Cc3cc(COC(=O)[C@H](N)Cc4cn(C)c5ccccc45)n(C)c3)c2c(=O)n1-c1ccccc1]

Step 1: (S)-2-amino-N-(1-(8-((5-(hydroxymethyl)-1-methyl-1H-pyrrol-3-yl)ethynyl)-1-oxo-2-phenyl-1,2-dihydroisoquinolin-3-yl)ethyl)pyrazolo[1,5-a]pyrimidine-3-carboxamide (40 mg, 0.072 mmol, 1 equiv.) was reacted with N^{α}-(tert-butoxycarbonyl)-1-methyl-D-tryptophan (35 mg, 0.107 mmol, 1.5 eq.), EDC hydrochloride (19 mg, 0.101 mmol, 1.4 eq.), and DMAP (5 mg, 0.036 mmol, 0.5 eq.) in dry DMF (2.4 mL, 0.03 M) according to General Procedure 7.

Step 2: The desired product was obtained as a solid (4-((3-((S)-1-(2-aminopyrazolo[1,5-a]pyrimidine-3-carboxamido)ethyl)-1-oxo-2-phenyl-1,2-dihydroisoquinolin-8-yl)ethynyl)-1-methyl-1H-pyrrol-2-yl)methyl N^{α}-(tert-butoxycarbonyl)-1-methyl-D-tryptophanate after a 4 h stirring in HCl (4 M in dioxane, 0.05 M) according to general procedure 13.

### Example 4

### Nanomedicine of PI3Kγ Inhibitor Combined with αPD-1 for Cancer Treatment

### Materials and Methods

Mice and cell lines. Female mice including the BALB/c mice and FVB/NJ mice were purchased from the Jackson Laboratory (Bar Harbor, ME, USA). The transgenic MMTV polyoma middle T oncoprotein (PyMT) mice were generated by crossing FVB/NJ females (Stock No. 001800) with hemizygous FVB/N-Tg (MMTV-PyMT) 634Mul/J males (Stock No: 002374). All animal experiments were performed following the Guiding Principles (approval reference number is PRO00009633) of the University Committee on Use and Care Animals at the University of Michigan.

The RAW 264.7 (TIB-71™) and 4T1 cell lines (CRL-2539™) were purchased from the American Type Culture Collection (Rockville, DS, USA). All cell types were maintained in the Dulbecco's Modified Eagle Medium (DMEM, Gibco, NY, USA) supplemented with 10% heat-inactivated fetal bovine serum (FBS) at 37° ° C. and 5% CO₂.

Preparation and characterization of Nano-PI. PTX (12 mg) and IPI-549 (8 mg) were dissolved in 1 mL of chloroform and then added dropwise into 100 mg of mouse serum albumin dissolved in 20 mL milli-Q water to generate a milky emulsion using a rotor-stator homogenizer. The Nano-PI nanosuspension was obtained after running 5-6 cycles at 30000 psi on a high-pressure homogenizer (Nano DeBEE) at 4° C. The organic solvent was removed using a rotary evaporator at 25° C. After filtering (0.22 μm), the Nano-PI suspension was lyophilized and stored at −20° C. Nano-P was prepared following the same procedures but with a PTX to mouse albumin ratio of 1:5. The size distribution and morphology were measured by Dynamic Light Scattering (DLS) and JEOL2010F transmission electron microscopy (TEM). The drug concentration in Nano-PI were detected by LC-MS/MS. The encapsulation efficiency (EE), drug loading capacity (DL) and drug recovery yield (Y) were calculated by the equations below.

$$EE ( \% ) = \frac{Wd - Wf}{Wd} \times 100 \% \qquad ( Eq 1 )$$

$$DL ( \% ) = \frac{Wd}{Wn} \times 100 \% \qquad ( Eq 2 )$$

$$Y ( \% ) = \frac{Wt}{Wa} \times 100 \% \qquad ( Eq 3 )$$

where the W_{d} represents the amount of drug in Nano-PI suspension; W_{f} represents the amount of free drug: Wₙ represents the amount of Nano-PI suspension: Wₜ represents the total amount of drug in the Nano-PI suspension after process: Wₐ represents total drug added. The free drug was separated from Nano-PI formulation by a Nanosep centrifugal devices (MWCO 3 KDa) with the centrifugation speed of 10, 000 rpm/min for 10 min.

Macrophage polarization. BMDMs were isolated from the femurs and tibias of BALB/c mice (female, 7 weeks old). Briefly, after euthanizing the mice, the femurs and tibia of the hind legs were collected, and the bone marrow cells were gently flushed with pre-cold RIPA 1640. After centrifugation, cells were re-suspended in complete DMEM containing 2 mM L-glutamine, 10% FBS, 10 ng/mL macrophage colony-stimulating factor (M-SF) (PeproTech Inc.), 50 U/mL penicillin, and 50 μg/mL streptomycin, and seeded into sterile plastic petri dishes (10 mL) at a density of 5×10⁶ cells/dish. The medium was replaced on day 3 and on day 7 and the BMDM cells were harvested and used for further experiments. BMDMs and RAW 264.7 cells were stimulated with LPS (100 ng/ml), IFNγ (50 ng/mL), IL-4 (20 ng/mL), and IL-13 (10 ng/mL), respectively, to generate M1/M2 macrophages. Macrophage morphology was observed using an inverted fluorescence microscope (Olympus). In addition, the cells and the supernatant medium in each well were collected, and the expression of CD80, INOS, and CD206 was separately measured using western blotting, and the secretion of cytokines, including TNF-α, TGF-β, IL-12, and IL-10, were detected by ELISA.

3D multicellular tumor spheroids (MICs), 4T1 cells and M2 macrophages derived from RAW264.7 cells were mixed in the ratio of 7:3 and then seeded into an ultralow-attachment 96 well plate (Corning) at a density of 5,000 cells/well and cultured for 30 h to form the tumor spheroids. These tumor spheroids were separately incubated with 300 μL complete medium supplemented with PTX (5 μm), gemcitabine (5 μM), doxorubicin (5 μM), and IPI-549 (5 μM) as well as the combination of IPI-549 and PTX (2.5 μM+2.5 M), IPI-549 and gemcitabine (2.5 μM+2.5 μM), and IPI-549 and doxorubicin (2.5 μM+2.5 μM) for 14 days. The volumes ([major axis]×[minor axis]²/2) of the MTCs were monitored every other day using an Olympus IX83 motorized inverted microscope with cellSens Dimension software (Olympus), and images were captured using a CellInsight CX5 High-Content Screening (HCS) Platform (Thermo Fisher) at 4× magnification.

Pharmacokinetics and tissue distribution of PTX and IPI-549 delivered by Nano-PI in MMTV-PyMT mice. PyMT mice (female, 14-15 weeks old) with 10-16 spontaneous tumors each were randomly assigned to three groups (n=12) and administered the following formulations: PTX/IPI-54 (IV), Nano-P (IV) plus IPI-549 (PO). Nano-P (IV) plus IPI-549 (IP) or Nano-PI (IV) at the PTX and IPI-549 dosage of 5 mg/kg and 2.5 mg/kg, respectively. PTX and/or IPI-549 were dissolved in 10% dimethyl sulfoxide (DMSO) in polyethylene (PEG) 400 and mixed with 50% sterile saline (0.9%, w/v), and Nano-P and Nano-PI were suspended in sterile saline. Blood was collected at 0.5, 4, 7, and 24 h post administration, and plasma was isolated after centrifugation (13,300 rpm for 10 min). Subsequently, mice were euthanized, and major organs including the liver, spleen, lung, lymph node, fat pad, and tumor were collected, weighed, and homogenized. For accurate detection of drug distribution, we randomly dissected 10 tumors and 8 fat pads at different locations from each mouse. Subsequently, the contents of PTX and IPI-549 in the tissue homogenate and plasma were detected by LC-MS using an ABI-5500 Qtrap (Sciex) mass spectrometer with an electrospray ionization source which was interfaced with a Shimadzu HPLC system with an Xbridge C18 column (50×2.1 mm ID, 3.5 μm). Results are represented as the amount of PTX or IPI-549 normalized per g/ml of tissue.

Anticancer efficacy in MMTV-PyMT transgenic mice. PyMT mice (9-10 weeks old) with spontaneous tumors were randomly assigned to six groups (n=17), and the total tumor size of each group was in the range of 80-110 mm³. The mice were then treated with vehicle, Nano-P, Nano-P plus α-PD1, IPI-549 plus α-PD1, Nano-P plus IPI-549 and α-PD1, Nano-PI and Nano-PI plus α-PD1 IPI-549 was dissolved in 10% DMSO in 40% PEG 400, mixed with 50% sterile saline, and orally administered daily at 15 mg/kg or injected intraperitoneally at 5 mg/kg every three days. Nano-P and Nano-PI suspended in sterile saline were intravenously administered at PTX and IPI-549 dosages of 10 mg/kg and 5 mg/kg, respectively. α-PD1 (100 μg/mouse) was administered (IP) on days 66, 69, and 72 after birth. To assess the advanced properties of Nano-PI mediated oncotherapy, PyMT mice (11-12 weeks old) with an average tumor size of 150 mm³ were randomly assigned to 3 groups (n=17) and then intravenously administered with the vehicle, PTX/IPI-549 (dissolved in 10% DMSO+40% PEG 400+50% sterile saline), and Nano-PI at the PTX and IPI-549 dosage of 5 mg/kg and 2.5 mg/kg, respectively Bodyweight, tumor number, and tumor volume were measured and recorded every three days. Three (normal dose batch)/four days (half dose batch) after the last administration, three mice from each group were euthanized, and the tumor and lymph nodes were collected for flow cytometry analysis. On day 85 (normal dose batch) and 111 (half dose batch), another four mice from each group were euthanized, and the lungs were harvested, washed, and fixed in the Bosin fixative (Sigma). After repeated rinsing, lung organs with metastatic tumor nodules were photographed. Then the excised lung organs were fixed, sectioned, and stained with H&E, followed by imaging using an inverted fluorescence microscope to investigate further inhibition of tumor lung metastasis.

Anti-tumor efficacy in 4T1 orthotopic breast cancer mice. An orthotopic breast cancer model was established in female BALB/c mice. Briefly, 4T1 cells and M2 macrophages (viabilities >96%) derived from RAW 264 7 cells were mixed (3:1, rat) and injected into the mammary fat pad of mice (5×10⁷ cells/mL). When tumor sizes reached ˜120 mm³, mice were randomly assigned to seven groups (n=11) and received different treatments. The administration was performed as a single treatment or combined with the following regimen for each drug three times. IPI-549 was dissolved in 10% DMSO in 40% PEG 400, mixed with 50% sterile saline, and orally administered daily or injected intraperitoneally at 5 mg/kg. Nano-P and Nano-PI were dissolved in sterile saline and intravenously administered at the PTX and IPI-549 dosages of 10 mg/kg and 5 mg/kg. Anti-PD1 antibodies (α-PD1) (100 μg/mouse) were administered (IP) on days 5, 8, and 1 1 after tumor inoculation. Body weights and tumor volumes ([long axis]×[short axis]2/2) were measured and recorded every three days. Three days after the last administration, three mice from each group were euthanized, and the tumor and lymph nodes were collected for flow cytometry. Thirteen days after the last administration, all the mice were sacrificed, and the tumors were dissected, weighed, photographed, and stored at −80° C. The tumor inhibition ratio (TIR) was calculated using the following equation: (1−Wₐ/Wᵥ)×100%, where Wₐ and Wᵥ represent average tumor weights of the administration groups and vehicle groups, respectively. For histological testing, lung tissues were harvested, fixed in 4% paraformaldehyde (PFA, w/v), and paraffin-embedded sections (5 μm) were prepared. After staining with hematoxylin and eosin (H&E), breast cancer lung metastasis was analyzed by inverted fluorescence microscopy.

Fluorescence-activated Cell Sorting (FACS) of immune cells in tumors and lymph nodes. PyMT mice (15 weeks old) with more than 10 tumors were euthanized, and the tumors were dissected for preparation of single-cell suspension following the method mentioned above. The cells were then washed, counted, and incubated with SYTOX Green (30 nM) for 30 min at room temperature in the dark. Then, the live cells (negative) were sorted out and collected using a Sony MA900 sorter equipped with lasers of 488 nm excitation and 530/30 bandpass.

Tumor re-challenge study in PyMT transgenic mice with tumor remission. The live cells were sorted using FACS from single cell suspensions isolated from PyMT mouse tumors. Then, 100 μL of single cell suspension (5×10° cells/mL) was implanted into the mammary fat pad of FVB/NJ female mice and PyMT mice 132 days after the last administration of Nano-PI combined with α-PD1 (210 days after birth). After 8 days, tumor volumes were measured and recorded every four days for the first three times, and then changed every three days in the following days. At the end, the mice were euthanized, and the lymph nodes, spleen, lung, BM, and blood were collected to prepare single-cell suspensions using the aforementioned methods. The memory T cells and B cells in the lymph nodes, spleen, lung, BM, and blood of PyMT mice and FVB/NJ mice were detected by flow cytometry analysis

Flow cytometry analysis. Single cell suspensions obtained from the fat pad, lymph node, tumor, spleen, lung, blood, and bone marrow of the PyMT mice, FVB/NJ mice, and BALB/c mice were incubated with FcR-blocking reagent (BD Biosciences) for 15 min at 4° C. and then stained with fluorescently labelled antibodies with appropriate dilutions including; anti-CD44-Alexa Fluor® 647, anti-F4/80-Pacific Blue, anti-CD31-Spark YG™ 570, anti-CD45-Spark blue 550, anti-CD45-Pacific blue, anti-F4/80-PE, anti-CD169-PE, anti-CD3-Alexa Fluor® 488, anti-CD3-Spark blue 550, anti-CD19-PE, anti-CD103-Pacific blue, anti-CD103-Alexa Fluor®-647, anti-CD19-Alexa Fluor® 488, anti-CD335-PE/Dazzle™ 594, anti-CD103-Alexa Fluor® 488, anti-CD11C-Pacific blue, anti-CD80-Alexa Fluor®-647, anti-CD80-Spark blue 550, anti-F4/80-Alexa Fluor®-647, anti-CD11 b-Alexa Fluor 594, anti-CD169-Alexa Fluor®-647, anti-CD206-Alexa Fluor® 488, anti-CD80-Alexa Fluor 488, anti-CD206-Alexa Fluor®-647, anti-CD86-PE/Dazzle™, anti-CD3-Alexa Fluor 488, anti-CD19-PE, anti-CD197-Alexa Fluor®-647, anti-CD44-Pacific Blue, anti-CD80-Alexa Fluor®-594, anti-CD 103-Alexa Fluor®-594, anti-CD73-Pacific Blue, anti-PD-L2-APC-633, anti-CD3-Spark YG™570, anti-CD19-Brilliant violet 421™, anti-CD8-Alexa Fluor®-594, anti-CD4-Alexa Fluor®-532, and anti-CD3-Alexa Fluor®-594 for 30 min on ice. All antibodies were purchased from Biolegenda, eBioscience™, and R&D Systems. For intracellular staining, cells were fixed, permeabilized with permeabilization staining buffer (eBioscience), and then incubated with anti-FoxP3-Alexa Fluor®-647. The stained cells were acquired on a Bio-Rad ZE5 Flow Cytometer equipped with four lasers (405 nm, 488 nm, 561 nm, and 640 nm) and twenty-one fluorescent detectors using BD FACSDiva software (BD Biosciences). All data analyses were performed using the flow cytometry analysis program FCS Express 7 (De Novo software). Dead cells and doublets were excluded based on forward and side scatter and fixable viability dye.

Confocal microscopy. The tumor and lymph nodes were dissected from PyMT mice (14 weeks old) and cryosections (10 μm thick) were prepared for staining of M2 macrophages. Subsequently, the sections were fixed and pre-incubated with 5% FBS for 30 min at room temperature, incubated with anti-F4/80-Alexa Fluor®-647 and anti-CD206-FITC for 12 h at 4° C., mounted in ProLong™ Diamond Antifade Mountant with DAPI (Molecular Probes™, P36971) and imaged using a Nikon AlSi confocal microscope equipped with 405 nm, 488 nm, 561 nm, and 640 nm lasers. Nano-PI encapsulating the Oregon Green 488-labeled PTX and IPI-549 was prepared following a similar method for drug distribution. PyMT mice (13 weeks old) were intravenously administered Nano-PI at dosages of Oregon Green 488-labeled PTX and IPI-549 50 mg/kg and 25 mg/kg, respectively. At 5 h post administration, the tumor and lymph node were dissected to obtain cryosections and incubated with the following antibodies; anti-F4/80-Alexa Fluor®-647, CD206-Alexa Fluor®-488, anti-CD80-Alexa Fluor®-594, anti-CD31-Spark YG™ 570, anti-CD3-Spark YG™570, anti-CD19-Brilliant Violet 421™, and anti-CD45R/B220-Brilliant Violet 421™ for 12 h at 4° C. The tumor and lymph node sections were mounted in ProLong™ Diamond Antifade Mountant with DAPI (Molecular Probes™) and ProLong™ Diamond Antifade Mountant (Molecular Probes™, P36961), respectively. The drug distribution mediated by Nano-PI was analyzed using a Nikon AlSi confocal microscope.

CyTOF analysis of all immune cells in tumor tissues and lymph nodes. The immune profiles of the tumor and lymph nodes were detected using CyTOF analysis Single-cell suspensions (3 million cells) of tumors and lymph nodes from PyMT mice treated with different formulations were prepared and then fixed and stained for CyTOF analysis as described previously using an optimized cocktail of 40 metal-conjugated antibodies designed to identify the changes in cell subsets within tumors and lymph nodes. CyTOF antibody conjugation and data acquisition were performed as described previously. Briefly, antibodies were conjugated to lanthanide metals (Fluidigm) using the Maxpar Antibody Labeling Kit (Fluidigm). Single-cell suspensions prepared from PyMT mouse tumor and lymph node samples were prepared as described above. Unstimulated cell suspensions were washed once with heavy-metal-free PBS and stained with 1.25 μM Cell-ID Cisplatin-195Pt (Fluidigm) at room temperature for 5 min. Fc receptors were blocked with TruStain FcX (anti-mouse CD16/32, Biolegend), and surface staining was performed on ice for 60 min in heavy-metal-free PBS with 0.1% BSA, 2 mM EDTA, and 0.05% sodium azide. The cells were then fixed with 1.6% paraformaldehyde for 20 min at room temperature and then permeabilized with Invitrogen permeabilization buffer (Thermo Fisher Scientific) for 30 min at room temperature before intracellular antibody staining at room temperature for 60 min in permeabilization buffer. Cells were left in 62.5 nM Cell-ID Intercalator Iridium-191/193 (Fluidigm) in 1.6% paraformaldehyde in PBS overnight at 4° C. until ready for acquisition on CyTOF Helios system (Fluidigm). A signal-correction algorithm based on the calibration bead signal was used to correct for any temporal variation in the detector sensitivity. Data were acquired on a CyTOF Helios system (Fluidigm) and cell populations were analyzed by gating with FlowJo/FCS Express software. Cell populations were analyzed by gating with FlowJo/FCS Express software. Global analysis using SPADE/tSNE was performed for unsupervised clustering analysis based on the expression of marked genes in different subsets of immune cells.

CyTOF gating scheme to show the immune cell populations. PyMT transgenic mice with spontaneous metastatic breast cancer were euthanized and tumor and lymph nodes were isolated 3 days post the last round of the treatments in FIGS. 16D-16F. Single cell suspensions were selected from the cell populations and dead cells were excluded. The CD45⁺CD3⁺ T cells were selected and the subtype T cells (CD4 and CD8) were gated out of the CD3 T cells. The TEM (CD62L⁻CD44⁺) and TCM (CD62L⁺CD44⁺) were gated out of the CD4⁺5CD3⁺CD4⁺ T cells or CD45⁺CD3⁺CD4⁺ T cells. The immunosuppressive IIM-3 T cells (CD3⁺TIM-3⁺) were selected from the total CD3 T cells. NK cells (CD3⁺CD49b⁺) were different population with T cells and the CD3⁺CD49b⁺ cell populations were gated as NKT cells. B cells (CD45⁺CD19⁺CD45R⁻B220⁺) were selected from the total immune cells. In addition, the macrophages (CD11b⁻Ly-6G⁺F4/80⁺) were selected and then divided into M1 phenotype (CD80⁺CD206⁻) and M2 phenotype (CD80⁻CD206⁺) macrophages. The activated DCs (CD103⁺CD11b⁻) were selected from total DCs (CD11C⁺IA-IE⁺). All the immune cell populations were analyzed and quantified. Relative panels are listed in Table 1.

TABLE 1
Heavy metal labeled antibody panels for CyTOF analysis.

| Channel | Target | Clone | Vendor |
|---|---|---|---|
| 112Cd | CD19 | 6D5 | Life Technologies |
| 141Pr | IFNg | XMG1.2 | Biolegend |
| 142Nd | CD86 | GL-1 | Biolegend |
| 143Nd | CD80 | 16-10A1 | Biolegend |
| 144Nd | Siglec-F | E50-2440 | Biolegend |
| 145Nd | CD4 | RM4-5 | Biolegend |
| 146Nd | CD45R (B220) | RA3-6B2 | Biolegend |
| 147Sm | CD206 | C068C2 | Biolegend |
| 148Nd | CD103 | AF1990 | Novus Biologicals |
| 149Sm | CD8 | 53-6.7 | Biolegend |
| 150Nd | mPDCA-1 (CD317) | 129C1 | Biolegend |
| 151Eu | CD49b (DX5) | DX5 | Biolegend |
| 152Sm | Ly-6C | HK1.4 | Novus Biologicals |
| 153Eu | IFNb | 7F-D3 | Abcam |
| 154Sm | CD11c | N418 | Biolegend |
| 155Gd | IA-IE (MHCII) | M5/114.15.2 | Biolegend |
| 156Gd | CD25 | 3C7 | Biolegend |
| 158Gd | TIM-3 | RMT3-23 | Biolegend |
| 159Tb | Ly-6G | 1A8 | Biolegend |
| 160Gd | Il-4 | 11B11 | Biolegend |
| 161Dy | Il-17a | TC11-18H10.1 | Biolegend |
| 162Dy | TCRγδ | GL3 | Biolegend |
| 163Dy | Il-17f | 316016 | Biolegend |
| 164Dy | Il-10 | JES5-16E3 | Biolegend |
| 165Ho | CD115 | AFS98 | Biolegend |
| 166Er | Cxcr5 | 614641 | Novus Biologicals |
| 167Er | FR4 | TH6 | Biolegend |
| 168Er | NOS2 | 5C1B52 | Biolegend |
| 169Tm | Ly-6A/E (Sca-1) | D7 | Biolegend |
| 170Er | CD62L | MEL-14 | Biolegend |
| 171Yb | CD44 | IM7 | Biolegend |
| 172Yb | CD11b | M1/70 | Biolegend |
| 173Yb | PD-1 | RMP1-30 | Biolegend |
| 174Yb | CTLA-4 | UC10-4B9 | Biolegend |
| 175Lu | F4/80 | BM8 | Biolegend |
| 176Yb | GmzB | GB11 | Abcam |
| 209Bi | CD3 | 145-2C11 | Biolegend |
| 89Y | mCD45 | 30-F11 | Fluidigm |
| 195Pt | Cisplatin | Live/Dead | Fluidigm |
| 191/193Ir | DNA Intercalator | DNA | Fluidigm |

Statistical analysis. All experiments were carried out in triplicates unless otherwise noted specifically, and all results are represented as the means±SD. Statistical analyses were performed using Student's unpaired t-test for comparisons between two groups, and multiple comparisons were conducted using one-way analysis of variance (ANOVA) with the Bonferroni test (SPSS software, version 12.0. SPSS Inc.). All statistical analyses were calculated using GraphPad Prism 8 and OriginPro 8 software, and statistical significance is donated as P<0.05.

Drug release test. Nano-PI and free drug dissolved in DMSO (400 μL, PTX 6.6 mg/mL, IPI 3.7 mg/mL) were sealed in dialysis bag (MWCO, 3.5 kDa) and immerged in 20 mL of plasma at 37° C., under shaking. At predetermined time intervals, 600 μL of sample was withdrawn and replaced with equal volume of fresh release medium. The concentration of PTX and IPI-549 in the samples was determined by LC-MS/MS. The accumulative release amount of PTX and IPI-549 was calculated accordingly.

Mass spectrometry (MS) imaging. Nano-PI was intravenously administered in the PyMT mice (female, 10-11 weeks old) at the dosage of PTX 100 mg/kg and IPI-549 50 mg/kg. After 4 hours, the mice were euthanized, and the tumors and lymph nodes were dissected to prepare frozen sections for MS imaging. The sections at 10 μm thick were prepared using a Cryostat (Leica CM 1950) and mounted on Indium tin oxide (ITO) coated glass slides (Fisher Scientific) and then dried in a desiccator for about 30 min. The sections were then sprayed with matrix (10 mg/mL 2′,5′-dihydroxyacetophenone in 90:10 (v/v) acetonitrile/water+0.1% LC-MS grade trifluoroacetic acid (TFA)) using an HTX™ sprayer (HTX Technologies, LLC) and dried for 15 min. Matrix applied tissues sections were analyzed by the MALDI source (MassTech Inc, Columbia, MD) coupled with Orbitrap IDX (Thermo Fisher) mass spectrometer. The data was visualized, and images were generated by Bruker SCILS Lab software.

Cellular drug distribution of Nano-PI in memor and lymph node. PyMT mice (female, 13 weeks old) with 6-10 spontancous tumors were randomly assigned to two groups (n=4) and intravenously administered with the free drug combination PTX/IPI-549 or Nano-PI at the PTX and IPI-549 dosage of 100 mg/kg (PTX-OG488, 10 mg/kg) and 30 mg/kg, respectively. PTX and IPI-549 were dissolved in 10% dimethyl sulfoxide (DMSO) in polyethylene (PEG) 400 and mixed with 50% sterile saline (0.9%, w/v), and Nano-PI were suspended in sterile saline. The tumor and lymph nodes tissues were dissected and prepared the single cell suspensions or cryosections with 10 μm thickness. The single cell suspensions from different mice were incubated with fluorescently labelled antibodies with appropriate dilutions including anti-F4/80-Pacifice Blue, anti-CD44-Alexa Fluor® 647, and anti-CD169-Alexa Fluor®-647. The stained cells were acquired on a Bio-Rad ZE5 Flow Cytometer equipped with four lasers (405 nm, 488 nm, 561 nm, and 640 nm) and twenty-one fluorescent detectors using BD FACSDiva software (BD Biosciences). All data analysis was performed using the flow cytometry analysis program FCS Express 7 (De Novo software). Dead cells and doublets were excluded based on the forward and side scatter and Fixable Viability Dye. And the sections from different mice were fixed and pre-incubated with 5% FBS for 30 min at room temperature, followed by further incubation with anti-F4/80-Alexa Fluor®-647 and anti-CD31-Spark YG™ 570 for 12 h at 4° C. Then sections were mount in ProLong™ Diamond Antifade Mountant with DAPI (Molecular Probes™, P36971) and imaged using a Nikon Alsi confocal equipped with lasers of 405 nm, 488 nm, 561 nm, and 640 nm.

Flow cytometry to analyze immune cell infiltration in tumor and lymph node. MMTV-PyMT transgenic mice (9-10 weeks old) were randomly assigned to 3 groups (n=4) when the total tumor size was in the range of 80-110 mm³. On day 66 after birth, the mice were then treated with vehicle, Nano-P plus IPI549 (IP) and α-PD1, Nano-PI plus α-PD1 every three days and for a total of 5 times with the PTX and IPI-549 dosages of 10 mg/kg and 5 mg/kg, respectively. IPI-549 was dissolved in 10% DMSO in 40% PEG 400, mixed with 50% sterile saline, and injected intraperitoneally at 5 mg/kg. Nano-P and Nano-PI suspended in sterile saline were intravenously administered with PTX and IPI-549 dosages of 10 mg/kg and 5 mg/kg, respectively. α-PD1 (100 μg/mouse) was administered (IP) on days 66, 69, and 72 after birth. Three days after the last administration, three mice from each group were euthanized, and the tumor and lymph nodes were collected for flow cytometry analysis. The single cell suspensions from tumor and lymph nodes were incubated with anti-CD45-Spark blue 550, anti-CD45-Pacific blue, anti-F4/80-Alexa Fluor®-647, anti-F4/80-PE, anti-CD169-PE, anti-CD3-Alexa Fluor® 488, anti-CD3-Spark blue 550, anti-CD19-PE, anti-CD103-Pacific blue, anti-CD103-Alexa Fluor®-647, anti-CD19-Alexa Fluor® 488, anti-CD335-PE/Dazzle™ 594 for 30 min on ice. The stained cells were acquired on a Bio-Rad ZE5 Flow Cytometer equipped with four lasers (405 nm, 488 nm, 561 nm, and 640 nm) and twenty-one fluorescent detectors using BD FACSDiva software (BD Biosciences). For each sample, 2 million cells were imported into Flow Cytometer. All data analyses were performed using the flow cytometry analysis program FCS Express 7 (De Novo software). Dead cells and doublets were excluded based on forward and side scatter and fixable viability dye.

Immunofluorescent staining of tumor and lymph node after treatments to identify the macrophages phonotype changes. MMTV-PyMT transgenic mice (9-10 weeks old) with transgenic spontaneous tumors were randomly assigned to 7 groups (n=4) when the total tumor size was in the range of 80-110 mm³. On day 66 after birth, the mice were then treated with vehicle. Nano-P, Nano-P plus α-PD1, IPI-549 (15 mg/kg. PO) plus α-PD1, Nano-P plus IPI-549 (IP) and α-PD1, Nano-PI, Nano-PI plus α-PD1 every three days and in total 5 times, respectively. IPI-549 was dissolved in 10% DMSO in 40% PEG 400, mixed with 50% sterile saline, and injected intraperitoneally at 5 mg/kg. Nano-P and Nano-PI suspended in sterile saline were intravenously administered at PTX and IP1-549 dosages of 10 mg/kg and 5 mg/kg, respectively. α-PD1 (100 μg/mouse) was administered (IP) on days 66, 69, and 72 after birth. Three days after the last administration, three mice from each group were euthanized, and the tumor and lymph nodes were collected, fixed and pre-incubated with 5% FBS for 30 min at room temperature, followed by further incubation with anti-F4/80-Alexa Fluor®-647, CD206-Alexa Fluor®-488, and anti-CD80-Alexa Fluor®-594 for 12 h at 4° C. Then sections were mount in ProLong™ Diamond Antifade Mountant with DAPI (Molecular Probes™ P36971) and imaged using a Nikon Alsi confocal equipped with lasers of 405 nm, 488 nm, 561 nm, and 640 nm.

To verify the stability of the compounds and formulations for nebulization treatments, the compounds, or formulations thereof (e.g., nanoformulation freeze-dried powder), will be resuspended with saline to make the solution for testing. The solution will be incubated at 4 C, 20° C. and 37° C. for 0, 2, 5, 10, 15 and 20 min, respectively. A small drop of each sample will be withdrawn at the varying temperature and timepoints and mixed with a methanol/acetonitrile mixture (1:1, v/v) with 10% water containing 5-(2-Aminopropyl)indole (5-IT, 20 nM) in a volume ratio of 1:4. The concentration the compounds or any breakdown products will be analytically detected (e.g., using LC-MS)

To verify the stability of the compounds and formulations for intravenous injections, the compounds, or formulations thereof (e.g., nanoparticle or liposome compositions), will be incubated with plasma (e.g., from mice, rat, hamster, and human) for 0, 5, 10, 30, 60 min and 2 hrs at 37 C, respectively. After the indicated time points, the sample will be filtered, dried, and reconstituted prior to analytical analysis of the compounds of any breakdown products.

Tumors and Lymph Nodes of PyMT Transgenic Mice with Spontaneous Metastatic Breast Cancer Showed an Increased M2 Macrophage Infiltration.

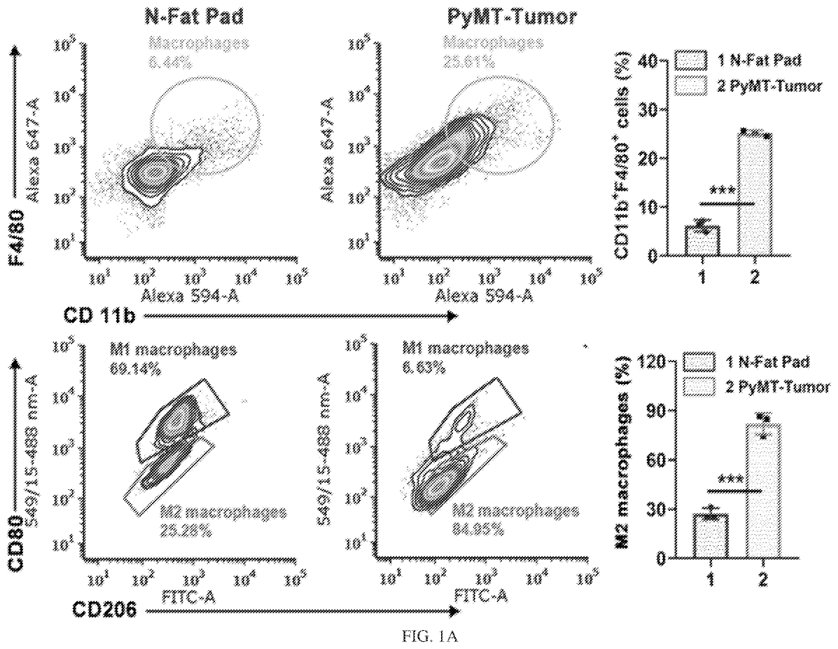

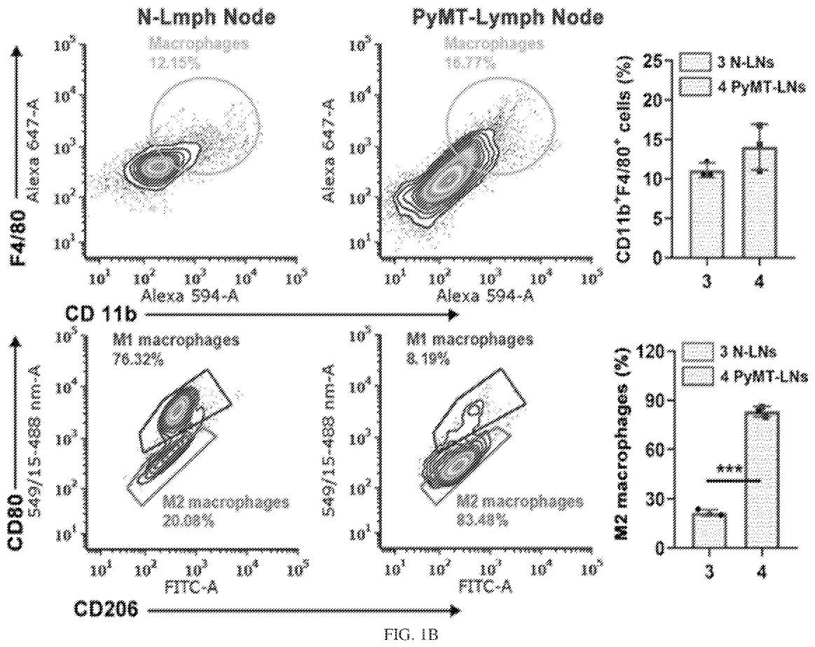

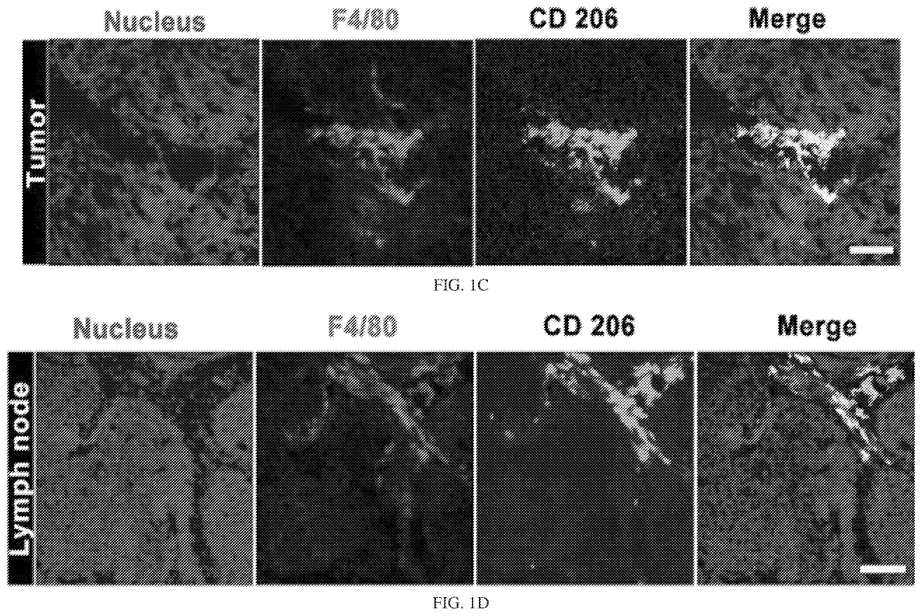

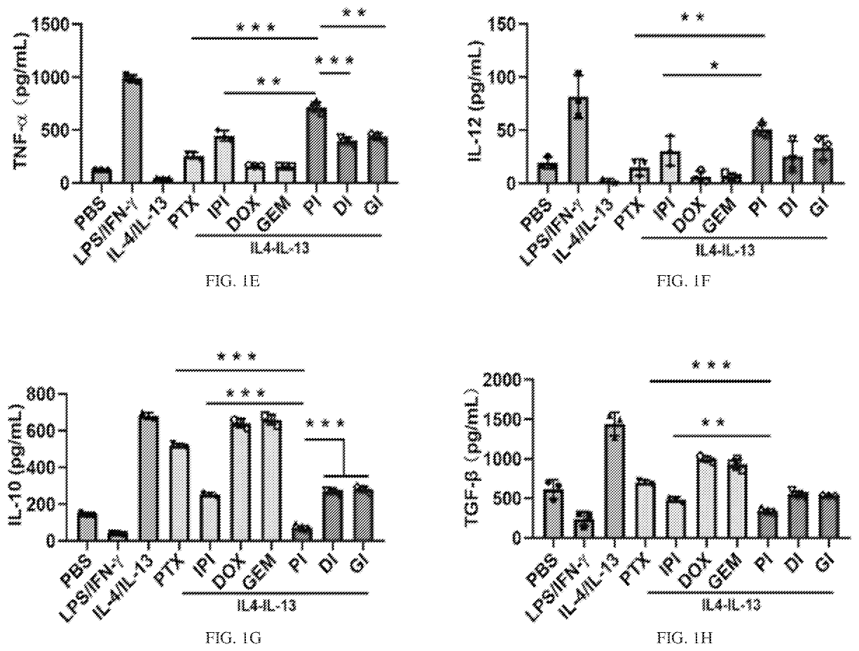

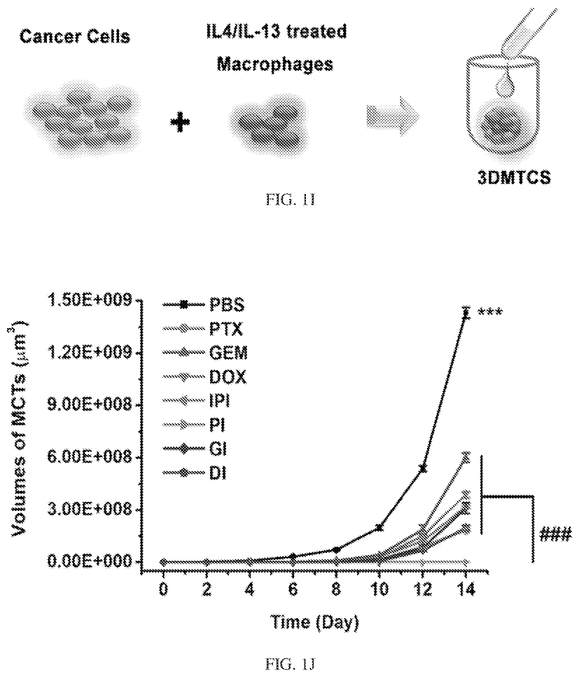

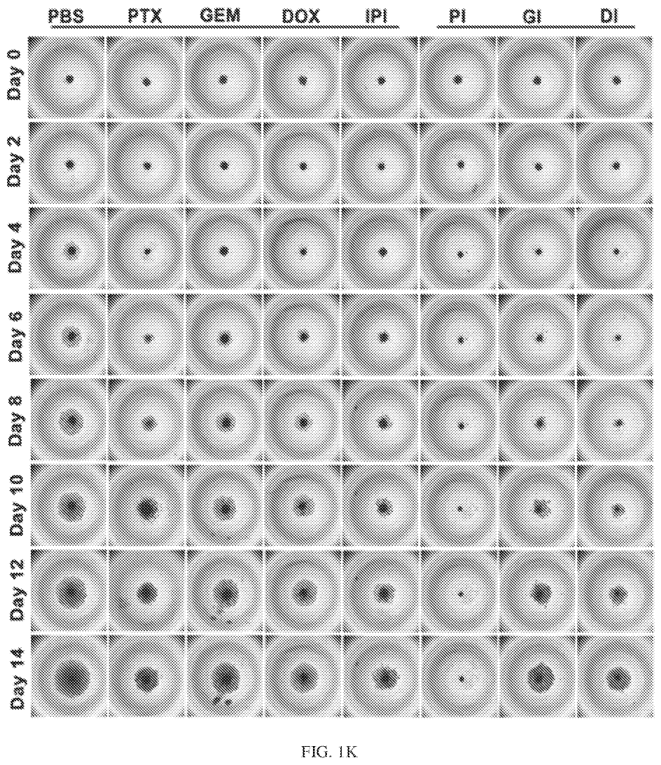

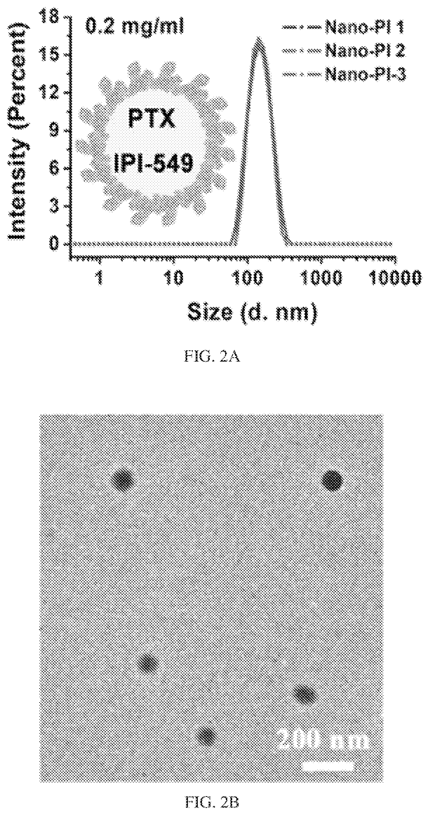

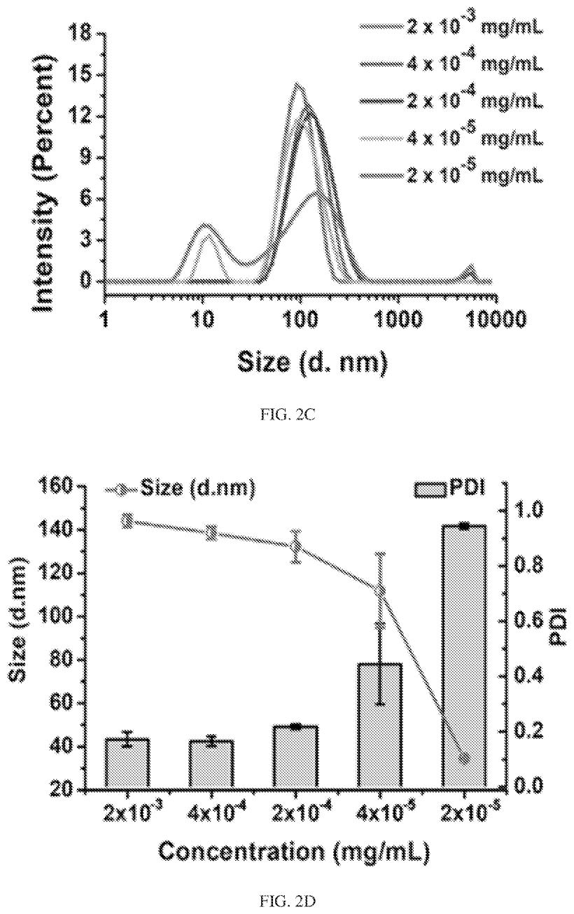

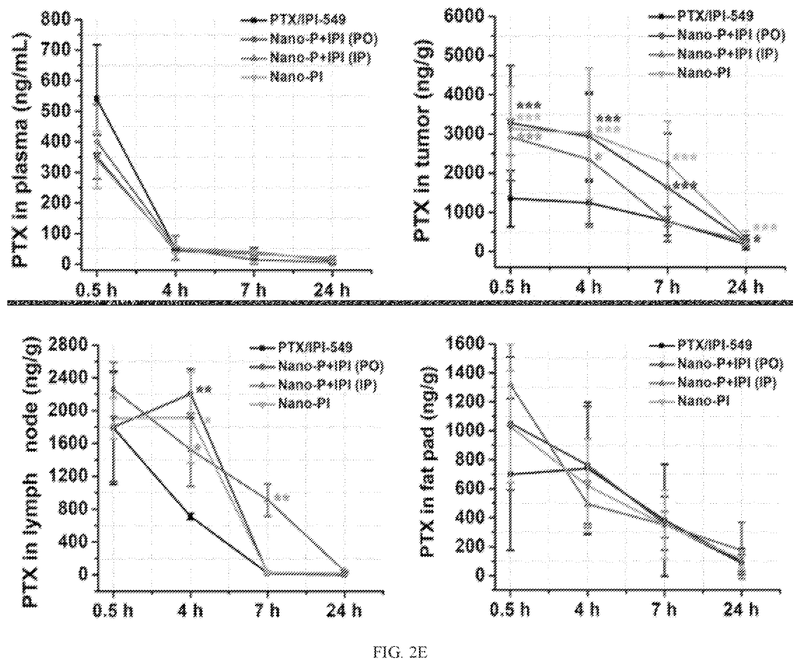

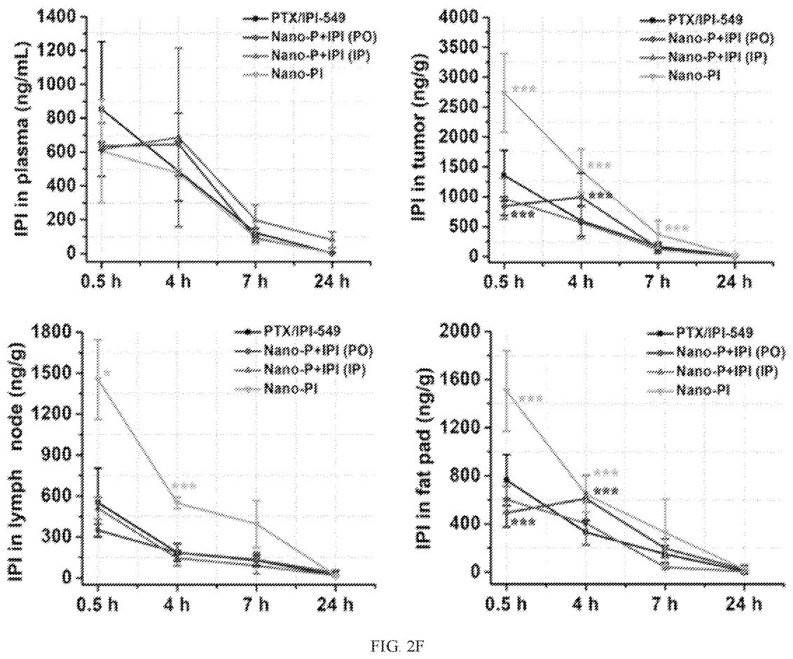

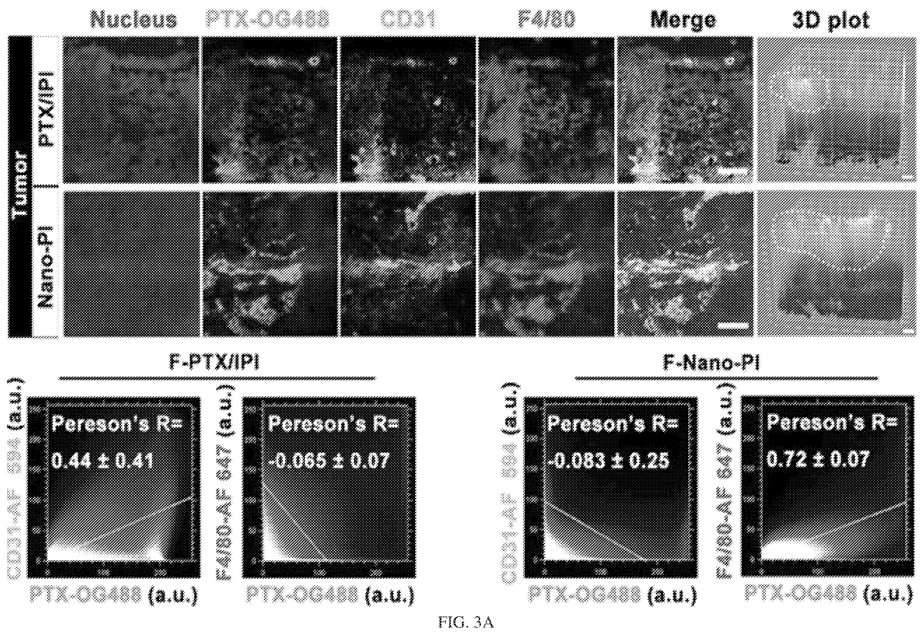

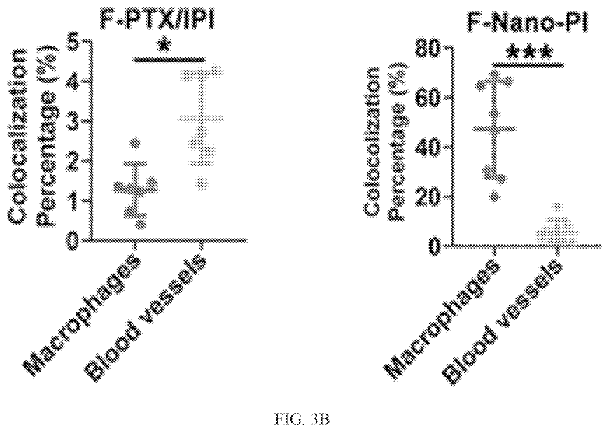

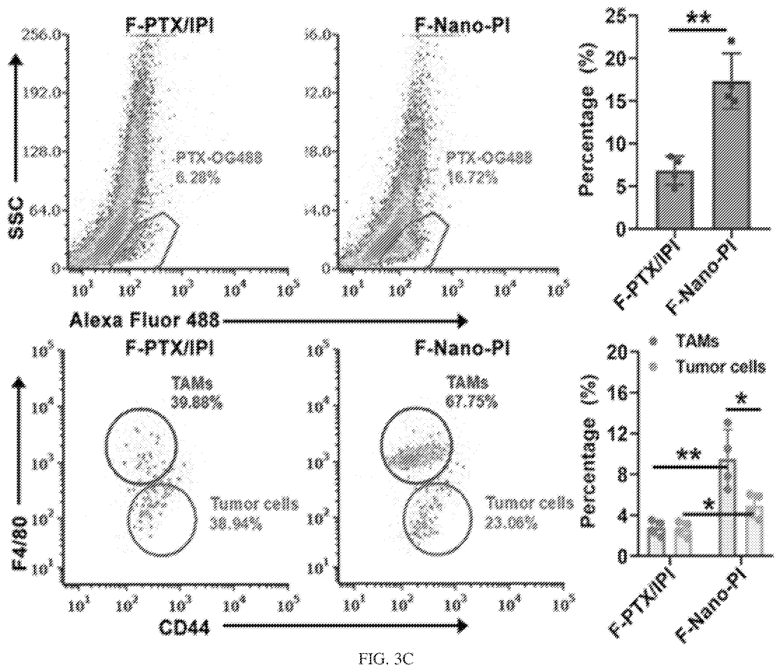

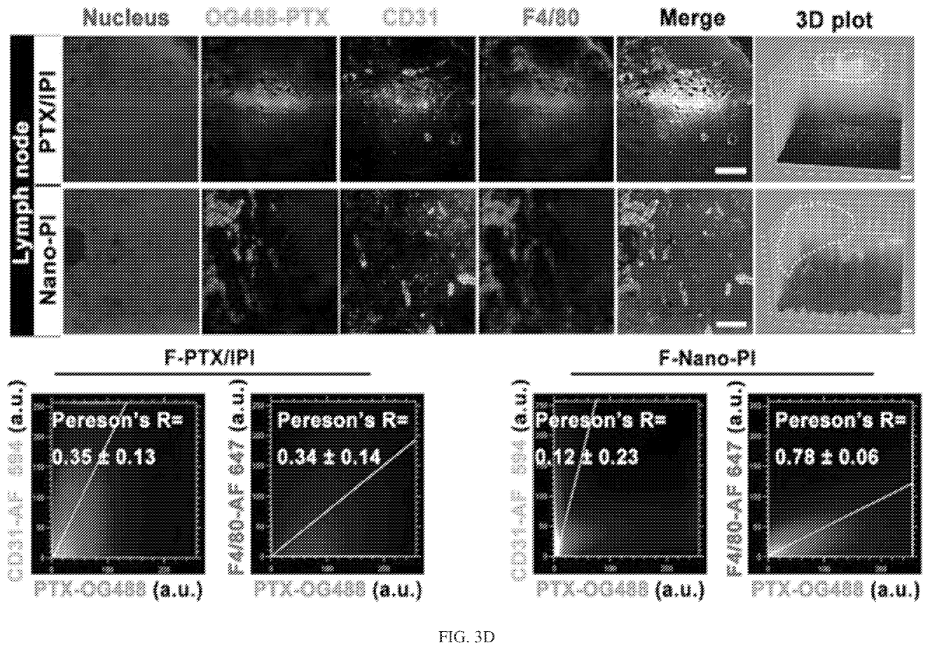

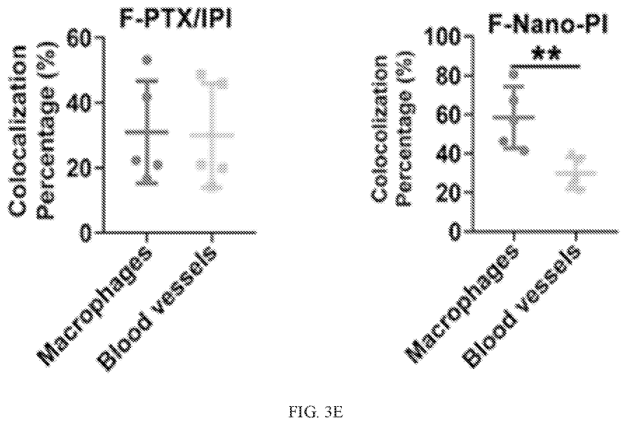

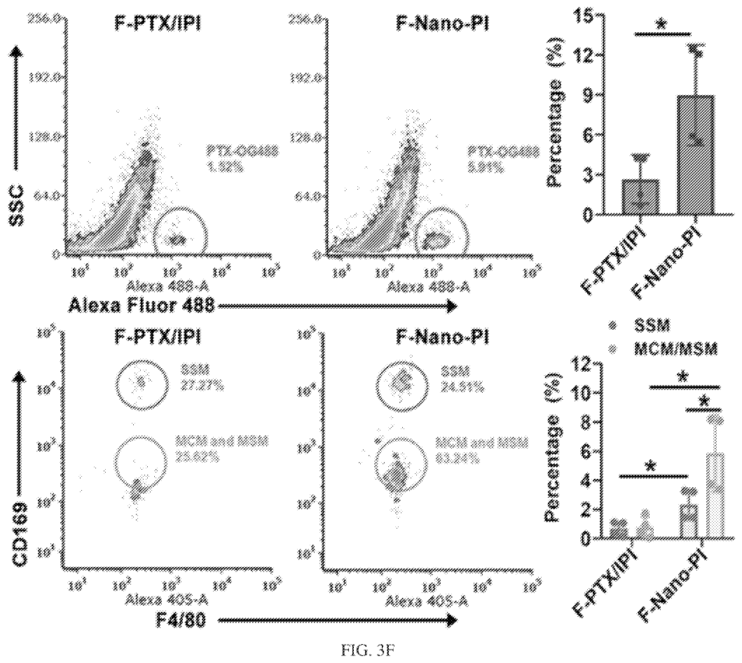

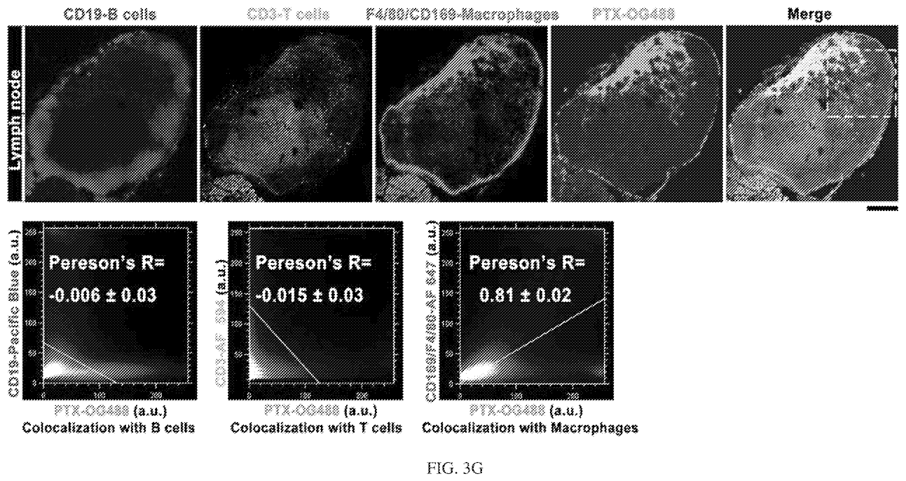

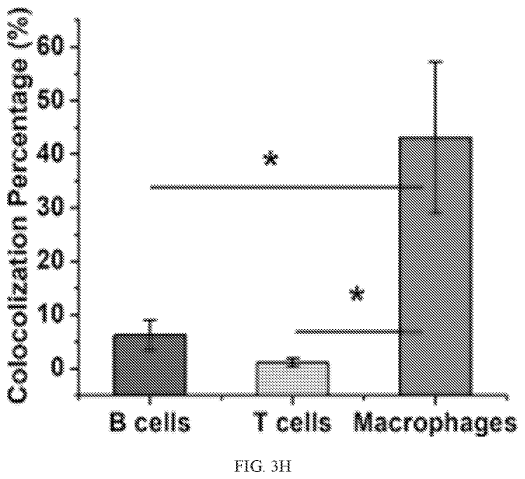

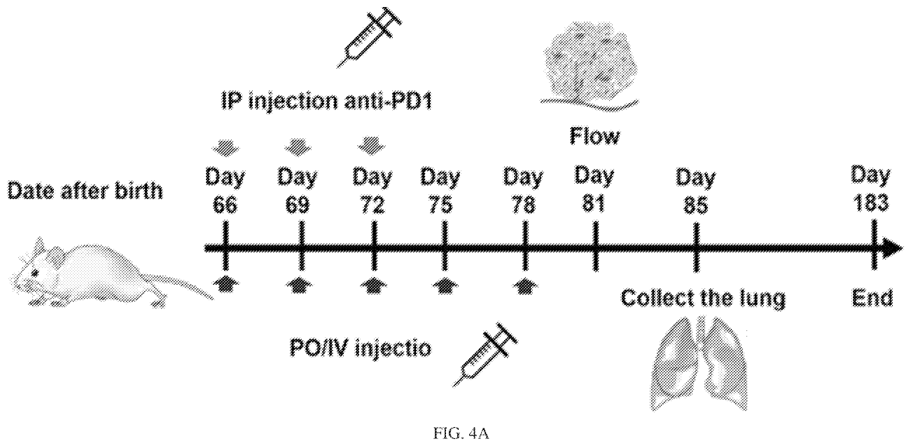

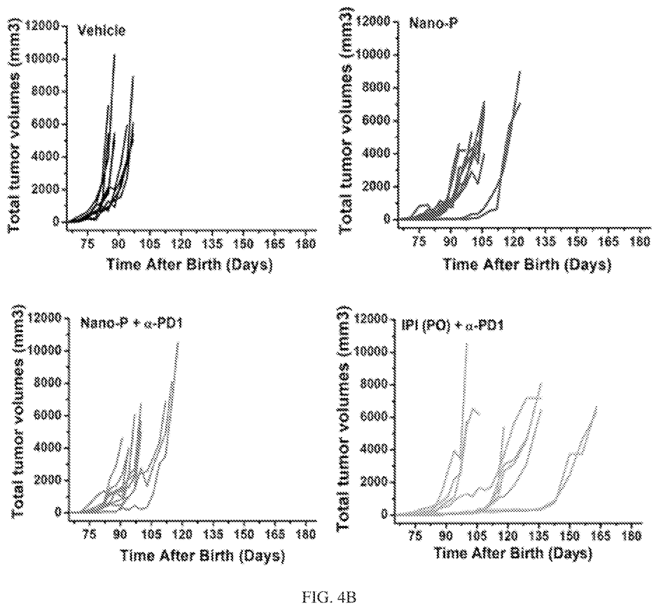

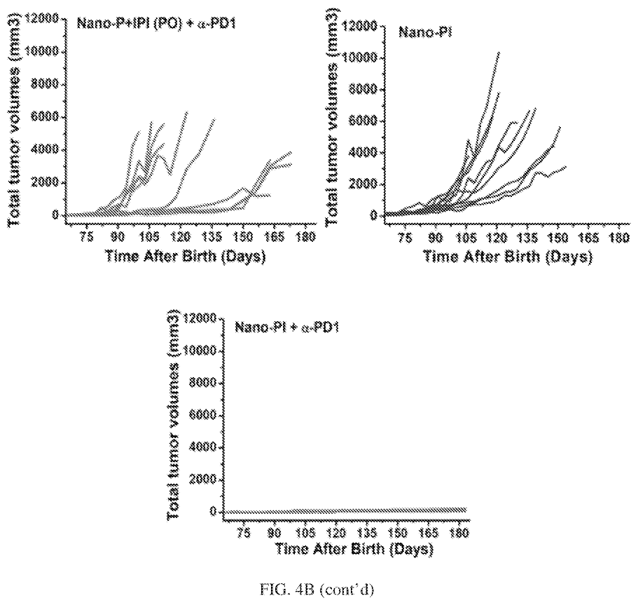

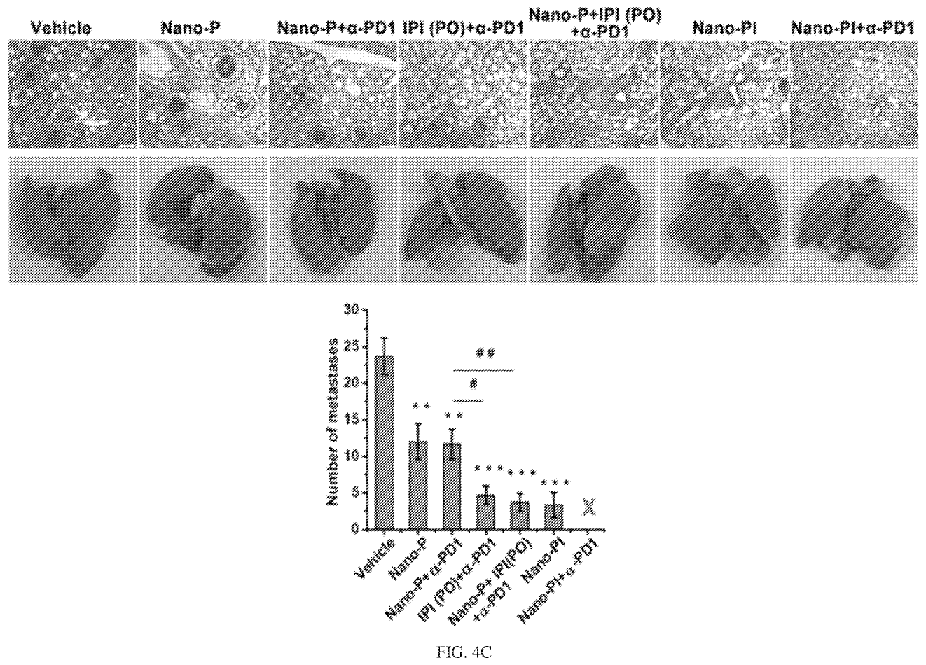

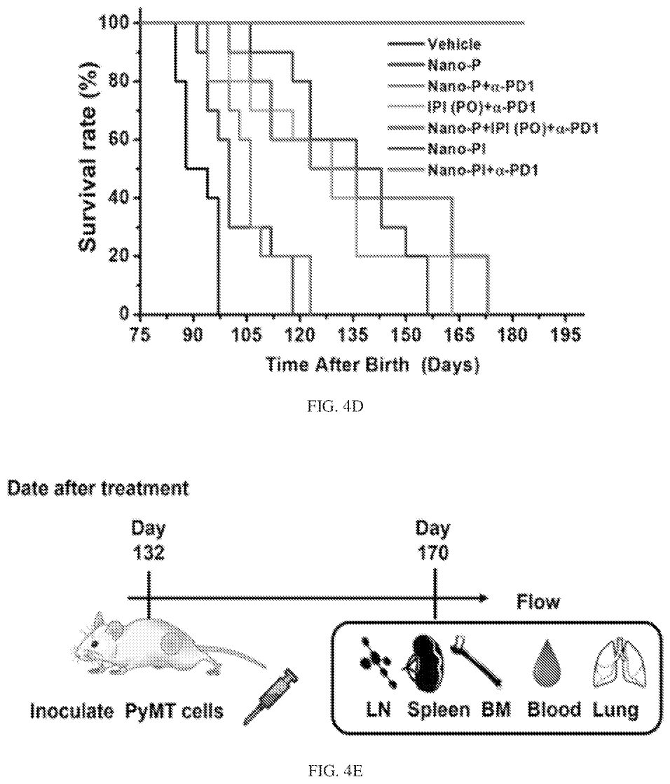

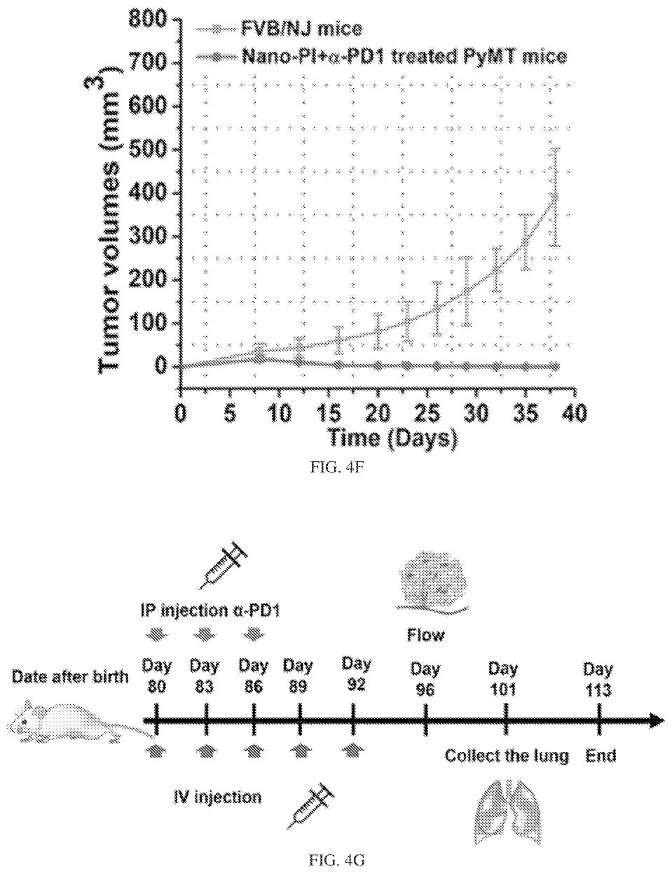

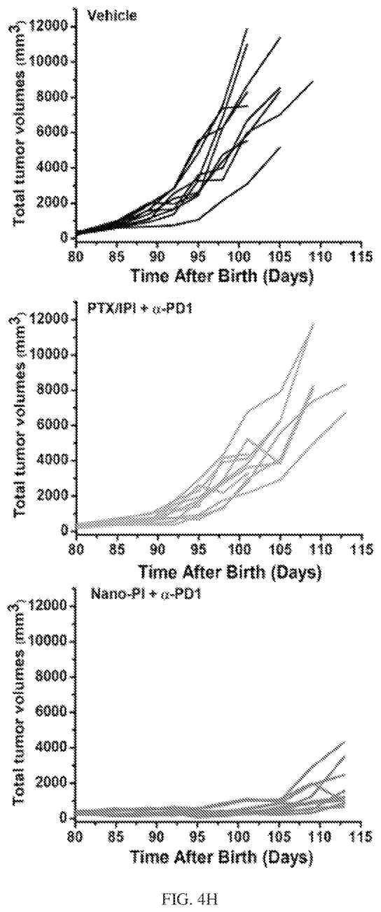

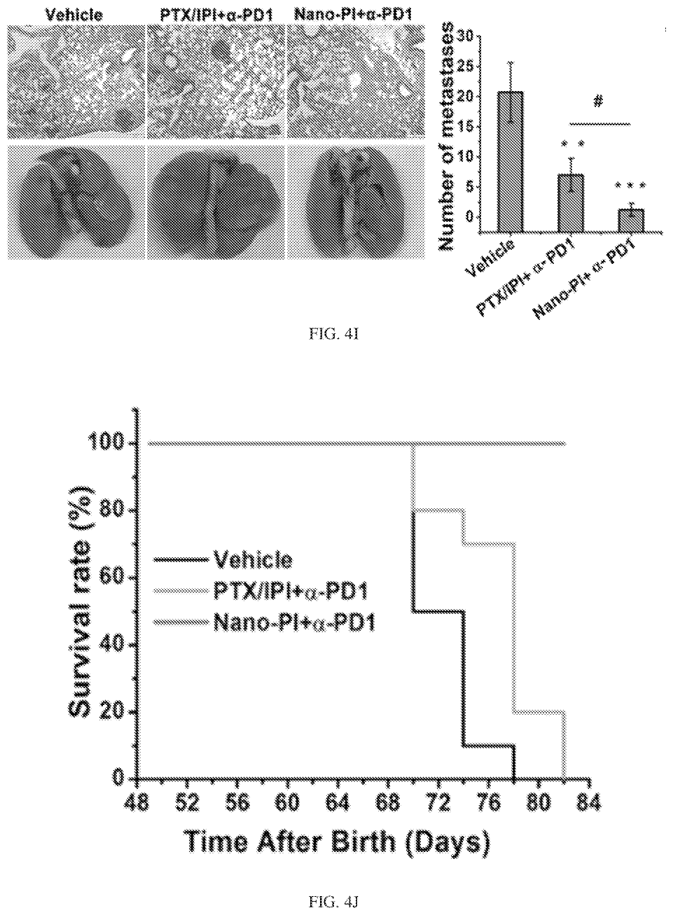

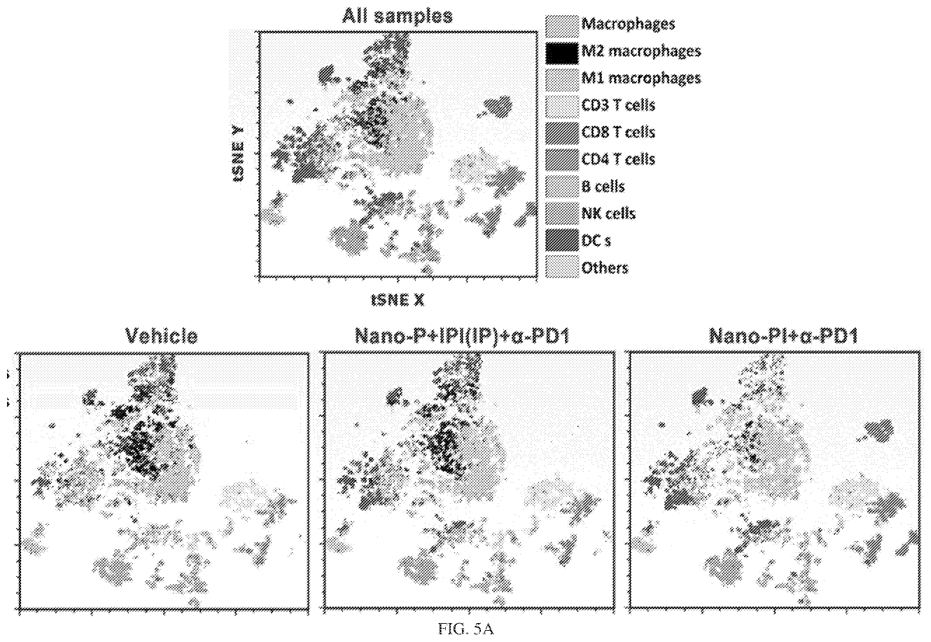

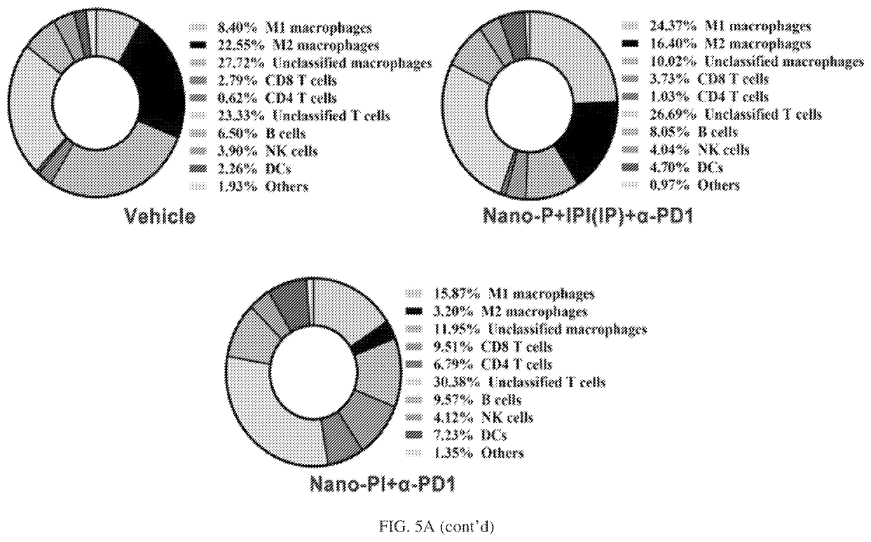

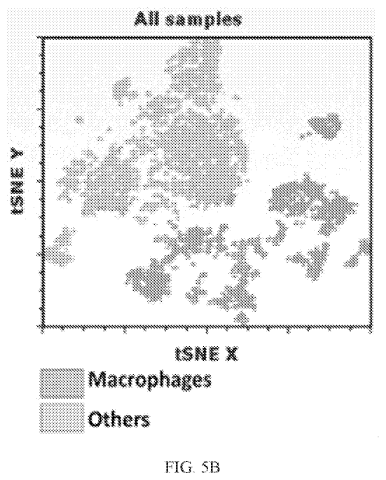

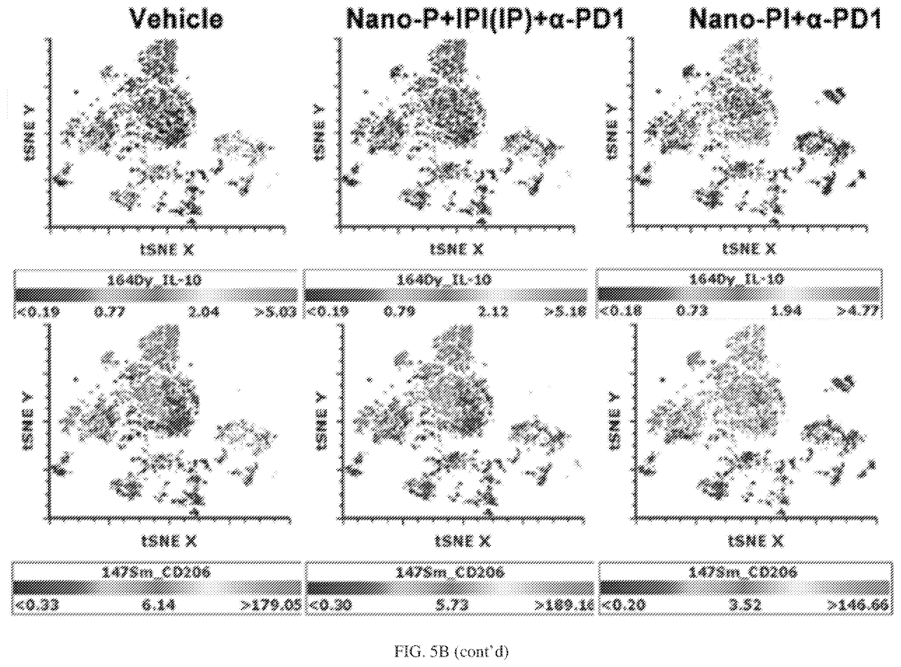

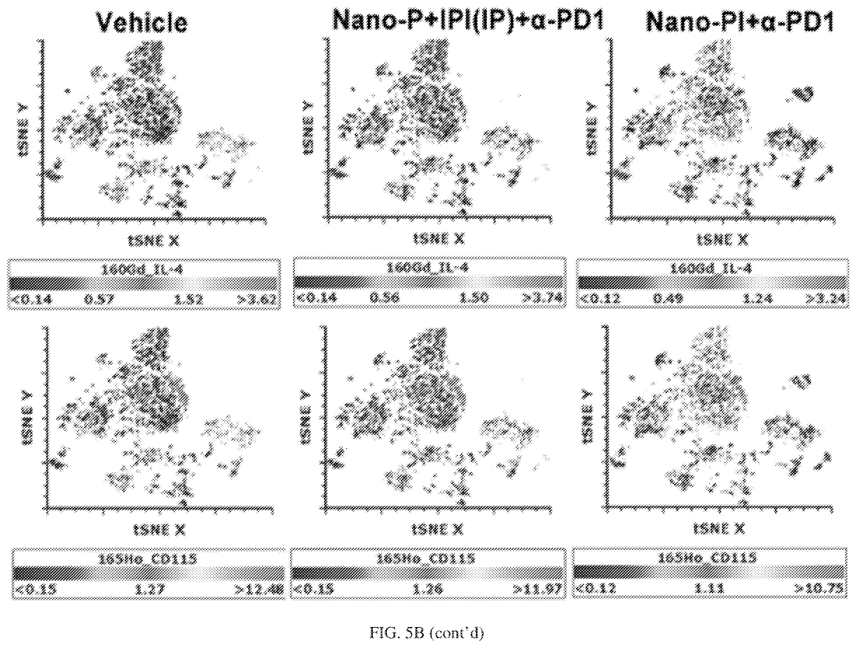

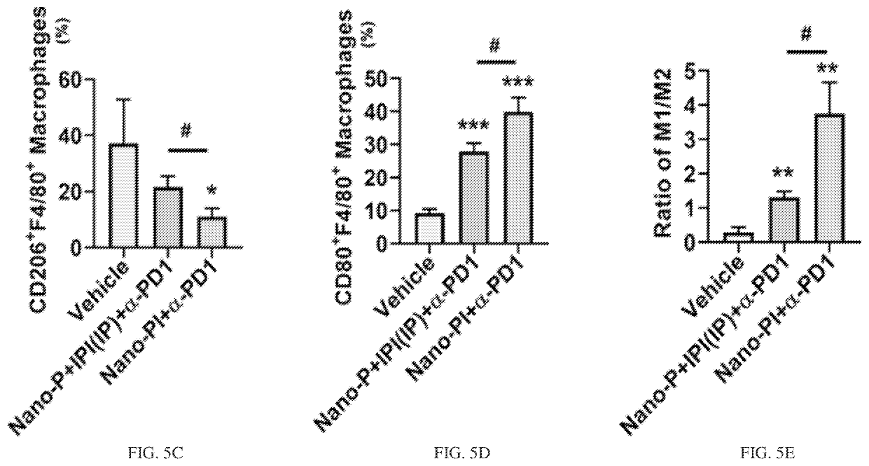

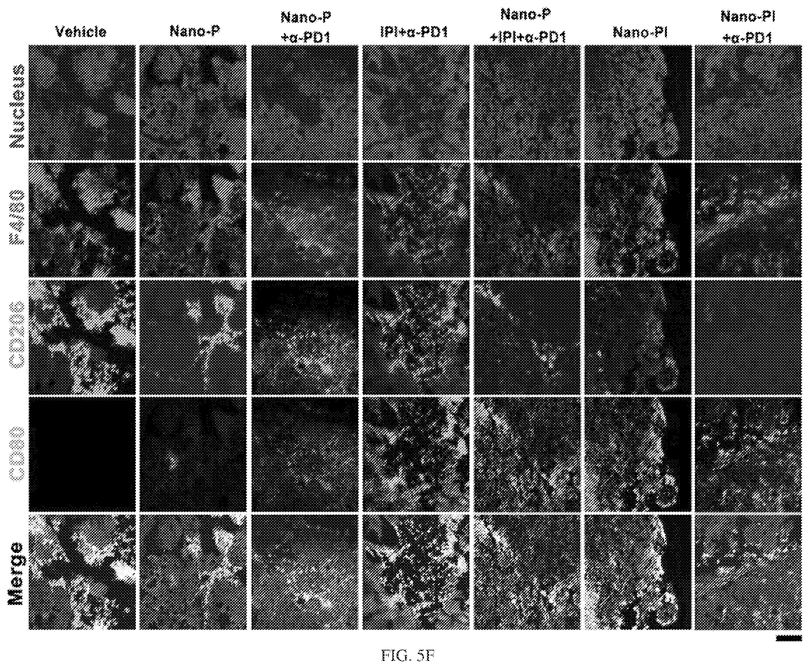

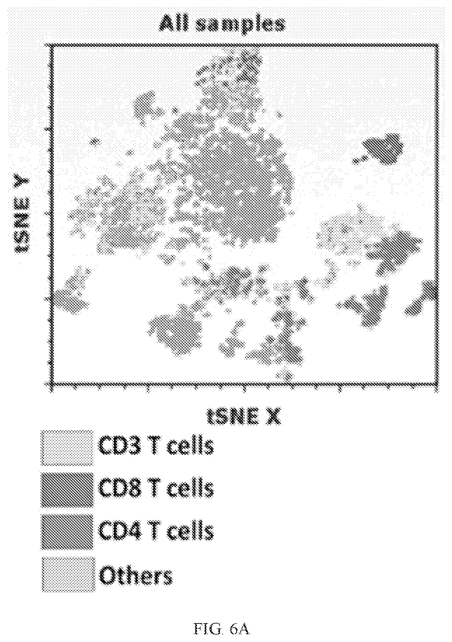

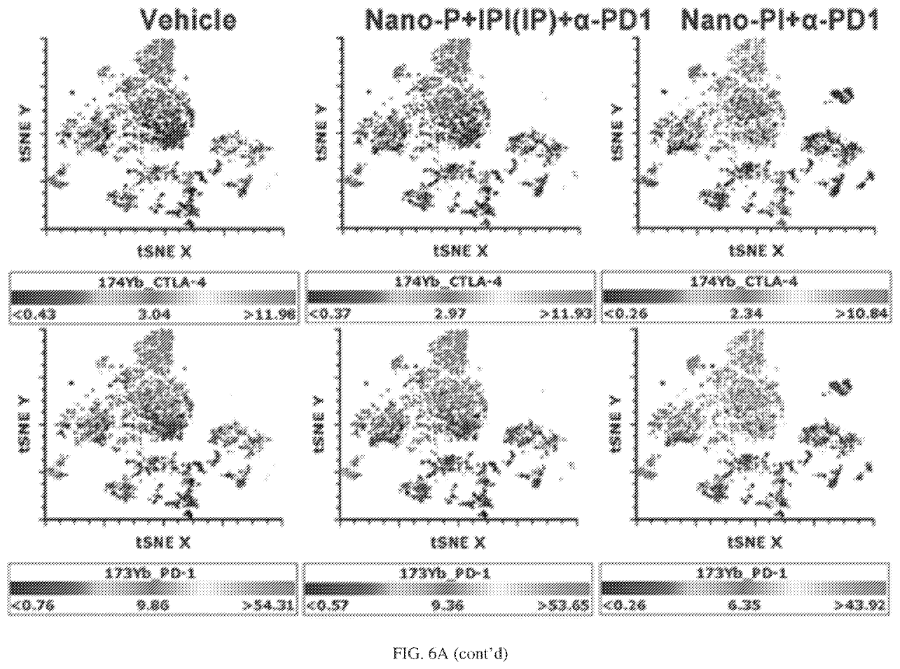

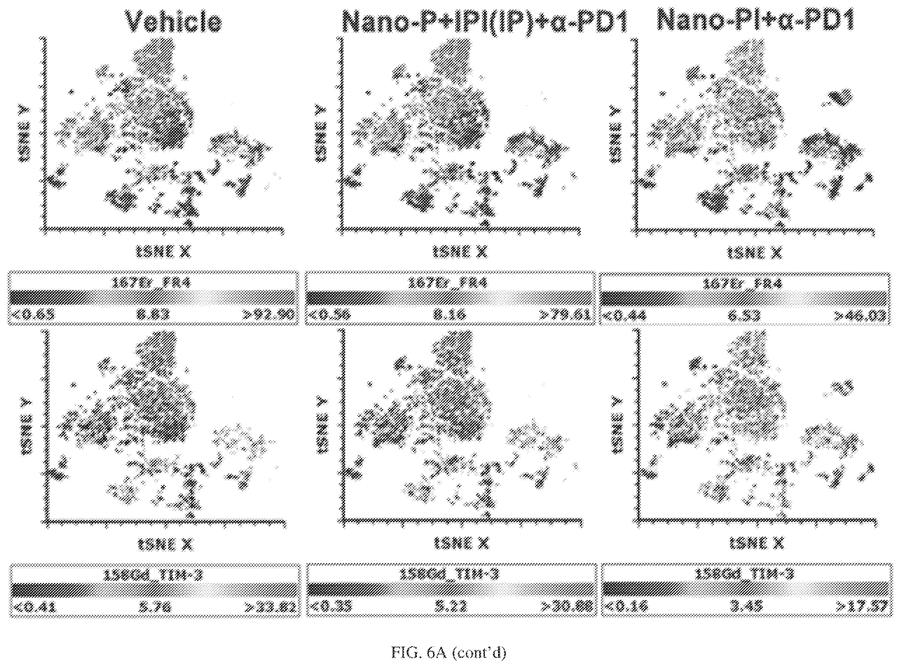

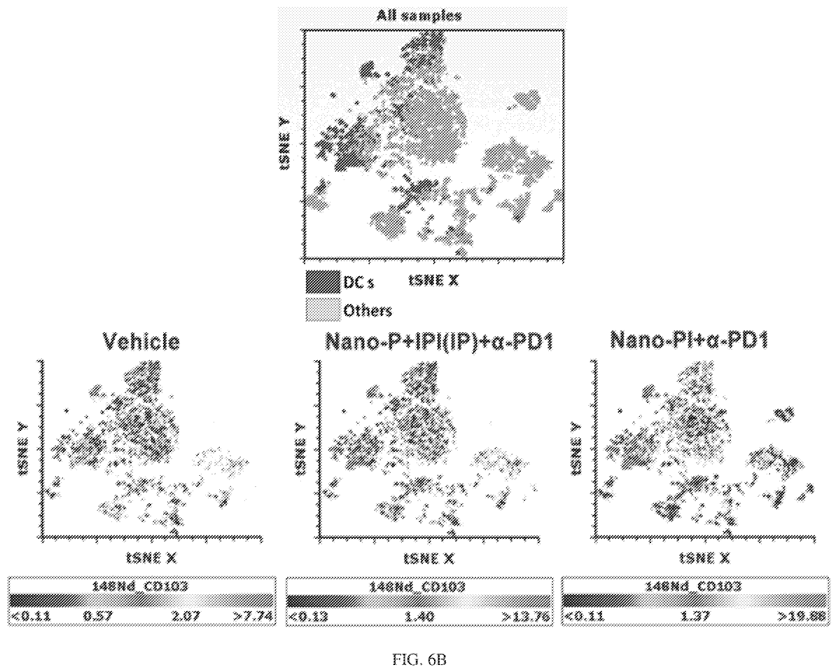

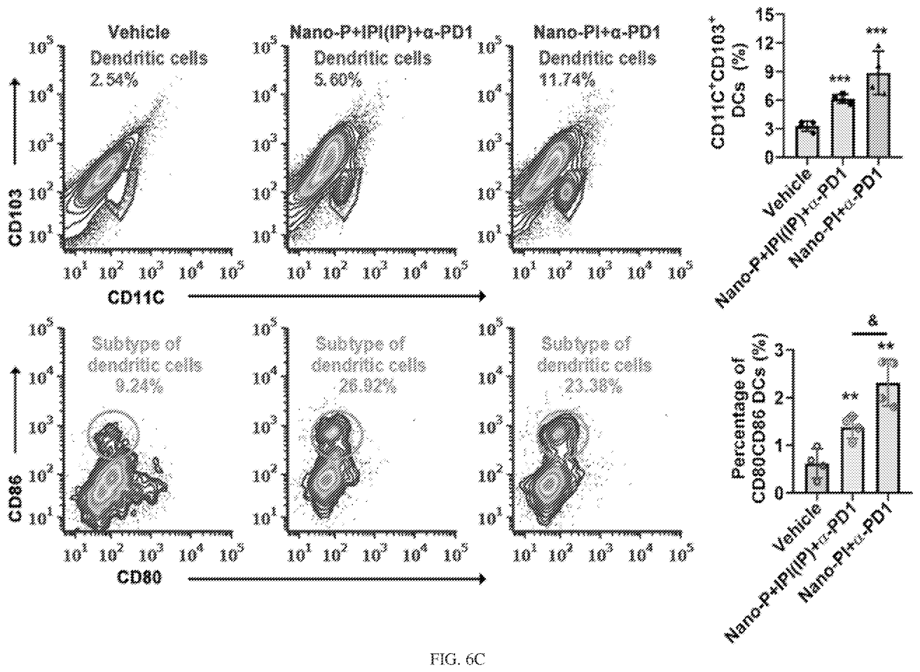

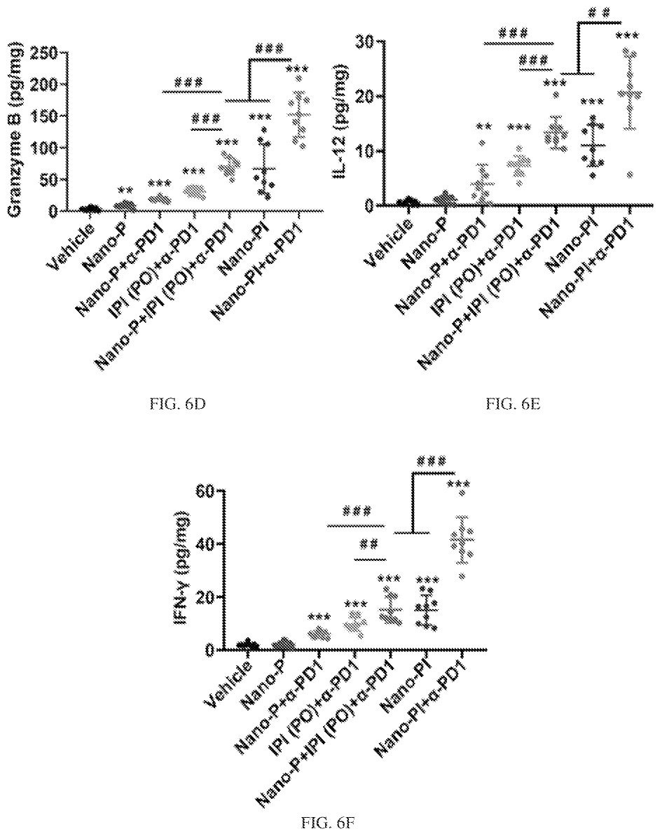

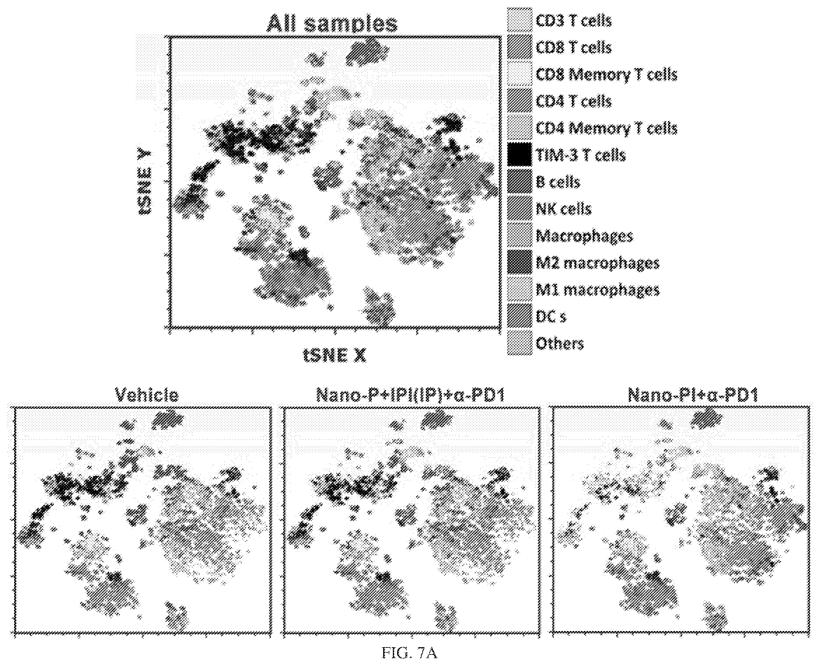

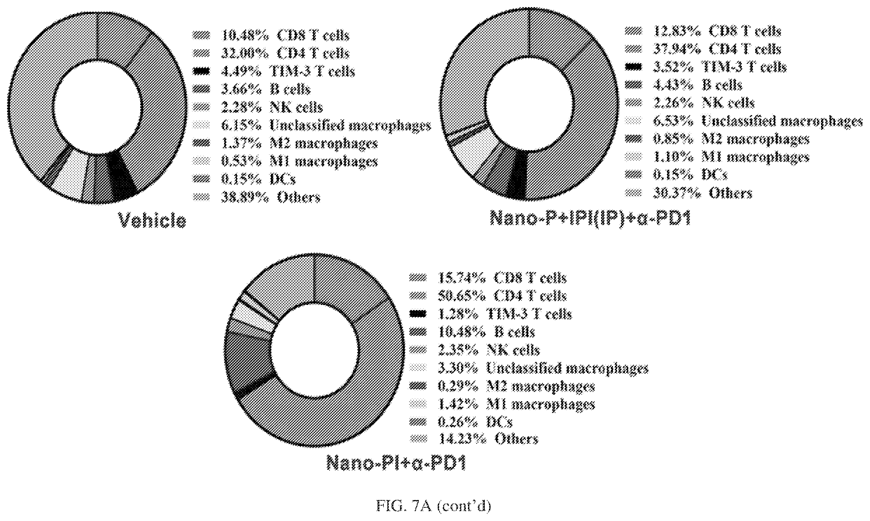

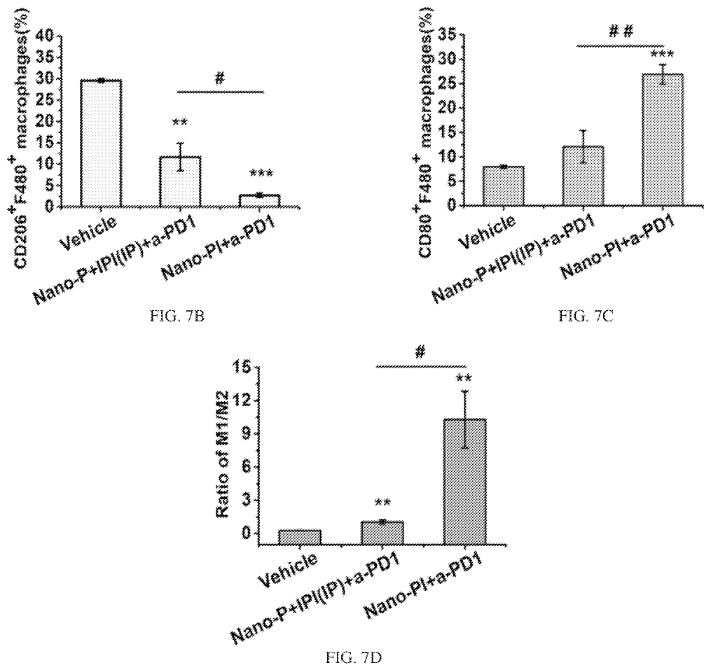

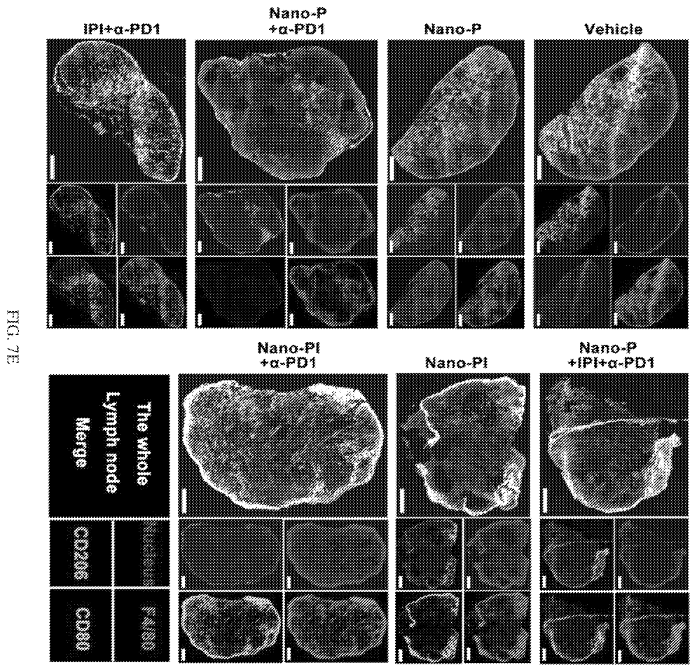

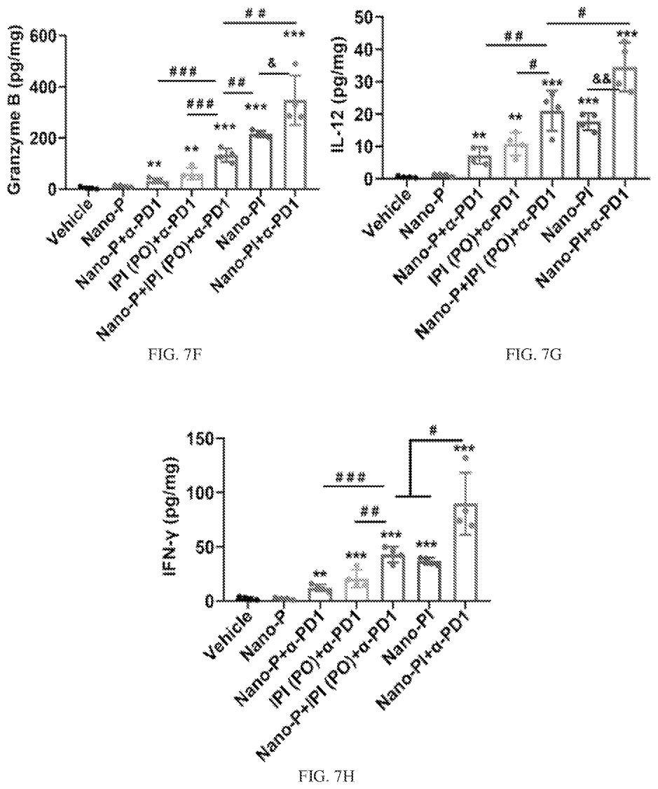

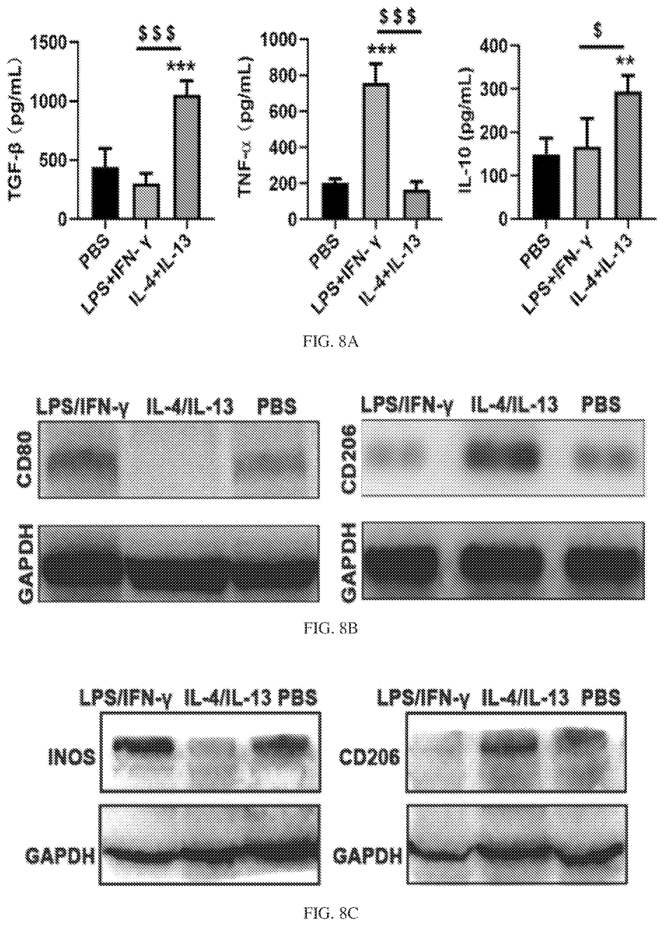

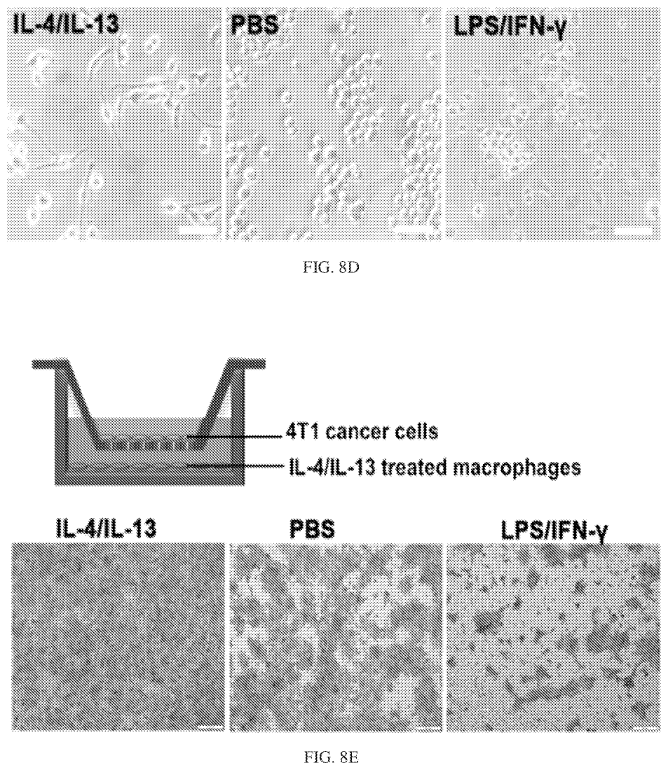

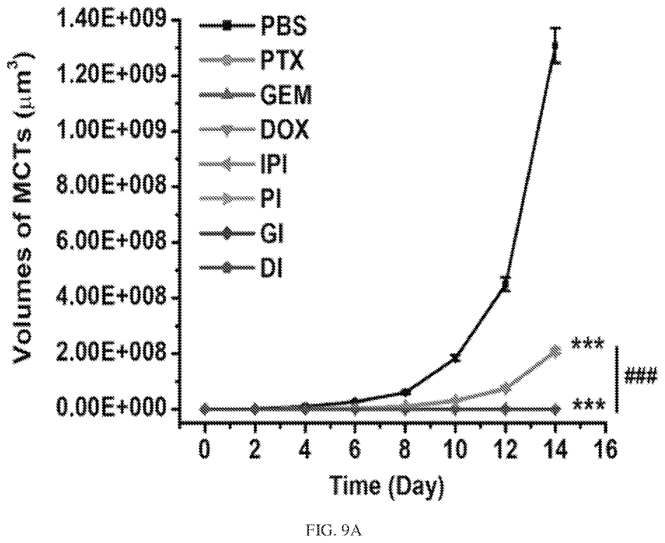

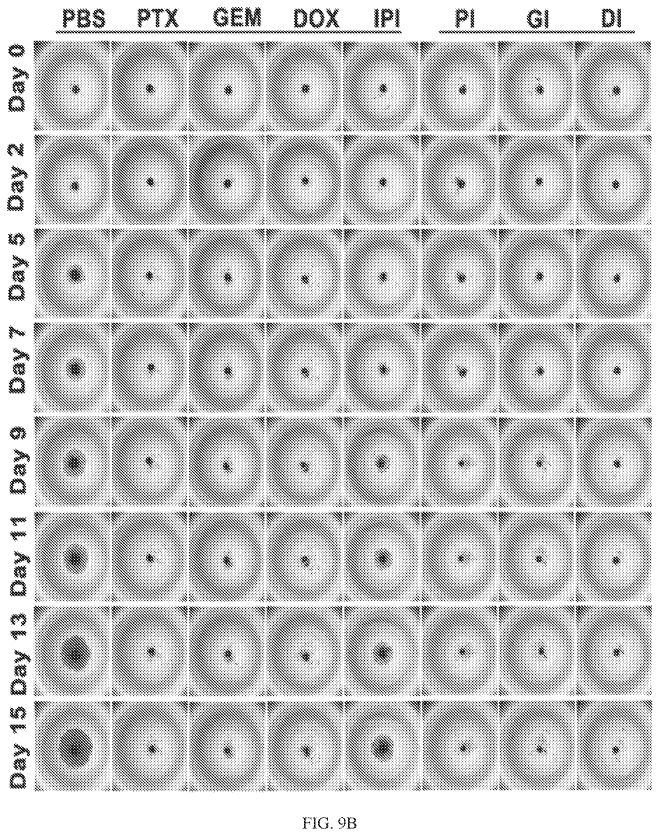

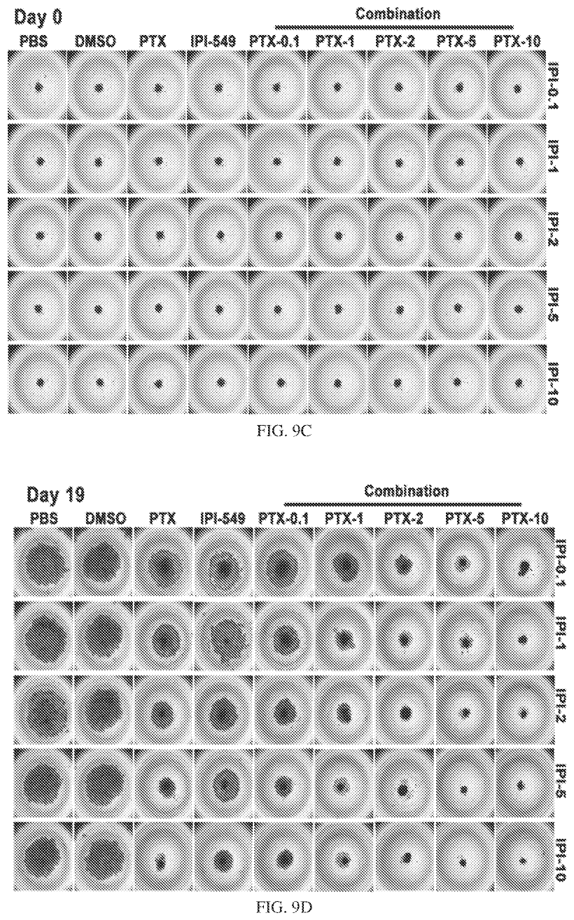

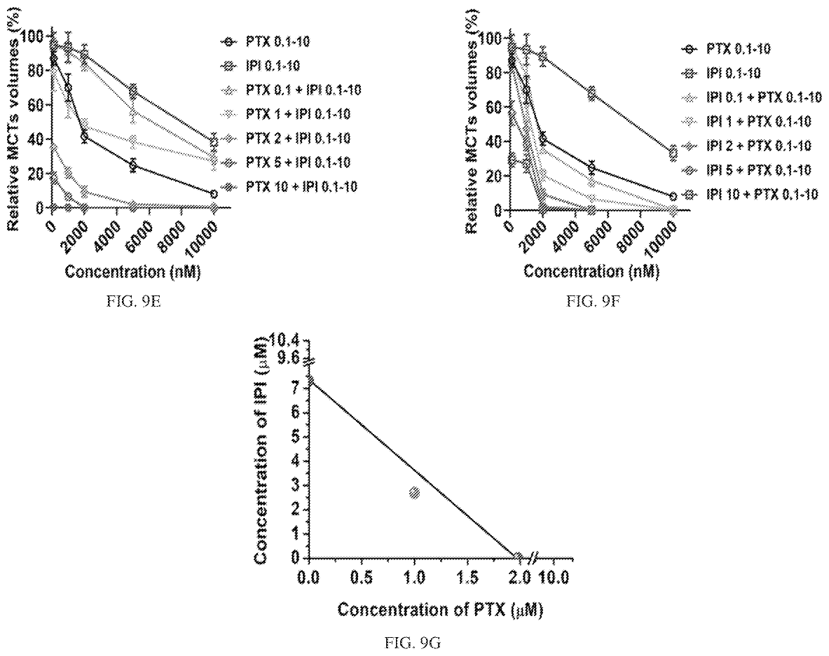

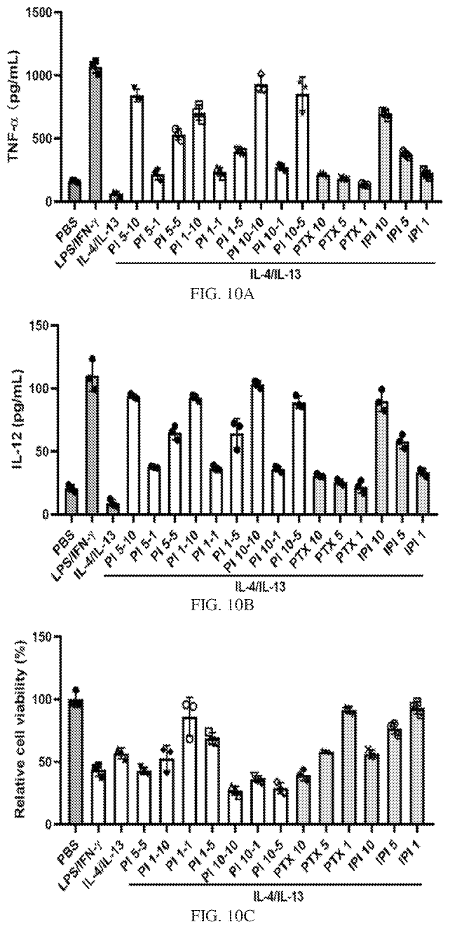

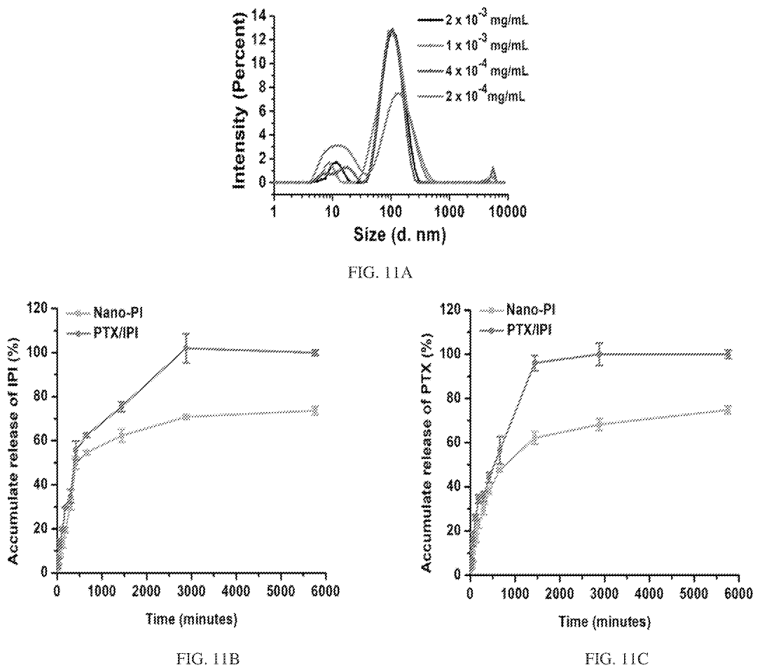

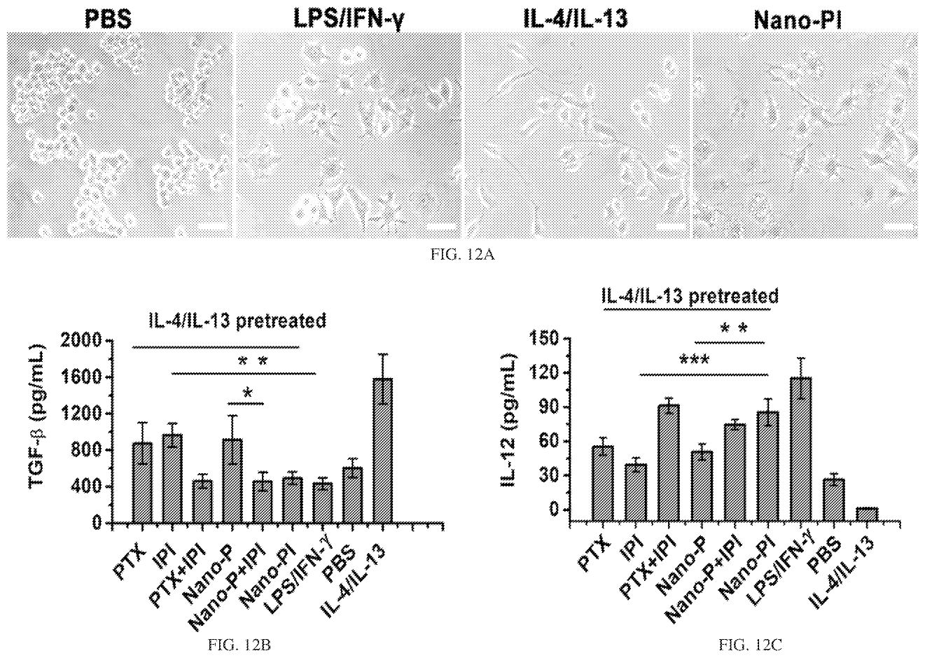

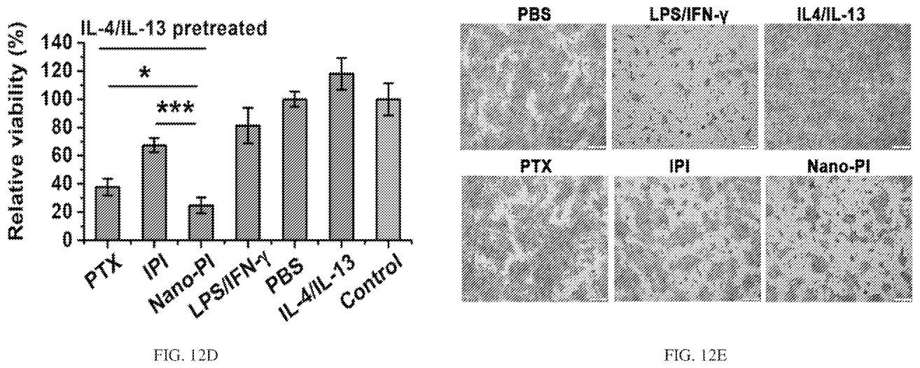

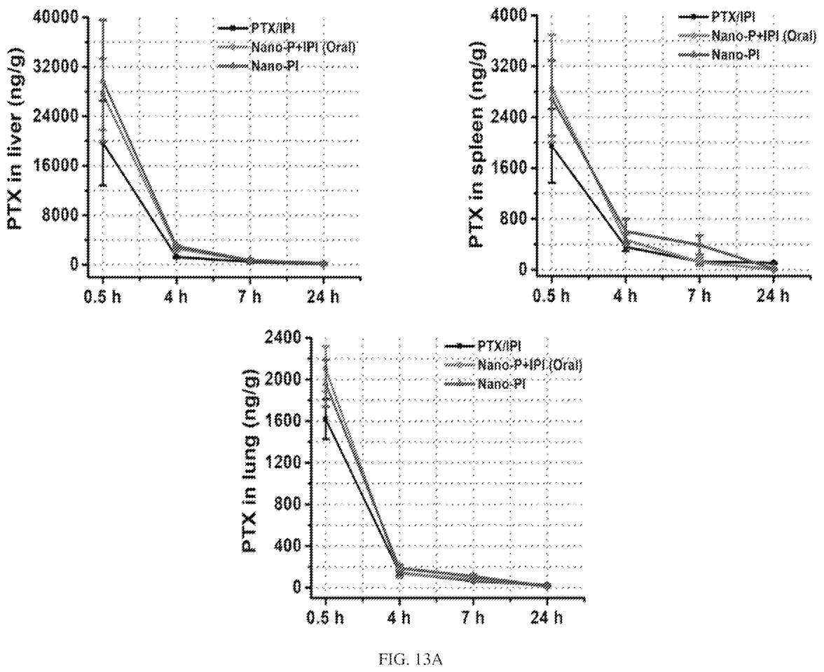

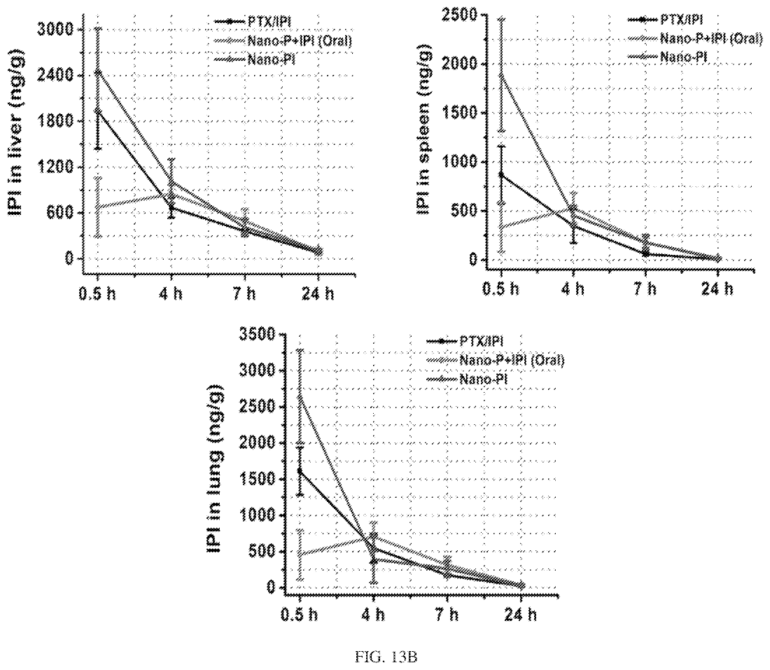

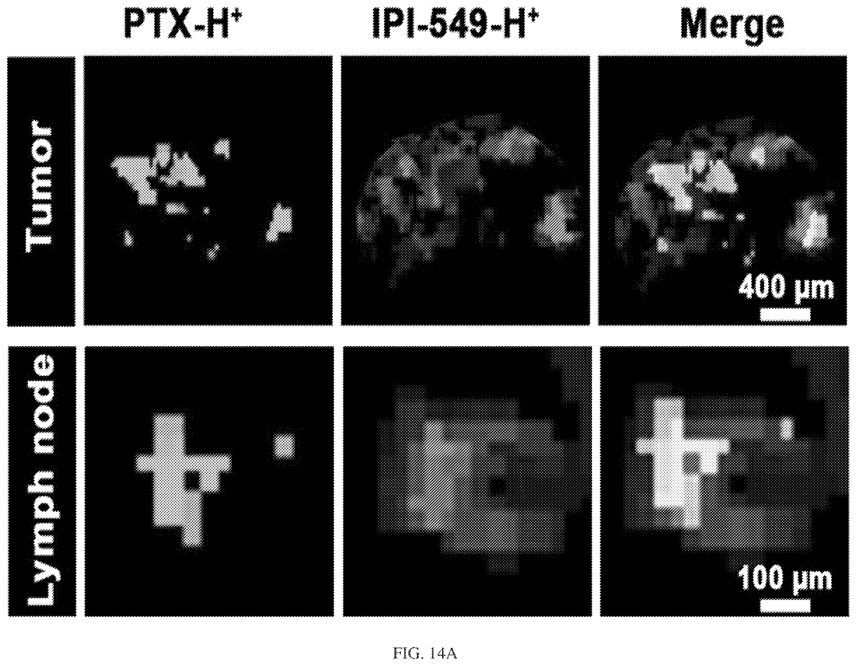

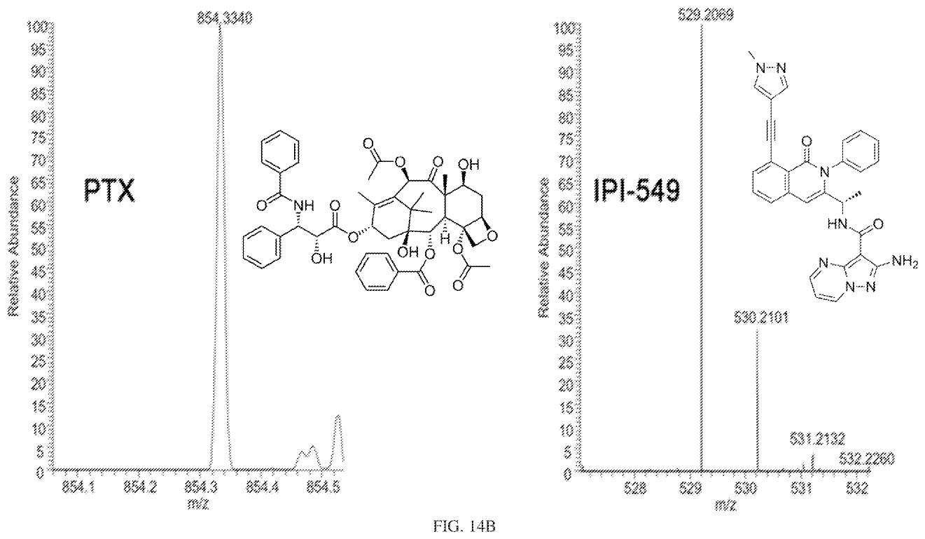

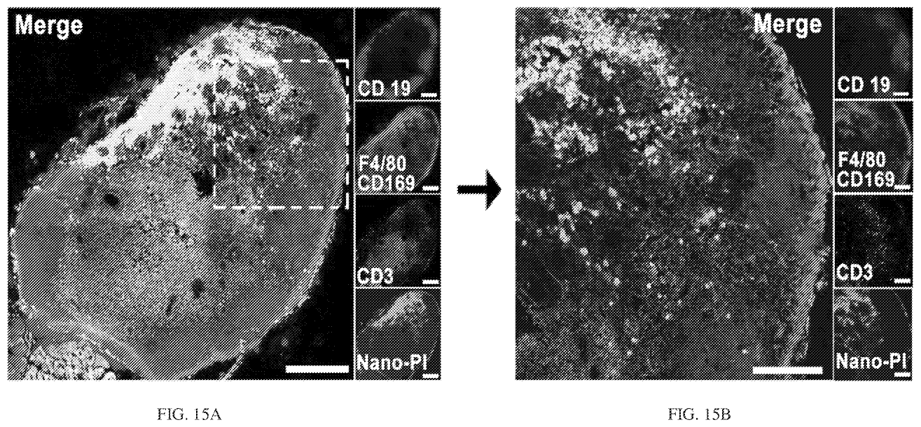

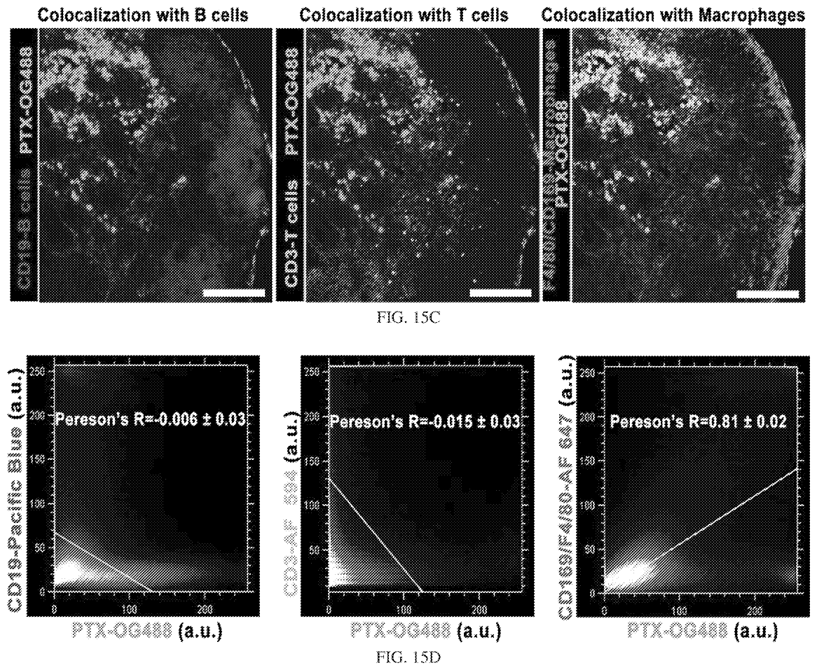

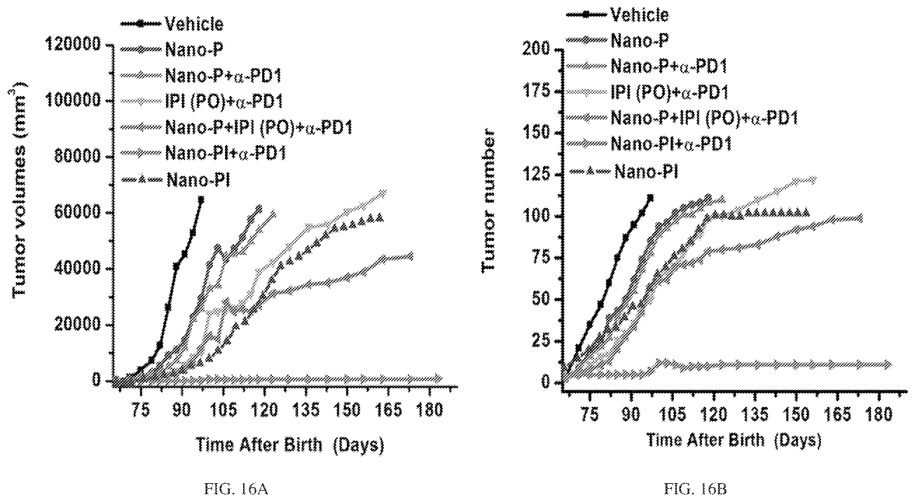

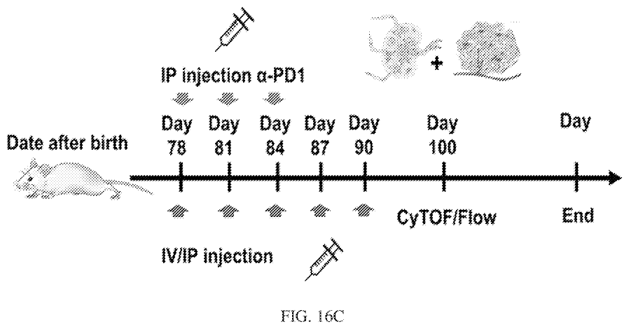

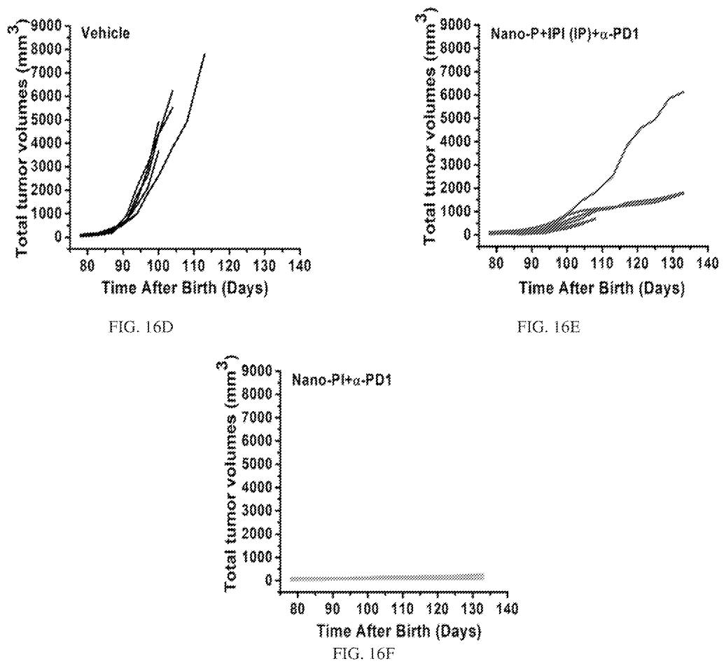

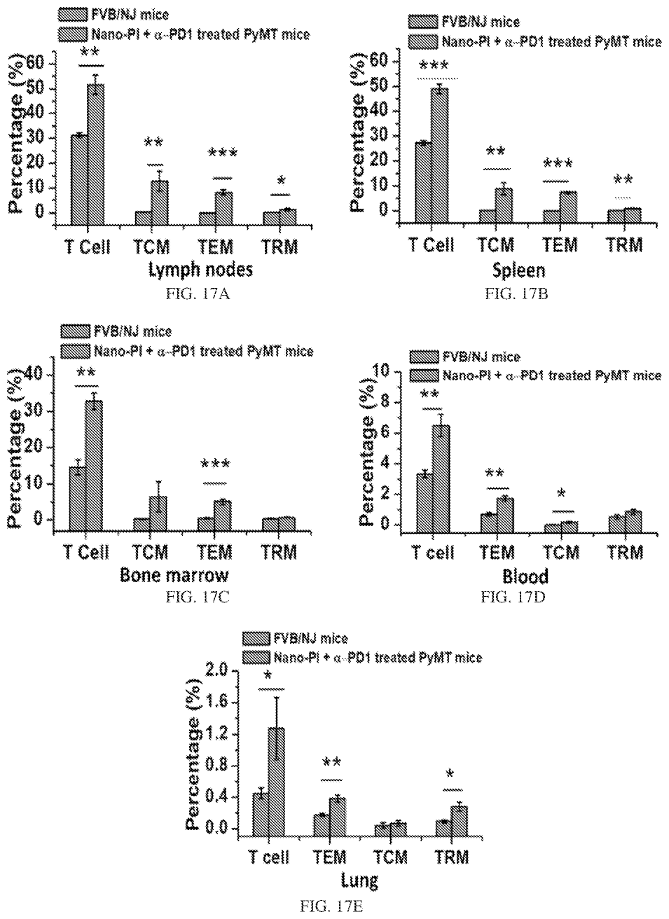

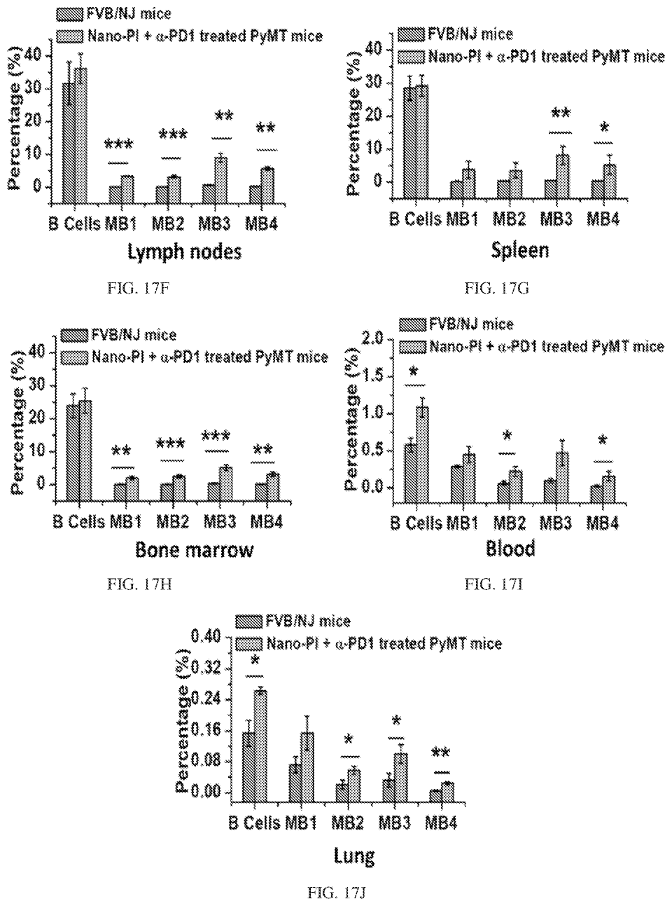

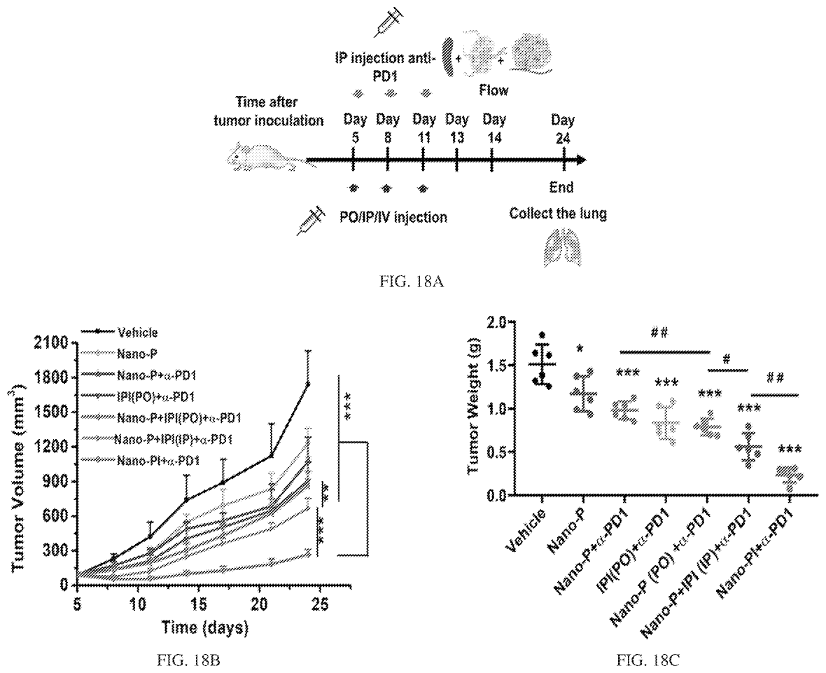

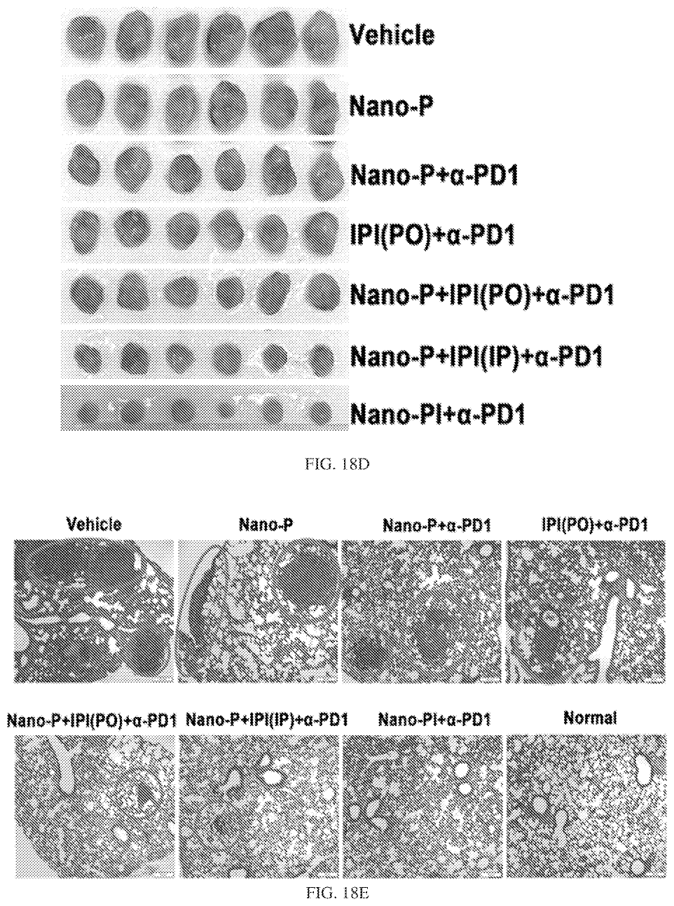

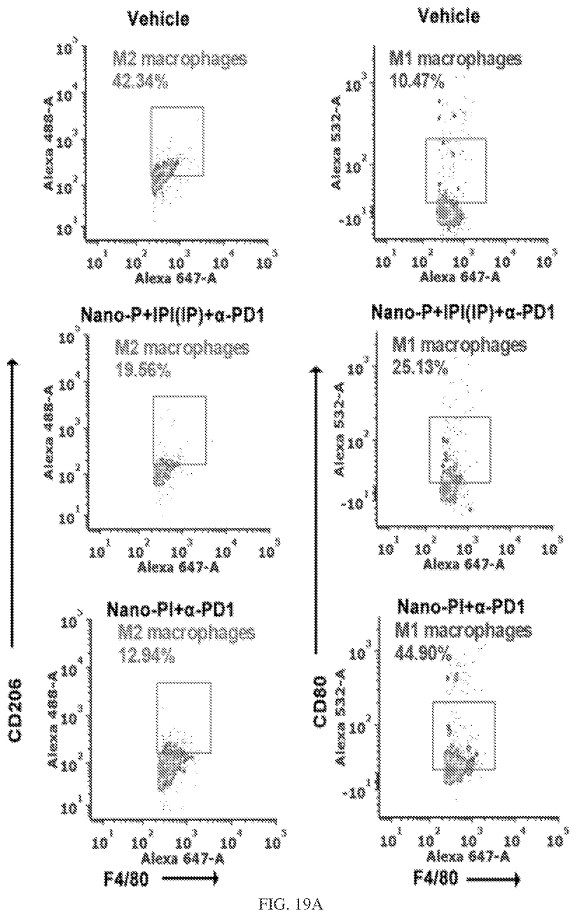

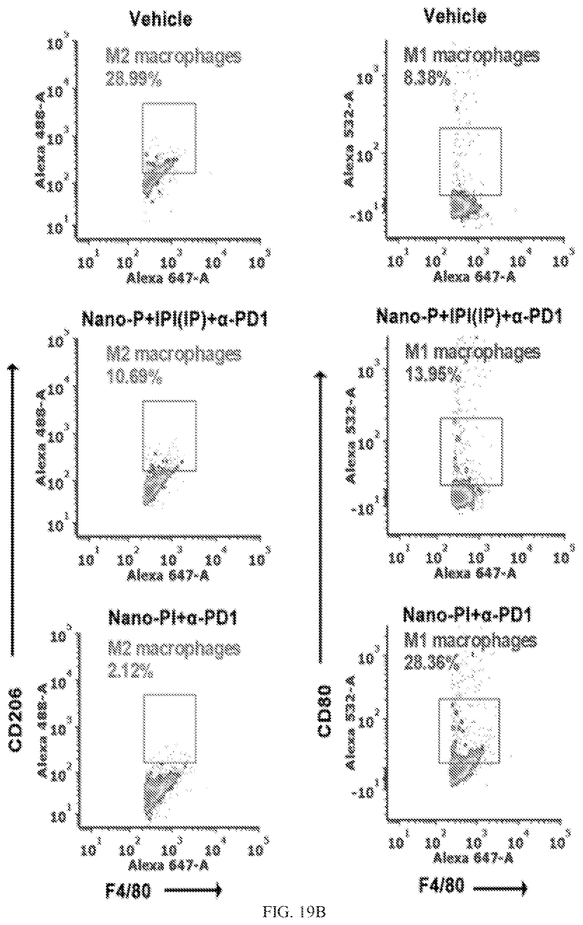

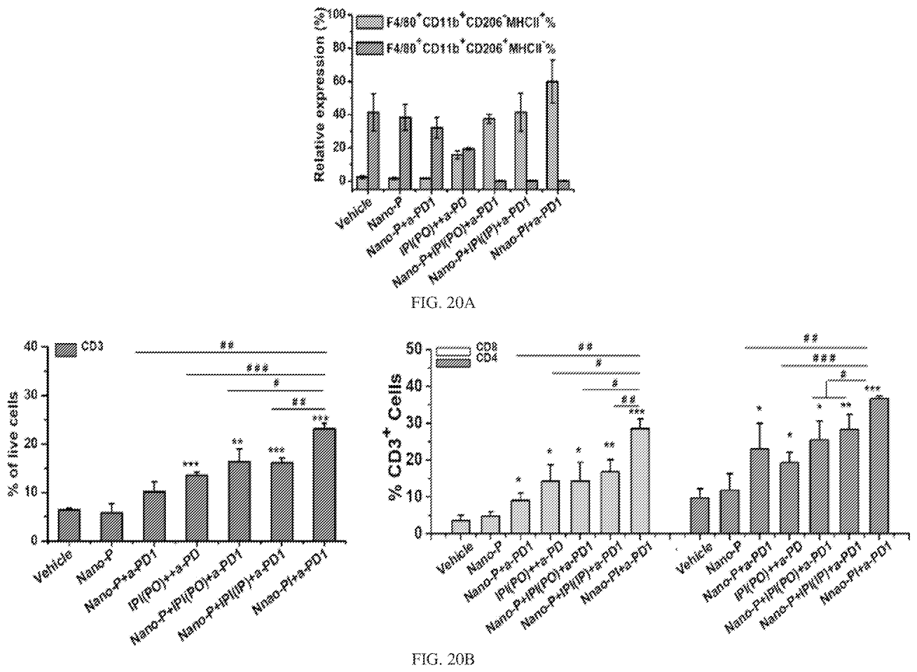

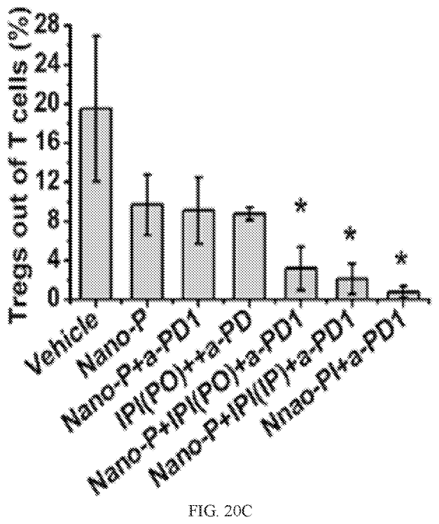

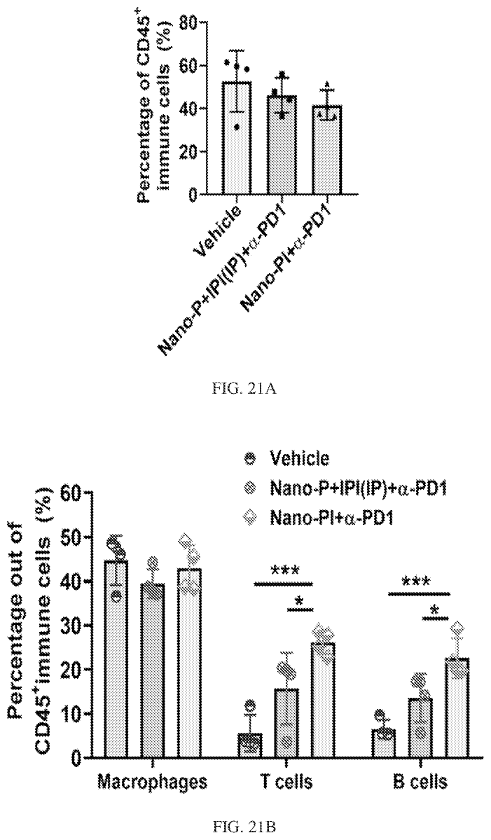

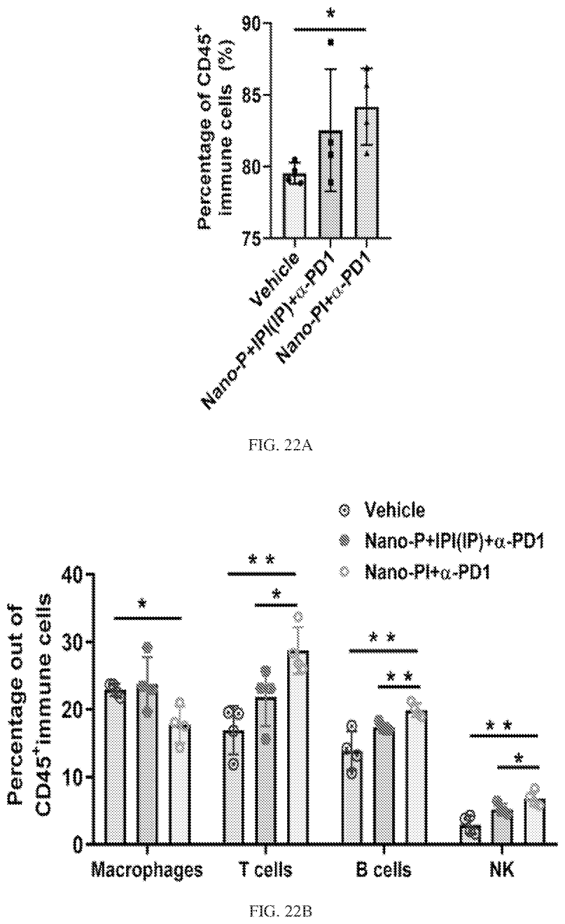

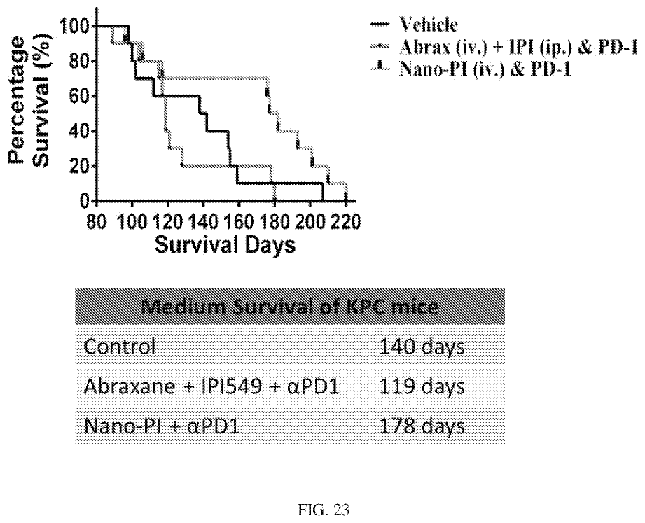

We monitored the total macrophage population, M1 and M2 macrophage frequencies in both tumor and lymph nodes of MMTV-PyMT transgenic mice (FVB/NJ) with spontaneous breast cancer and lung metastasis. Macrophage infiltration was observed in breast tumor lesions (25.1±0.5%) compared to that in the normal fat pad (6.2±1.0%), whereas the M2 phenotype was 3-fold higher in tumors (82.0±5.3%) than in fat pads of control mice (27.3±2.8%) (FIG. 1A). Additionally, M2 macrophages were 3.9-fold higher in the lymph nodes of tumor-bearing mice than in those of naive mice (83.2±2.5% vs, 21.5±1.6%, P=0.001), although the total number of macrophages in the lymph nodes from tumor-bearing mice and normal mice was similar (FIG. 1B). These results were validated through immunofluorescence staining, which showed higher amounts of M2 macrophages in both the tumors and lymph nodes (FIGS. 1C-ID). This increase in M2 macrophages in both tumors and lymph nodes in the PyMT spontaneous metastatic breast cancer model is consistent with clinical observations in patients with breast cancer. Thus, our observations suggest that the modulation of M2 macrophages in both tumors and lymph nodes may lead to better treatment outcomes in metastatic breast cancer.

Enhanced M2 to M1 Macrophages Repolarization and Inhibition of Tumor Spheroid Growth by the Combination of IPI-549 with PTX.

Since previous studies reported that chemotherapy may either increase recruitment of M2 macrophages or induce M2 to M1 macrophage polarization, screening was conducted for an optimal combination of IPI-549 and chemotherapeutic drugs that could effectively repolarize M2 macrophages to the M1 phenotype (FIG. 8) Bone-marrow-derived macrophages (BMDMs) were pretreated with IL-4 and IL-13 to polarize them to M2 macrophages and then treated with various combinations of drugs. As shown in FIGS. 1E-1H, the combination of IPI-549 with PTX enhanced M2 to M1 macrophage polarization as depicted by an increase in M1 markers (TNF-α and IL-12) and a decrease in M2 markers (IL-10 and TGFβ) in comparison with either IPI-549 or PTX alone. However, combinations of IPI-549 with other chemotherapeutic agents (doxorubicin and gemcitabine) did not show such an effect.

We also established three-dimensional (3D) multicellular tumor spheroids (MCTs) by co-culturing breast cancer 4T1 cells with M2 macrophages derived from RAW264.7 cells at a ratio of 7:3 to further investigate the synergistic effect of IPI-549 and PTX against cancer cell growth in the presence of M2 macrophages (FIG. 1I). The combination of IPI-549 and PTX completely inhibited 3D MCTs growth (FIGS. 1J-1K), whereas the combination of IPI-549 with doxorubicin and gemcitabine did not show any obvious improvement compared with a single drug treatment. In addition, a single agent alone, such as IPI-549. PTX, doxorubicin, or gemcitabine, showed limited inhibition of 3D MCTs growth (FIGS. 1J-1K). In addition. PTX and IPI-549 demonstrated a synergistic effect in inhibiting MCTs growth in a co-culture of 4T1 cancer cells and M2 macrophages, in comparison with single drug treatment alone at the different concentrations as indicated by a combination index (CI) of 0.7 (FIGS. 9C-9G). However, the combination of IPI-549 with different chemotherapeutic agents, such as PTX, doxorubicin and gemcitabine, did not show improvement in inhibiting 3D tumor spheroids (established by 4T1 cells alone without macrophages) compared to the single drug treatments (FIGS. 9A-9B). These data suggest that the synergistic antitumor effect of IPI549 and PTX is macrophages-dependent.

### Nanoformulation of IPI549 and PTX (Nano-PI) Enhanced the Accumulation of IPI-549 and PTX in Macrophages Located in Both Tumors and Lymph Nodes.

To repolarize immunosuppressive macrophages, the small molecules should be delivered to the macrophages in both lymph nodes and tumors. Free IPI-549 and PTX have limited accumulation in tumor and lymph nodes and thus have low macrophage distribution within these tissues. Previously, we have shown that albumin nanoparticles of PTX (Abraxane, Nano-P) can specifically accumulate in TAMs. Therefore, we designed the albumin nanoparticles co-encapsulated with IP1-549 and PTX (Nano-PI). We hypothesized that the molecular interaction between the two drugs might have helped to encapsulate them together due to the existing high binding affinity between PTX and albumin, whereas IPI-549 alone cannot be encapsulated in the albumin nanoparticle. Characterization of Nano-PI revealed a diameter of 143.5+2.0 nm with a polydispersity index (PD1) of 0.125+0.0158 and spherical morphology (FIGS. 2A-2B). The drug loading capacities in Nano-PI suspension were 8.1+0.04% (PTX) and 4.3+0.04% (IPI-549); whereas the encapsulation efficiencies of Nano-PI were 88.0+2.8% (PTX) and 83.8+5.4% (IPI-549). The drug recovery yield after processing was 90.7+4.7% (PTX) and 73.3+11.9% (IPI-549). The final drug ratio in Nano-PI was 2:1 (paclitaxel: IPI-549 w/w), which exerted a combination effect on macrophage repolarization and tumor growth inhibition (FIGS. 9-10). Nano-PI was stable within the dilution to PTX concentration of 2×10⁻⁴ mg/ml (in PBS) and to 4×10⁻⁴ mg/ml (in PBS containing 10% fetal bovine serum (FBS)), respectively (FIGS. 2C-2D and FIG. 11A). The in vitro drug release profiles of Nano-PI in plasma at 37° ° C. showed that both PTX and IPI-549 were released more slowly as compared to free drug (FIGS. 11B-11C). Furthermore, Nano-PI promoted macrophage repolarization from the M2 to M1 phenotype, leading to a more potent inhibition of cancer cell migration than PTX or IPI-549 alone (FIG. 12).

Next, we evaluated whether intravenous administration of Nano-PI could enhance drug accumulation in the tumors and lymph nodes in MMTV-PyMT transgenic mice with spontaneous breast cancer. Liquid chromatography tandem mass spectrometry (LC-MS) was performed to quantify the drug concentration in tissues (FIGS. 2E-2F. FIG. 13). As shown in FIGS. 2E-2F and Table 2, Nano-PI resulted in an increased accumulation of both PTX and IPI-549 in tumors and lymph nodes compared to that of free drug (PTX/IPI, IV). This was demonstrated by a 2.4-fold and 2.2-fold increased area under the curve (AUCₜᵢₛₛᵤₑ) of PTX and IPI-549 in tumors, and a 2.0-fold and 2.2-fold increased AUCₜᵢₛₛᵤₑ in lymph nodes as compared with free drug. In addition, Nano-PI increased the accumulation of IPI-549 in tumor and lymph node by 2.3- and 2.5-fold (AUCₜᵢₛₛᵤₑ) in comparison with the clinically used combination of PTX albumin nanoformulation (Nano-P. I.V.) and IPI-549 (P.O) (Nano-P/IPI, P.O.) or Nano-P (I V.) plus IPI-549 (I.P.) (FIGS. 2E-2F and Table 2).

TABLE 2
AUC_{0-24 h} (ng*h/ml for plasma or ng*h/g for tissue) of PTX and
IPI-549 in different tissues calculated from data in FIG. 2E

| | PTX | | | | IPI-549 | | | |
|---|---|---|---|---|---|---|---|---|
| | Plasma | Tumor | Fad pad | Lymph node | Plasma | Tumor | Fad pad | Lymph node |
| PTX/IPI | 1886.6 | 16950.5 | 8376.0 | 6081.5 | 4757.8 | 6360.5 | 4092.6 | 4047.5 |
| Nano- | 1414.1 | 34720.5 | 8908.5 | 10820.6 | 4298.6 | 6328.1 | 5059.3 | 3715.0 |
| P + IPI(PO) | | | | | | | | |
| Nano- | 1309.5 | 33385.6 | 9236.1 | 19026.9 | 4814.2 | 5060.2 | 2955.2 | 3546.0 |
| P + IPI(IP) | | | | | | | | |
| Nano-PI | 1450.2 | 41349.7 | 8521.2 | 10321.7 | 3880.1 | 14036.7 | 8482.2 | 8716.3 |

To evaluate whether Nano-PI efficiently delivered the drug to macrophages in both tumors and lymph nodes, we prepared Nano-PI encapsulated with fluorescent OG488-labeled PTX and IP1549 (F-Nano-PI). We then visualized drug distribution in different types of cells in both tumors and lymph nodes after intravenous administration by confocal imaging and flow cytometry. F-Nano-PI (green) predominantly distributed in macrophages in tumors as indicated by the colocalization with macrophages(red) (FIGS. 3A-3B, FIG. 13). Flow cytometry analysis further proved that F-Nano-PI increased drug accumulation in tumors (from 6.9+1.5% to 17.3+2.8%) as compared to free drug (F-PTX/IPI). Free drugs were evenly distributed in tumor cells (39.88%) and TAMs (38.94%); whereas the F-Nano-PI were distributed more in TAMs (67.75%) than tumor cells (23.06%) (FIG. 3C, FIG. 14).

In lymph nodes, Nano-PI also enhanced drug accumulation in macrophages as indicated by the strong co-localization of F-Nano-PI (green) with macrophages (red) but not with B or T cells (FIGS. 3D-3G, FIG. 15). Further, F-Nano-PI was distributed more in medullary sinus macrophages (MSMs) and medullary cord macrophages (MCMs) than subcapsular sinus macrophages (SSMs) (FIGS. 3F-3H). In contrast, the distribution of the free drug (PTX-OG488) in lymph nodes was lower and less colocalized in macrophages. Collectively, our data suggested that Nano-PI enhanced the delivery of PTX and IPI-549 to lymph nodes and tumors especially to the macrophages in these tissues in comparison with free drugs.

Nano-PI Combined with α-PD1 LED to Complete Long-Term Remission and Eliminated Lung Metastasis in PyMT Transgenic Mice

The anti-tumor efficacy of Nano-PI in combination with α-PD1 treatment was evaluated in transgenic MMTV-PyMT mice with spontaneous breast cancer and lung metastasis (32, 38-40). The MMTV-PyMT mice grew 1-15 mammary tumor foci and developed multiple lung metastases lesions in 8-10 weeks. They were sacrificed when the largest tumor lesion reached 2 cm in diameter. Drug treatment began when mice were 9-10 weeks old when the multifocal mammary adenocarcinomas had reached a total volume of 80-110 mm³ for all lesions (FIG. 4A). Combination treatment of Nano-PI (PTX 10 mg/kg and IPI-549 5 mg/kg via five I.V, doses) and α-PD1 antibodies [100 μg/mouse, three doses by intraperitoneal (I.P.) injection] eradicated tumor growth and achieved complete tumor remission (100% complete response. CR) over 183 days after birth (FIG. 4B and FIGS. 16A-16B), eliminated lung metastasis (FIG. 4C), and resulted in 100% mouse survival (FIG. 4D). The average total tumor volume per mouse in the vehicle group reached 12020.4+2404.1 mm³ on an average of 88 days after birth, and the median survival time was 88 days (50% mice died or were sacrificed as pre-determined to reach the endpoints) (FIG. 4B). The current clinically tested regimen, IPI-549 (P.O.) combined with Nano-P (I.V.) and α-PD1 (I.P.), resulted in delaying tumor growth and a median survival of 123 days but did not achieve complete cancer remission (0% CR). Other treatment groups including Nano-P (IV). Nano-P (I.V.) plus α-PD1. IPI-549 (P.O.) plus α-PD1, and Nano-PI (I. V.) only, did not achieve complete tumor remission (FIGS. 4A-4D). The antitumor efficacy of Nano-PI plus α-PD1 was confirmed again using the same dosing regimen as described above compared with the three-drug combination of Nano-P, IPI-549 (I.P.), and α-PD1 (FIG. 16C). Nano-PI plus α-PD1 exhibited complete tumor remission (>130 days), whereas the combination of Nano-P and IPI-549 (I.P.) and α-PD1 showed only a partial response (FIGS. 16D-16)

To test the long-term efficacy of Nano-PI plus α-PD1 in the mice with tumor remission, we next performed tumor re-challenge implantation on day 210 after birth on the PyMT mice with tumor remission by implanting (S.C.) 5×10⁶ cells/mL. PyMT cancer cells in the mammary fat pads and without further treatment (FIG. 4E). The mice with long term tumor remission that were initially treated with Nano-IP and α-PD1 completely rejected tumor growth from the implanted PyMT cancer cells for 38 days, whereas naive mice (FVB/NJ mice, control group) showed tumor growth of ˜400 m³ (FIG. 4F). It is worth noting that these data did not suggest treatment of Nano-PI combined with α-PD1 cure the PyMT spontaneous breast cancer because we observed endogenous tumor lesion started to growth around 200 days after birth. Additionally, we detected the total memory T and B cells populations in the peripheral blood, bone marrow, lymph nodes, spleen, and lung at the end of the tumor re-challenge experiments (248 days after birth). Compared with the control group, the mice with long-term tumor remission and prior treatment had higher central memory T cells (TCM, CD3⁺CD62L⁺CD44⁺) and effector memory T cells (TEM, CD3⁺CD62L⁻CD44⁺) (FIGS. 17A-17E). Furthermore, we also measured four memory-related B cell subsets: MB1 (CD19⁺CD73⁺CD80⁺). MB2 (CD19⁺CD73⁺PD-L2⁺), MB3 (CD19⁺PD-L2⁺CD80⁺), and MB4 (CD19⁺CD73⁺CD80⁺PD-L2⁺) (FIGS. 17F-17J). Mice with long-term tumor remission also had higher amounts of these four subtypes of B cells in the lymph nodes, spleen, bone marrow, peripheral blood, and lungs. These data suggest that Nano-PI combined with α-PD1 may have induced higher total memory cells, which probably contribute to its long-term anticancer efficacy. However, it is worth noting that we only measured total immune memory cells, but not antigen-specific memory cells due to unknown antigens in this model.

We further tested the anti-tumor efficacy of Nano-PI at a lower dose in comparison with a combination of free PTX and IPI-549 by intravenous administration (five doses). Nano-PI (PTX 5 mg/kg, IPI-549 2.5 mg/kg. I.V.) plus α-PD1 (100 μg/mouse, three doses by I.P.) or the combination of free PTX (5 mg/kg. I.V.) and IPI-549 (2.5 mg/kg. I.V.) plus α-PD1 (100 μg/mouse, three doses by I.P.) were administered to MMTV-PyMT mice (FIG. 4G). Compared to combination of free PTX and IPI-549, Nano-PI inhibited tumor growth (FIG. 4H), lung metastasis (FIG. 4I) and resulted in 100% survival at the end of observation (FIG. 4J). Finally, we also evaluated the anticancer efficacy of Nano-PI and α-PD1 in an orthotopic metastatic breast tumor model by implanting 4T1 cells into the mammary fat pad of BALB/c mice. Nano-PI (three doses, once every three days, 10 mg/kg PTX, 5 mg/kg IPI-549) in combination with α-PD1 exhibited the most anticancer efficacy on tumor growth and lung metastasis, compared with either single administration of Nano-P or IPI-549 or a combined administration (FIG. 18).

Nano-PI Combined with α-PD1 Remodeled Tumor Immune Microenvironment by Promoting M2 to M1 Macrophages Repolarization, Increasing CD4⁺ and CD8⁺ T Cells, Decreasing Tregs, and Preventing T Cell Exhaustion in Tumors

To explore the details of how Nano-PI remodeled the tumor immune microenvironment, we first utilized cytometry by time of flight (CyTOF) to monitor tumor immune cell profiles in MMTV-PyMT spontaneous breast cancer 10 days after the last treatment. Nano-PI plus α-PD1 altered the M1 and M2 frequencies resulting in 5-fold reduction in M2 macrophages and a 2-fold increase in M1 macrophage population (M1: 15 87%, M2 3.2%), in comparison with intravenous injection of Nano-P plus IPI-549 and α-PD1 (M1: 24.37%, M2: 16.4%) or the vehicle group (M1: 8.4%, M2: 22.55%) (FIG. 5A). Nano-PI plus α-PD-1 reduced expression of immunosuppressive M2-macrophages markers (CD206, CD115) and decreased expression of the anti-inflammatory cytokines IL-10 and IL-4 in immune cells (FIG. 5B). In addition, flow cytometry analysis from the same mouse experiments showed that Nano-PI plus α-PD1 treatment decreased M2 macrophages by 4-fold and increased M1 macrophages by 3- to 4-fold in tumor tissues (FIGS. 5C-5E, FIG. 19A). The immunofluorescence staining further demonstrated that the Nano-PI plus α-PD1 induced the most efficient M2 to M1 macrophage repolarization in tumors in comparison with other treatment groups, where Nano-PI plus α-PD1 treated tumors had very low M2 macrophages as indicated by the minimal staining of M2 macrophage marker, but an increase of M1 macrophages in tumors (FIG. 5F). Moreover. Nano-PI at a lower dose (Nano-PI, PTX 5 mg/kg, IPI-549 2.5 mg/kg) was also confirmed to repolarize macrophages from M2 to M1 in orthotopic breast cancer using 4T1 breast cancer mice (FIG. 20A) and MMTV-PyMT (FIG. 19A).

In addition, we also analyzed the immune cell infiltration in tumors of PyMT mice by flow cytometry (2 million cells per sample) 10 days after the last treatment. Although drug treatments did not change the total number of immune cells (CD45+) infiltrated in tumors, Nano-PI plus α-PD-1 group and Nano-P plus IPI-549 and α-PD1 group decreased macrophage infiltration to 20% and 17%, respectively, compared to 27% of macrophage infiltration in vehicle treated group. Further, the Nano-PI plus α-PD-1 treated group and Nano-P plus IPI-549 and α-PD1 treated group increased T cell infiltration to 14% and 7.4%, respectively compared to 2% in the vehicle treated group. Finally, the Nano-PI plus α-PD-1 treated group and Nano-P plus IPI-549 and α-PD1 treated group increased B cell infiltration to 8.9% and 6.8%, respectively compared to 3.2% in the vehicle treated group (FIG. 21).

To investigate how Nano-PI plus α-PD1 treatment altered T-cell immunity in the tumors of both PyMT mice (FIG. 6A) and 4T1 orthotopic breast cancer mice (FIGS. 20B-20C), we first performed analysis of subtypes of T cells by CyTOF. The results revealed that Nano-PI plus α-PD1 treatment resulted in a 6.6- and 11-fold increase of CD4⁺ T cells, 2.5- and 3.4-fold increase of CD8⁺ T cells, compared to Nano-P combined with IPI-549, α-PD1 and vehicle treatment groups, respectively (FIG. 6A). Furthermore, Nano-PI plus α-PD1 treatment decreased Tregs in tumors by 20-fold (0.8%) compared with the vehicle group (19 5%) in PyMT mice. Similar results by flow analysis were also observed in orthotopic breast cancers using 4T1 cells (FIG. 20C). Finally, Nano-PI plus α-PD1 treatment prevented T cell exhaustion in the tumor tissues confirmed by the decreased expression of exhaustion markers (CTLA-4, PD-1, TIM-3, and FR4) (FIG. 6A).

In addition, Nano-PI plus α-PD1 treatment also increased DCs cell percentages among immune cells (FIG. 5A) and CD103⁺ positive DCs, which helped induce CD8⁺ T cell-mediated anti-tumor immunity (FIG. 6B). Further characterization of dendritic cells in tumors by flow cytometry revealed that Nano-PI plus α-PD1 treatment increased the total DCs (CD11C+CD103⁺) population 8.9+2.0% compared to 3.2+0.5% in vehicle treated group. This treatment also elevated activated DCs (CD80+CD86+) percentage by 1.7 to 3.8-fold compared with Nano-P plus IPI-549 and α-PD land vehicle groups, which are crucial for T-cells activation. (FIG. 6B). Finally. Nano-PI and α-PD1 treatment also increased the concentration of granzyme B. IL-12, and IFN-γ in tumors, indicating a high amount of activated antigen-presenting cells (DCs or macrophages) and the cytotoxic T cells (FIGS. 6D-6F).

Nano-PI Combined with α-PD1 Remodeled the Immune Microenvironment in Lymph Nodes by Promoting M2- to M1-Macrophage Polarization, Increasing CD4⁺ and CD8⁺ T Cells, Increasing B Cells, Decreasing TIM 3⁺ T Cells

We next analyzed the immune cell subpopulation alterations in the lymph nodes of MMTV-PyMT mice 10 days post-treatment by CyTOF and flow cytometry. The CyTOF analysis showed that Nano-PI plus α-PD1 treatment decreased M2 macrophages frequency by 3-fold and increased M1 macrophages frequency by 2.5-fold compared to vehicle group (FIG. 7A). Flow cytometry analysis further confirmed these findings and showed a 5- to 14-fold decrease in M2 macrophages and a 2- to 3-fold increase in M1 macrophages compared to the Nano-P plus free IPI-549 treatment group and vehicle control treatment group (FIGS. 7B-7D, FIG. 19B). In addition. Nano-PI plus α-PD1 treatment increased CD4+T and CD8⁺ T cells frequencies and decreased TIM-3 positive T cells frequency in the lymph nodes compared with both the Nano-P with IPI-549 and α-PD1 treated group and the vehicle treated group (FIG. 7A). Furthermore, Nano-PI plus α-PD1 treatment also increased B cells frequency by more than 2-fold in the lymph nodes as compared to the other groups (FIG. 7A).

In addition, the total number of each immune cell subpopulation in lymph nodes of PyMT mice 10 days post-treatment were also monitored using flow cytometry with 2 million cells from each sample. The data showed that total number of each immune cells subpopulation in the lymph nodes were altered after treatment of Nano-PI plus α-PD1 compared to Nano-P plus IPI-549 and α-PD1 or vehicle treated groups (FIG. 22). Macrophage infiltration, as a percentage of total cells, was slightly decreased from 18.2% (control) to 16.4% (Nano-P. IPI and α-PD1) and 12.2% (Nano-PI plus α-PD1) in the lymph nodes. T cell infiltration, as a percentage of total cells, was increased from 13.4% (control) to 18.9% (Nano-P, IPI and α-PD1) and 24.4% (Nano-PI plus @PD-1) in the lymph nodes. Furthermore, B cell infiltration was also enhanced from 8.7% (control) to 13.9% (Nano-P. IPI and α-PD1) and 16.5% (Nano-PI plus α-PD1) in the lymph nodes. Finally. NK cell infiltration, as a percentage of total cells, was increased from 1.3% (control) to 4.0% (Nano-P, IPI, and α-PD1) and 5.0% (Nano-PI plus α-PD1) in the lymph nodes (FIG. 22).

Furthermore, confocal microscopic images of the whole lymph node showed that the Nano-PI plus α-PD1 treatment group decreased M2 macrophages and increased M1 macrophages as compared to other treatment groups and the vehicle treated group (FIG. 7E). Finally, the Nano-PI plus α-PD1 treated group increased anti-tumor cytokine production (granzyme B, IL-12, and IFN-γ) in the lymph nodes compared with the other treatment groups (FIG. 7F-7H). These results indicate that the Nano-PI with α-PD1 treatment successfully remodeled the microenvironment of lymph nodes that contributed to their anticancer efficacy in PyMT mice.

### Pharmacokinetic Data

Plasma pharmacokinetics parameters are shown in Table 3.

TABLE 3
The plasma pharmacokinetics parameters of different formulations.

| | | | | | | | | Nano- |
|---|---|---|---|---|---|---|---|---|
| | Nano-P + | | | Nano- | Nano-P + | | | P + IPI |
| | IPI (P.O.) | PTX/IPI | Nano-PI | P + IPI(IP) | IPI(P.O.) | PTX/IPI | Nano-PI | (IP) |
| | PTX | PTX | PTX | PTX | IPI | IPI | IPI | IPI |
| Administration route | IV | IV | IV | IV | PO | IV | IV | IP |
| Tlast: hr | 24 | 24 | 24 | 24 | 24 | 24 | 24 | 24 |
| Clast: ng/ml | 12.2 | 8.0 | 15.4 | 6.6 | 2.5 | 0.7 | 0.4 | 80.7 |
| T_max_obs: hr | 0.5 | 0.5 | 0.5 | 0.5 | 4 | 0.5 | 0.5 | 4 |
| C_max_obs: ng/ml | 350.4 | 539.8 | 340.6 | 401.0 | 645.5 | 853.6 | 606.1 | 686.7 |
| C_0: ng/ml | 470.1 | 757.7 | 449.5 | 534.3 | 0.0 | 925.0 | 627.8 | 0.0 |
| Adjusted_r2 | 1.0 | 1.0 | 1.0 | 0.5 | 0.9 | 1.0 | 1.0 | 0.6 |
| Lambda_z: hr−1 | 0.1 | 0.1 | 0.1 | 0.1 | 0.3 | 0.3 | 0.3 | 0.1 |
| MRT: hr | 8.3 | 4.4 | 10.7 | 3.6 | 3.8 | 3.4 | 3.5 | 10.2 |
| N_lambda_z | 3 | 3 | 3 | 4 | 3 | 3 | 3 | 3 |
| Dose: mg/kg | 5 | 5 | 5 | 5 | 2.5 | 2.5 | 2.5 | 2.5 |
| AUC_last: (ng*hr)/ml | 1414.1 | 1886.6 | 1450.2 | 1309.5 | 4298.6 | 4757.8 | 3880.1 | 6114.2 |
| AUC_infinity: (ng*hr)/ | 1604.3 | 1974.3 | 1729.3 | 1356.0 | 4308.5 | 4759.9 | 3881.1 | 7026.5 |
| ml | | | | | | | | |
| AUMC_last: (ng*hr*hr)/ | 5842.3 | 5536.6 | 6691.8 | 3444.8 | 15896.4 | 15944.7 | 13611.5 | 39762.1 |
| ml | | | | | | | | |
| AUMC_infinity: (ng*hr*hr)/ | 13382.2 | 8595.2 | 18433.6 | 4888.6 | 16172.2 | 15999.9 | 13639.0 | 71975.7 |
| ml | | | | | | | | |
| T_half: hr | 10.8 | 7.6 | 12.5 | 4.9 | 2.7 | 2.1 | 2.0 | 7.8 |
| CL: L/(kg*hr) | 3.1 | 2.5 | 2.9 | 3.7 | 0.6 | 0.5 | 0.6 | 0.4 |
| V_z: L/kg | 48.7 | 27.6 | 52.3 | 26.1 | 2.3 | 1.6 | 1.8 | 4.0 |
| V_ss: L/kg | 26.0 | 11.0 | 30.8 | 13.3 | | 1.8 | 2.3 | |

Nano-PI Combined with α-PD1 Improved Survival of KPC Mice with Metastatic Pancreatic Cancer.

FIG. 23 shows survival rate of KPC mice with pancreatic cancer after treatment (n=10): mouse serum albumin (vehicle), Abraxane (Abrax, IV 10 mg/kg)+IPI549 (IP, 15 mg/kg)+α-PD1 (PD1, IP, 100 μg); and Nano-PI (PTX 10 mg/kg, IPI-549 5 mg/kg) plus α-PD1 (PD-1, IP 100 g). The Nano-PI was given intravenously once every three days for five doses. α-PD1 was administered intraperitonially once every three days for 3 doses (100 μg/mouse). IPI-549 was given intraperitoneally once every three days for five doses.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art and may be made without departing from the spirit and scope thereof.

What is claimed is:

1. A composition comprising: an effective amount of a phosphatidylinositol 3-kinase (PI3K) inhibitor, or a pharmaceutically acceptable salt thereof; and an albumin nanoparticle.

2. The composition of claim 1, wherein the PI3K inhibitor is a Class I PI3K inhibitor.

3. The composition of claim 2, wherein the PI3K inhibitor is an isoform-selective PI3K inhibitor.

4. The composition of claim 1, wherein the PI3K inhibitor is a compound of formula (III): 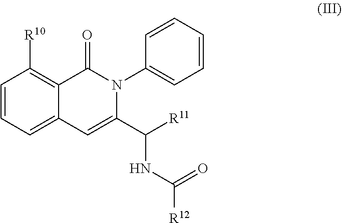
or a pharmaceutically acceptable salt thereof, wherein:
R¹⁰ is selected from —C≡C—R^{x}, C₁-C₄ alkyl, C₂-C₄ alkenyl, C₂-C₄ alkynyl, halo, cyano, C₁-C₄ alkoxy, C₁-C₄ alkylamino, and di-C₁-C₄-alkylamino;
R¹¹ is selected from C₁-C₄ alkyl and hydrogen;
R³ is an 8- to 10-membered bicyclic heteroaryl having 1, 2, or 3 nitrogen atoms, wherein the heteroaryl is optionally substituted with 1 or 2 substituents selected from amino, C₁-C₄ alkyl, and halo; and
R^{x} is selected from a 5- or 6-membered monocyclic heteroaryl having 1 or 2 heteroatoms independently selected from N and S, aryl, hydrogen, and C₁-C₄ alkyl, wherein the heteroaryl and aryl are optionally substituted with 1 or 2 substituents selected from C₁-C₄ alkyl.

5. The composition of claim 4, wherein R¹⁰ is —C≡C—R^{x}, and R^{x} is a 5-membered monocyclic heteroaryl having two nitrogen atoms, which is substituted with one C₁-C₄ alkyl.

6. The composition of claim 4 or claim 5, wherein R¹¹ is methyl.

7. The composition of any one of claims 4-6, wherein R¹² is a pyrazolo[1,5-a]pyrimidine substituted with one amino group.

8. The composition of any one of claims 4-7, wherein the compound of formula (III) is:

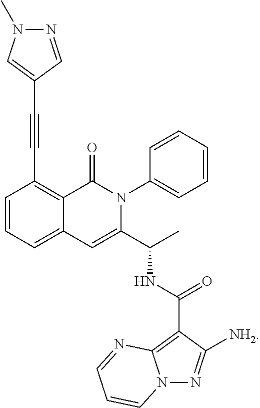 [SMILES: C[C@H](NC(=O)c1c(N)nn2cccnc12)c1cc2cccc(C#Cc3cnn(C)c3)c2c(=O)n1-c1ccccc1]

9. The composition of any of claims 1-8, wherein the nanoparticle has a diameter between 50 and 200 nm.

10. The composition of any of claims 1-9, wherein the albumin nanoparticle encapsulates the PI3K inhibitor.

11. The composition of any of claims 1-10, wherein the albumin is human serum albumin or albumin from animal species.

12. The composition of any of claims 1-11, wherein the composition further comprises a chemotherapeutic agent.

13. The composition of claim 12, wherein the albumin nanoparticle encapsulates the chemotherapeutic agent.

14. The composition of claim 12 or 13, wherein the chemotherapeutic agent is paclitaxel.

15. A compound of formula (I): 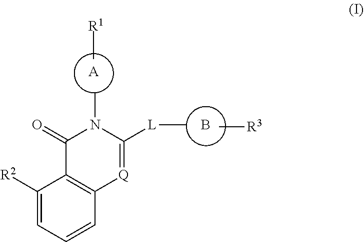
or a pharmaceutically acceptable salt thereof, wherein:
Q is CH or N;
A is aryl or a 5- or 6-membered monocyclic heteroaryl having 1, 2, 3, or 4 heteroatoms independently selected from N, O, S, and P;
R¹ is selected from hydrogen, halo, C₁-C₄ alkyl, C₃-C₆ cycloalkyl, C₁-C₄ haloalkyl, —OR^{a1}, —N(R^{b1})(R^{c1}), —SO₂R^{d1}, —SO₂N(R^{e1})(R^{f1}), and —NHSO₂R^{g1}, wherein R^{a1}, R^{b1}, R^{c1}, R^{d1}, R^{e1}, R^{f1}, and R^{g1} are each independently selected from hydrogen, C₁-C₄ alkyl, and C₁-C₄ haloalkyl;
R² is selected from hydrogen, halo, C₁-C₄ alkyl, C₁-C₄ haloalkyl, C₁-C₄ alkoxy, C₁-C₄ haloalkoxy, and a group of formula (II): 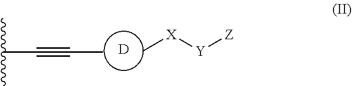
wherein:
D is a monocyclic heteroaryl or monocyclic heterocyclyl, each of which is optionally substituted with a C₁-C₄ alkyl group;
X is a bond, —C(O)—, —NH—, or —C(O)NH—;
Y is —(CR^{a2})ₙ-G²-, wherein each R^{a2} is independently selected from H and C₁-C₄ alkyl, or wherein two R^{a2} together with the carbon atom(s) to which they are attached form a C₃-C₇ cycloalkyl; G² is a bond, cycloalkylene, or heterocyclylene; and n is 0, 1, 2, or 3;
Z is —OR^{b2}, —SR^{c2}, —N(R^{d2}) (R^{e2}), or —CH₃, wherein R^{b2}, R^{e2}, R^{d2}, and R^{e2} are each independently selected from hydrogen, aryl, arylalkyl, C₁-C₄ alkyl, —C(O)—C₁-C₄₀ alkyl, —C(O)—C₂-C₄₀ alkenyl, and a group of formula (IIa): 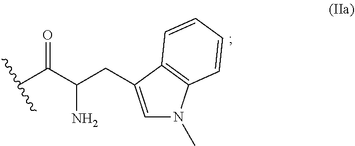 [SMILES: *C(=O)C(N)Cc1cn(C)c2ccccc12]
and
R³ is selected from hydrogen and a group -L³-E, wherein:
L³ is a bond, C₁-C₂ alkylene, —CH═CH—, —C≡C—, —C(O)—, —O—, —NH—, —S—, —C(O)O—, —C(O)NH—, —C(O)S—, arylene, cycloalkylene, heteroarylene, or heterocyclylene, or wherein L³ comprises a combination of any two of such groups; and
E is a bicyclic heterocyclyl or bicyclic heteroaryl, each of which is optionally substituted with 1, 2, 3, 4, or 5 substituents independently selected from halo, C₁-C₄ alkyl, C₃-C₆ cycloalkyl, C₃-C₆-cycloalkyl-C₁-C₄-alkyl, C₁-C₄ haloalkyl, oxo, —OR^{a3}, —N(R^{b3})(R^{c3}), —SO₂R^{d3}, —SO₂N(R^{e3})(R^{f3}), and —NHSO₂R^{g3}, wherein R^{a3}, R^{b3}, R^{e3}, R^{d3}, R^{e3}, R^{f3}, and R^{g3} are each independently selected from hydrogen, C₁-C₄ alkyl, and C₁-C₄ haloalkyl;
L is —(CR^{a4}R^{b4})ₘ-G⁴-, wherein:
R^{a4} and R^{b4} are independently selected from hydrogen and C₁-C₄ alkyl;
m is 0, 1, or 2; and
G⁴ is a bond, —NHC(O)—, —NH—, —O—, or —S—; and
B is a bicyclic heteroaryl or bicyclic heterocyclyl, each of which is optionally substituted with 1, 2, 3, 4, or 5 substituents independently selected from halo, C₁-C₄ alkyl, C₃-C₆ cycloalkyl, C₁-C₄ haloalkyl, optionally substituted aryl, —OR^{a5}, —N(R^{b5})(R^{c5}), —SO₂R^{d5}, —SO₂N(R^{e5})(R^{f5}), and —NHSO₂R^{g5}, wherein R^{a5}, R^{b5}, R^{c5}, R^{d5}, R^{e5}, R^{f5}, and R^{g5} are each independently selected from hydrogen, C₁-C₄ alkyl, and C₁-C₄ haloalkyl;
wherein when R³ is hydrogen, R² is a group of formula (II) and Z is not —CH₃; and
wherein when R² is hydrogen, halo, C₁-C₄ alkyl, C₁-C₄ haloalkyl, C₁-C₄ alkoxy, or C₁-C₄ haloalkoxy, R³ is a group -L³-E.

16. The compound of claim 15, wherein the compound is a compound of formula (Ia): 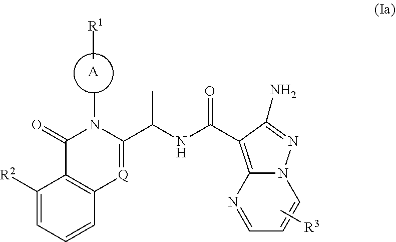
or a pharmaceutically acceptable salt thereof.

17. The compound of claim 16, or a pharmaceutically acceptable salt thereof, wherein A is phenyl and R¹ is hydrogen.

18. The compound of claim 15, wherein the compound is a compound of formula (Ib): 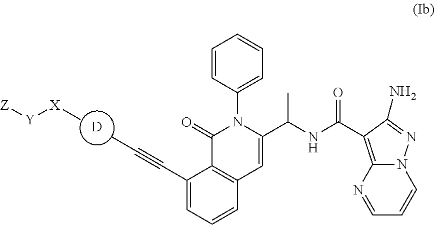
or a pharmaceutically acceptable salt thereof.

19. The compound of any one of claims 15-4, or a pharmaceutically acceptable salt thereof, wherein D is a five-membered monocyclic heteroaryl or a 4- to 6-membered monocyclic heterocyclyl, each of which independently comprises 1, 2, 3, or 4 heteroatoms independently selected from N, O, S, and P.

20. The compound of claim 5, or a pharmaceutically acceptable salt thereof, wherein D is selected from pyrrole, pyrazole, imidazole, imidazoline, oxazole, oxathiazole, oxadiazole, azetidine, pyrroline, pyrrolidine, and piperidine.

21. The compound of claim 5, or a pharmaceutically acceptable salt thereof, wherein D has a structure selected from:

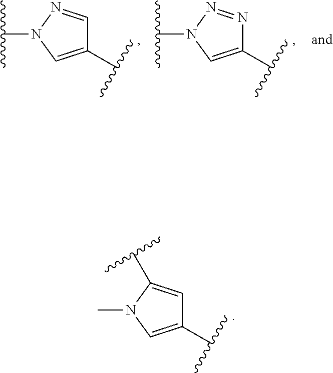

22. The compound of any one of claims 15-7, or a pharmaceutically acceptable salt thereof, wherein X is a bond or —C(O)—.

23. The compound of any one of claims 15-8, or a pharmaceutically acceptable salt thereof, wherein Y is —(CR^{a2})ₙ—CH₂—, wherein n is 0 or 1, and wherein each R^{a2} is hydrogen, or wherein the two R^{a2} groups, together with the carbon atom to which they are attached, form a cyclopropylene ring.

24. The compound of claim 9, or a pharmaceutically acceptable salt thereof, wherein the group —X—Y—Z has a formula selected from:

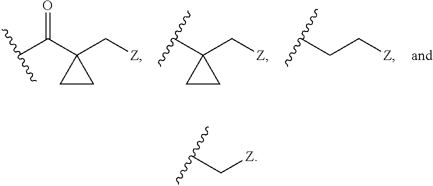

25. The compound of claim 10, or a pharmaceutically acceptable salt thereof, wherein Z is —OR^{b2}, wherein R^{b2} is selected from hydrogen, —C(O)—C₁-C₄₀ alkyl, —C(O)—C₂-C₄₀ alkenyl, and a group of formula (IIa).

26. The compound of claim 11, or a pharmaceutically acceptable salt thereof, wherein Z is Z is —OR^{b2}, wherein R^{b2} is selected from hydrogen, —C(O)—C₁₅-C₂₀ alkyl, —C(O)—C₁₅-C₂₀ alkenyl, and a group of formula (IIa).

27. The compound of claim 15, wherein the compound is a compound of formula (Ic): 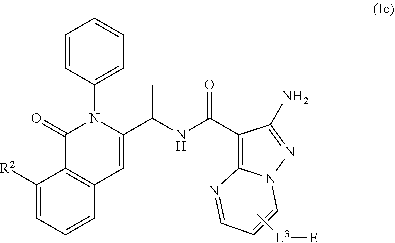
or a pharmaceutically acceptable salt thereof.

28. The compound of claim 13, or a pharmaceutically acceptable salt thereof, wherein R² is selected from halo and a group of formula (II).

29. The compound of claim 14, or a pharmaceutically acceptable salt thereof, wherein R² is halo.

30. The compound of any one of claims 13-15, or a pharmaceutically acceptable salt thereof, wherein L³ is a bond, —CH₂—CH₂—, —CH═CH—, —C≡C—, —C(O)NH—, or a 5-membered heteroarylene having 1, 2, or 3 nitrogen atoms.

31. The compound of any one of claims 13-16, or a pharmaceutically acceptable salt thereof, wherein E has a formula: 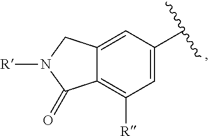
wherein R′ and R″ are independently selected from C₁-C₄ alkyl, C₃-C₆-cycloalkyl-C₁-C₄-alkyl, C₁-C₄ haloalkyl, and —NHSO₂R^{g3}, wherein R^{g3} is C₁-C₄ alkyl.

32. The compound of claim 17, or a pharmaceutically acceptable salt thereof, wherein R′ is C₃-C₆-cycloalkyl-C₁-C₄-alkyl, and R″ is selected from C₁-C₄ alkyl, C₁-C₄ haloalkyl, and —NHSO₂R^{g3}, wherein R^{g3} is C₁-C₄ alkyl.

33. The compound of claim 15, wherein the compound is selected from: 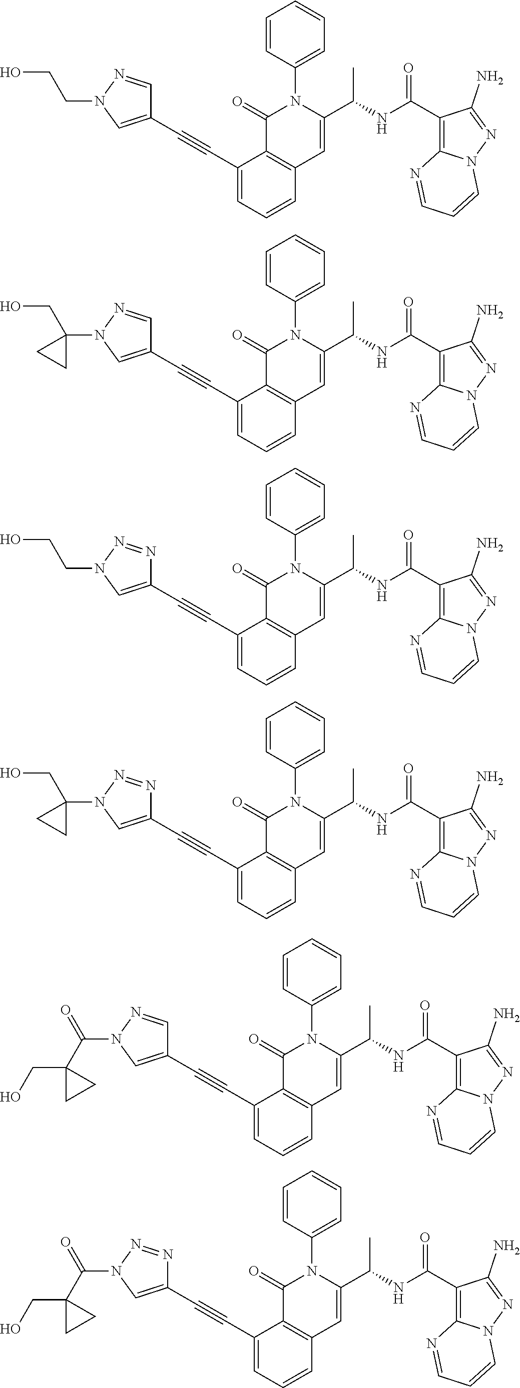 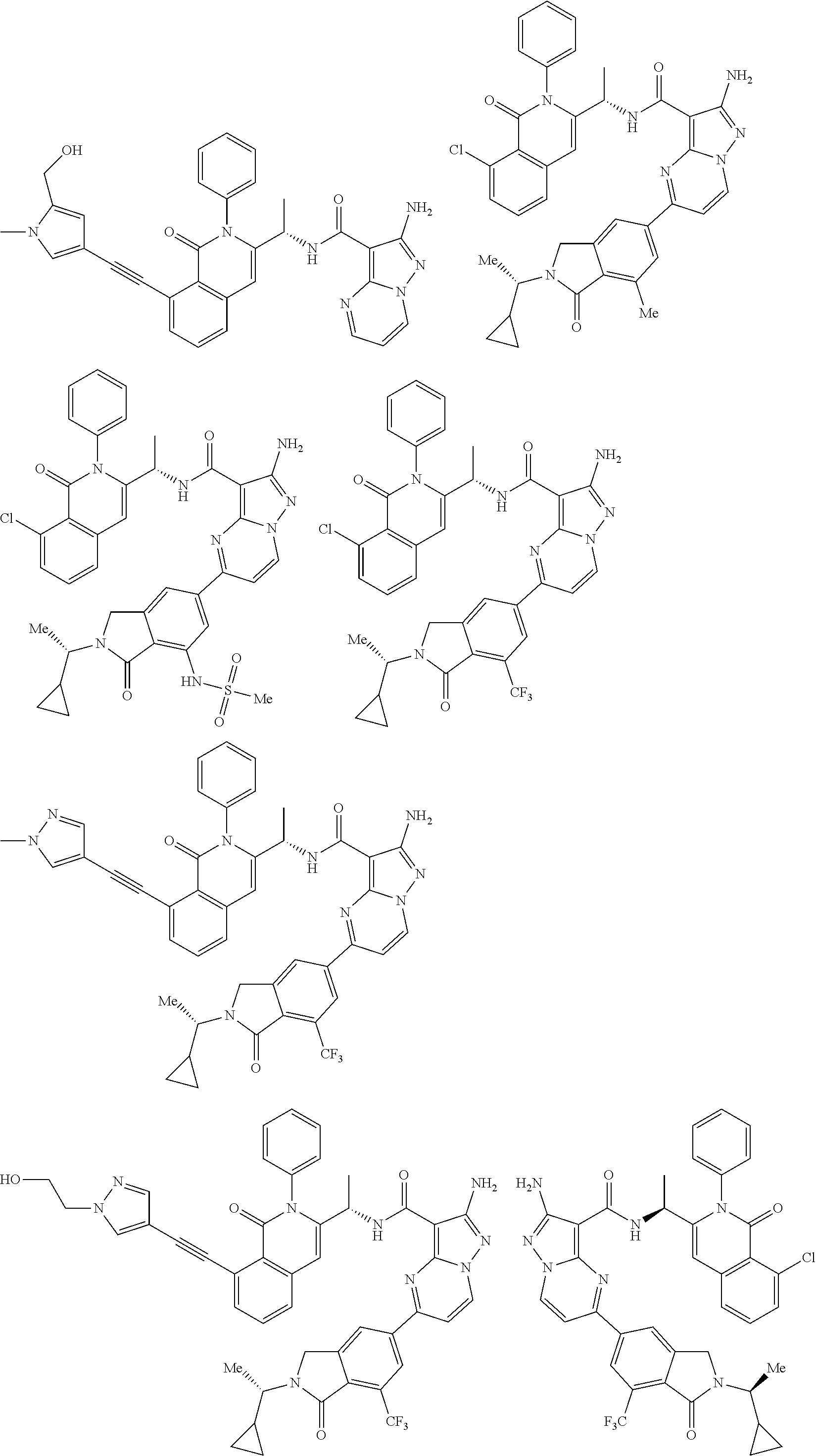 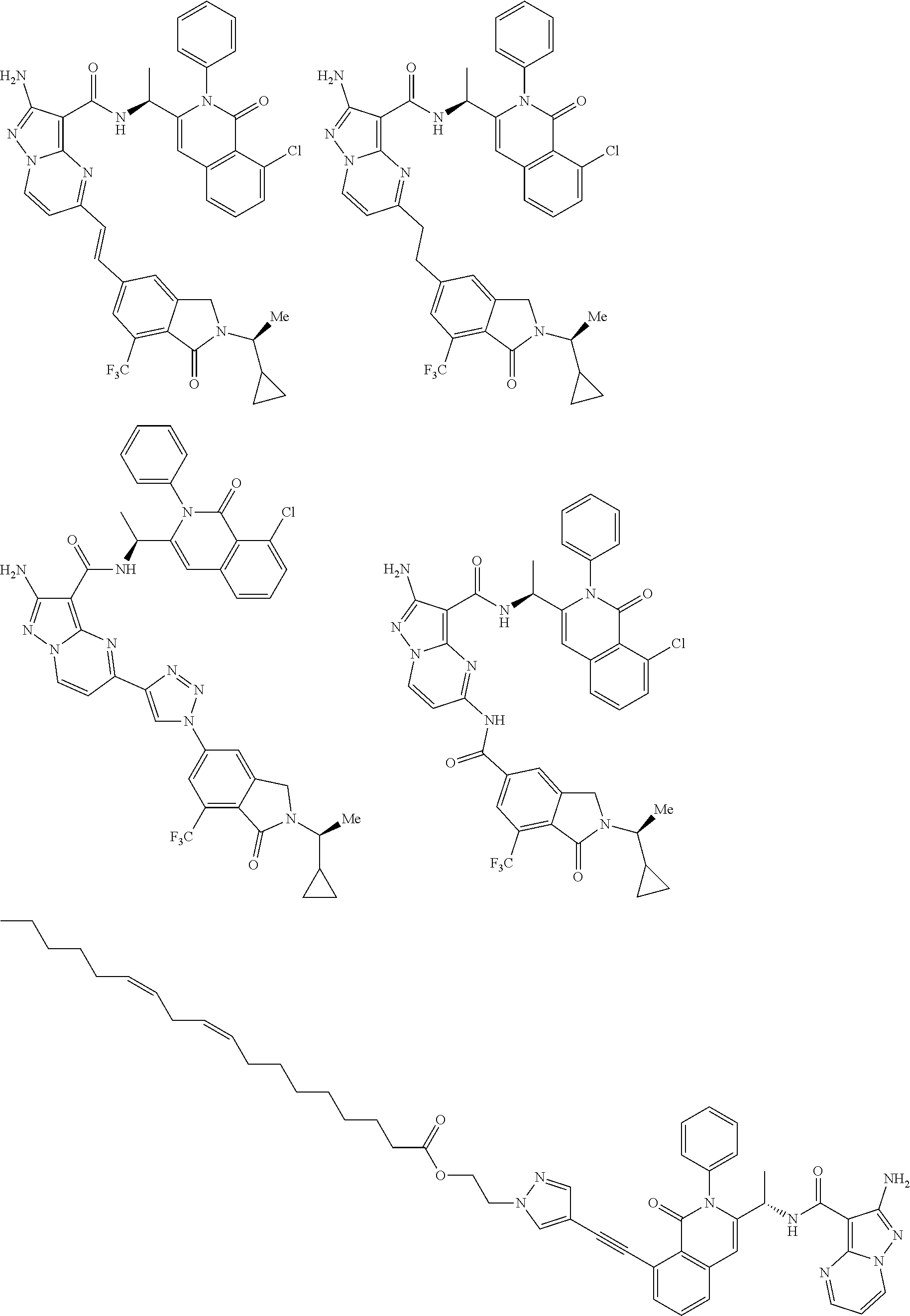 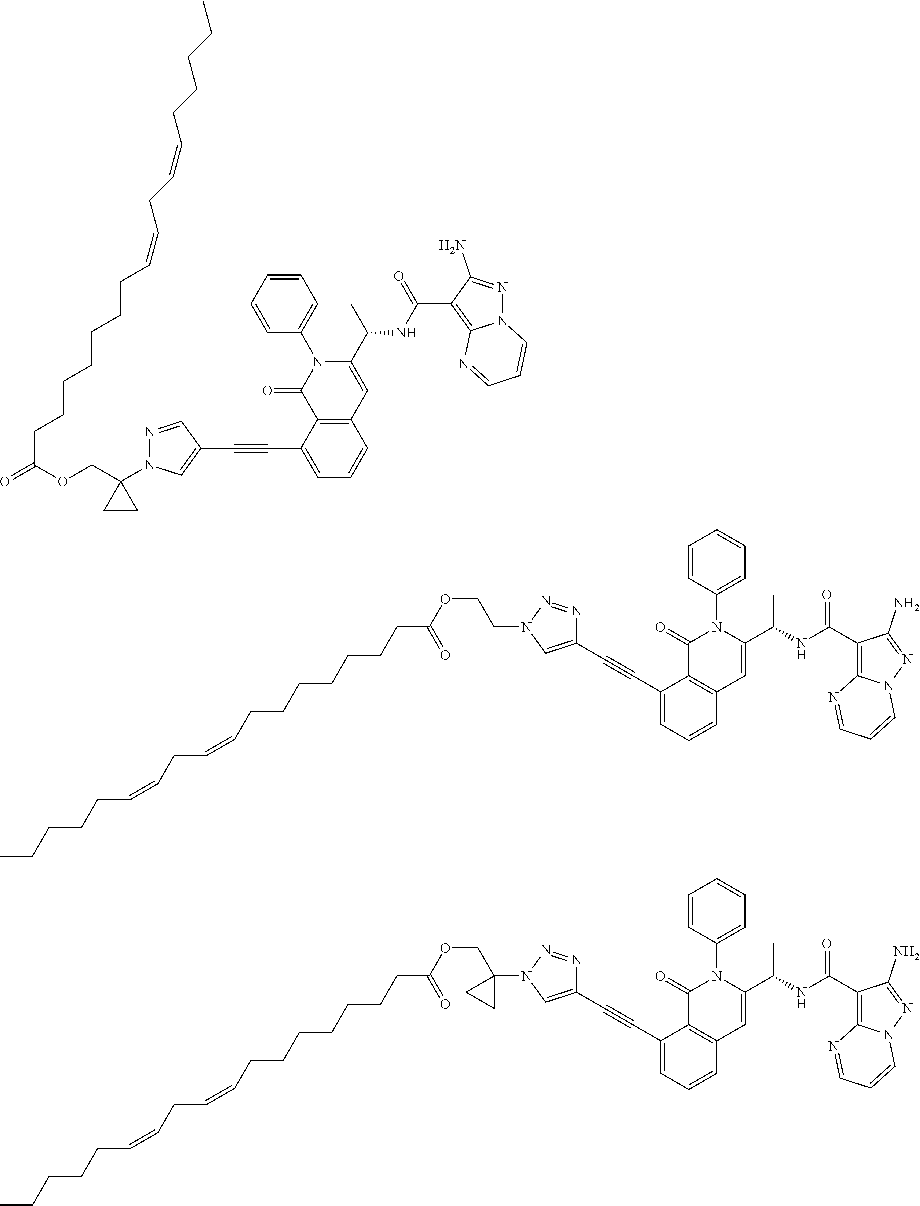 [SMILES: CCCCC/C=C\C/C=C\CCCCCCCC(=O)OCC1(n2cc(C#Cc3cccc4cc([C@H](C)NC(=O)c5c(N)nn6cccnc56)n(-c5ccccc5)c(=O)c34)cn2)CC1 ; CCCCC/C=C\C/C=C\CCCCCCCC(=O)OCCn1cc(C#Cc2cccc3cc([C@H](C)NC(=O)c4c(N)nn5cccnc45)n(-c4ccccc4)c(=O)c23)nn1 ; CCCCC/C=C\C/C=C\CCCCCCCC(=O)OCC1(n2cc(C#Cc3cccc4cc([C@H](C)NC(=O)c5c(N)nn6cccnc56)n(-c5ccccc5)c(=O)c34)nn2)CC1] 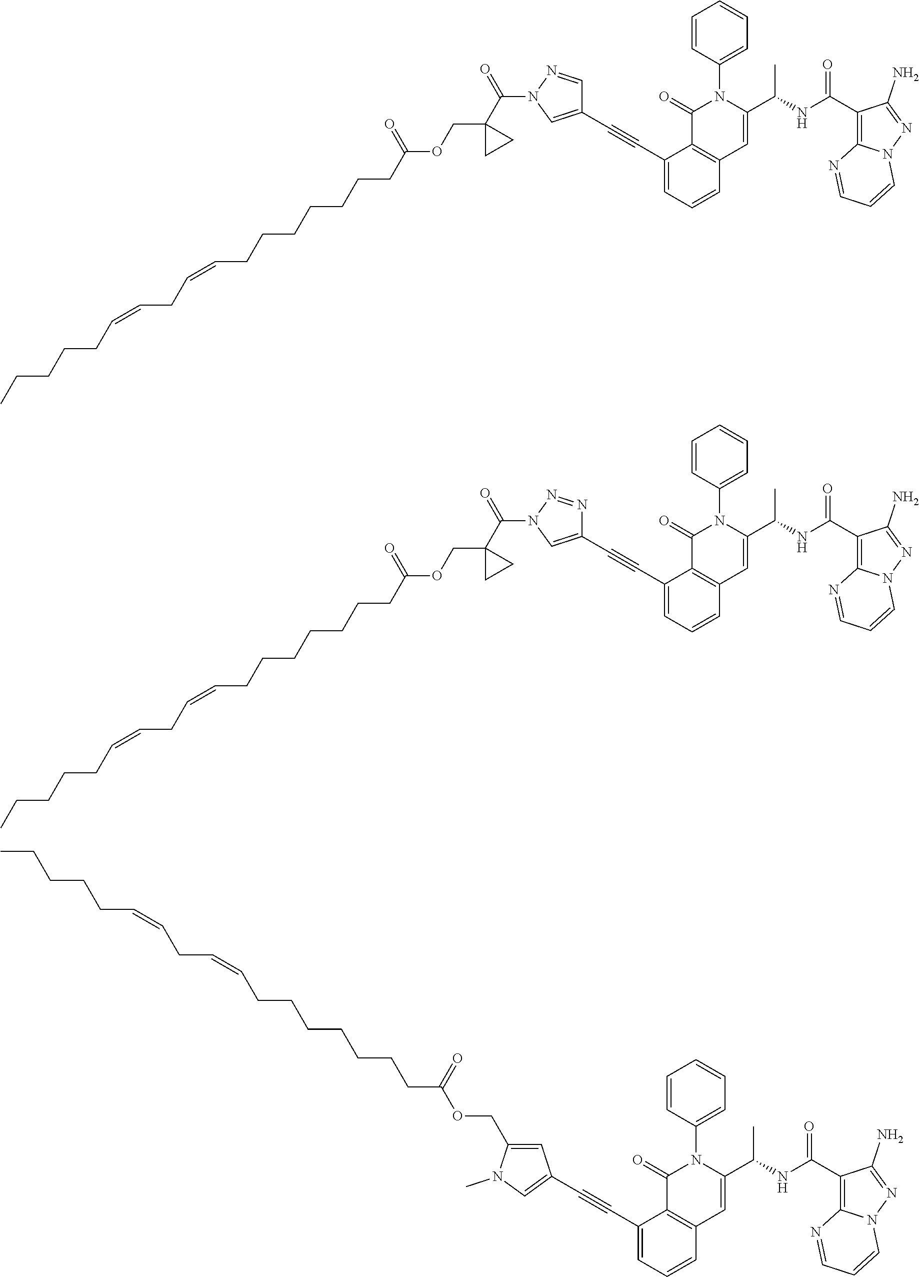 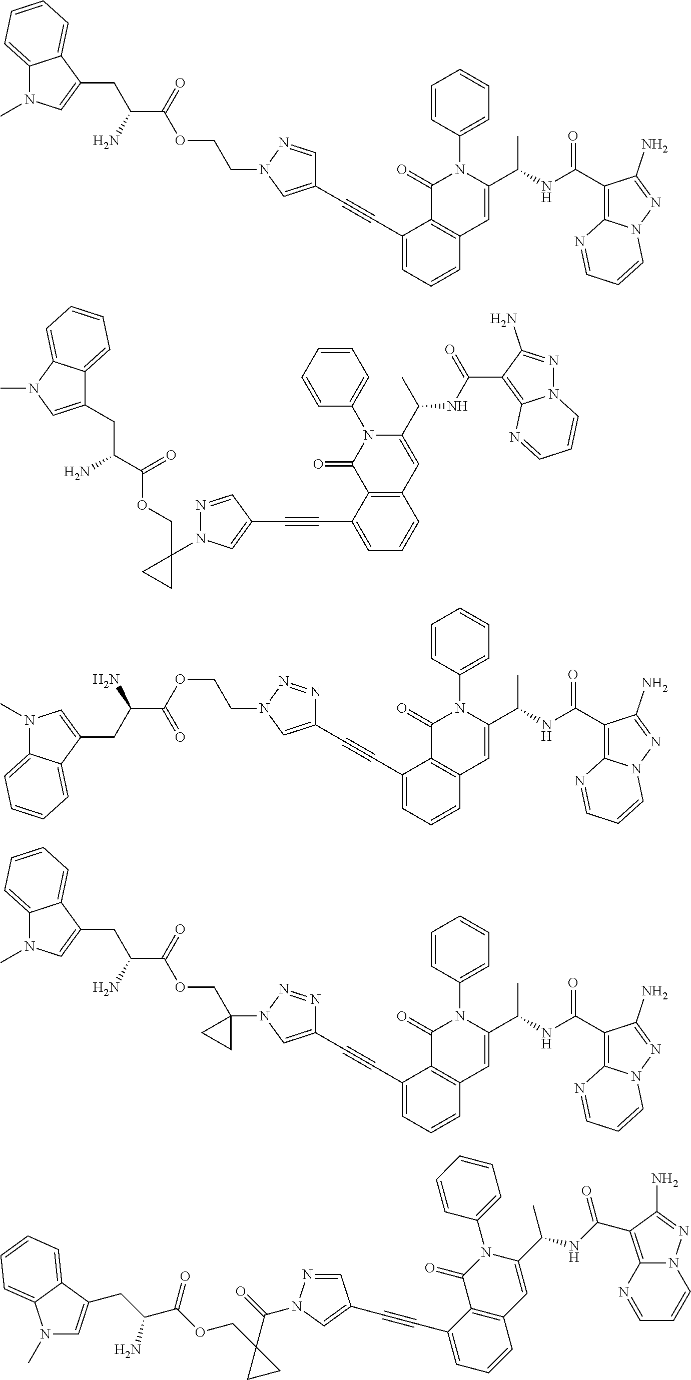 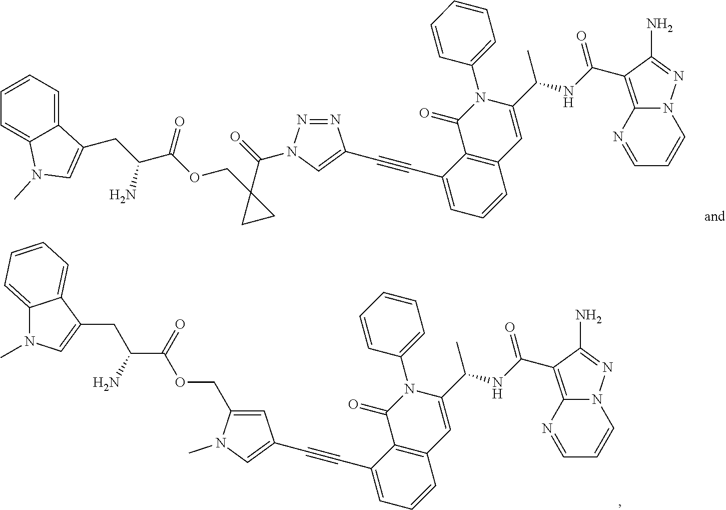 [SMILES: C[C@H](NC(=O)c1c(N)nn2cccnc12)c1cc2cccc(C#Cc3cn(C(=O)C4(COC(=O)[C@H](N)Cc5cn(C)c6ccccc56)CC4)nn3)c2c(=O)n1-c1ccccc1 ; C[C@H](NC(=O)c1c(N)nn2cccnc12)c1cc2cccc(C#Cc3cc(COC(=O)[C@H](N)Cc4cn(C)c5ccccc45)n(C)c3)c2c(=O)n1-c1ccccc1]
and pharmaceutically acceptable salts thereof.

34. A pharmaceutical composition comprising an effective amount of a compound of any one of claims 15-33, or a pharmaceutically acceptable salt thereof.

35. The pharmaceutical composition of claim 34, wherein the composition further comprises an albumin nanoparticle.

36. The pharmaceutical composition of claim 35, wherein the nanoparticle has a diameter between 50 and 200 nm.

37. The pharmaceutical composition of claim 35 or 36, wherein the albumin nanoparticle encapsulates the compound.

38. The pharmaceutical composition of any of claims 35-37, wherein the albumin is human serum albumin or albumin from animal species.

39. The pharmaceutical composition of any of claims 34-38, wherein the composition further comprises a chemotherapeutic agent.

40. The pharmaceutical composition of claim 39, wherein the chemotherapeutic agent is paclitaxel.

41. A method of treating or preventing a disease or disorder in a subject comprising administering to the subject an effective amount of a composition of any of claims 1-14, or a compound of any one of claims 15-40, or a pharmaceutically acceptable salt thereof.

42. The method of claim 41, wherein the method further comprises administration of an immunotherapy.

43. The method of claim 42, wherein the immunotherapy comprises administration of a PD-1 or PD-L1 antibody.

44. The method of any of claims 41-43, wherein the disease or disorder comprises cancer, an autoimmune disease or disorder, or an inflammatory disease or disorder.

45. The method of any of claims 41-44, wherein the disease or disorder is cancer.

46. The method of claim 44 or 45, wherein the cancer comprises a solid tumor or hematological cancer.

47. The method of any of claims 44-46, wherein the cancer is metastatic cancer.

48. The method of any of claims 44-47, wherein the disease or disorder is breast cancer, pancreatic cancer, lung cancer, lymphoma.

49. The method of any of claims 44-48, wherein the method suppresses or eliminates cancer metastasis, decreases tumor growth, prevents tumor recurrences, or any combination thereof.

50. The method of any of claims 41-49, wherein the compound or the composition is administered by subcutaneous injection.

51. The method of any of claims 42-50, wherein the immunotherapy is administered at the same time, preceding, or following the compound or the composition.

52. The method of any of claims 42-51, wherein the immunotherapy is administered by subcutaneous injection.

53. The method of any of claims 41-52, wherein the subject is a human.
